(19) United States
(12) Patent Application Publication
(10) Pub. No.: US 2005/0074834 A1
(43) Pub. Date: Apr. 7, 2005

(54) Method and system for classifying a scenario

(75) Inventors: Frank W.R. Chaplen, Albany, OR (US); William H. Gerwick, Corvallis, OR (US); Goran Jovanovic, Corvallis, OR (US); Wojtek J. Kolodziej, Corvallis, OR (US); Jim Liburdy, Philomath, OR (US); Phil McFadden, Corvallis, OR (US); Brian Kevin Paul, Corvallis, OR (US); Thomas K. Plant, Philomath, OR (US); Janine E. Trempy, Corvallis, OR (US); Nicolas Roussel, Corvallis, OR (US); Corwin Willard, Monroe, OR (US); Rosalyn H. Upson, Eugene, OR (US); Andrzej Pacut, Warsaw (PL)

Correspondence Address: KLARQUIST SPARKMAN, LLP, 121 SW SALMON STREET, SUITE 1600, PORTLAND, OR, 97204, US

(73) Assignee: The State of OR acting by & through the State Board of Higher Educ. on behalf of OR State Univ.

(21) Appl. No.: 10/801,389

(22) Filed: Mar. 12, 2004

Related U.S. Application Data
(63) Continuation-in-part of application No. PCT/US02/29085, filed on Sep. 12, 2002.
(60) Provisional application No. 60/322,004, filed on Sep. 12, 2001.

(51) Int. Cl. G06F 19/00; G01N 33/48; G01N 33/50; C12Q 1/04

(52) U.S. Cl. 435/34; 702/19

(57) ABSTRACT

Living cells can be used to identify or quantify bioactive conditions, including without limitation, chemicals, biological pathogens, and environmental conditions, such as pH, in samples based on changes in, for example, cell color, morphology and/or physiology. Such changes can be directly detected or detected with the aid of instrumentation. One embodiment of the method comprises exposing a system to a bioactive condition, such as a chemical agent, a biological pathogen, an environmental condition, such as pH, etc., and combinations of such conditions. The system then exhibits a response to the bioactive condition. The response of the system, or a portion thereof, to the bioactive condition is then represented, such as by digital images. The method then involves attempting to classify a scenario by database comparison. Classification can be in terms of numeric or non-numerical classifiers. Typically, the system comprises living cells. Living cells useful for practicing the method experience a detectable change in response to an interaction with a bioactive condition. A likely living cell for use with the method and apparatus of the present invention is a chromatophore. The present method has a number of uses, including classifying unknown drug candidates, classifying unknown toxins, classifying chemical warfare agents, etc. The method a can be implemented using a computer program encoding the method. Moreover, a computer-readable medium is described on which is stored a computer program having instructions for executing the method. A cytosensor apparatus also is described.

### CROSS REFERENCE TO RELATED APPLICATIONS

The present application is a continuation-in-part of pending international application No. PCT/US02/29085, and further claims the benefit of the earlier filing dates of U.S. provisional patent application No. 60/322,004, filed Sep. 12, 2001, U.S. utility application No. 09/952,216, filed on Sep. 12, 2001, and U.S. utility application No 10/024,654, filed on Dec. 17, 2001, all of which prior applications are incorporated herein by reference in their entirety.

### STATEMENT OF GOVERNMENT SUPPORT

This invention was made, at least in part, using funds provided by the Defense Advanced Research Projects Agency, contract Nos. N66001-99-C-8631 and N660001-01-C-8047, Office of Naval Research grant No. N00014-96-1-0566, and NSF grant No. BES-9905301. The United States government may have rights in this invention.

### FIELD

The present disclosure concerns a method for determining the identity of an unknown scenario, such as exposure of ensembles of biological elements to bioactive conditions, an analytical approach for classifying the data concerning the scenario, a method for making apparatuses useful for practicing the method, and apparatuses for practicing the method.

### BACKGROUND

Biosensors are devices that use homogeneous or heterogeneous mixtures of biological elements to detect conditions of interest. The material presented by B. Alberts et al. suggests that living systems are not irreducible entities but are mixtures of interacting biological elements, such as metal ions, metabolites, small peptides, proteins, nucleic acids etc. (B. Alberts, A. Johnson, J. Lewis et al., Molecular Biological of the Cell, 3^{rd} Edition, Garland Science, New York, 1994). The use of arrays of biological elements improves the classification of substances in a number of fields of endeavor, including toxicology, pharmacology, medical diagnostics, environmental monitoring, scientific discovery and others. In the practice of medicine, biosensors already play a substantive role in glucose monitoring—using mixtures of molecules that have physiological functions (enzymes, peroxides); and the many types of antibody tests currently produced for medical diagnostics and forensics exemplify a major category of biosensor that take advantage of random mixtures of biological elements to classify analytes.

Biosensors that include living cells as sensing elements are often referred to as cytosensors. Cytosensors are “function-based” sensors because they typically are used to detect substances according to the effect of the substances on the functional activities of biological elements in cells. In that respect, cytosensors are similar to animal testing, with both cells and animals providing many functional targets of opportunity for detecting biological activities for a variety of applications. Consequently, cytosensor measurements can be configured to replace live animal tests. Functional targets (such as enzymes, receptors, membranes, cytoskeletal structures, cellular organelles, second messenger signals, cytoplasmic elements, ensembles of cells, tissues and others) can be partially specified or unspecified at the time a measurement is performed and can remain unspecified even for a standardized test. In addition, classes of analytes can be identified with and without identification of any analytes. For example, irritants in cosmetics can be detected with a cytosensor by a standardized reaction without knowledge of a reaction mechanism. Such a test need not identify any specific irritants, but may indicate the presence of irritants. Such methods can serve as alternatives to animal testing in applications such as food and drug testing.

There are known apparatuses that operate based on the response of a test compound, including patented apparatuses. For example, Okun et al., U.S. Pat. No. 5,919,646, describes an apparatus and method for real-time measurement of cellular response of a test compound. Elving, U.S. Pat. No. 4,985,353, discloses detection of a toxin produced by Bordetella pertussis based on a color change in a fish scale. Lerner et al., U.S. Pat. Nos. 5,462,856 and 6,051,386, disclose methods for identifying chemicals that act as agonists for a G-protein-coupled, cell-surface receptor. Danosky and McFadden, Biosensors & Bioelectronics 12, 925-936 (1997) describe the activities of fish chromatophores. Weaver, U.S. Pat. No. 4,401,755, discloses a process for encapsulating a biologically active compound with a gel material, allowing the material to gel.

J. Ayers et al. (1998) reports the development of biomimetic autonomous robots with control architectures that operate analogously to the modular neuronal components that underlie locomotion and feeding patterns of underwater animals (J. Ayers, P. Avracky, N. McGruer et al., A modular behavioral-based architecture for biomimetic autonomous underwater robots, In: Proc. Of the autonomous vehicles in mine countermeasures symposium, Naval Postgraduate School, In Press, 1998). L. H. Hartwell et al. (1999) report that cellular functions, such as signal transmission, are carried out by ‘modules’ made up of many species of interacting molecules. General principles that govern the structure and behaviour of modules may be discovered with help from synthetic sciences such as engineering and computer science, from stronger interactions between experiment and theory in cell biology, and from an appreciation of evolutionary constraints (L. H. Hartwell, J. J. Hopfield, S. Liebler, A. W. Murray, From molecular to modular cell biology, Nature, 402 (6761 Suppl):C47-52, Dec. 2, 1999).

D. Bray postulated that proteins in cells have as their primary function the transfer and processing of information and stated that biological systems are “trained by environmental cues with distributed information processing and communication paradigms (D. Bray, Protein molecules as computational elements in living cells, Nature, 376, 307-312, 1995). This observation is representative of paradigms for reasoning and language that have been used by the field of artificial intelligence for developing learning systems. Adaptive, probabilistic networks have been used extensively for data classification. Additional summaries of prior art in the fields of artificial intelligence and application of neural networks to data analysis and representation can be found in Luger (Luger, G. F. and Stubblefield (1997) Artificial Intelligence, 1^{st} Edition, Addison-Wesley, Pearson Education, New York).

A. L. Barabasi and R. Albert (1999) report that systems as diverse as genetic networks or the World Wide Web are best described as networks with complex topology with the scale-free, power-law distribution of network connectivities and modularity being a common feature (A. L. Barabasi and R. Albert, Emergence of Scaling in Random Networks, Science, 286, 509-512, 1999). Dynamic systems with small world properties (scale-free or random network structures) display improved signal propagation and computing power (Watts, D. J. and Strogatz, S. H., Collective dynamics of “small-world” networks. Nature 393(6684), 440-442, 1998). H. H. McAdams et al. teach a method for the structured modeling of biological systems by analogy with methods used to model electrical circuits by electrical circuit logic components (H. H. McAdams, A. P. Arkin and L. Shapiro, System and Method for Simulating Operation of Biochemical Systems, U.S. Pat. No. 5,914,891, 1999).

### SUMMARY

Major improvements in the the functional classification of bioactive agents using biosensors can derive from the identification of underlying structural motifs in cellular regulatory and transformative networks and an understanding of how these fundamental ensemblic activities are used for cellular computing. Furthermore, computational analogies found in single-cell systems can hold across scales and be used for the development of biosensors for analyzing the function of sub-cellular to landscape scale systems that incorporate biological elements as sensors, signal processors, data storers or communicators.

Several embodiments of a method for classifying a scenario are described. One embodiment of the method comprises exposing a system to a bioactive condition, such as a chemical agent, a biological pathogen, an environmental condition, such as pH, etc., and combinations of such conditions. The system then exhibits a response to the bioactive condition. The response of the system, or a portion thereof, to the bioactive condition is then represented, such as by digital images. The method then involves attempting to classify a scenario by database comparison. Classification can be in terms of numeric or non-numerical classifiers. The database can be integrated into the system, a database generated by the system, or can be a database provided by a third party, or any combination thereof. Moreover, the database can be available to the system directly, or may be available via remote access.

The system may comprise living cells, and such living cells may be immortal. Living cells can be used to identify or quantify bioactive conditions, including without limitation, chemicals, biological pathogens, and environmental conditions, such as pH, in samples based on changes in, for example, cell morphology and/or physiology. Such changes can be directly detected or detected with the aid of instrumentation. For example, cells can be arranged so that visual inspection is adequate to identify the presence of bioactive conditions, or, alternatively, an optical measurement system or other instrumentation can be provided.

Examples of living cells include, without limitation, chromatophores, 12 pheo-chromocytoma cell, HeLa cell, 3T3 cells, COS7 cells, HEPG2 cells, microbial communities, tissues, and combinations thereof. The system also can be higher order organisms, such as fish and mammals. Living cells useful for practicing the method experience a detectable change in response to an interaction with a bioactive condition.

One feature of this particular embodiment of the method involves representing a response of the system by determining data sufficient to determine a numerical feature space vector. A database is then provided for comparison. The database can be generated by exposing a system to known scenarios to determine a numerical feature space vector. The data obtained is then transformed, such as by determining software expert parameters based on extracted data; and weighting the expert parameters. Integrated experts also can be tuned, such as by using adaptive expert calibration. Understanding the functional organization of cellular regulatory and transformative networks will therefore result in optimal feature selection and data reduction efficiency.

The described embodiments of the method can be used to attempt to classify the bioactive condition by database comparison. This may involve exposing the system to one or more scenarios to provide sufficient data to generate a characteristic signature for the bioactive condition. Data is then extracted, and the locations of data clusters in feature space are calculated to represent the characteristic signature of the bioactive condition. The location of data clusters in feature space representing the characteristic signature of the bioactive condition is then compared to data clusters representing known bioactive conditions. From this information, it is possible to determine a likelihood that a bioactive condition is a known bioactive condition. For example, the likelihood can be determined using a Mahalanobis distance for the unknown scenario relative to at least one known scenario cell reaction model space. The calculation of relative location of data clusters can be done with software experts. Attempting to classify a complex scenario also can comprise synthesizing a complex scenario from known scenarios present in the database. The scenario generated by the bioactive condition is then compared to the complex scenario, and a determination made concerning the likelihood that a complex scenario is the scenario.

In these embodiments, the known scenarios can be simplex scenarios, and furthermore each simplex scenario may be an elicitor. In such situations, the elicitor can consist of bioactive conditions and the protocol utilized to apply the bioactive conditions to the system. The bioactive conditions may have known or unknown effects on the system. Unknown bioactive agents, in combination with one or more elicitors, may be repeatedly tested. Each simplex scenario typically provides information regarding effect on the system of the bioactive conditions. The method can be useful to provide information about the manner in which the bioactive condition affects the system.

Where the system comprises living cells, the numerical feature space vector may contain parameters selected from the group consisting of nucleic acid composition (e.g., 16S-RNA profiling; DNA/mRNA microarray analysis); membrane lipid fatty acid composition (e.g., phospholipids fatty acid profiling); metabolic activity (e.g. community level physiological profiling); cellular secretions; chemical measurements (e.g. organic pollutants, nutrient discharges, heavy metals, dissolved oxygen, pH, redox, chlorine ion concentration and combinations thereof); physical measurements (temperature, ultra-violet radiation intensity, light intensity, changes in physical properties of semiconductor nanoparticles); and combinations thereof. The physical measurements made may provide a measure of, for example, cell morphology and turbidity changes.

Again, where the system is a live cell, a numerical feature space vector may include measurements of cellular processes selected from the group consisting of gene expression (e.g., DNA microarray analysis, recombinant marker analysis, green fluorescent protein analysis, enzymatic activity, such as recombinant luciferase activity, immunodetection, such as western blotting, lateral flow immunodetection, immune precipitation, and combinations thereof, semiconductor nanoparticle analysis, and combinations thereof); cell regulation (e.g., changes in protein phosphorylation patterns, such as may be measured by 2D gel electrophoresis); metabolism (e.g., measuring growth medium composition, metabolite secretion patterns, protein secretion patterns, intracellular measurements of metabolite levels); and combinations thereof. Physical methods for making suitable measurements include, without limitation, gas chromatography, liquid chromatography, mass spectrometry, nuclear magnetic resonance spectrometry, gel electrophoresis, and combinations thereof.

A likely living cell for use with the method and apparatus of the present invention is a chromatophore, such as a fish chromatophore, e.g., a Betta chromatophore, such as may be selected from the group consisting of B. splendens, B. schaumnestbauer, B. bellica, B. coccina, B. farciata, B. foerrchi, B. rmaradgina, B. maulbruter, B. anabatoidcr, B. balunga, B. brederi, B. macractoma, B. picta, B. pugrrax, B. rubra, B. taeniata, and B. unimaculata),. Other types of chromatophores also can be used, such as a frog chromatophore, e.g., a Xenopus chromatophore. Where the living system is a chromatophore, the numerical feature space vector likely includes color change measurements, such as measurements of changes in the refracted wavelength of light, color intensity, etc. Other examples of cell features that can be monitored include, again without limitation, cell morphology, cell area, cell motility or any combination thereof. For color change measurements, images can be acquired of the chromatophore to reflect response of the chromatophore to a scenario. These responses are then converted to hue, saturation, and value histograms. Probabilistic segmentation is then applied to assign a probability that a histogram data point belongs to a particular group of data points in the histogram to isolate data points representing response of the chromatophore to the scenario from extraneous information. This provides probabilistic clusters for chromatophore responses of interest for segmenting each of the images into image classes. The images are divided into plural subfields, and numerical information is extracted for each subfield from the image segments, such numerical information representing features of the images having information concerning the chromatophore response to the scenario. Numerical data is generated for each subfield for each chromatophore response monitored, and a model fitting procedure is applied to the set of numerical data and identifying model parameters, where the model parameters may represent a single cell response or plural cell responses. At least a portion of the model parameters is used to define a feature model space having scenario clusters for known materials.

The method can be used where the known scenario is a complex scenario, and such a complex scenario may comprise multiple simplex scenarios. Moreover, each simplex scenario may be an elicitor. The elicitor may consist of a known bioactive condition and the protocol utilized to apply the bioactive conditions to the system. The bioactive condition may have known or unknown effects on biological pathways, such as cell signaling pathways that control cell function. The complex scenario may comprise simplex scenarios that represent information regarding the mechanism of action of known bioactive conditions. Classifying the scenario with respect to the complex scenario can provide information about mechanism of action of the bioactive condition.

Where the system comprises plural live cells, database information can be obtained by serially exposing plural live cells to a known scenario. Images are acquired of the cells to reflect response of the cells to the scenario. These responses are converted to hue, saturation, value histograms. Probabilistic segmentation is applied to assign a probability that a histogram data point belongs to a particular group of data points in the histogram to isolate data points representing response of the cells to the scenario from extraneous information, thereby providing probabilistic clusters for cell responses of interest for segmenting each of the images into image classes. The images are divided into plural subfields, and numerical data sets for each subfield are extracted from the image segments, such numerical information representing features of the images having information concerning the cellular response to the scenario. A model fitting procedure, e.g., a parametric nonlinear dynamic model, is applied to each data set and corresponding model parameters are extracted. And, at least a portion of the model parameters are used to define a feature model space having scenario clusters for known materials. In these embodiments, attempting to classify the scenario comprises repeatedly exposing the cells to an unknown scenario, and repeating steps required for providing a model space having statistical expert information concerning the unknown scenario. Distances between clusters representing unknown scenarios and known scenarios are then calculated, and a likelihood determined that an unknown scenario is a known scenario by combining such distances by expert voting.

The present method has a number of practical uses, including classifying unknown drug candidates, classifying unknown toxins, etc. And, the method can be useful for comparing orthogonal biological system responses within a numerical feature space vector.

A particular embodiment comprises providing an apparatus comprising a digital camera for recording images, a receiver for receiving live cells, an injection port for injecting a material into contact with the cells, and a computer for processing information concerning response of the cells to the material. Live cells are then exposed to a known material, and images are acquired of the cells at a first time to a second time through a predetermined time period. These images are analyzed to isolate cell features from extraneous information by converting response changes to hue, saturation, value histograms versus time. Probabilistic segmentation is applied to assign the probability that a histogram data point belongs to a particular group of data points in the histogram to isolate data points representing response of the cells to the material from extraneous information, thereby providing probabilistic clusters versus time for cell responses of interest for segmenting each of the images into image segments. The images are divided into plural subfields, and numerical information is extracted for each subfield from the image segments, such numerical information representing features of the images having information concerning response of the cells to the material. Numerical information is then generated versus time for each subfield for each cell response monitored to provide plural expert curves representing cellular response to the material versus time. Parametric nonlinear auto-regressive external input models are applied to each of the plural expert curves to describe such curves using a predetermined number of parameters. At least a portion of the parameters are used to define a feature model space having scenario clusters for known materials. Cells are then exposed to an unknown material, and such steps are repeated as required for determining expert parameters. The expert parameters are weighted, and normalized to provide a virtual expert. Thereafter, a determination is made concerning the likelihood that an unknown scenario is a known scenario.

A cytosensor apparatus also is described, comprising a vessel defining an inlet for cells that provides an inlet for at least one bioactive unit or at least one test compound for functionally contacting the at least one bioactive unit and at least one test compound. A detector also is provided for detecting changes in the bioactive unit. The apparatus typically utilizes a computer for controlling the apparatus and analyzing changes detected by the detector. The apparatus also may include a signal processing system coupled to the detector, and an analyzer that converts a digital output from the signal processing system into a result.

The method also can be implemented using a computer program encoding the method. Moreover, a computer-readable medium is described on which is stored a computer program having instructions for executing embodiments of the disclosed method.

### BRIEF DESCRIPTION OF THE DRAWINGS

FIGS. 1A-1B illustrate pigment aggregation in chromatophores obtained from fish of the genus Betta prior to and after exposure to norepinephrine, respectively.

FIGS. 2A-2B illustrate the appearance of Betta chromatophores prior to and after an exposure of a few hours duration to cholera toxin (CTX).

FIGS. 3A-3B illustrate the appearance of a jewel cichlid scale (from Hemichromis bimaculatus) before and after, respectively, exposure to diisopropyl fluorophosphate (DFP).

FIG. 3C illustrates eight scales from a jewel cichlid viewed in reflected light. Four scales were exposed to DFP and four scales were unexposed.

FIG. 4A shows iridophore patches of a jewel cichlid scale before and after exposure to DFP.

FIG. 4B is a graph of color changes of an iridophore patch as a function of time following exposure to DFP. FIG. 4C is a color-space rendering of a color trajectory of the iridophore patch of FIG. 4B following DFP exposure.

FIG. 5A illustrates a segmentation of colors from iridophore patches before (top) and after (bottom) exposure to DFP.

FIG. 5B is a graph of response of the iridophore patch of FIG. 5A as a function of time to various concentrations of DFP.

FIGS. 6A-6C illustrate pigment aggregation in Betta chromatophores after exposure to norepinephrine (NE) without pre-incubation with CTX, with pre-incubation with a threshold concentration of CTX, and with pre-incubation with a substantial concentration of CTX, respectively.

FIGS. 7A-11A illustrate the appearance of Betta splendens chromatophores prior to exposure and FIGS. 7B-11B illustrate the appearance of Betta splendens chromatophores after exposure, respectively, to various strains of bacteria.

FIGS. 12A-12B illustrate the appearance of cultured chromatophores before and after, respectively, exposure to another strain of bacteria.

FIG. 13 illustrates the appearance of a scale that includes several types of chromatophores after exposure to one or more analytes.

FIGS. 14A-14D are photographs of chromatophores after exposure to bioactive conditions.

FIGS. 15A-15B are photographs of Betta splendens and Hemichromis bimaculatus chromatophores, respectively, cultured on polystyrene using a common method.

FIGS. 16A-16C are photographs of several Betta splendens chromatophore color variants unexposed to analytes.

FIG. 17 is a dark field photograph of chromatophores from a violet variant of Betta splendens without exposure to analytes.

FIGS. 18A-18C are photographs of an extruder used for encapsulation.

FIG. 19 is a schematic diagram of a cytosensor.

FIG. 20 is a cross sectional schematic diagram illustrating a camera unit for a cytosensor.

FIG. 21 is a lighting system and chamber holder for use with a cytosensor.

FIG. 22 is a plan view of the lighting system and chamber holder of FIG. 21.

FIG. 23 is a cross sectional view of a chamber 130 used with certain embodiments of the described cytosensor.

FIG. 24 is an exterior assembled view of the chamber 130 of FIG. 23.

FIGS. 25A and 25B are alternative embodiments of a chamber 130 for microfluidic flow.

FIGS. 26A-26B illustrate a cell chamber.

FIG. 27 is a schematic diagram of one portion of a fluid interconnect.

FIGS. 28A-28B illustrate another portion of the fluidic interconnect of FIG. 27.

FIG. 29 is a schematic diagram illustrating a microlamination method for defining a cell chamber.

FIGS. 30A-30C are schematic diagrams of multi-analyte reaction cells that include analyte reservoirs.

FIG. 31 is a photograph of a mold for a reaction chamber.

FIG. 32 is a schematic diagram of an optical detection system.

FIG. 33 is a flow chart setting forth steps used to practice a soft classification method according to the present invention.

FIGS. 34A-34B are photographs of an erythrophore prior to and after exposure to norepinephrine in a solution containing calcium ions.

FIGS. 35A-36B are photographs of an erythrophore prior to and after exposure to norepinephrine in a solution lacking calcium ions.

FIG. 36 provides dose-response curves for erythrophores exposed to channel blockers verapamil, nifedipine, and deltiazem, with and without added calcium ions.

FIG. 37 is a graph of erythrophore response to verapmil.

FIG. 38 provides curves illustrating erythrophore response to BAPTA/AM and ionomycin.

FIG. 39 illustrates erythrophore response to high and low concentrations of ryanodine.

FIG. 40 is a block diagram of a method of analyzing cytosensor data.

FIG. 41 is a graph illustrating data produced by embodiments of the disclosed method.

FIG. 42 is a schematic diagram of an encapsulation apparatus.

FIG. 43 is a photograph of a ferromagnetic micro-ball.

FIGS. 44A-44C illustrate operation of a Y-branch.

FIGS. 45A-45B are photographs of masks for a round coil and a square coil, respectively.

FIG. 46 is a photograph of a coil made using the mask of FIG. 38A.

FIGS. 47A-47B are photographs of a micro-ball valve orifice and catch plate, respectively.

FIG. 48 is a graph of misalignment error as a function of bonding pressure.

FIG. 49 is a graph of diodicity as a function of flow rate for micro-ball valves.

FIGS. 50A-50E illustrate construction of a channel assembly.

FIG. 51 is a three dimensional representation of feature spaces defined by response of chromatophores to B. cereus strains in the absence of elictors.

FIG. 52 is a three dimensional representation of 12 dimensional feature spaces defined by response of chromatophores in the presence of various elictors.

FIG. 53 is a three dimensional representation of feature space defined by L-15 medium in the absence of elicitors, indicating that cluster separation in the presence of L-15 for bacterial strains BC 5 and BC 6 is poor.

FIG. 54 is a three dimensional representation of feature space defined by MSH in the presence of elictors demonstrating that cluster separation in the presence of MSH for bacterial strains BC 5 and BC 6 is good.

FIG. 55 is a three dimensional representation of feature space defined by MSH in the presence of elictors demonstrating that cluster separation in the presence of MSH for bacterial strains BC 5 and BC 6 is good.

FIG. 56 is a three dimensional representation of feature space defined by MSH and forskolin demonstrating that forskolin also allows differentiation of BC 5 and BC 6 but not as efficiently as MSH.

FIGS. 57A and 57B compare the response pattern from cells to clonidine in a 2-well plate versus a microfluidic flow device.

FIG. 58 is a representation of 12-dimensional feature space defined by an elicitor panel as disclosed herein providing a cluster map for BC 1.

FIG. 59 is a representation of 12-dimensional feature space defined by an elicitor panel as disclosed herein providing a cluster map for BC 5.

FIG. 60 is a representation of 12-dimensional feature space defined by an elicitor panel as disclosed herein providing a cluster map for BC 6.

FIG. 61A-B show four simplex scenarios corresponding to each of the elicitors present in an elicitor panel.

FIG. 62 provides a mechanistic interpretation of the results of FIG. 61.

FIG. 63A-63F illustrate the response of chromatophore cells to Bacillus cereus ATCC49064 after the chromatophore cells first have been preincubated with Lactococcus for 30 minutes before the addition of Bacillus cereus.

FIG. 64A illustrate a software agent model and device schematic for a system input that illustrates a conceptual model for the modular organization of certain cellular regulatory activities that uses concepts for data organization and analysis.

FIG. 64B illustrate a software agent model and device schematic for a system input that illustrates a conceptual model for the modular organization of certain cellular regulatory activities that uses concepts for data organization and analysis.

FIG. 64C illustrates an embodiment of a biosensor using integrated ensembles of biological elements (DNA aptamer and antibodies) and data analysis modalities (knowledge base and associated inference engine) for sample classification and identification.

FIG. 65 illustrates N-Dimensional Segmentation of Mechanism Space with Elicitors (N=1). where each simplex scenario is defined by a unique reference node and is represented by a vertical column, rows represent secondary nodes, and not all secondary nodes are included in each simplex scenario.

FIGS. 66A-D illustrate application of an elicitor panel using the reference node approach (in FIG. 66A, chromatophores were exposed to 1 μM cirazoline, 100 nM clonidine or 100 μM H89 1200 seconds following media control causing aggregation in each instance; in FIG. 66B chromatophores were exposed to 1 μM cirazoline, 100 nM clonidine or 100 μM H89 1200 seconds after initial exposure to 100 μM forskolin. Effect of clonidine and cirazoline is not detectable following exposure to forskolin, whereas H89 exposure still results in cell aggregation; FIG. 66C is a schematic diagram illustrating GPCR signal transduction pathways mediated through adenylate cyclase and cAMP. Forskolin is an adenylate cyclase activator; cirazoline is an α₁-adrenergic receptor agonist; clonidine is an α₂-adrenergic receptor agonist; H-89 is a PKA inhibitor; in FIG. 66D 1 μM cirazoline (green), 1 nM clonidine (red) or 100 μM H89 (yellow) were added to chromatophore cultures 12000 seconds following exposure to 100 μM forskolin and 1 mM PMSF (yellow); no aggregation occurred in response to clonidine and cirazoline exposure, PKA demonstrated increased sensitivity to H89 inhibition in the presence of PMSF (yellow), and control cultures (blue) showed decreased sensitivity).

FIG. 67 is a projection of the feature spaces defined by L-15 Medium (A) and the full elicitor panel (B) using Principle Component Analysis (PCA) (agents include the three Bacillus strains and PMSF; the L-15 representation demonstrates improved classification in the presence of elicitors; the integration of probabilistic graph theoretical concepts with the use of will result in analytical tools with capable of determining the impact of unknown compounds).

FIG. 68 compares results from two different biosensor systems, referred to as Gemini and Mercury.

FIG. 69 is a network graph depicting non-conditional and conditional network structures for Gs and Gq pathways.

FIG. 70 illustrates integration of structural motifs identified in cellular regulatory systems with Bayesian network approaches and Markov Models.

FIG. 71 is is a graph (minutes versus cell area) illustrating typical output showing change in cell area with respect to time.

### DETAILED DESCRIPTION

### I. Introduction

Every element in biological systems, broadly defined, has functionalities associated with the transfer and processing of information on the basis of individual or ensemble activities. Spatiotemporal organizational motifs integrate modules into small world, scale-free, hierarchical networks for improved communication efficiency, computing power and system robustness. Self-organization can provide increased flexibility for system formation and responses to the loss or degradation of ensemblic elements. Exposure to environmental cues results in the formation of data structures or functional modules for system activities. Knowledge regarding the existing environment or possible environments within which the system exists is represented through the iterative selection of system elements (system evolution) and training or calibration runs, all organized within a modular framework for maximum plasticity and response efficiency.

A method for determining the identity of an unknown agent or action, referred to herein as a scenario, or other information concerning the scenario, such as biological mechanistic information, is described, along with embodiments of a system useful for implementing the method. The method typically involves generating data, and then analyzing that data according to ensemble methods. The method can be automated for implementation by a computer system operating computer readable medium for rapid data acquisition, display and analysis.

### II. Definitions

For convenience, several terms used to describe examples are defined. These terms are provided to aid the reader, and should not be interpreted to have a narrower scope than would be understood by a person of ordinary skill in the art.

A bioactive material is a material that elicits a physiological, morphological, or other response from a living system either alone or in combination with other materials.

An analyte is a substance to be detected or quantified and in some examples is a bioactive agent that is a chemical agent or a living organism such as fungi, bacteria, virus, molds, protists, animal cells, or other animals that elicits a response from a living cell.

A cytosensor is a biosensor apparatus that uses living cells to detect analytes. In some examples, a cytosensor includes at least one cell and changes produced in the cell by an analyte or combination of analytes is detected by methods such as visual inspection, or other methods. In other examples, changes produced in response to exposure to an analyte are detected by a statistical analysis of cell properties.

An ensemble is both a non-random mixture of biological elements and a collection of systems with well defined thermodynamic properties as defined in the field of statistical thermodynamics.

An ensemble or voting data analysis method is one that is not predicated on the use of statistical methods in the usual sense.

### III. Design Paradigms for Improved Biosensor Design

Biosensor units consist of ensembles of biological elements including, but not limited to, higher organisms, tissues, multiple cell types, cells, sub-cellular organelles and molecules of biological origin. Improved biosensor systems can be developed using design paradigms for software agents taken from the field of artificial intelligence and underlying structural motifs used by biological systems for organizing regulatory and transformative networks, with resulting improvements in the organization of sensing, signal processing and communication activities.

Every element involved in cellular networks (nucleic acid, protein, metabolites, etc.) has roles in cellular sensing, information processing, and communication activities. Elements self-organize into functional modules under master regulatory elements as the system responds to environmental cues, providing maximum flexibility for module formation and compensation for loss or degradation of individual elements. Spatiotemporal organizational motifs provide functional modules with a small world, scale-free network topology for improved communication efficiency, computing power and system robustness. The working examples provide specific examples of organizational motifs found in biological and other systems. Overlapping cellular regulatory and transformative networks are plastic and “trained” by their local environment. Short term responses involve the formation of functional modules using materials at hand without significant changes at the genetic level. Longer term responses involve significant remolding of the physicochemical organization of the cell and can be measured through changes in gene expression patterns or the complement of available regulatory and transformative activities at the system level.

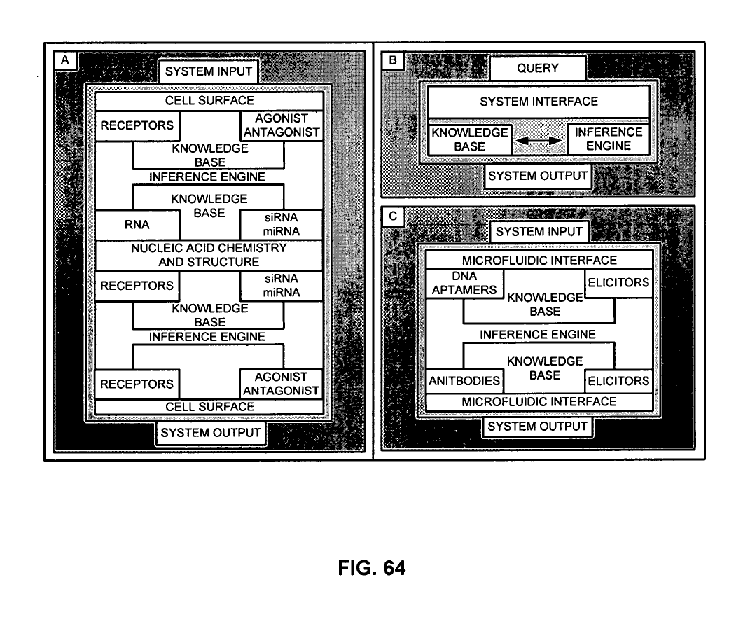

FIG. 64A shows a conceptual model for the modular organization of certain cellular regulatory activities that uses concepts for data organization and analysis (inference engine and knowledge base) taken from the field of artificial intelligence as shown in FIG. 64B. An inference engine, consisting of statistical models, maps system inputs (sample data) to outputs (data representation) in consultation with training data present in the knowledge or data-base. The data dimensionality of sample inputs is reduced as necessary at the system interface through extraction of numerical and non-numerical features. Optimal feature selection is important for effective and robust data classification. Data structures in the database provide frameworks for representing prior knowledge and integrating current data (obtained through sample or other analyses) in order to generate system responses to environmental cues. Knowledge bases and inference engines are integrated into the physical structure of the biological elements that form the functional modules, representing both prior and current knowledge regarding the local environment. Application of primary elictors (well-defined effectors of cell function) defines functional modules or data structures specific for analyses of interest. Secondary elictors are used to provide additional information within the context(s) provided by the primary elicitor(s) for given scenario(s).

FIG. 64C shows an embodiment of a biosensor using integrated ensembles of biological element (DNA aptamers and antibodies) and data analysis modalities (knowledge base and associated inference engine) for sample classification and identification. Sensor structure is plastic and “trained” by the local environment created by the problem to be solved (classification of lipo-polysaccharide (LPS) from different species of gram-negative bacteria, for example). Short term responses to environmental cues (samples) will be governed by elicitors, known bioactive agents that act competitively at recognition sites in the molecular array and in the solution space above the array. This is analogous to the agonist/inverse agonist/partial agonist/G-protein receptor paradigm used by chromatophores to increase the classification dimensionality of the extracellular space and perform the analog to digital conversion necessary for intracellular signal processing. Longer term responses to environmental cues (applying analyte to different molecular sorbents already on hand, development of new molecular recognition sorbents for bioactive agents not included in the original training set) will involve a significant remolding of the physical structure of the sensor base. This is analogous to RNA and DNA translation/regulatory activation. Prior knowledge is represented through the selection of molecular recognition sorbents (evolved in the lab) and elicitors (also evolved in the lab) and data integrated into the database. Current knowledge regarding the environment (sample matrix) comes through sample matrix analysis (initial training) and sensor calibration with analyte standards (fine tuning or optimization). The combined elicitors and molecular sorbent represent the knowledge base for the sensor system. Data dimensionality significantly reduced as the organization and structure of the biosensor elements performs most of the processing prior to data analysis with soft classification.

### IV. Living Systems for Exhibiting a Response to Bioactive Materials or Conditions

A variety of living systems can be used to detect bioactive agents. Among the more useful cells for testing bioactive materials are chromatophore cells. Living cells in addition to chromophores can be used to practice the present method and system for classifying a scenario. For example, virtually any living cell that experiences a detectable change in response to an interaction with an analyte or analytes, which change can be measured, can be used. A partial list of additional living cells that can be used includes, without limitation, PC 12 pheo-chromocytoma cells, HeLa cells, small multi-cellular organisms, such as daphnia, brine shrimp, fish, rodents, and human beings.

1. Chromatophore Cells

Chromatophores are dermal cells that develop from the neural crests of animals such as fish, cephalopods, and amphibians. Within these cells are organelles called pigment granules or chromatosomes. The movement of the chromatosomes within a pigment cell alters the cell appearance. When the pigment granules are evenly dispersed, the cell appears dark; when the pigment is aggregated at the cell center, the cell appears pale. Pigment granules are transported along the system of microtubules and actin filaments within the chromatophore. In many species, changes in coloration can be controlled by the nervous and endocrine systems, with the response mediated by the chromatophore cell membrane receptors (F. W. R. Chaplen, R. H. Upson, P. N. McFadden, and W. J. Kolodziej, Fish Chromatophores as Cytosensors in a Microscale Device: Detection of Environmental Toxins and Bacterial Pathogens, Pigment Cell Research 15, 19-26, 2002). There are a number of different chromatophore types, which contain different pigments.

The optical properties of some chromatophores are determined by organelles that include light-absorbing pigments such as melanins (black), pteridines (red), and carotenes (yellow). The optical properties of other chromatophores are based on organelles that have periodic internal structures that iridescently interfere with incident light causing selective reflection of certain colors and transmission of other colors. Such organelles are referred to as iridescent and can change their hue, and hence the hue of the cell, in response to various stimuli. Any single chromatophore generally contains one type of organelle so that its appearance is determined by this type of organelle.

Classes of chromatophores are commonly assigned names based on a dominant color, for example, melanophores (black), erythrophores (red), and iridophores (yellow and/or iridescent). Subtypes of these classes include bluish-black melanophores, yellowish-red erythrophores, and others. The complex spectral properties of various animal skins are typically produced by combinations of chromatophores of several types.

One source of chromatophores is Betta splendens fish. These fish are readily available in many colors, and the fins typically include chromatophores of various types. Male Betta splendens are generally more noticeably colored and hence are superior to females as a source of chromatophores, but female Betta splendens fish can be used. Chromatophores can also be obtained from other fish species.

Betta fish are customarily classified as Asian labyrinth fishes in the family Belontiidae. The family Belontiidae includes five subfamilies (Belontinae, Macropodinae, Trichogasterinae, Sphaerichthyinae, and Ctenopinae). The subfamily Ctenopinae includes the genus Betta (see, for example, H. Pinter, Labyrinth Fish, Barron's Educational Press, New York (1986)). Betta that are listed by Pinter include: B. (Schaumnestbauer); B. bellica Sauvage, 1884; B. coccina Vierke, 1979; B. fasciata Regan, 1909; B. foerschi, Vierke, 1979; B. imbellis I. adiges, 1975; B. maragdina Ladiges, 1972; B. splendens Regan, 1909; Betta Maulbruter; B. avabatoidcs Bleeker, 1850; B. balunga Herre, 1940; B. brederi Myers, 1935; B. macractoma Regan, 1909; B. picta (Cuvier u. Valenciennes, 1846); B. pugnax (Cantor, 1850); B. rubra Perugia, 1893; B. taeniata Regan, 1909; and B. unimaculata (Popta, 1905). Betta fish and fish of the whole subfamily Ctenopinae are sources of useful chromatophores. Some useful fish include freshwater zebrafish, south American cichlids, African cichlids, saltwater damsels, goldfish, gouramis, and others.

Early research used simple comparisons of paired digital images to measure pigment dispersion and aggregation in melanophores from the African clawed frog, X. laevis. While this approach results in basic information about the responsiveness of the cells to a given stimulus, an examination of the kinetics of chromatosome movement is more useful for characterizing the strength of a reaction and the nature of the response. Later research demonstrated that measurement of the dynamics of chromatosome movement provided a wealth of information about the biological response of cells to systemic perturbations.

Disclosed embodiments of the current system can acquire digital images at a rate of up to one frame per second throughout the course of a sample run. Moreover, certain embodiments utilize sophisticated image segmentation software that is capable of identifying individual cells and tracking particular features, such as apparent cell area.

2. PC-12 Pheo-Chromocytoma Cells

Exposing Pheo-chromocytoma cells to bioactive conditions, such as analytes, can effect the development of outgrowths, such as dendrites, by such cells. Various such outgrowths are readily recognizable and distinguishable, and the size of such outgrowths can be measured quite accurately. As a result, by exposing PC 12 pheo-chromocytoma cells to particular bioactive conditions, and thereafter monitoring a change in the size and/or morphology of such outgrowths, a quantifiable change from a state prior to administration of the bioactive conditions to a state subsequent to administration of the bioactive conditions can be obtained. Any quantified change can thereafter be processed according to embodiments of the method of the present invention, or analyzed using the system and /or software of the present invention, to provide information concerning the bioactive conditions.

Based upon the example of PC 12 Pheo-chromocytoma cells, it will be understood by a person of ordinary skill in the art will understand that additional neuronal cell lines, including both rodent and human cell lines, can be used for feature detection as a result of an exposure to a bioactive condition. Such cells can be primary cells, i.e., animal-derived cells that are used a single time for the desired analysis. Examples of primary neuronal cells that can be used include cells from the spinal cord, the hippocampus, the cerebellum, and other regions of the brain. Particular cells lines that can be used according to the present invention, without limitation, include MG108-15, N1E-115, Neuro-2A, and combinations of such cells.

3. HeLa and Other Cancer Cell Lines

Cancer cell lines also are useful for detecting the presence of a bioactive condition or providing information concerning such bioactive condition. One example of a human cancer cell line that can be used is HeLa cells, which are immortal cancer cells derived from cervical cancer. HeLa cells are useful for studying gene expression and protein production.

HeLa cells can be functionally admixed or placed in proximity to a bioactive condition. Thereafter, RNA produced by the HeLa cells can be isolated and quantified using, for example, DNA microarrays. This provides a quantifiable change as a result of exposure to a bioactive condition since RNA quantity produced as a result of DNA transcription of a particular gene in a first state prior to effective exposure to the bioactive condition and a second state that is subsequent to exposure to the bioactive conditions provides an indication of the living cell's response to the bioactive conditions. Thus, quantified detection of RNA is a feature that can be analyzed according to the present method. Similarly, in addition to quantification of nucleic acids produced by exposure to a bioactive condition, protein production as a result of similar exposure also can be quantified.

In addition to HeLa cells, additional cell lines that can be used in a similar fashion include 3T3, COS7, HEPG2, all of which are mammalian cells.

4. Single Cell Organisms

Simple, single-cell organisms also can be used. Examples of such organisms include protozoans, such as amoebae and parameciums, euglena, etc. One example of a feature that can be quantified in such organisms subsequent to exposure to an analyte is cytoplasmic streaming. Cytoplasmic streaming involves a change from a fluidic state to a gel-like state and vice-versa within the organism. Cytoplasmic streaming can be used by the organism for cell motility.

The degree of cytoplasmic streaming correlates with exposure to external conditions, including environmental conditions, such as temperature and pH, as well as to exposure to bioactive conditions. Again, as a result of such exposure, a first state of the organism representing a particular degree or absence of cytoplasmic streaming to a second state subsequent to exposure to the bioactive condition can be quantified and hence used as a feature for analysis in the method of the present invention.

Additional examples of simple organisms and their reaction to analytes that can be used include stentors, which are bell-shaped organisms. These organisms change the shape of their exterior surface for various purposes, including encompassing material for ingestion. An analyte can be effectively applied to the stentor, and then the speed with which the exterior morphology of the stentor bell changes can be quantified and used as a feature.

5. Multi-Cellular Organisms

One example of such a multi-cellular organism that can be used is daphnia, which are commonly referred to as water fleas. The Environmental Protection Agency has adopted procedures for using such organisms to monitor the quality of water samples. A number of features of such organisms can be monitored to determine a change from a first state prior to administration of, or otherwise exposure to, a bioactive condition to a second state subsequent to administration of or exposure to a bioactive condition. Moreover, the frequency and/or amplitude of the change of the morphology also can be monitored and subsequently analyzed according to the method or using the software of the present invention. One example of such a morphological change is a swelling in the abdomen of the daphnia as a result of exposure to a bioactive condition.

6. Complex, Multi-Cellular Organisms

More complex organisms also can be used. Examples include flathead minnows and rainbow trout, rats, mice, humans. The EPA currently uses lethal-dosage (LD₅₀) tests with such organisms to detect the presence of analytes or to monitor the quality of water samples. The EPA's method requires that a certain number of fish die so that an LD₅₀ can be determined. However, such fish exhibit other visually detectable responses to exposure to analytes that can be monitored prior to death of the fish. These features include color, swimming velocity, swimming acceleration, buoyancy, direction bias, mouth movements (gulp, cough), gill ventilotory actions (amplitude and frequency), gill coloration, fin projection (e.g., limp fins), and startle response.

### V. Testing Bioactive Materials

Living cells described above can be used to detect the presence of, and may be used to provide information concerning the nature of, a wide range of bioactive conditions, including bioactive compounds, organisms, or toxins, and prior knowledge of the type or structure of the bioactive compound is unnecessary. For example, chromatophores respond to analytes derived from medical, forensic, or pharmaceutical specimens such as neurotransmitters, norepinephrine, adenosine, dopamine and analogs thereof such as LSD, cocaine, serotonin analogs, hormones such as MSH (1 nM), melanophore concentrating hormone (MCH) and analogs thereof, intracellular signal transduction agents such as nitric oxide, forskolin (10 nM), cAMP, cGMP, calcium ion, protein kinase A, and analogs thereof; pharmaceutically active agents such as caffeine (100 μM), alpha-2 adrenergic agonists (yohimbine), pertussis toxin, dibutyryl cAMP, dibutyryl cGMP, including prescription drugs, off-the-shelf drugs, and illicit drugs; toxic agents such as chemical warfare agents (for example, diisopropyl fluorophosphate (DFP)); agricultural chemicals such as paroxon; chemical toxins in food and water; biological toxins in food and water such as cholera toxin; toxin-producing bacteria (Bacillus cereus, Salmonella enteriditis); bacterial cells, fungal cells, yeast, protists, and animal cells, such as PC12 neuronal cells.

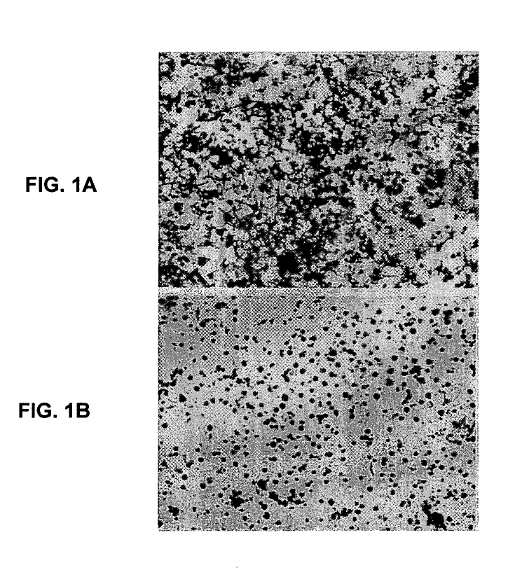

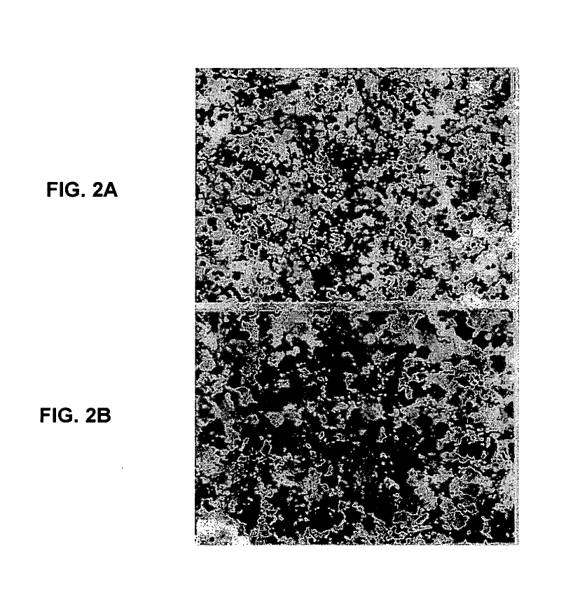

Chromatophore response to some of these analytes is illustrated in the accompanying figures. FIGS. 1A-1B illustrate the appearance of Betta chromatophores before exposure and after a few seconds of exposure, respectively, to a 1 nM solution of norepinephrine. Referring to FIG. 1B, aggregation of chromatophore pigment is apparent. FIGS. 2A-2B illustrate chromatophore appearance before and after, respectively, exposure to 100 pM solution of cholera toxin. Pigment aggregation is evident.

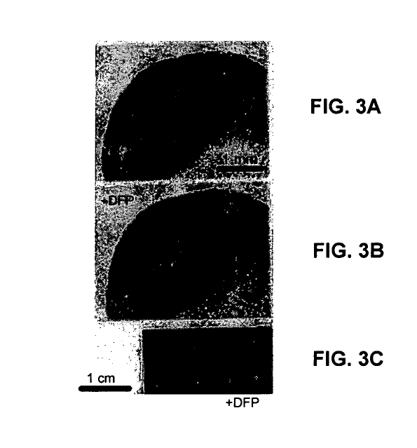

FIGS. 3A-3B illustrate the appearance of a jewel cichlid scale from Hemichromis bimaculatus before and after exposure to diisopropyl fluorophosphate (DFP), respectively, at a concentration of about 100 μM. FIG. 3C illustrates the appearance of eight scales from a jewel cichlid viewed by reflected light on a black background. Four scales on the right were exposed to DFP while the four scales on the left were unexposed.

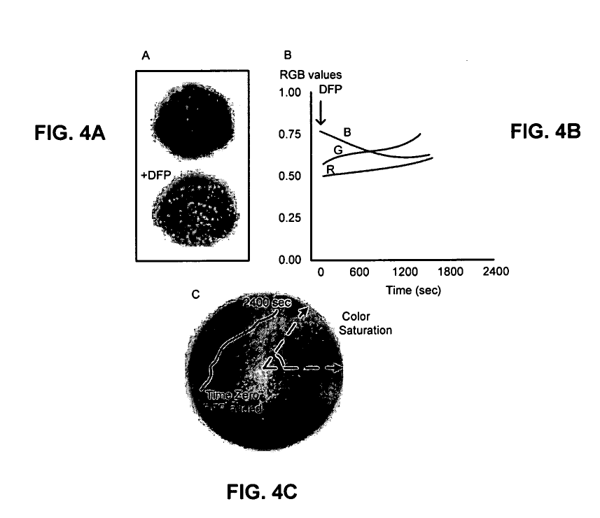

FIGS. 4A-4C also illustrate changes in jewel cichlid chromatophores in response to DFP exposure. FIG. 4A shows iridophore patches of a jewel cichlid scale, before and after exposure to DFP. FIG. 4B contains graphs of red (R), green (G), and blue reflectances (B) of an iridophore patch as a function of time after exposure to DFP. FIG. 4C contains a color-space rendering of a color trajectory of the iridophore patch following DFP exposure.

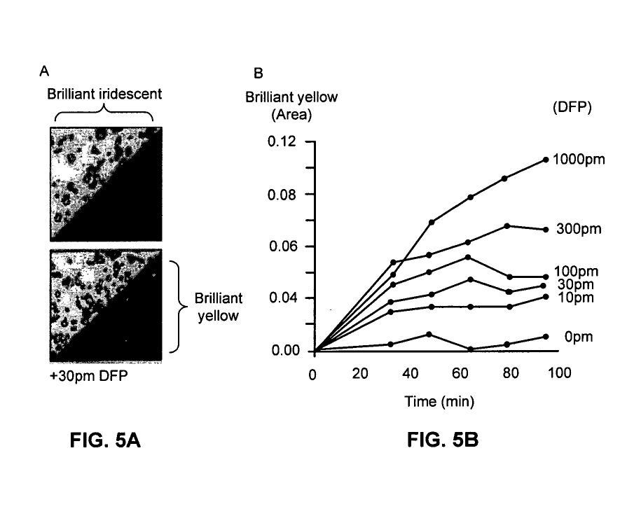

FIGS. 5A-5C further illustrate DFP exposure on iridophore patches. FIG. 5A contains a segmentation of colors from an iridophore patches before (top) and after (bottom) exposure to 30 μM DFP. The upper-left corners of the images show the segments that are more brilliant than a cut-off intensity (i.e., those that are the most iridescent among the population of chromatophores). The lower-right corners show the sub-segment of brilliant iridophores that were yellow in hue. FIG. 4B is a graph of brilliant yellow area as a function of time for exposure of iridophores to various concentrations of DFP.

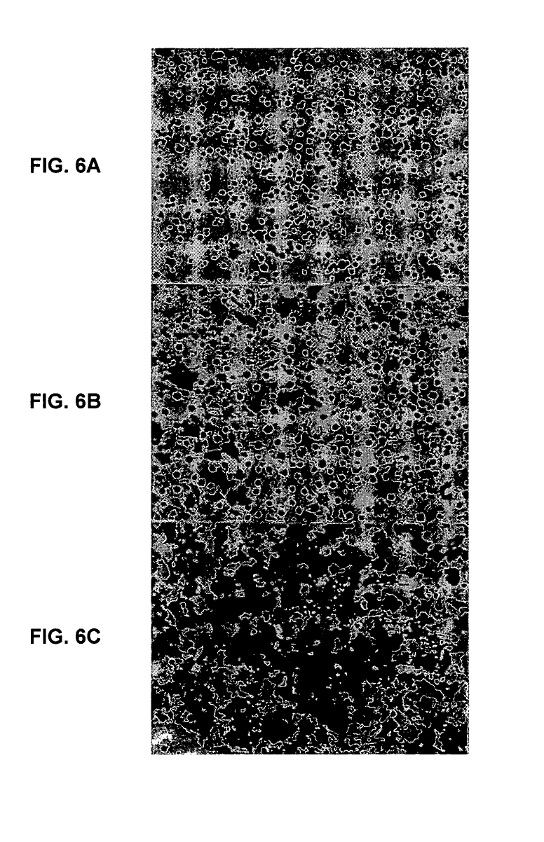

FIGS. 6A-6C illustrate pigment aggregation in Betta chromatophores after exposure to norepinephrine without pre-incubation with cholera toxin (CTX), with pre-incubation with a threshold concentration of CTX, and with pre-incubation with a substantial concentration of CTX, respectively. The pre-incubation period lasted a few hours, and then the chromatophores were exposed to norepinephrine (NE). As is evident, pre-incubation with CTX impaired pigment aggregation in response to NE.

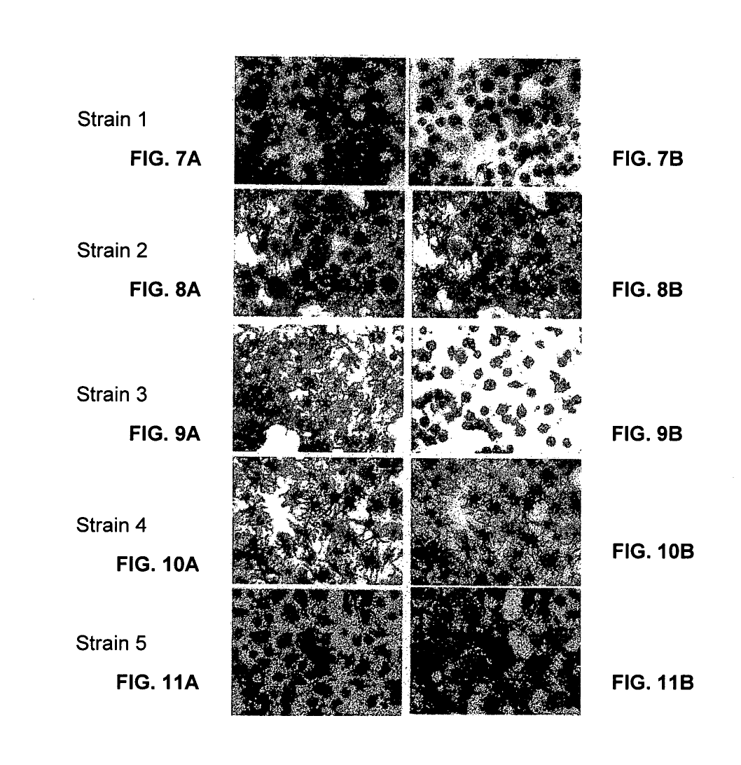

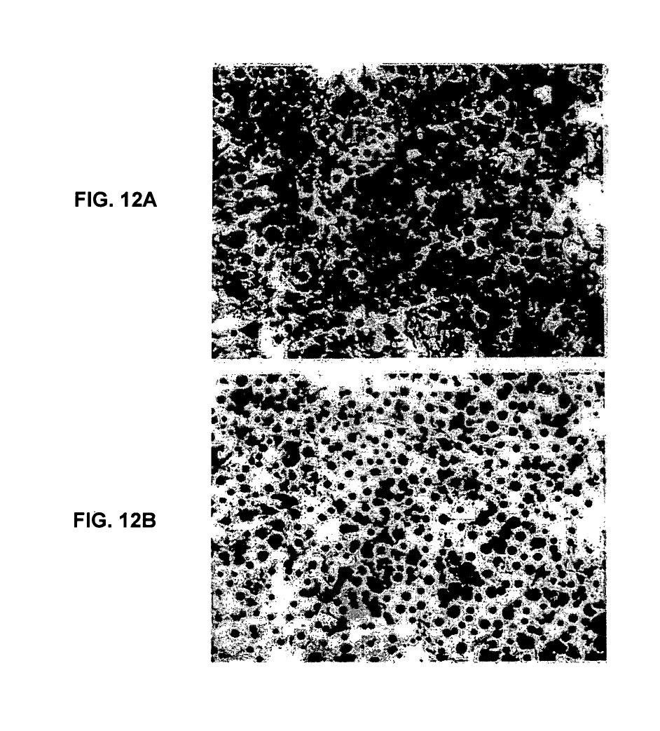

Chromatophores can be used to detect bacterial exposures. For example, FIGS. 7A-11B illustrate the appearance of Betta splendens chromatophores exposed to various strains of bacteria. FIGS. 12A-12B illustrate pigment aggregation produced by exposing cultured chromatophores to yet another strain of bacteria. FIG. 12A illustrates the appearance prior to exposure and FIG. 12B illustrates the effects of exposure to bacteria.

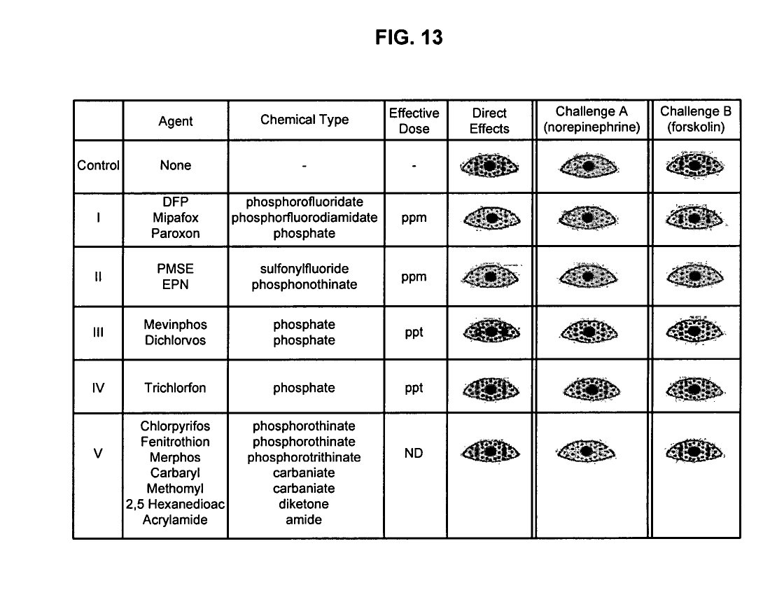

Additional breadth of sensitivity to bioactive compounds can be achieved by using more than one type of chromatophore in a cytosensor. For example, FIG. 13 illustrates the effects of some bioactive conditions on various types of chromatophores that are present in fish scales. The bioactive conditions are divided into categories I-V and representative agents (listed in the second and third columns of FIG. 13) associated with these categories are used to exposure chromatophores at concentrations of up to a few parts per million (ppm) or parts per trillion (ppt). A fourth column lists an approximate concentration at which a chromatophore response was apparent to an observer. Effective doses noted as ppm indicate that concentrations of less than about 300 ppm were effective and those noted as ppt indicate that a response was evident at concentrations as low as about 1 ppt. The appearance of chromatophores is illustrated in the last three columns. Three types of chromatophores (black melanophores, red erythrophores, and a variably hued iridescent patch) are included. The sizes of the melanophore and erythrophores symbols indicate whether pigment was dispersed or aggregated as a result of exposure; the color of the central iridophore patch corresponds to an overall color of the patch as a result of the exposure. The fifth column illustrates scale appearance after response to an agent listed in the first column. The sixth and seventh columns illustrate appearance of scales after exposure to an agent, followed by a challenge with norepinephrine or forskolin, respectively.

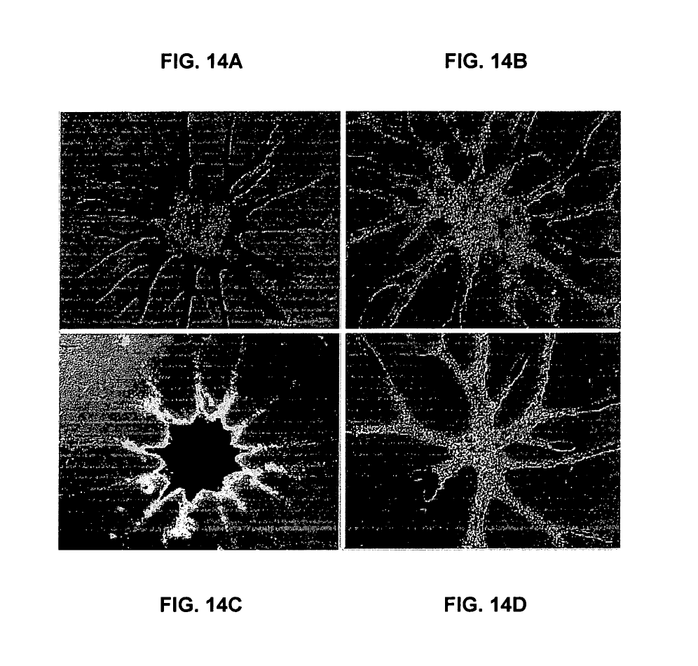

FIGS. 14A-14D are photographs of chromatophores after exposure to bioactive conditions. Differences in appearance can be associated with different bioactive conditions.

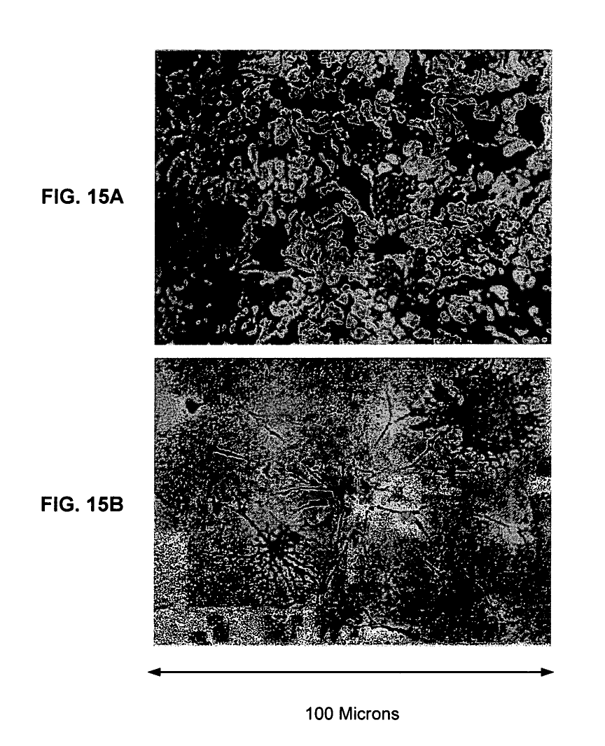

Chromatophores obtained from different species can exhibit different plating densities and morphologies. FIGS. 15A-15B are photographs of Betta splendens and Hemichromis bimaculatus chromatophores, respectively, cultured on polystyrene using a common method. The photographs were obtained at the same magnification.

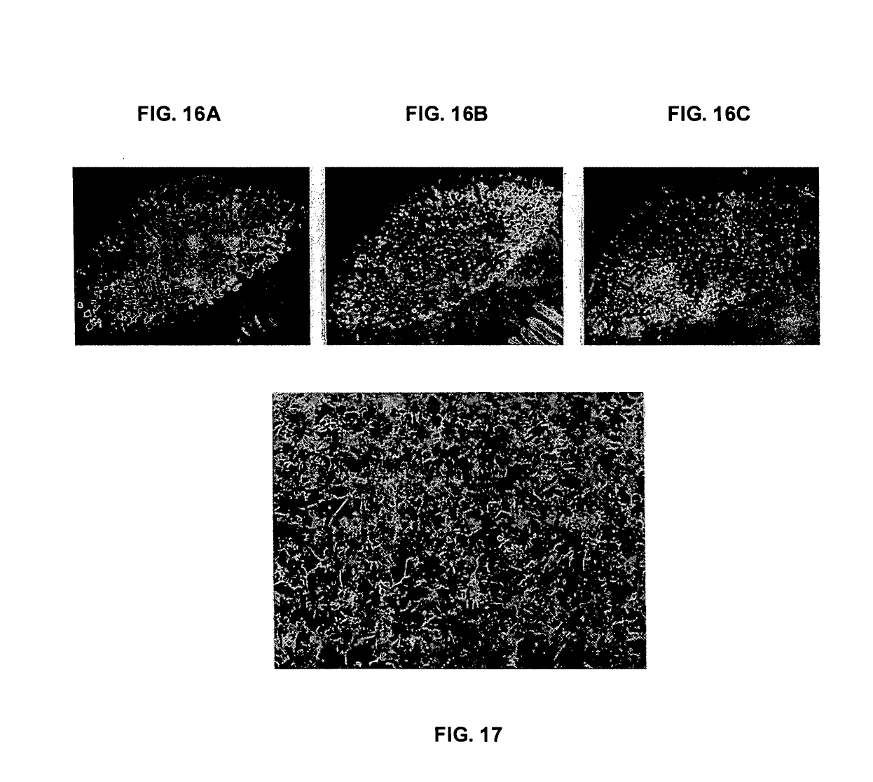

FIGS. 16A-16C are photographs of several Betta splendens chromatophore color variants unexposed to an analyte. FIG. 17 is a dark field photograph of chromatophores from a violet variant of Betta splendens unexposed to an analyte.

As shown in the figures described above, chromatophores have morphological and color characteristics that are ideal for visual, microscopic, or instrumented analysis. As a specific example, Betta chromatophores are small in diameter in comparison with other fish chromatophores that are can be as large as 30 microns or more in effective diameter. (Effective diameter is defined as a length of a minor axis of an ellipse that circumscribes 90% of the visible plasma membrane of a chromatophore. Because chromatophores are often dendritic, cell diameter can be otherwise difficult to determine.) In addition, Betta chromatophores tend to be uniform in size and can be densely packed to facilitate detection of color changes either visually or electronically. (See FIG. 15A.) Thus, dense, information-rich populations occupy small areas and can be interrogated with microscopic analysis and/or video recording. As a specific example, 1000 Betta splendens chromatophores can be situated in an area of less than 1 mm². In contrast, 1000 Nile tilapia chromatophores require an area of about 100 mm². At higher densities Nile tilapia chromatophores were observed to not thrive. Multispectral data collection is facilitated using Betta splendens chromatophores because uniform populations of colors are readily prepared. FIGS. 16A-16C illustrate the appearance of Betta splendens scales FIG. 17 shows cultured Betta splendens chromatophores.

1. Isolating Chromatophores and Viability of Isolated Chromatophores

The following example protocol was used to isolate chromatophores from a blue-green Betta splendens variety, a mostly black Nile tilapia, and a multicolored Hemichromis bimaculatus. Betta chromatophores were cultured in FSL Medium (described below) and survived in a 24-well culture dish for 30 days. The chromatophores remained fully responsive to norepinephrine (NE) during this time with cell numbers dropping by less than half at the end of 30 days. Most of this loss was attributable to the cell feeding method (the medium was suctioned away and the cells were then flooded with fresh medium). The remaining Betta chromatophores were healthy, responsive, and displayed normal morphologies. Any overgrowth of non-chromatophore cells, such as epithelial cells and fibroblasts, can be reduced or eliminated by differential centrifugation procedures that reduce these cell types during an initial plating of cultures.

In contrast, similarly prepared cultures of Nile tilapia melanophores were generally deteriorated at 30 days, with 90% of the cells either lost during medium changes, or remaining as non-responsive, morphologically abnormal remnants. The multispectral chromatophores of Hemichromis bimaculatus also exhibited substantial deterioration after less than 3 weeks in culture.

In addition, scales and fin slices from Betta splendens survive as active explants for at least 4 weeks, a time that is approximately as long as the longest survival time of chromatophores in explants from other tested fish species (Nile tilapia, Hemichromis bimaculatus, and zebrafish). Betta chromatophores can also survive exposure to broad temperature ranges. For example, Betta erythrophores were found to be tolerant of temperatures of up to 30° C. for up to 1 week. Shorter exposure periods of 2 hours at temperatures up to 35° C. did not affect viability. However, 12 hours at 35° C. caused Betta erythrophores to shown signs of deterioration. These upper temperature tolerance limits are a few degrees higher for Bettas than for chromatophores from Nile tilapia that cannot withstand 30° C. temperatures for sustained periods. Betta chromatophores are also relatively insensitive to changes in salinity and osmolarity. Evidence for this is described below in conjunction with experiments in which Betta splendens chromatophore response to bacteria was monitored. In these experiments it was found that the Betta chromatophores could be shifted into an FSL medium that was diluted by at least a 1:1 ratio with bacterial culture medium. Bacterial culture medium is different in both ionic composition and osmolarity from FSL. Similarly, tests of chemical agents have often entailed adding pure water as a diluent to FSL, and the ensuing decrease in ionic strength and osmolarity by at least 20% did not alter the responsiveness of the chromatophores in subsequent testing. Thus, Betta chromatophores can be effectively deployed in instrumentation and protocols that involve substantial changes in ionic strength and osmolarity.

2. Entrapping and Immobilizing Chromatophores

Chromatophores can be entrapped, encapsulated, etc. using a variety of techniques. In a particular example, a suspension of isolated chromatophores was mixed with alginate solution containing 1% to 2.5% weight percent of sodium alginate in de-ionized water. The alginate solution can contain ferromagnetic material (10% to 30% by weight) to facilitate manipulation of beads containing chromatophores within a cytosensor device. The mixture was extruded into beads via an extrusion device 100 shown in FIGS. 1 8A-18C with a method described below in Example 2. The diameter of the extruded beads can be adjusted to any desired diameter, typically in the range 100 μm to 2,000 μm, by controlling the flow of the cell-alginate mixture and the flow of the shearing fluid. Entrapped chromatophores typically do not spread into a morphology characteristic of substrate-anchored living cells. The beads containing entrapped chromatophores can then be used in a cytosensor device.

In another method, the chromatophores can be immobilized on a substrate such as a glass or plastic substrate. Typically, a suspension of isolated chromatophores is brought into contact with glass or polymer beads that have outside diameters in a range of about 100 μm to 2000 μm. The surface of these beads can be treated with attachment factors (acid washing for glass beads for roughness enhancement, collagen and/or fibronectin) to promote cell adhesion. The beads can contain ferromagnetic material (10%-30% by weight) to facilitate bead manipulation within a cytosensor device. See, for example, Example Embodiment 12. The chromatophores typically attach weakly to the bead surface within a few minutes, and then more tightly during the next hour. During the next 24 hours the cells spread into morphologies typical of substrate-anchored living cells. The beads containing immobilized chromatophores can then be used in a cytosensor device.

3. Exposing Living Cells to a Bioactive Condition

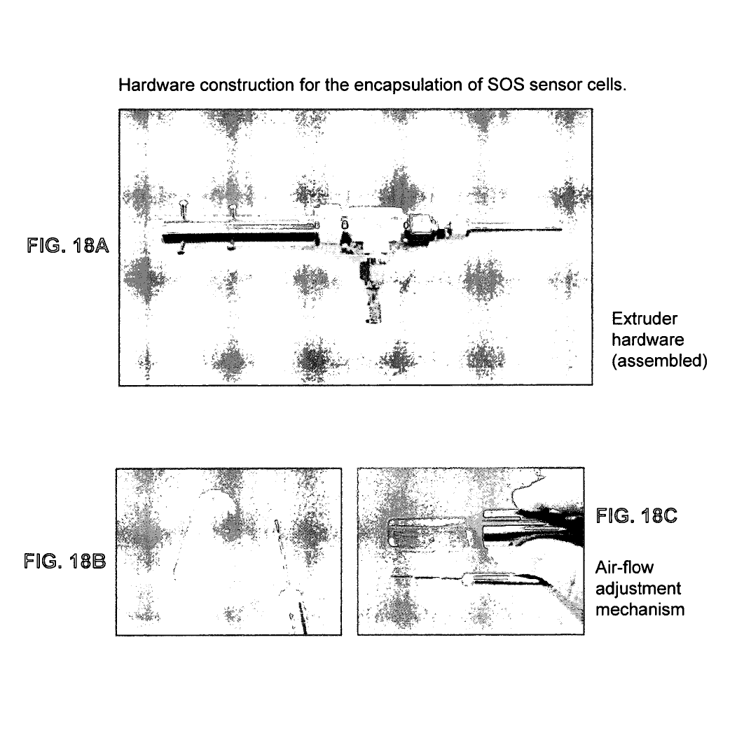

Exposure of living cells, such as chromatophores, to a bioactive condition can be performed outside or inside a cytosensor. These methods are referred to as ex-situ and in-situ, respectively. Ex-situ mixing can be done by mixing the bioactive condition, such as chemicals, fungi, bacteria, virus, mold, protists, animal cells, or animals with an alginate solution (such as one embodiment described in the working examples), with an analyte and a suspension of chromatophores. The analyte/chromatophore/alginate mixture can be extruded into beads. The extrusion is done in the device such as the one shown in FIGS. 18A-18C. Bead size can be adjusted to a size suitable for the injection of the beads into microchannels of a cytosensor device. Alternatively, the encapsulated chromatophores can be visually examined.

In some cases, the resulting beads can be encapsulated with a polymer that allows bioactive conditions to pass into the bead such that the bioactive agent is maintained in physiological contact with the chromatophore. The encapsulation of the bead also functions to maintain the moisture content and nutrient content of the alginate bead such that the chromatophore is maintained in a stabilized environment. Suitable encapsulating agents include for example poly-L lysine, poly-D-lysine, and poly-lysines in fractional mixtures with collagen. The permeability of the membrane for chemicals of different molecular weights may be adjusted separately by adjusting the concentration of polylysine solution, molecular weight of polylysine, and reaction time between polylysine and alginate.

When used in conjunction with a cytosensor, the encapsulated beads can be loaded into a cytosensor device and manipulated as described below. Once the semi-permeable membrane (i.e., poly-L lysine) is formed, transport of fungi, bacteria, virus, mold, protists, animal cells, and/or other animals between the interior of the capsule and the exterior of capsule is prevented. The fungi, bacteria, virus, mold, □immers□, animal cells, and/or other animals can be encapsulated either alive or dead. The transport of chemicals across the membrane (from inside the capsule to the capsule environment, or from the capsule environment into the capsule) depends on the molecular weight of the chemical and the cut-off molecular weight of the membrane, and can be suitably selected.

In-situ methods of exposing chromatophores to analyte involve, for example, first forming alginate beads as described herein and then placing the analyte in functional contact with the beads and allowing the alginate, which surrounds the beads, to dissolve. More specifically, entrapped chromatophores in the form of the alginate beads as described below, can be loaded into the cytosensor device and placed/retained in microchannels, a cell chamber, or an observation chamber. The alginate beads are then exposed to an analyte and the bead is dissolved/disrupted. The beads can be dissolved/liquefied with the use of a suitable complexing agent for polyvalent metal ions. The analyte is thus placed in functional contact with the chromatophore, and this creates an in-situ mixture of analyte and chromatophores at a desired location within the cytosensor device.

Another representative method of in-situ presentation of encapsulated chromatophores and analyte involves performing all of the steps inside a cystosensor. Encapsulation of the chromatophores and, for example, an environmental analyte is performed inside a cytosensor device. A sample of analyte is injected into a receiving chamber of the cytosensor device that already contains admixture of liquid alginate and chromatophores. Such an admixture can be produced by inserting entrapped chromatophores as described below, followed by dissolution of the beads with a complexing agent.

In yet another example, a method of creating an admixture of liquid alginate, chromatophores and analyte, involves introducing liquid alginate containing chromatophores and an analyte either sequentially or simultaneously into a mixing chamber of a cytosensor device. The mixing of the two streams occurs partially by the dissolution of one liquid stream into the other and by the shear created by relative motion of the two fluids. The admixture is then extruded through a coaxial microchannel surrounded with a microchannel annulus carrying a sheeting fluid. The sheeting fluid provides shear for the detachment of the alginate bead, and also provides polyvalent metal ions for the cross-linking of alginate. This method repeats the same steps as discussed above for ex-situ mixing except that it is performed inside the cytosensor device within the microchannel structure. The entrapped analyte and chromatophores in the form of alginate beads are then pushed by the sheeting fluid flow into a washing/encapsulation chamber where the bead is captured by a capture dot as described in the working examples.

The flow of sheeting fluid is replaced with a washing fluid and subsequently with a lysine solution. The lysine solution forms the capsule around the bead just as described in the encapsulation procedure. After another washing, the complexing agent is introduced to liquefy the content of the capsule. Encapsulation is complete and the capsule is ready to be sent to an observation chamber.

This process is repeated with new analyte samples thus providing continuous formation of capsules containing analyte captured at different times and/or locations. Such methods of mixing of analyte and chromatophores are referred to as in-situ snapshot methods.

### VI. Cytosensor Apparatus

1. General Discussion

Cytosensors described herein typically detect an analyte or quantify an analyte concentration based on a morphological, color, or other change in a biological element. In a specific embodiment, a cytosensor includes a group of distinct elements that are either fluidly connected or electronically connected such that they facilitate the capture of data derived from a change in a biological element, such as color change in a chromatophore. The elements of the cytosensor system can be distinct structures or can be integrated such that the entire cytosensor is, or plural, modular cytosensors are, contained within a single housing.

In a representative embodiment, a cytosensor is configured so that analytes are delivered in an aqueous solution or other solution. Typically, an analyte is suspended or dissolved in a liquid medium, such as an aqueous medium. Such compositions may be formulated to include osmotically balanced salts, a physiological pH, and further may be formulated to be free of caustic components that can corrode or otherwise degrade the cytosensor. It is generally desirable to present the analyte in a solution that is substantially free of particulates that can clog or foul cytosensor fluid transfer components. Typically, the analyte solution is filtered using an in-line filter that admits bacteria (1 micron) but removes particles larger than about 20 microns.

Representative cytosensor fluid handling systems include a syringe configured to deliver analyte into a carrier fluid stream flowing in tubing. The carrier fluid and the analyte are directed to biological material, such as chromatophores, that are retained in a chamber. The chamber typically includes an optical window or is generally transparent. In a representative example, plastic tubing of about 0.25 mm inside diameter is configured to receive an analyte volume of about 1 microliter delivered by a glass or stainless steel syringe injected at a rate of about 0.1 microliter/sec.

Other representative examples deliver analytes using wicking or capillary flows. For example, a wick material such as cellulose fiber is configured to draw an analyte sample into a biological material chamber. The analyte can be applied by inserting a portion of the wick into an analyte volume or by releasing a volume of analyte onto the wick. Variables, such as wick diameter, analyte volume, and flow rates can be selected for a particular application. Wick-based flows are advantageous for applications in which pumps and pump power sources are to be avoided.

In other examples, an analyte volume enters a cytosensor prior to being encapsulated. The procedure is similar to the syringe delivery of the analyte described above, except the analyte is mixed with chromatophores and a gelling agent.

Biological materials, such as chromatophores, can be provided in animal parts such as fish scales or fins, or individual chromatophores can be provided. Scales can be isolated by procedures similar to those described in the working examples. The scale (or scales) is then positioned in a chamber, enclosed under a flow of medium, and viewed by, for example, light microscopy or other imaging device, such as a digital camera. Fish scales may be conveniently retained by placement on a porous or fibrous material such as a nylon fabric or cellulose.

Biological materials, such as chromatophores, can be delivered into a chamber at a flow-rate sufficient to allow such material time to settle to the floor of the chamber. The floor of the chamber can be treated with attachment factors (e.g., collagen and fibronectin) to promote cell adhesion. The biological materials typically attach weakly to the floor within a few minutes, and then more tightly during the next hour, at which time the flow-rate can be increased if needed. Over the next 24 hours the cells spread into morphologies typical of substrate-anchored living cells. At this time, the cells are ready for testing under a flow of medium.

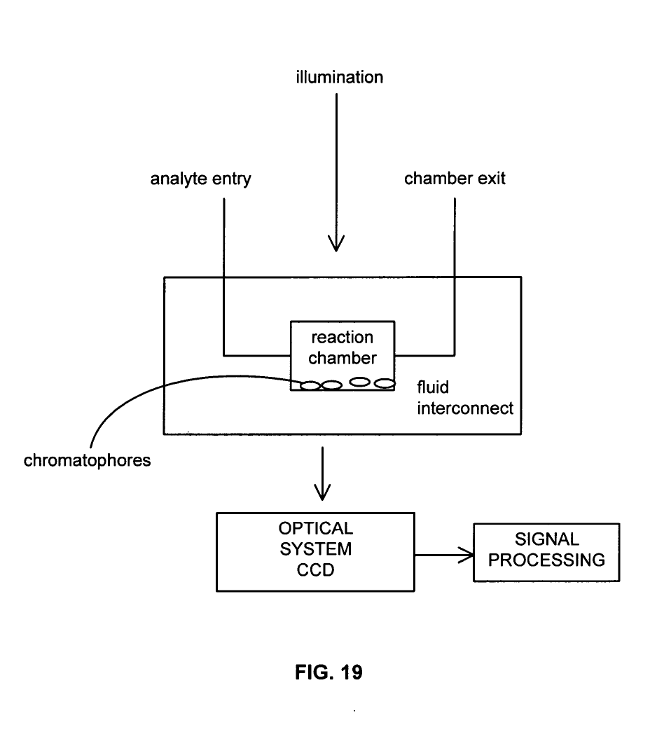

Certain embodiments of the cytosensor apparatus are particularly useful for implementing embodiments of the described method that utilize some visually quantifiable response to an analyte, and even more typically, to monitoring plural responses to the administration of plural analytes. Such embodiments typically include a chamber housing for housing a responder and for introducing a scenario into the housing in a manner that provides a visual response in a responder material also housed in the housing. FIG. 19 schematically illustrates such a cytosensor embodiment that includes an analyte entry tube that delivers an analyte to a reaction cell that contains selected chromatophores. The reaction cell defines a reaction chamber that includes a sidewall and one or more transparent windows, and the chromatophores are held stationary in the reaction chamber. In a specific embodiment, the analyte entry tubing is 0.25 mm inside diameter TEFLON tubing, and a 1 microliter analyte volume is introduced into the reaction chamber. The analyte can be introduced with a syringe, a syringe pump, a peristaltic pump, or other pump. Alternatively, the analyte can be introduced by wicking with, for example, cellulose fibers, or using a capillary flow. The chamber housing is effectively coupled to an imaging system, particularly a digital imaging system, for obtaining images of response by the responder to the scenario. A detector is positioned to detect response of the responding material to the scenario. The detector is used as in input device for providing data to at least one computer. If only one computer is used, then that computer typically is capable of integrating several functions, including image capture, processing, display, networking, and general input and output. An output device for analyzing and/viewing data, such as acquired images either singularly or in any desired series, is coupled to the computer. A working embodiment utilizes a touch screen for both displaying desired information concerning data acquisition or system operation and for inputting operator commands.

2. Description of Particular Embodiments

A. Chamber Housing and Camera System

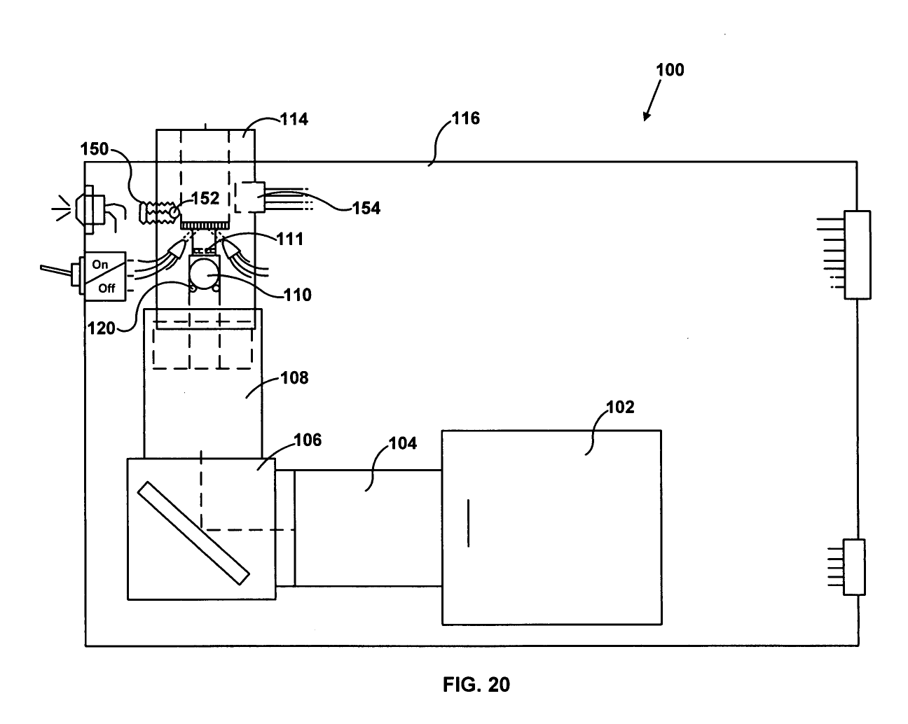

FIG. 20 is a schematic, cross sectional diagram of one embodiment of a cytosensor having a camera unit 100 comprising a camera 102, a first optional extender 104, an optional mirror system 106, an optional second extender 108, lens 110, aperture control 111, and a chamber housing 114. For the illustrated working embodiment of cytosensor 100, camera 102 was a commercially available camera, i.e., a Pulnix camera, model No. TMC-7DSP, which is a single chip camera with DSP color processing. The Pulnix camera can output data in VBS (NTSC or PAL), Y/C or S-Video, and RGB. The camera sensitivity is 2 lux at F1.4. Moreover, the electronic shutter rate can be varied. The images are 768 horizontal pixels by 494 vertical pixels.

Camera system 102 includes several integrated parts, some of which are optionally used. For example, extenders 104 and 108 are used to provide a proper focal length from the lens 110 to camera 102. The illustrated working embodiment 100 used optional, variably sized first extender (e.g., 25+mm in one working embodiment) 104 and optional, variably sized second extender (e.g., 5 mm in one working embodiment) 108. The chamber holder 114 is securely coupled to the extension 108, such as by being threadedly coupled to the extension.

Camera system 100 is housed in housing 116. To conserve space, the illustrated embodiment utilized mirror system 106 to change a linear arrangement of camera components to an angled geometry. Thus, a person of ordinary skill in the art will realize that the mirror system 106 is optional.

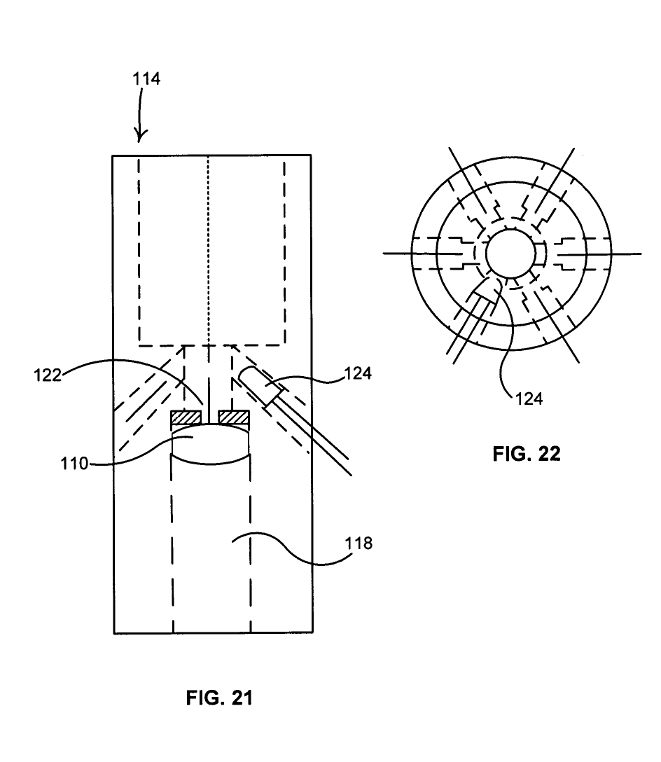

The camera system 100 also includes a light system 114. A number of working embodiments of lighting systems have been made, and the choice of a particular lighting system depends on a variety of factors, including batch versus continuous operation of the cytosensor, lighting from below the living cells or above the living cells, wavelength of the irradiating light used, etc. FIG. 21 is a cross sectional, schematic view of a chamber holder 114 and light system used with one embodiment of a cytosensor. FIG. 22 is a plan view of the chamber holder 114 and lighting system illustrated in FIGS. 20 and 21. Chamber 114 includes a lens-receiving chamber 118 for housing lens 110 therein. As shown in FIG. 20, lens 110 is positioned appropriately using O-ring 120. Lens 110 is positioned adjacent a pin hole 122, which is used to establish a depth-of-field for viewing living cells.

Chamber holder 114 also is machined in the illustrated embodiment to tightly receive plural light sources, such as plural LEDs 124. The illustrated embodiment used 6 LEDs, but other numbers of LEDs could be used as well. Also, the illustrated embodiment typically used an LED system that emits white light, although working embodiments of the cytosensor apparatus also have used variably colored LEDs.

LEDs 124 are positioned below a chamber assembly, which is described in more detail below. Moreover, the illustrated embodiment also illustrates that LEDs 124 are angularly positioned within chamber housing 114 relative to a surface receiving living cells in the chamber. Positioning the LEDs at an angle, approximately a 458 angle in the illustrated embodiment, provides a number of advantages over illuminating the living cells at some substantially zero angle, from either below or above the living cells. The cytosensor includes a light system cable connector for coupling the light system to a control computer.

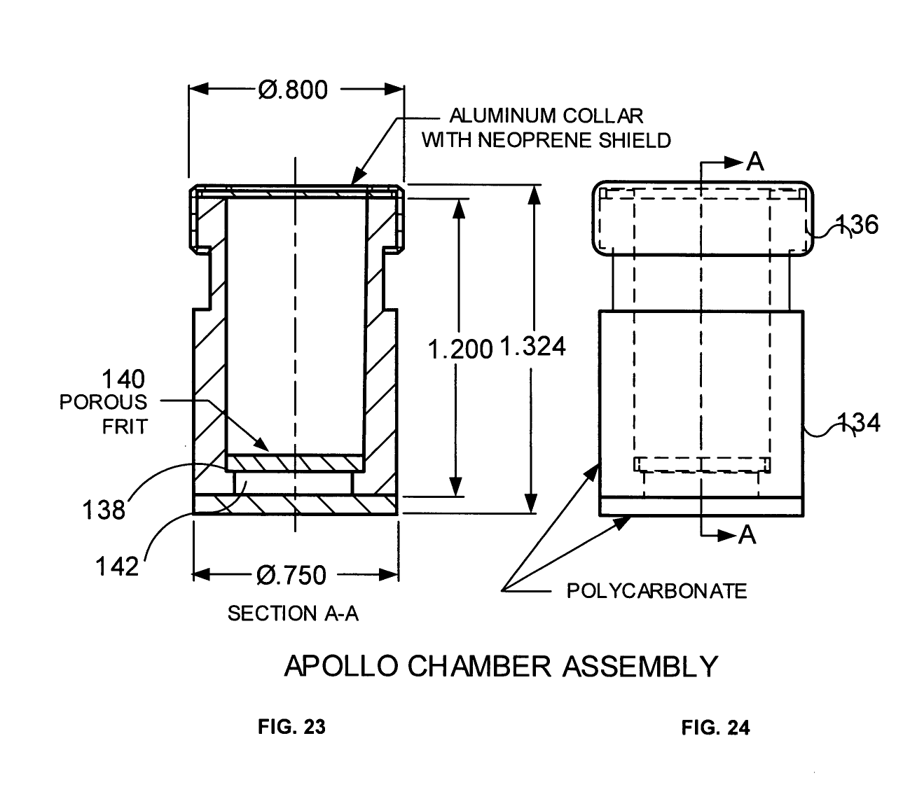

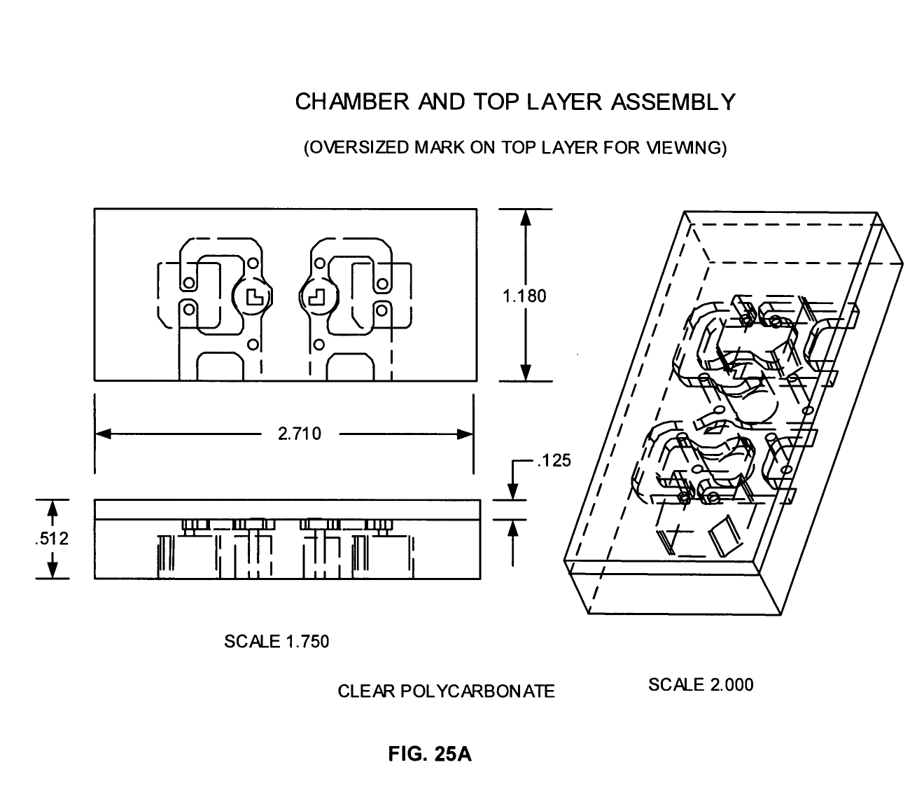

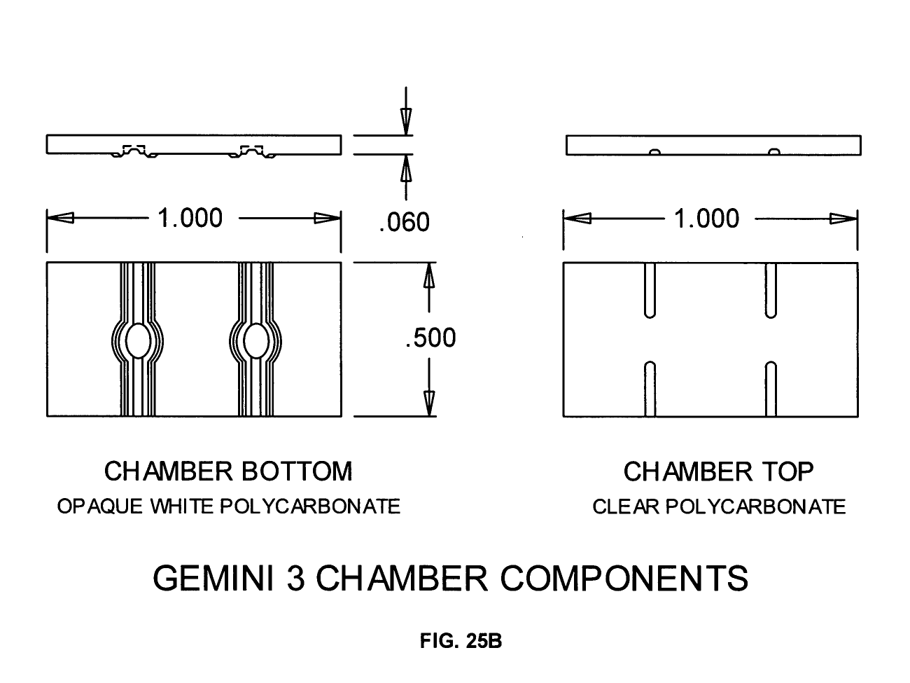

FIG. 23 is a cross sectional view of a chamber 130 used with certain embodiments of the described cytosensor, and FIG. 24 is an exterior assembled view of the chamber 130. Chamber 130 was made from polycarbonate because it can be transparent, and also is easy to machine. Chamber 130 included a transparent bottom wall 132, side wall 134, and top 136. Top 136 comprised an aluminum collar having a neoprene shield in a working embodiment. Bottom wall 132 was secured to side wall 134 by solvent welding. Chamber 130 was machined to include wall ledge 138. A porous frit is positioned on ledge 138, thereby defining a living cell receiving portion 142. Living cells can be placed in cell receiving portion 142, and then chamber 130 inserted into chamber holder 114.

With reference to FIG. 20, it can be seen that chamber holder 114 is designed to receive chamber 130 therein. The illustrated embodiment of chamber holder 114 includes some structure useful for securing the chamber 130 therein, and possibly useful for orienting the chamber 130 correctly within holder 114. For example, the illustrated embodiment includes spring-biased set screw 150. Screw 150 includes a ball end 152 that mates with a ball receiving portion machined into chamber 130. Sensor 154 senses the presence of chamber 130, and thus can initiate operation of a control computer to image changes induced in the living cells by an analyte, to quantify the change resulting from exposure to the analyte, and to analyze such data according to the statistical method described.

A person of ordinary skill in the art will realize that various structures can be useful for making functioning chambers 130. For example, the chamber 130 can be used to receive bulk materials or can be configured as a microfluidic chamber. Furthermore, chamber 130 can be designed for batch operation, i.e., for a one-time use of living cells, or can be configured to provide continuous mixing of the living cells and analytes in the chamber for viewing by the camera.

Chamber 130 includes some structure for introducing an analyte, such as a drug candidate, agent or toxin, to living cells that respond to the analyte. For example, certain embodiments includes a septum, or septum-like device, through which a syringe needle was inserted to flow desired material into the chamber. This process also can be automated, such as by using one or more pumps, e.g., syringe pumps to continuously or intermittently pump desired materials into appropriate material receiving portions of chamber 130.

FIGS. (26A-26B) illustrate one embodiment of a reaction cell. A reaction chamber is defined in a laminated stack of sheets of a transparent, adhesive-backed material such as MELINEX. The reaction chamber is typically rectangular, with representative dimensions of 2 mm by 3 mm by 0.4 mm. The 0.4 mm depth of the chamber is generally selected as an integer multiple of the thickness of the sheets laminated to form the reaction cell. Inlet and outlet apertures of about 0.5 mm diameter are provided for introduction of and extraction of analytes and other reagents.

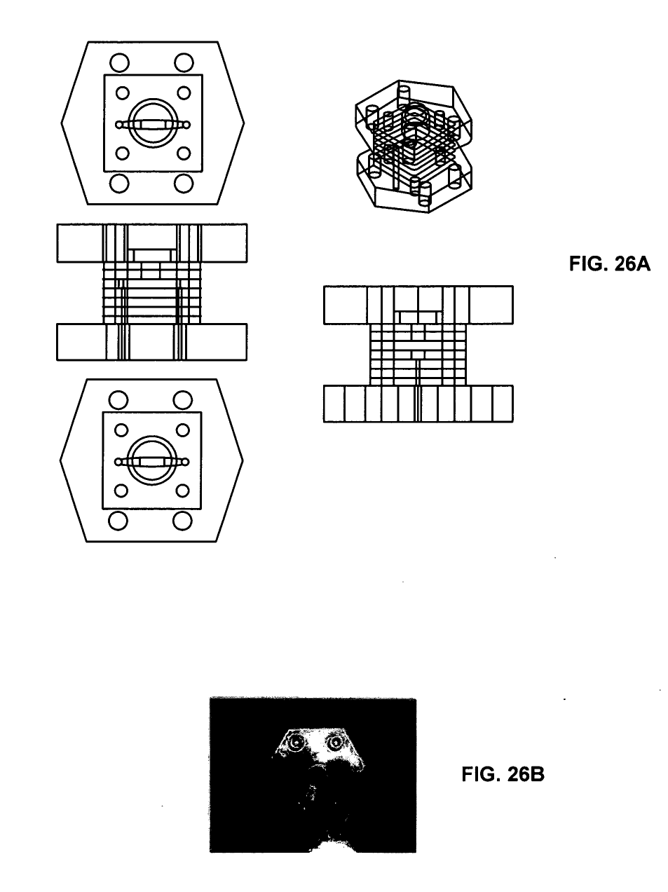

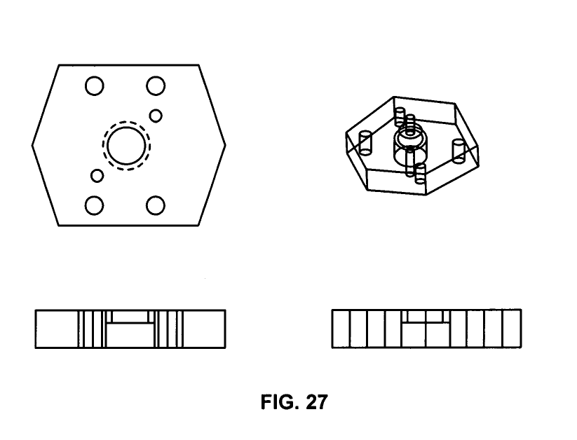

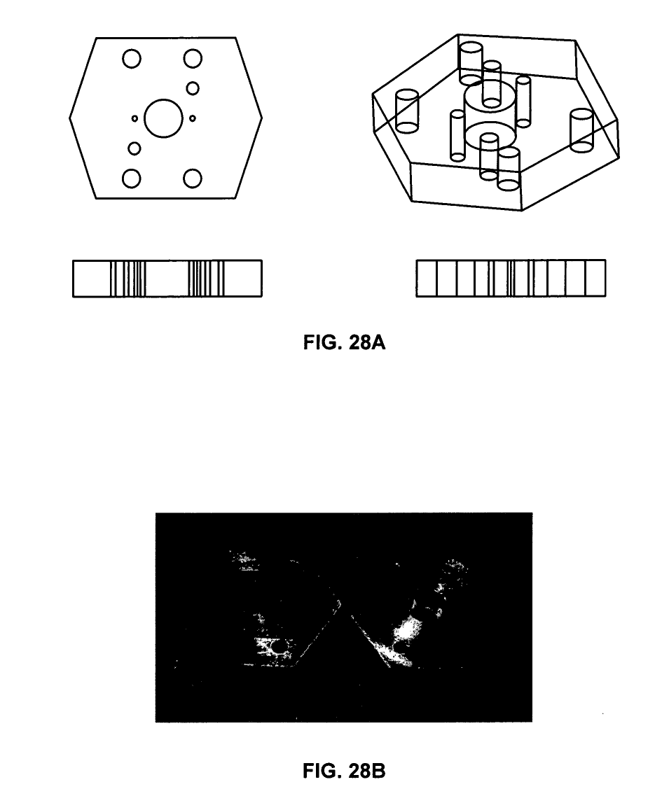

As shown in FIG. 26A, the reaction cell is mounted to a fluid interconnect. The fluid interconnect includes a top plate and a bottom plate having central apertures to permit illumination of the chromatophores. These plates are illustrated in FIGS. 27 and 28A-28B. Mounting holes are provided for attaching the fluid interconnect to the reaction cell, and fluid inlets/outlets are provided as well. Bolts (not shown) are used to connect the top and bottom plates, sandwiching the reaction cell.

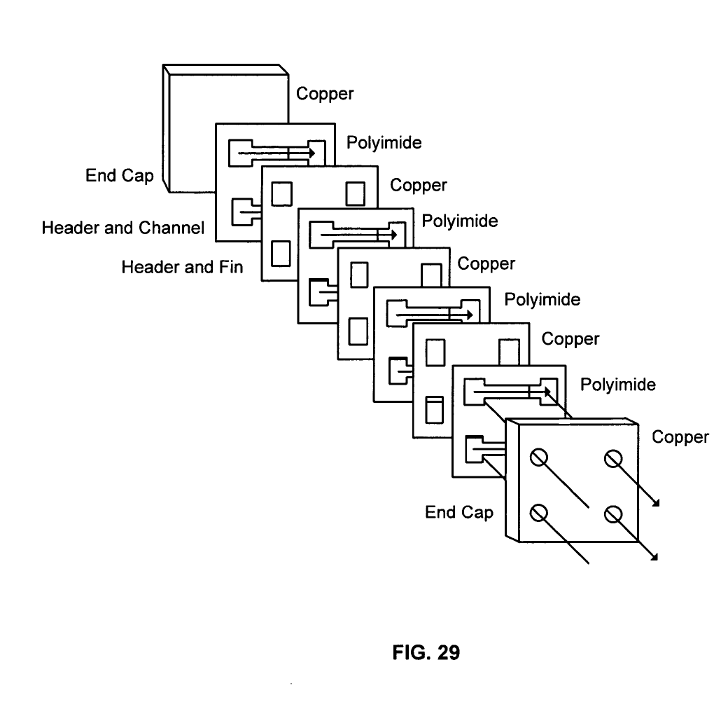

FIG. 29 is a diagram of an alternative reaction cell that defines two reaction chambers. A stack of polyimide layers and copper layers are laminated by heating the stack to that the polyimide layers bond the copper layers. The stack is terminated on a first surface with a first end cap, and on a second surface with a second end cap that includes inlet/output apertures for any necessary analytes or other materials. Reaction cells that include fewer than or more than two reaction chambers can be fabricated similarly.

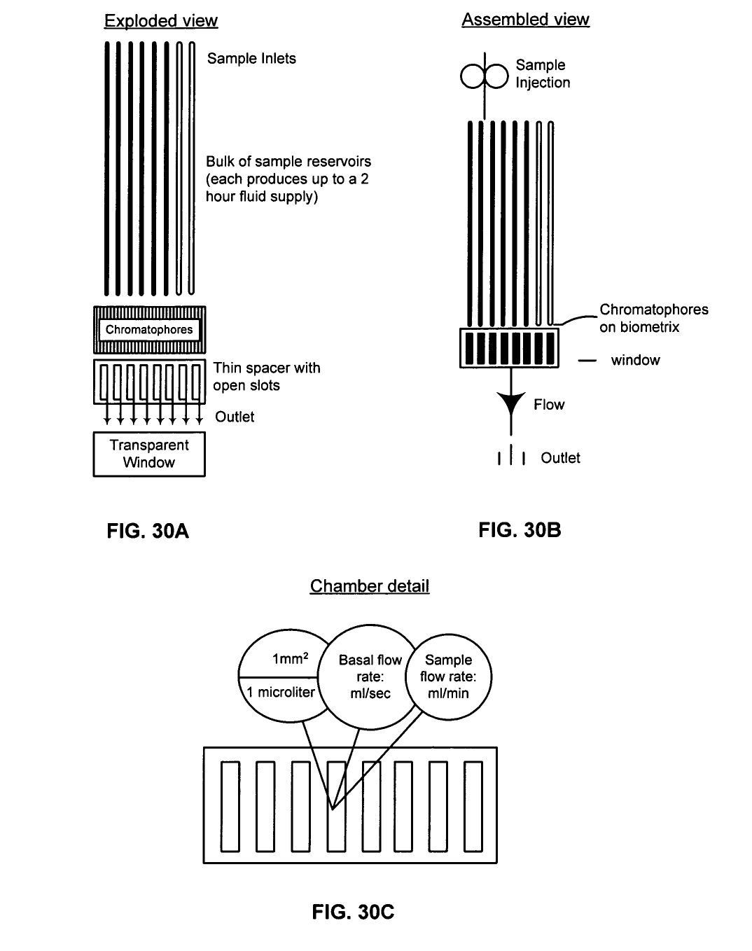

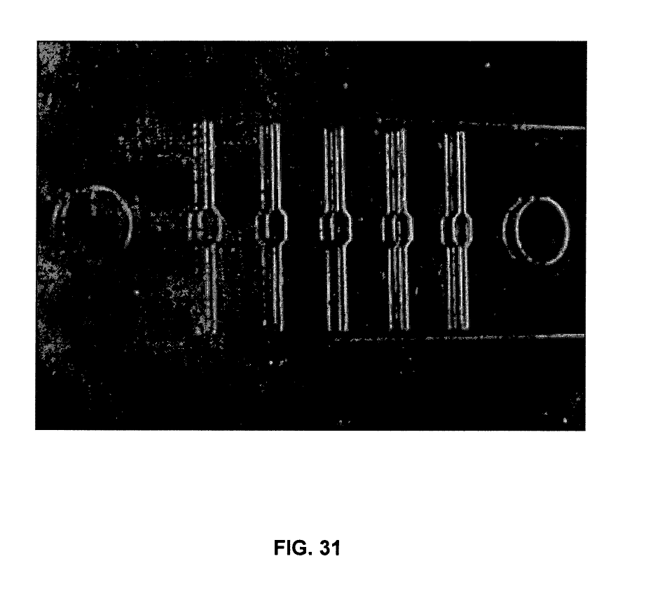

FIGS. 30A-30C are schematic diagrams of a multi-analyte reaction cells that include analyte reservoirs and FIG. 31 is a digital image of a micromold for fabrication of reaction chambers.

B. Control Computer

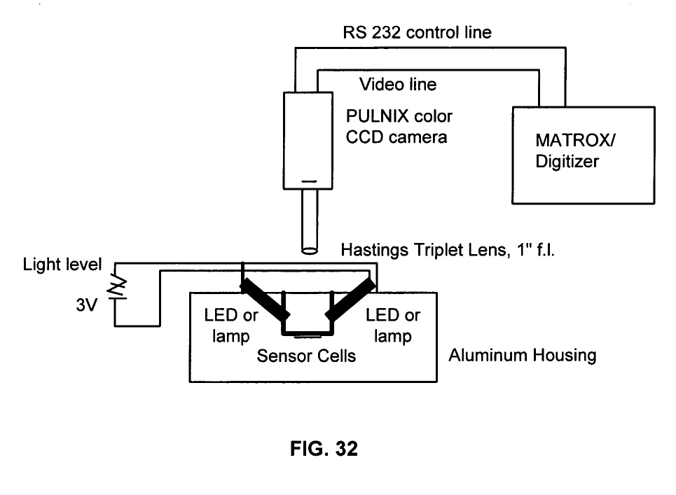

Working embodiments of the cytosensor are coupled to a control computer 200 using a camera cable 202 as illustrated in FIG. 32. Camera cable 202 includes a connector for the image system and a video port connector for coupling to a video port of the control computer 200.

A working embodiment of the apparatus used a commercially available Matrox 4Sight-II system as the control computer 200. It will be realized that other control computers can be used, or multiple computers can be used with the described system. However, the following provides a general discussion of the control computer hardware, which provides integrated video capture, processing and display and software.

Control computer 200 includes a mother board, up to three PC/104-Plus™ boards, a single optional hard drive and/or a flash disk for data storage, or two optional hard drives and a fan. Commercially available versions of control computer 200 can be obtained with either an embedded Intel® Celeron® (128 MB DIMM, 10 GB hard drive) or Pentium®III processor coupled to an Intel® 440BX processor bridge. Current embodiments operate at about 850 MHz. Standard and non-standard analog and digital video acquisition is providing using Camera Link™ and IEEE 1394 IIDC. Input ports include USB ports, RS-2323 and RS-422/RS-485 ports. Control computer 200 includes two serial ports, one of which is configurable for RS-232 pr RS-422/RS-485 operations, as well as a parallel port and two USB ports for optional devices, such as a keyboard and command device, such as a mouse. Such systems run Microsoft® software, including Windows® NT® embedded 4.0, Windows® 2000, Windows® or Windows® XP embedded. The system also includes a Matrox G450 graphics controller to provide graphics control and varied graphic control functions, such as non-destructive graphic overlay on live video and dual head display for a primary analog or digital output along with a TV or a secondary analog output.

C. Touchscreen Control Panel and Display

Data acquired and processed by the system described can be displayed on a touch screen control panel.

One or more light sources such as LEDs, a laser diode or other laser, or lamps are situated to illuminate the chromatophores. In an embodiment, illumination from the light sources is collected with a lens that directs the collected illumination to the chromatophores. Light reflected by or transmitted through the chromatophores is collected with a lens and directed to a imaging system that includes a CCD or other camera. Typically, the chromatophores are imaged onto the CCD. The CCD supplies an electrical image signal (typically an analog NTSC video signal) to a digitizer that produces a digital image of the electrical image signal that is supplied to a computer. Alternatively, an image sensor that provides a digital image directly can be used. The computer receives the digital image and stores the digital image in a tagged image file format (TIFF) or other convenient format for processing. Images are generally obtained at a rate of about 2/sec. The apparatus is configurable for measurement of transmitted or reflected light. Typically, transmitted or reflected light is suitable for melanophores, erythrophores, and xanthophores, but for some chromatophores such as iridophores, reflected light is superior. The apparatus is also configurable for measurements of fluorophores.

Images are typically acquired in a red-green-blue (RGB) color representation in which values (r, g, b) for each of the colors R, G, B are assigned to each pixel. Other representations can be used, such as a hue-saturation-intensity representation, or other representations. After conversion into the selected color representation, the image data is segmented into areas corresponding to one or more predefined colors or color ranges that are based on the selected chromatophores. A color segment is defined as a predetermined number of adjacent pixels having a color within a predetermined range. This procedure is described in detail below.

In a specific example, the image sensor is a 1/2½ interline transfer CCD color camera. Color camera variables include the number of the CCD arrays (e.g.. 3-CCD arrays with RGB filters), CCD formats (typically ⅓″, 1/2½nd 1″), and resolution (e.g., the number of horizontal and vertical pixels N, M, respectively). For example, a CCD camera can have N=768 columns and M=494 rows of pixels, or 379,392 total pixels. and the CCD cell size is 8.4 μm (H)×9.8 μm(V). The camera produces an analog RGB signal that is digitized using a multi-channel frame grabber having sampling rates up to 30 MHz and a data transfer rate of 130 MB/second. A digital color camera can also be used. Such a camera typically has a variable speed shutter (1/60 sec. to 1/10,000 sec.) and can be operated both synchronously and asynchronously.

D. Apparatus Assembly

The touch screen includes three cables, a first monitor cable for touch screen video output that is coupled to the control computer using a video output port, to a serial port for touch screen command, and via a power cable to a power source. The imaging system is coupled to the control computer by connecting a camera cable into the video outlet on the imaging device, and to the control computer to a video input port. The camera shutter speed initially is set to zero. The operating software is then loaded, such as by a network connection.

The camera and imaging system are then assembled. The imaging device, such as a digital camera, is coupled to a camera fixture. Components are then coupled to the camera, including an extender, lens, aperture control, and small extender. The light system is then coupled to the last extender, and the detector inserted into the light system. The detector is oriented such that a scenario input system, optionally the septum and syringe described above for a working embodiment, is oriented for best practical use. The detected is then covered with a chamber housing. The assembled device is now ready to receive indicator materials, such as chromatophores, and scenarios.

E. Apparatus Software

After connecting the hardware, launch Cytosoft software, which performs the following functions. First, the light control driver is installed, followed by establishing initial parameters.

A program (Cytograb) is then used grab a sequence of images and save them onto the hard drive in a directory automatically created by Cytograb. After completing a system operation, the program automatically transfers the files to a server, if the transfer services are setup.

The program is started, and at first, their four buttons are presented, New, Light Switch, Options and Exit.

New:

Select New to run a new experiment. After clicking on this button, the program will go to the operation screen where there are 3 buttons; pause, stop, and stop and delete.

Pause—to pause the experiment. After clicking this button, it will change to resume automatically and vice versa.

Stop—to stop recording the current experiment.

Stop and delete—to stop recording the current experiment as well as delete the corresponding files and directory on the hard drive.

Light Switch:

Manual switch to turn the light on and off.

Options:

After clicking on the options button, a new window will pop up with the following options:

Camera type—Color RGB/Black and White.

Auto light switch—check this box to have the light turn on during image captures.

Sampling interval—the period of time between captures in milliseconds.

Max. number of images—how many pictures will be taken (this also corresponds to how long the experiment will run).

Max. time—length of the experiment in minutes (this corresponds to how many pictures will be taken).

Exit:

After pressing the Exit button, the program will ask the question “Do you want to SHUTDOWN the system completely?” and give 3 options to choose from:

Yes—The program will shutdown the Matrox system completely.

No—The program will only close Cytograb, not the system.

Cancel—The program will return to the initial Cytograb screen.

A Cytosoft program allows the user to grab, playback and analyze the images during the experiment. The images will be saved to or loaded from the same directories as with Cytograb. The program loads a new window onto the screen with 4 sections: Control, Information, Data Display, and Sidebar.

Control Section:

The Control Section lets the user setup and control the experiment. When the program starts, there are 6 buttons, New, Open, Light Switch, Replay, Setup and Exit.

New:

Select new to run a new experiment. After clicking on this button, the program will go to another control section where there will be 2 buttons: pause and stop.
Pause—to pause the experiment. After clicking this button, it will change to resume automatically and vice versa.
Stop—to stop the experiment; after clicking stop, check the “Delete files” box if you also want to delete the files from the hard drive.
   Open:

Click Open to playback previously recorded experiments. Select the directory that corresponds to the desired experiment.

Replay:

To start replaying the movies after setting up the directory

Light Switch:

Manual switch to turn the light on and off.

Setup:

After clicking on the setup button, a new window will pop up with the following options

Camera type—Color RGB/Black and White.

Auto Light Switch

To turn on light on automatically only during image captures (=blinking light).

Sampling Interval

The period of time between captures in milliseconds.

Max. Number of Images

How many pictures will be taken (this also corresponds to how long the experiment will run).

Max. Time

Length of the experiment in minutes (this corresponds to how many pictures will be taken)

Exit:

After pressing the Exit button, the program will ask the question “Do you want to SHUTDOWN the system completely?” and give 3 options to choose from:
Yes—The program will shutdown the Matrox system completely.
No—The program will only close Cytosoft, but not the system.
Cancel—The program will return to the first page of Cytosoft.
   Information Section:

This section displays information regarding the current actual playback or acquisition. It shows the acquired or loaded (for playback) frame, the actual processed one, remaining frame, the corresponding time elapsed, the duration, the remaining time and date of record.

Data Display Section:

This window displays various data during the experiment, each corresponding to a tab in the sidebar section (next).

Sidebar Section:

Using sidebar tabs, there are 6 ways to display data: Live video, First grab, Current grab, Inspector (all or single quadrants) and Results,:

Live Video:

To display the live video from the camera connected to the Matrox system.

First Grab:

To display the first grabbed image of the experiment (both during acquisition and playback).

Current Grab:

To display the current grabbed image. In the acquisition mode, it is the last image acquired by the camera. In playback mode, it is the last loaded image from the hard drive.

Inspector (or Statistics):

To display “on-the-fly” graphs of the area features and model for each quadrant of processed images. The user can choose between 2 views:

All quadrants—displays all the quadrants for the selected data (area feature or model)

Single quadrant—displays only one large quadrant at a time (the user can use Up, Down, Right and Left to move from one quadrant to another)

Results (or Detector):

To quantify the probability of agent concentration, based on the models computed in each quadrant. These results are updated only at the end of the current experiment and will show the name of the agent with the probability value. A Match level bar at the bottom shows the current level of probability. The color corresponds to the level of probability and the number shows the exact value of the probability (it will be displayed if and only if it is more than 10%).

F. Operation

After the Cytosoft software is loaded, begin data capture as follows. First, initiate Setup to configure setup. The lighting system of the cytosensor is then actuated by clicking Auto light switch. A new experiment can then be initiated. For use with syringes, the syringe plunger is removed (e.g., 1 ml barrel, 25 gauge needle) and the needle inserted, just through the septum. A second syringe (containing up to 0.5 ml of sample) is inserted all the way through septum, next to but not immediately adjacent to first syringe. Mixing can be achieved raising the plunger all the way up then injecting quickly, thereby mixing the agent with media.

### VI. Experimental Approaches for Knowledge Base Generation

1. Reference Node

The application of primary elicitors defines reference nodes, which in turn defines the functional module or knowledge base structure for a particular application. A network node is defined as a unique interaction between cellular signal transduction network elements. An important checkpoint or network node is a unique interaction that can be perturbed through the addition of effectors. A key concept is that of a reference node. A reference node is an important checkpoint that when acted upon by an effector provides a reference or fixed point in mechanism space with certain useful properties for assigning mechanistic interpretations to the action of biologically active agents. The selection of a reference node is strictly functional and dependent both on the experimental determined properties of a given important checkpoint and on the ability of the reference node to simplify problem solutions. A reference node may or may not map to master regulatory elements in cellular or other biological networks. Careful selection of reference nodes might result in a significant increase in efficiency with respect to utilization of material or computational resources or might aid in the resolution of analytical problems such as singularities in the solution matrix.

Reference nodes become the principal building blocks formulating elicitor panels that effectively monitoring the information flows through cellular signal transduction pathways. Primary elicitors acting at primary nodes are used to define an N-dimensional reference plane in mechanism space that uniquely identifies a given simplex scenario.

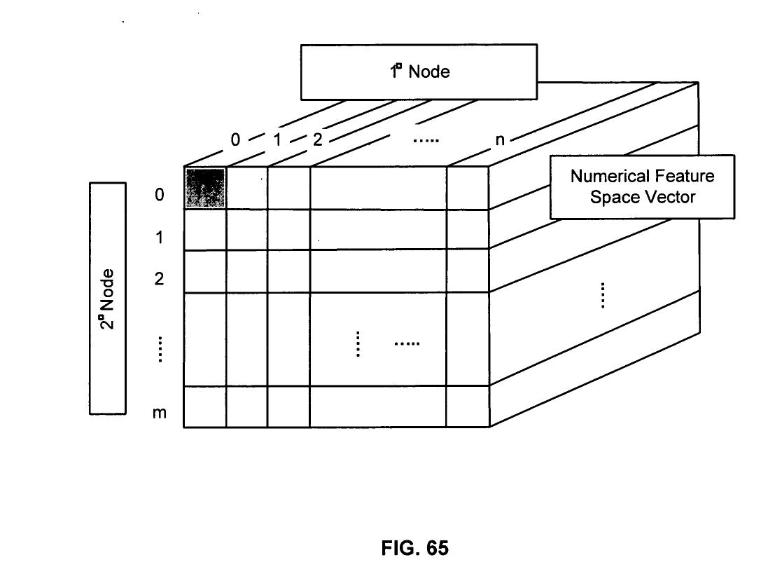

FIG. 65 shows mechanism space segmented with simplex scenarios defined through 1-dimensional reference planes or a single reference point. Secondary elicitors acting at secondary nodes further decompose mechanism space and along with mechanistic models supply the fine detail necessary for accurate mechanistic assignment to unknown agents. Effectors are categorized as direct or non-direct depending on whether or not they elicit a measurable cell response. Primary effectors might be direct or indirect whereas secondary effectors will generally be direct.

Proof-of concept for the mechanism space segmentation approach comprises exposing chromatophores to high concentrations of forskolin, to establish a reference point.

2. Experimental Demonstration of the Reference Node Concept

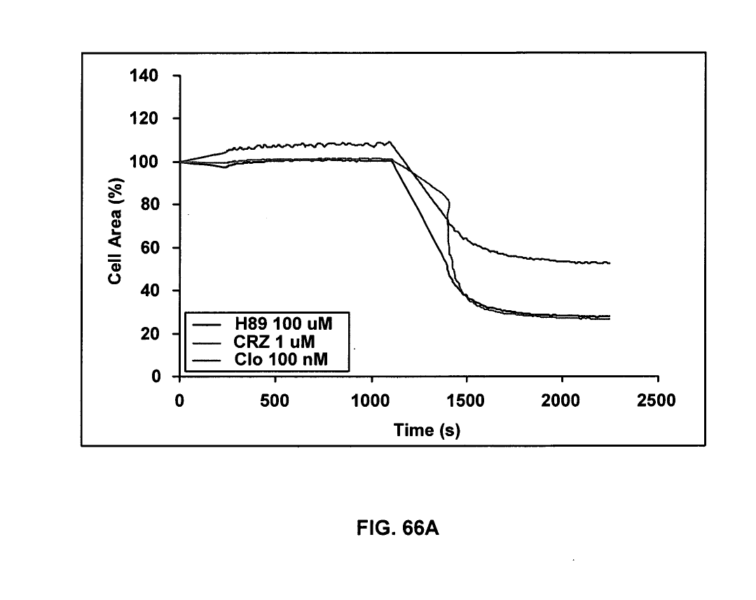

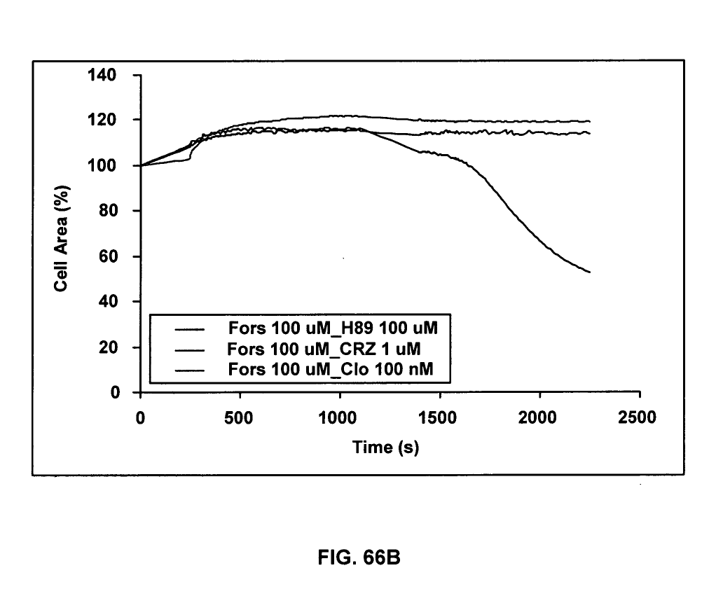

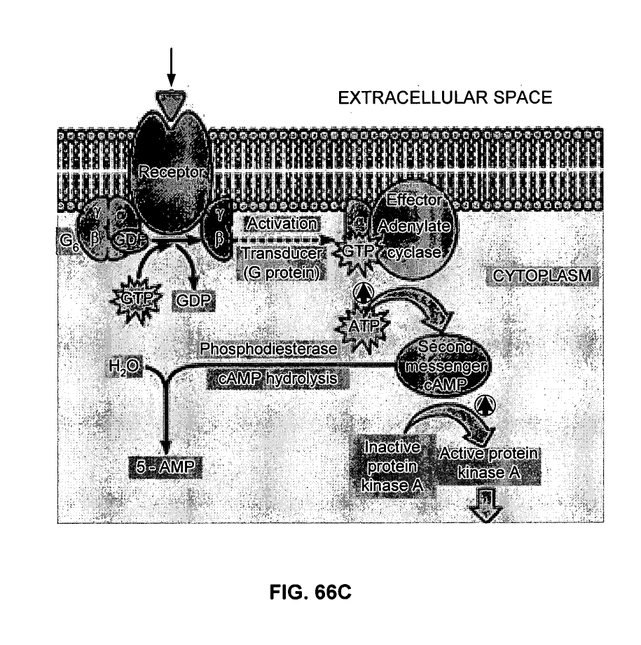

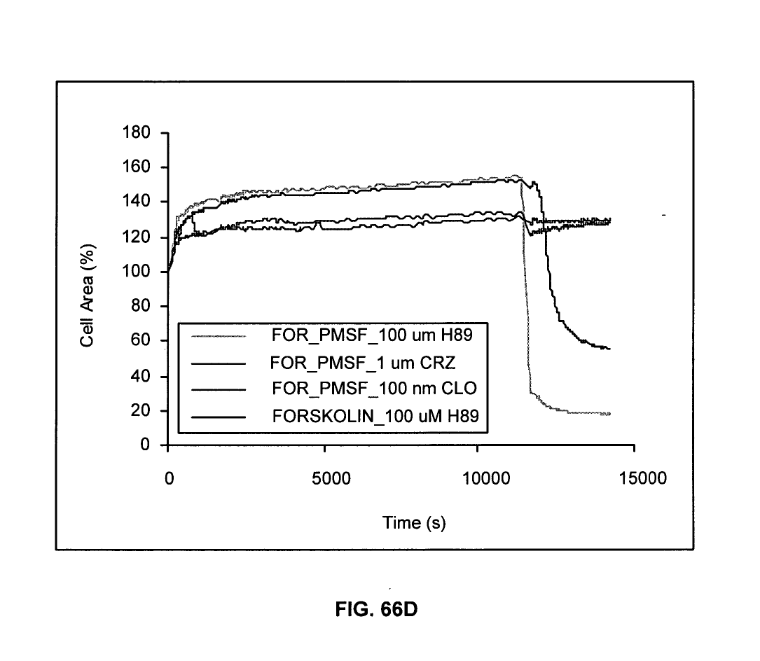

The usefulness of the reference node concept is demonstrated in the following example where forskolin was applied to fish chromatophores. FIG. 66 shows that exposure to forskolin establishes a reference point at adenylate cyclase, which decouples the upper segment of the α₁-adenergic and α₂-adenergic receptor-linked GPCR pathways. Secondary elicitors H89 (protein kinase A inhibitor), clonidine (α₁-adenergic agonist) cirrazoline (α₂-adenergic receptors agonist) were then utilized to provide additional information about the interaction of phenylmethlysulfonyl fluorise (PMSF) with the chromatophores. FIG. 66A shows that chromatophores aggregate in response to clonidine, cirazoline and H89 in the absence of forskolin. FIG. 66B shows that the system is unresponsive to clonidine and cirazoline in the presence of forskolin and continues to be responsive to H89, which the schematic in FIG. 66C shows is the next regulatory checkpoint following adenylate cyclase in the GPCR pathway.

Selection of forskolin as a primary elicitor simplifies the determination of the mechanism of action of PMSF, a serine protease inhibitor and nerve agent analog. FIG. 66C shows that exposure to PMSF, a serine protease inhibitor, heightens the sensitivity of cells to H89. Application of PMSF in conjunction with forskolin therefore isolates the measurable impact of PMSF to other regions of the signal transduction network. Stimulus of the system by secondary elicitors provides additional information regarding the mechanism-of-action of PMSF. This is evident in the heightened sensitivity of PKA to H89 in the presence of PMSF while the upper segment of the pathway is decoupled by application of forskolin as shown in FIG. 66D.

3. Elicitor Set Experiments

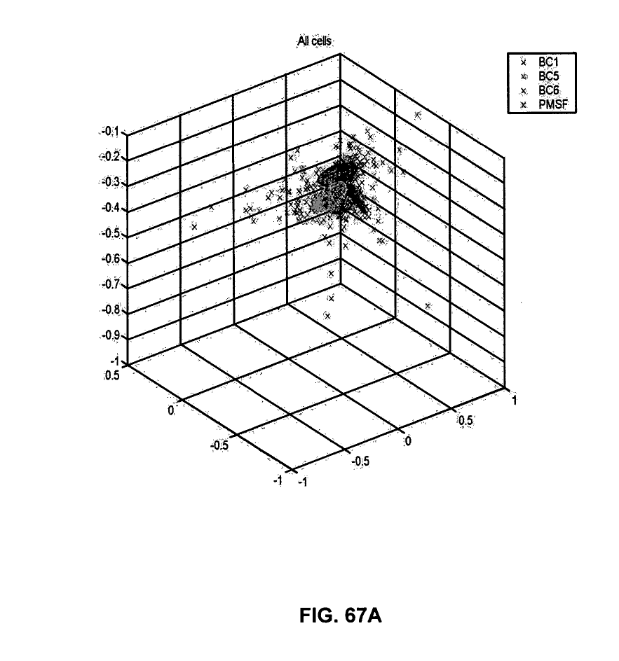

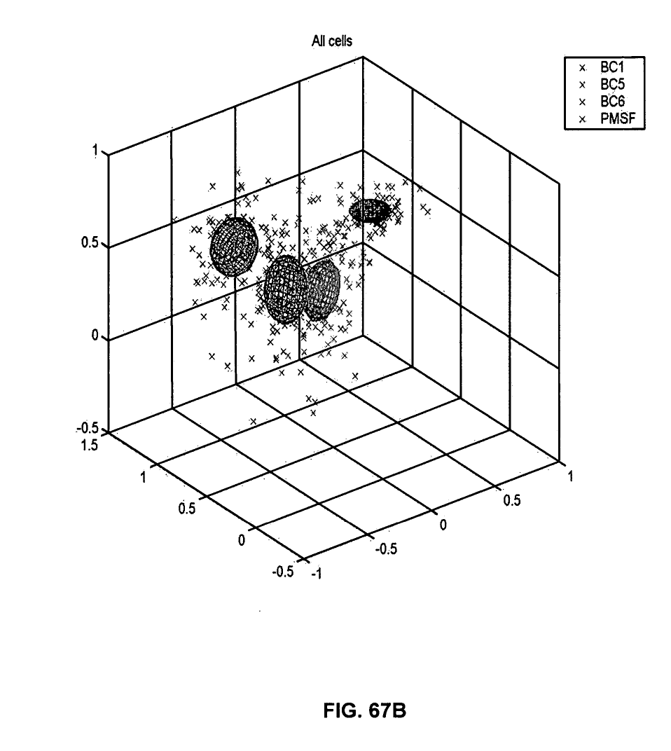

Elicitor set experiments are performed through the consecutive exposure of cells to unknown agents and known effectors of signal transduction pathways. The group of experiments defined by an elicitor set is considered a single “matrix” of experiments. Matrix 2 demonstrated the general elicitor set concept utilizing three strains of Bacillus cereus (BC1-49064, BC5-10987 and BC6-14579) each expressing different complements of bacterial toxins as detailed in Table 1. Matrix 3 added the chemical agent phenylmethylsulfonyl fluoride (PMSF), a serine protease inhibitor and nerve agent analog. Both matrices utilized chromatophores isolated from Betta splendens. Effectors for these experiments included forskolin, melanin stimulating hormone (MSH), clonidine and L-15 media. The “unknown” agents were tested against the elicitor panel formulated as detailed in Table 2. These experiments demonstrate that incorporation of elicitors into experimental methods results in a non-linear rescaling of feature space. The result is better cluster separation and improved classification based on cell response. FIG. 67 shows cluster separation for the three strains and PMSF in the presence of the full elicitor panel as compared to the separation that occurs in the absence of elicitors. Separation of clusters for BC 5 and BC6 is much improved with application of the elicitor panel and is discussed in detail herein.

TABLE 1
Complements of toxins expressed by each B. cereus strain.

| | | | | New heat | |
|---|---|---|---|---|---|
| Strain | Hbl Toxin | Nhe Toxin | bceT Toxin | + | Others |
| BC1-49064 | + | + | − | + | + |
| BC5-10987 | − | + | − | − | + |
| BC6-14579 | + | + | + | − | + |

TABLE 2
Elicitor panel composition. Elicitors are defined
as effector and application method.

| | | |
|---|---|---|
| Effector | Concentration | Rxn Time¹ (min) |

| | | | |
|---|---|---|---|
| Forskolin | 100 | μM | 15 |
| MSH | 10 | nM | 20 |
| Clonidine | 100 | nM, | 10 |
| | 50 | nM² | |

| | | |
|---|---|---|
| L-15 Media | Standard Composition | 10 |

¹Reaction time following exposure to test agent for 5 minutes
²50 nM clonidine was not utilized as part of Matrix 2.

4. Improvement in Signature Classification with Elicitors

Classification of BC5 relative to BC6 represented the more difficult classification problem within the set of “unknown” agents selected for Matrix 2 and 3 and are the focus of this discussion. The complement of toxins expressed by these two strains is shown in Table 1. Prior to the development of the elicitor set concept, the feature vector whose components consist of observable non-numerical features or labels, such as cell type (erythrophore, melanophore), and cell initial state (dispersed/aggregated, dendricity), was defined. Table 3 summarizes the classification efficiencies possible for various combinations of observable features in the absence of elicitors. The overall classification efficiency for both Matrix experiments (60%, 56%) is only slightly better than for a random guess. Classification efficiencies for different sub-sets of the feature vector were as high as 86%, indicating that the proper selection of physically observable features may significantly improve classification efficiencies. However, the consistency of classification for each feature set was highly variable between Matrix experiments. For example, selection of well-dispersed, high dendricity melanophores resulted in the highest classification efficiency for Matrix 2 (86%), whereas well dispersed, high dendricity erythrophores had the best classification efficiency for Matrix 3 (73%). Another example is the relative performance of melanophores and erythrophores, which changed between Matrix experiments. The reasons for the variability are unclear at this time, although the effects of genetic variation and unavoidable inconsistencies in experimental methods due to the human factor on experimental consistency are well-known. Much work is necessary in order to identify the optimal selection of labels for classifying different chemical and biological agents.

TABLE 3
Global Classification Accuracy for Bc5 and Bc6 in the absence of Elicitors

| | Matrix 2 | | Matrix 3 | |
|---|---|---|---|---|
| | All cells All initial cell states | | All cells All initial cell states | |
| | 60% | | 56% | |
| Non-Numerical Features | Erythrophore | Melanophore | Erythrophore | Melanophore |
| Well-dispersed - High | 64% | 86% | 73% | 60% |
| dendricity | | | | |
| Well-dispersed - Low | 64% | 70% | 60% | 65% |
| dendricity | | | | |
| Partially aggregated - High | 60% | 82% | 67% | 60% |
| dendricity | | | | |
| Partially aggregated Low | 60% | 71% | 60% | 51% |
| dendricity | | | | |
| All | 53% | 66% | 62% | 59% |

Elicitors are considered additional features in feature space for the purposes of general agent classification. The addition of elicitors to experimental methodologies significantly improves classification efficiencies. Table 4 shows classification efficiencies in the presence of the full elicitor panels for Matrices 2 and 3. The overall classification efficiency for both experiments has now improved to approximately 90%. Classifying cells in terms of physically observable cell states further enhanced the classification efficiency in a number of instances relative to absence of elicitors for the same cell class, but also significantly decreased classification efficiencies in others. In addition, the range of classification efficiencies (24%-100%) was far higher than in the absence of elicitors (51-86%). The reason for this observation is unclear at this time. However, the reaction of certain cell types to different effectors is greatly affected by genetic variation in chromatophores isolated from B. splendens and this may be a factor. Despite the variation observed with individual cell classes both intra- and inter-Matrix experiments, the overall classification efficiency for both the non-elicitor and elicitor results is highly consistent from Matrix to Matrix

TABLE 4
Global Classification Accuracy of BC5 and BC6 in the Ppresence of Elicitors

| | Matrix 2 | | Matrix 3 | |
|---|---|---|---|---|
| | All cells All initial cell states | | All cells All initial cell states | |
| | 89% | | 91% | |
| Initial Cell State | Erythrophore | Melanophore | Erythrophore | Melanophore |
| Well-dispersed High dendricity | 47% | 50% | 54% | 60% |
| Well-dispersed Low dendricity | 100% | 98% | 71% | 59% |
| Partially aggregated High | 24% | 67% | 46% | 95% |
| dendricity | | | | |
| Partially aggregated Low | 100% | 100% | 62% | 66% |
| dendricity | | | | |
| All | 95% | 92% | 97% | 94% |

### VIII. Data Analysis

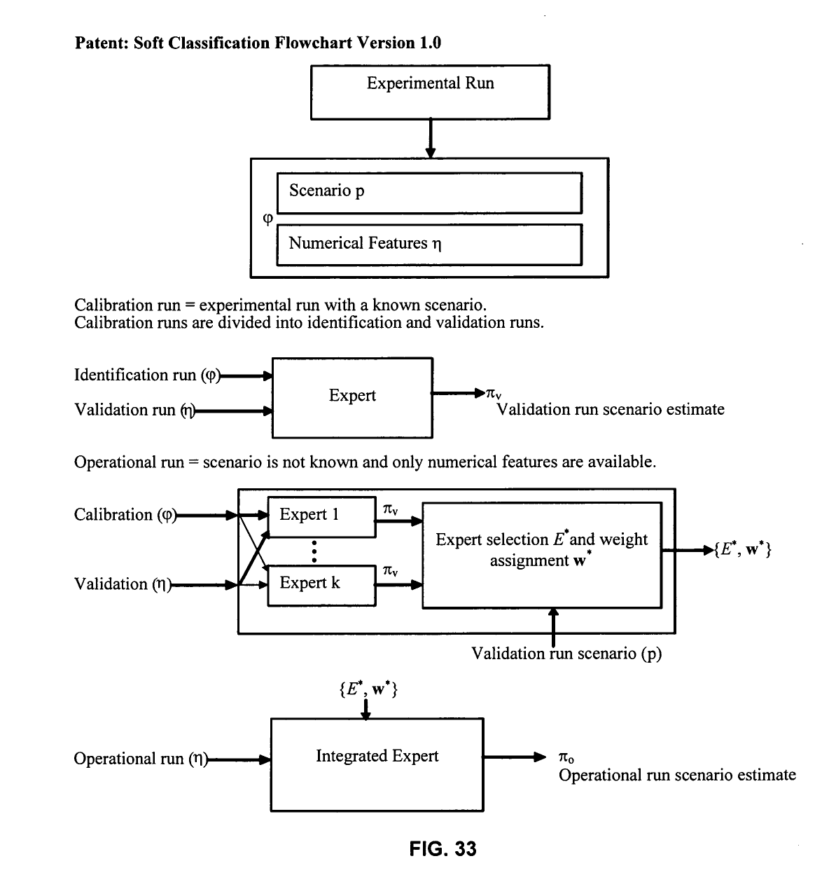

This embodiment of the present method is useful for classifying and analyzing data where large data sets are obtained. A flowchart highlighting steps used to practice the method and optionally to be implemented by software is provided as FIG. 33. Because of the high dimensionality of the data, multidimensional entity features are used, which are useful for representing both numerical and non-numerical features, such as ordinal or nominal information, and perhaps more abstract components such as distributions or functions. For known classification methods, each feature is represented only numerically, but in the presence of non-numerical features, and standard statistical approaches fail.

The present embodiment of the disclosed method can analyze data that includes both numerical and non-numerical features. This embodiment involves using a few basic components, including a feature space, integrated expert, adaptive expert calibration and soft classification.

Each experimental run is represented by a multidimensional entity of features, part or all of which is observable and known to an operator. Two basic types of runs are used, calibration runs, where the entire feature entity is observed, and operation runs, where only part of the feature entity is observed.

A single run of an apparatus described herein results in a time sequence of images or frames, and a sequence of frames is referred to as a movie. A movie provides a visual record of a response system, such as living cells, or plural different types of cells, behaviour, to exposure to an agent. Different magnifications can be used. For example, at low magnification, each movie frame may be divided into nₛ sub-images. For high magnification, individual cells may be isolated where the cell count is nₛ. Within each sub-image, a vector of numerical quantities is calculated, and may include, without limitation, cell area (average), color, cell morphology, size of outgrowths, etc. Using the frame acquisition time stamp these numerical vectors are indexed to form a multi-dimensional time series. Each expert e, e=1, . . . N_{E}, selects a different subset of this multi-dimensional time series components. The selected subset is not modeled by a discrete-time nonlinear dynamic system. The underlying model parameters form a vector \hat{ }ₑ. Each sub-image produces a separate model thus resulting in \hat{ }ₑ^{k}, k=1, . . . , nₛ numerical vectors. The types of agents or ordinal features, which takes values related to the agent strength (impact on living organism). For instance, a chemical agent, such as clonidine, could have various concentrations, indicated as low, medium or high. Likewise, a bacterial pathogen, such as Bacillus cereus, could have variable toxicity, indicated as being weak or strong.

1. Feature Space

The values of ordinal and nominal features are referred to as labels. With n_{f} ordinal and nominal features, the total number of labels is equal to nₗ=Σ^{nf}_{f=1}m_{f} where m_{f} is the number of values of f-th features. In the simplest situation, a single label is assigned to each non-numerical features. In a general case, multiple labels may be assigned to a single non-numerical feature, with weights that represent confidence in this assignment. Thus, the feature value is represented by a distribution. Due to distribution additivity, the sum over the weights of any single feature can be interpreted as a confidence in the entire single feature assignment.

All the labels can be grouped in a label vector, which may serve as a single aggregated ordinal/nominal features. The weights assigned to all n_{f} labels constitute a distribution of this vector features, called the scenario p. In particular, the value of the scenario p for a particular label l is denoted by pₗ. A single experimental run is represented by the scenario and a numerical feature vector. This numerical vector may vary in the repeated runs having the same scenario. The feature space is defined here as a collection of such representations resulting from all experimental runs. Each scenario induce a labeling in the numerical part of the feature space. A special but important case of scenario for which the distribution is concentrated on a single label will be called the simplex scenario. Labeling induced by simplex scenarios divide the numerical features space into subsets. A complex scenario can be viewed as a combination of simplex scenarios. Soft classification is understood as a creation of a scenario.

For example, if there are a number of non-numerical features nₗ=2, and with m₁=3, m₂=2, the number of labels is nₗ=5. The scenario is given by a vector
p=p_{low clonidine concentration}
p_{medium clonidine concentration}
p_{high clonidine concentration}
p_{weak Bacillius cereus concentration}
p_{strong Bacillius cereus concentration}
   of nₗ=5, coefficients. For every simplex scenario all coefficients except one are equal to zero, i.e., p=0, 0, 1.3, 0, 0
   corresponds to the value of 1.3 assigned to high level of clonidine concentration.
   2. Integrated Expert and Adaptive Calibration

An expert is defined as a mapping from the numerical feature space to the set of scenarios. This mapping is realized through assigning to a numerical vector a set of mixing (weighting) coefficients corresponding to all the labels. The above numerical vectors is in general only a portion of the feature numerical component. Here an arbitrary number of nₑ experts working in parallel create an integrated expert. A process of adaptive calibration is needed to properly fuse the individual expert outputs.

For example, if nᵢ apparatus calibration experimental runs with a given scenarios {p(i), I=1, . . . , nᵢ}. For each expert e, eε{1, . . . , nₑ}, a single run with scenario p(i), produces nₛ model parameter vectors Λ^{ik}ₑ, k=1, . . . , nₛ corresponding to the respective sub-images. Let Cⁱₑ denote the set of Λ^{ik}ₑ, k=1, . . . , nₛ representing the calibration run with the scenario p(i). Cⁱₑ is called a calibration cluster. Similarly each operation run with a scenario p(0) produces cluster C⁰ₑ. The expert e calculates a probabilistic distance between C^{k}ₑ and C^{j}ₑj, kε{0, 1, . . . , nᵢ}:
d^{jk}ₑ=P(//C^{k}ₑ−C^{j}ₑ//)
Where P( ) is a non-increasing, non-negative valued function and //C^{k}ₑ−C^{j}ₑ// denotes a standard distance between the clusters C^{k}ₑ and C^{j}ₑ. In the expert e “opinion” the scenario of urn p(k) is given by the following estimate
pₑ(k)=M({p(I), d^{ik}ₑ}, iε{1, . . . nᵢ})
where combined weighted scenarios.

To illustrate the above a special case is considered, where the probabilistic distance between two clusters is calculated using the Mahalanobis metric. Let the vectors Λⁿ, nε{1, . . . , nₛ}, form a cluster C. Define the cluster “mean” Λ and cluster covariance C as:
Λ=1/nₛΣ^{ns}ₙ₌₁Λⁿ
and
C=1/nₛΣ^{ns}ₙ₌₁Λⁿ(Λⁿ)′−ΛΛ′
Then write C(Λ, C). The modified Mahalanobis metric δ=//C(Λ¹, C¹)−C(Λ2, C²)//, where dimΛ¹=dimΛ²=m can e defined in many ways, for example:
δ=/¹₀(Λ¹−Λ²)′(pC¹+(1−p)C²(Λ¹−Λ²)dp
or
δ=max_{0≦p≦1}(Λ¹−Λ²)′(pC¹+(1−p)C²(Λ¹−Λ²)

Modeling Λⁿ as the realizations of Gaussian random variable we have δ=Chi-Square distribution with m degrees of freedom. In this case a reasonable probabilistic distance can be calculated as d=P(δ)=1−X²ₘ(δ).

This process begins with collecting the calibrations runs and dividing the resulting set of features into identification and validation subsets I and V of nᵢ and nᵥ elements respectively. Note that I and V do not have to be mutually exclusive. Denote by p\hat{ }ₑ(i) the scenario, and by p\hat{ }_{e,l}(i) the scenario value for l-th label, as estimated by e-th expert for the i-th identification run, where e=1, . . . , nₑ, l=1, . . . , nₗ, I=1, . . . , nᵢ. For the i-th run, the actual scenario and its value for l-th label are denoted by p(i) and pₗ(i), respectively. The experts' estimates of the i-th identification run scenario are merged together by a function π_{E;w} to form an integrated expert, namely
p\hat{ }_{E,w}(i)=π_{E;w}(p\hat{ }ₑ(i), e εE)
Where E⊂{1, . . . , nₑ} is a selection of experts and function π_{E;w} is assumed to be known up to the parameter vector w. Both E and w are found by an adaptive calibration process.

The adaptive calibration process searches for a best selection of experts E* and the optimal value of parameters w*. The search begins with an initial selection of experts E. Match function Q_{I} is defined as
Q_{I}(E;w)=Σ_{iεI}cᵢq(pE;w(i),p(i))
Where the weights cᵢ describe a relative importance of i-th experiment, and the function q measures a disparity between two scenarios. The problem of adaptive calibration, for a given selection of experts E, is to minimize Q_{I} as a function of w. Denote w*(E) the optimal parameter value for a given E. In the next step the expert's selection E is evaluated according to the following match criteria
Q(E;w)=Σ_{iεV∪I}cᵢq(pE;w*(E)(i),p(i))
Any discrete minimization method can be used to improve the expert selection, and iterate with the identification step. This process ends with E*=arg min E⊂{1, . . . , n_{E}} and w*(E*)=arg inf_{w}Q_{I}(E*;w). In particular, a complete search can be applied efficiently for small values of ne. A complete search can be avoided by first eliminating the least performing experts and minimizing Q(E) only over the remaining set. The above steps yield the best integrated expert π*=pE*,w*(E*).

For example, assume that there are n calibration runs with the corresponding scenarios {p(1) . . . , p(nᵢ))}. Let d^{jk}ₑ denote a probabilistic distance between the runs with the scenarios p(k) and p(j) as calculated by the expert e. Thus, for each fixed k the expert e gives a distribution D^{k}ₑ of probabilistic distances
D^{k}_{e={. . . ,d}^{n,k}ₑ}), k=1. . ., nᵢ
The normalized probabilistic distance is given by
δ^{jk}ₑ,=d^{n,k}ₑ/Σⁿⁱᵢ₌₁d^{ik}ₑ,
Let pe(k)=(pe,l(k), l=1, . . . , nᵢ) denote the estimate of the p(k) scenario by the expert eεE. The re-norm-weighted mean, rₑεR is used to calculate p_{e,l}(k) as follows
P_{e,l}(k)=Σⁿⁱᵢ₌₁δ^{jk}ₑ(pₗ₍ᵢ))^{r}^{ₑ})1/rₑ
Let wₑ, eεE be a set of non-negative normalized weights
wₑ≧0, Σ_{eεE}wₑ=1
The integrated expert estimates pₗ(k) by combining p_{e,l}(k) as follows
pₗ(k)=(Σ_{eΣE}wₑ(p_{e,l}(k) )^{r})^{l/r}

The interpretation of pₗ(k) for different values of r is as follows: for r=1 it is the weighted arithmetic mean; for r=0, pₗ(k)=ΓI_{eεE}p_{e,l}(k)^{w}^{ₑ} it is the weighted geometric mean; and for r=−1 it is the weighted harmonic mean. For r=∞ it equals to maxeεE (p_{e,l}(k)), and for r=−∞ it equals mineεE (p_{e,l}(k)).

The quality of calibration of an integrated expert is measured by looking at how close the actual scenarios of the calibration runs are from those estimated by the integrated expert. The estimated scenario is denoted by pe(k)={pₗ(k), . . . , pₙⱼ)} and the actual scenario is denoted by p(k)={(pₗ(k), . . . , pₙᵢ(k)}k=1, . . . , ₙᵢ. The Kullback criterion is used here
q(p^{Λ}(k);p(k))=Σⁿ^{ᵢ}ᵢ₌ₗpₗ(k)log(p^{Λ}i(k)/pₗ(k))
Which is always non-negative and takes its minimal value of 0 if and only if p^{Λ}=p. A good integrated expert should give an estimated scenario p^{Λ} close to p and hence have the Kullback criterion value close to 0.

The weight wₑ of expert e used in the combination of expert opinions does not have the interpretation of ‘the probability that expert e is correct’. The fact that the experts' weights sum to 1 does not imply that precisely one expert can be correct. It is perfectly possible for two experts to give different distributions D^{k}ₑ, but for both to have ‘correct’ (in the sense of performance index) estimate pₑ(k) of the scenario pₑ(k). Hence, it is not possible to consider the correctness of the experts as ‘exclusive’. Instead, the experts' weights may be interpreted as scores. In the proposed model the choice of weights is made by looking at the performance that the integrated expert would have if he was to be scored as a standard expert, and choosing that values of weights that maximize this performance.

3. Soft Classification

Each operational run supply the numerical component of the feature vector to the optimally selected experts eεE*, that generate scenario estimates pₑ. These in turn are fused to form the integrated expert scenario estimate. Soft classification translates the scenario into weights for each value of every ordinal or nominal feature. Each weight measures the confidence in a particular feature taking a specific value in this operational run.

### IX. EXAMPLES

The following examples are provided to illustrate certain features of the present application. The scope of the application should not be limited by those features exemplified by such examples.

### Example 1

Betta chromatophores were generally cultured as described in this example. All steps in the culturing process were done using sterile technique in a tissue culture hood.

Selected fish are placed in a 4-liter anesthetic ice bath until dead, typically for at least 10 minutes, and are then washed by swirling with a large, blunt forceps in sterile water. The washed fish are then transferred into a plastic petri dish and the fins are removed using surgical scissors and fine forceps. The fins are transferred to another plastic petri dish containing phosphate buffered saline (PBS, 128 mM NaCl, 5.6 mM glucose, 2.7 mM KCl, antibiotic/antimycotic (1:100 dilution of GIBCO penicillin-streptomycin-fungizone 100× stock solution), 10 mM Na₂HPO₄ (pH 7.4), and 1.46 mM KH₂PO₄). The fin tissue is then diced with scissors into pieces of a selected size, typically 0.5-1.0 cm².

Skin is removed by placing the fin pieces in a 50 mL plastic tube containing 10 mL skinning solution (1 mM NaEDTA in PBS at a pH of 7.4). The tube is then placed in an orbital shaker at about 80-100 rpm for about 20 minutes and the skinning solution is changed about five or more times during this time. After the last skinning solution change, a filter-sterilized (0.2 μm filter) digestion solution (collagenase I (3 mg/mL)), and hyalouronidase (0.2 mg/mL) in 7 mL PBS is added. After 10-20 minutes, the digestion solution is removed using a transfer pipet and placed in a 15 mL plastic tube that is spun in a tabletop centrifuge for 2-3 minutes. The digestion solution (supernatant) is then transferred back to the 50-mL plastic tube containing the fin pieces and returned to the orbital shaker.

A pellet remaining in the bottom of the 15 mL tube contains individual cells from the fins, but the first pellet collected typically contains primarily epithelial cells and is discarded. This pelleting process is repeating at 15 minutes intervals and is repeated until substantially all of the chromatophores are collected.

Cells are recovered from the pellet by adding 7 mL of L-15 (25 mM HEPES, antibiotic/antimycotic, pH 7.4), mixing thoroughly, and re-spinning 1000×g for 2-3 minutes. The L-15 is then aspirated and the cells are resuspended in about 6 mL of fresh L-15. The volume of media used depends on the size of the pellet and the desired density of the cultures. Typically between 2 to 10 mL are used.

The cells are then plated in media that contains no serum because serum proteins compete with chromatophores for binding to the substrate. The substrate (typically a cell chamber surface, such as MELINEX, polycarbonate, glass, polystyrene) is coated with collagen IV (basement membrane collagen) at 0.5 to 5 μg/mL and fibronectin at 0.25 to 2.5 μg/mL and allowed to stand for a hour to allow adsorption to substrate. The substrate is then rinsed with PBS and let dry. Cells, after being plated onto substrate, are then covered and left to stand for about an hour to allow them to settle and attach.

After 1-2 hours in the hood the cultures now attached to the substrate and the reservoirs are filled with FSL cell culture medium (L-15, 5% fetal bovine serum, 25 mM HEPES, antibiotic/antimycotic prepared as a 1:100 addition of GIBCO penicillin-streptomycin-fungizone 100× concentrate at a pH of 7.4). The cultures can be stored at room temperature.

### Example 2

This example describers a representative encapsulation method. The on-line creation of microcapsules containing both an environmental sample and chromatophores can be done with an extruder, such as shown in FIG. 18, that receives feed-streams of a gel-forming material to produce sub-millimeter sized spheres containing chromatophores and sample.

Fish cells (chromatophores) from Betta fish are isolated (as described above) from the fish tail and fins and mixed with sterile alginate solution. Polymerized alginate beads of uniform size (˜400 μm) are obtained by extruding the alginate solution through the needle of an air-jet droplet generator (see FIG. 18) and collecting them in the CaCl₂ solution. The beads are subsequently encapsulated within a semipermeable poly-L-lysine (PLL) capsule. The interior of the microcapsules is liquefied with citric acid. This enables a liquid environment for the fish cells within a permeable capsule that will allow the diffusion of nutrients and toxins of certain molecular weight ranges.

The fish cells are pelleted by centrifugation and resuspended in 5 mL of 1.5-2.5% sodium alginate solution that has been filter sterilized using a 0.2-μm filter. The sodium alginate solution is made by dissolving alginate powder into warm saline solution (0.85 g NaCl in 100 mL distilled water). The alginate is sprinkled into the saline solution, a small amount at the time, with gentle mixing. Once it has dissolved (approximately 1-2 h), the viscous solution is allowed to cool, and then it is transferred into plastic tubes, capped, and stored in refrigerator until required.

The fish cells that were resuspended in sodium alginate solution are then extruded using either a syringe or an air-jet droplet generator into 100 mL of a 1.5% CaCl₂ solution. Ferromagnetic particles are added to the cell/alginate mixture prior to extrusion. The ferromagnetic particles help control motion of the microcapsules in the biosensor device using a magnetic field. See Example Embodiment 12 for description of magnetic particle manipulation.

The resulting beads are then transferred to a sterile 50-mL plastic centrifuge tube with a conical bottom. The beads are washed (10 times) with 30 mL of 0.1% 2-α-cyclohexylamino ethane sulfimic acid. A 0.1% CHES solution is prepared by adding 5 mL of CHES stock solution to 95 mL of 1.1% CaCl₂ solution, CHES stock solution is made by dissolving 2 g of CHES and 0.51 g of NaCl in 90 mL of distilled water, adjusting the pH to 8.2 with NaOH, and increasing the volume to 100 mL. After allowing the beads to settle (for up to 60 s), the volume of the supernatant is reduced with a vacuum aspirator.

A semipermeable capsule membrane is then formed by reacting the gel droplets with 30 mL of a 0.05% (w/v) poly-L-lysine (PLL) solution (PLL hydrobromide—SIGMA (Mw=30,000-70,000) in sterile saline. The resulting capsules are then allowed to settle, the excess PLL solution is aspirated off, and then the capsules are washed with 30 mL each of 0.1% CHES and 1.1% CaCl₂ and with two aliquots of saline. The capsules are then resuspended in 30 mL of 0.03% sodium alginate solution for 4 min to form an outer layer on the capsules and to neutralize free active groups on the PLL membrane.

The interior of the PLL microcapsule is liquified by suspending 5 mL of the capsules in 30 mL of a 0.05 M sodium citrate solution (2.58 g of sodium citrate and 0.85 g of NaCl in 200 mL of distilled water) for 6 minutes. The capsules are then washed several times in saline to remove any excess citrate and then rocked end-to-end for 30 min to allow the alginate to diffuse out of the capsules, and for the capsules to swell toward their equilibrium state.

The capsules are now ready to be incubated with various test compounds. Incubation permits bacteria to express toxins that are detected by chromatophores.

### Example 3

This example is directed to using more than one population of sensor cells. One or more cell chambers were micromachined in polycarbonate. In representative examples, 2 and 5 cell chambers were defined. Each of the chambers can be supplied from a common reservoir. Such a design allows flexibility in fluidic connections as well as in a number and use of chromatophore chambers. Supply lines or inlet channels can be independently controlled with valves or pumps to set desired flow rates for different analytes.

Syringe pumps situated upstream of the chambers can be used to apply the sample to successive chambers. Each chamber can include individual control of sample intake volumes and flow rate using valves or with pump control. A common downstream pumping mechanism can be used to maintain a basal flow of fluid to the bank of chambers. The downstream pump can be a syringe pump, a capillary wick, a negative partial pressure, or some other pumping system. All chambers are typically configured with a basal flow of fluid that can be achieved by either individual pumps or a common downstream pump. Such a pump can be a syringe pump, a wicking type pump mechanism, an electro-osmotic type, or some other pump. A multi-stream arrangement can be used to direct parallel and separate streams through a common chamber to achieve concurrent sampling in a common cell chamber. Multiple streams within a single chamber may be possible and thereby permit introduction of multiple analytes into a common chamber. By setting the proper flow conditions these streams can remain separate within the chamber. A simple two-stream prototype was used to evaluate the extent of fluid mixing by adjacent streams within a single chamber and showed that fluid mixing control is achievable.

An illumination system includes a light source such as a laser diode, light emitting diode, or lamp that emits radiation that is directed to the sensor cells. An optical system can be provided to shape and direct the radiation, and mirrors or beamsplitters can be configured so that a single light source illuminates more than one cell chamber. One or more cameras are arranged to receive radiation from the cell chambers. A single camera can be arranged to image cells in one or more cell chambers using relay optics, or several cameras can be used. Multitasking can be used to enable one or more computer systems to process the images collected from multiple chambers.

An encapsulation machine can be used to take periodic samples of analytes and package them for optical measurements, using a method of continuous encapsulation of sensor cells. After formation, capsules can be arranged in an order that allows each to be associated with the period of time that a respective sample was fed into the system. Controlled microfluidics and/or movements achieved by the use of magnetic fields on suitably doped capsules can be used to move the capsules to positions within an instrument where they can be optically observed. An optical detection system can be used to measure optical appearance of the chromatophores within the capsules.

Various types of illumination can be used to irradiate the capsules, including a fluorescent lighting arrangement based on innate fluorescence of some chromatophores (including erythrophores), or fluorescence of a marker introduced into the chromatophores.

An analyzer with parallel processing capabilities or other suitable hardware architecture can be used to keep track of the optical appearance of the capsules and determine whether a significant change occurs in any capsule. Given that each capsule is associated with a particular sample entry time, results can be associated with analyzer times at which biologically active agents are introduced.

A sensor cell feed line provides chromatophores in a form that makes them readily available for encapsulation and an analyte feed line receives a liquid sample that is directed to an encapsulation zone. The encapsulation zone is a portion of a cytosensor at which sensor cells and analyte are mixed together and, in conjunction with gel-forming and gel-dissolving components, capsules are formed.

### Example 4

### Cells are Ensembles of Biological Elements

This example highlights the fact that cells act as ensembles of biological elements. Using the trimeric G-protein mediated signaling pathways as an example, receptor-trimeric G-protein-adenylate cyclase complexes form at the cell surface, controlling cAMP levels, which then primarily modulate PKA activity. Localized function is achieved through binding of PKA to AKAP. Short term responses involve the spatiotemporal organization or reorganization of existing biological elements resulting in the aggregation or disaggreggation of melanosomes. Longer term responses involve remolding or reoptimization of the system through the generation and integration of new or modified biological elements, resulting in remolded signaling modalities. B. D. Gomperts et al. review signal transduction networks (B. D. Gomperts, I. M. Kramer and P. E. R. Tatham, Signal Transduction, Elsevier Academic Press, New York, 2002). More specific references for the G-protein mediated organelle movement are detailed below.

A. M. Preininger and H. E. Hamm review heterotrimeric guanosine triphosphate-binding protein structure and function. Trimeric gene proteins form complexes with various effectors and signaling partners in response to G-protein coupled receptor (GPCR)-ligand interactions and other binding events. The determination of the crystal structures of G proteins in various activational states and, more recently, in complexes with effectors and other signaling partners highlights the mechanisms involved in G-protein coupled receptor (GPCR) regulation (A. M. Preininger and H. E. Hamm, G-Protein Signaling: Insights from New Structures, Science's STKE, www.stke.org/cgi/content/full/sigtrans,2004/218/re3).

Adenylate cyclase and other cell surface effector proteins mediate signals between upstream receptor binding events and downstream regulatory cues involved in the digitization of incoming analog signals. V. J. Watts reviews the molecular mechanisms of adenylate cyclase activation. The nine-membrane bound isoforms of the enzyme adenylate cyclase (AC) are highly regulated by neurotransmitters and drugs acting through GPCRs to modulate intracellular cAMP levels (V. J. Watts, Molecular Mechanisms for Heterologous Sensitization of Adenylate Cyclase, J. Pharm. Exptl. Thera. 302(1), 1-7, 2002). G. G. Kelley and others report on the mechanisms and regulation of phospholipase-Cε, a recently discovered effector molecule with many activities distinct from those of other phospholipase isoforms, particularly with respect to the manner of involvement of GPCRs in its regulatory cascade (G. G. Kelley, S. E. Reks and A. V. Smrcka, Hormonal regulation of phospholipase Cε through distinct and overlapping pathways involving G12 and Ras family G-proteins, Biochem. J., 378, 129-139). Heterologous and homologous phosphorylation and dephosphorylation of effector and receptor complexes by PKA and receptor-specific kinases and other regulatory molecules result occurs in response in ligand concentration. High concentrations of ligand result in down regulation of receptors with high specifities for the binding ligand through phosphorylation by receptor specific kinases. Lower concentrations of ligand result in a rhythmic phosphorylation/dephosporylation of affected receptors digitizing incoming signals.

D. Diviani and J. D. Scott review the role of A-kinase anchoring protein (AKAP) kinase signaling complexes for localization of GPCR/AC/PKA mediated signaling pathways. Rab GTPases involved in GPCR mediated organelle movement are AKAPs (D. Diviani and J. D. Scott, AKAP signaling complexes in the cytoskeleton, J. Cell Sci., 114, 1431-1437). As with PKA, There are several classes of proteins that bind and target PKC to specific subcellular sites known in general as PKC anchoring proteins, or more specifically, STICKs, PICKs and RACKs. RACKS in particular associate PKC with specific membrane domains.

V. I. Gelfand et al. review the formation of signaling complexes involved in organelle transport in Xenopus laevis melanophores. The signaling cascades involve GPCRs, AC, protein kinase A (PKA), protein phosphatase 2A (PP2A), calcium modulated kinase II (CaMKII), Rab GTPases and myosin-5 (A. A. Nascimento, J. T. Roland and V. I. Gelfand, Pigment cells: A model for the study of organelle transport. Annu. Rev. Cell Dev. Biol., 19, 469-91). Reilin et al. demonstrate that PKC is also involved in the G-protein regulated movement of organelles (Reilin, A. R., Tint, I S, Peunove, N. I. et al. Regulation of organelle movement in melanophores by protein kinase A (PKA), protein kinase C (PKC), and protein phosphatase 2A (PP2A), J. Cell. Biol., 142(3), 803-13.

Addition of different hydrophobic tails to the various molecular players in the G-protein mediated and other signaling pathways aids in lipid membrane localization of particular signal transduction activities. M. A. del Pozo et al. (2004) report that regulation of lipid rafts by integrin signals may regulate the location of membrane domains such as lipid rafts and thereby control domain-specific signaling events in anchorage dependent cells (M. A. del Pozo, N. B. Alderson, A. B. Kiosses et al., Integrins regulate Rac targeting by internalization of membrane domains, Science, 303, 6 Feb. 2004). L. J. Pike reviews lipid rafts, membrane microdomains implicated in processes as diverse as signal transduction, endocytosis, and cholesterol trafficking, and implicated in the spatial organization of cellular functional modules (L. J. Pike, Lipid rafts: heterogeneity on the high seas, Biochem. J., 378(Pt. 2), 281-292, Mar. 1, 2004). S. Maslov and K. Sneppen show that links between highly interconnected proteins are systematically suppressed whereas links between a highly connected and low connected protein pairs are favored (S. Maslov and K. Sneppen, Specificity and Stability in Topology of Protein Networks, Science, 296, 910-913, 3 May 2002).

Those of ordinary skill in the art understand that the regulatory mechanisms involved in the control and localization of cell function are myriad and involve many molecules internal and external to the cell, including, but not limited to small metabolites, peptides, proteins, metal ions, and nucleic acids and that the detailing of current understanding of mechanisms involved in G-protein regulated pathways rather than other regulatory, transformative or as yet unidentified pathways does not limit this invention in any way.

Those of ordinary skill in the art will recognize that the general mechanisms of functional module formation described above for organelle transport also can be applied to many different activities at many different hierarchical levels of system organization. For example, J. R. Terman and A. L. Kolodkin report that Drosophilia Nervy, a member of the myeloid translocation gene family of A kinase anchoring proteins (AKAPs), regulates repulsive axon guidance by linking the cyclic adenosine monophosate (cAMP)-dependent protein kinase A (PKA) to the Semaphorin 1a (Sema-1a) receptor Plexin A (PlexA). Thus, Nervy couples cAMP-PKA signaling to PlexA to regulate Sema-1a-mediated axonal repulsion, revealing a simple molecular mechanism that allows growing axons to integrate inputs from multiple guidance cues (J. R. Terman and A. L. Kolodkin, Nervy links protein kinase A to plexin-mediated semaphoring repulsion, Science, 303, 1204-1207).

### Example 5

### Bioactive Compounds Can Act at Specific Molecular Targets in Cells

The following experiments with fish chromatophores provide evidence that biosensor cells can be used to detect bioactive compounds that act upon specified molecular targets. For example, one important class of compounds includes pharmacological agents that act upon the membrane channels that admit calcium ions into cells. Drugs based on the activation or inhibition of these membrane channels can be useful in treatments of various diseases and syndromes. To provide such chromatophore-based sensors, sensor cells are evaluated for the presence of a calcium ion channel. As a specific example, calcium-dependence of pigment transport in melanophores and erythrophores was evaluated.

Erythrophore cultures were prepared 3-10 days prior use as described herein. Dorsal, caudal, and anal fins were clipped from euthanized male Betta splendens and diced into 2.0-4.0 mm squares. The diced fins were washed in six solution changes of phosphate buffered saline (PBS) (137 mM NaCl, 2.7 mM KCl, 10 mM Na₂HPO₄, 1.6 mM KH₂PO₄, pH 7.4) containing 1 mM NaEDTA, and transferred into 7 ml of digestion solution (PBS containing 960 unit/ml collagenase I and 230 unit/ml hyalouronidase Worthington Biochemical Corp., Lakewood, N.J., USA). After a 30-minute incubation, dissociated erythrophores were separated from the digestion solution by centrifugation for 3 minutes at 300×g and resuspended in L15 media (Sigma-Aldrich Co., St. Louis, Mo., USA) supplemented with 5% fetal bovine serum. Erythrophores from three successive digestion solution incubations were pooled and suspended in complete L15 media at a concentration of 1×10⁵ cells/ml. Cells were plated on 15 mm diameter glass cover slips coated with 20 μg/cm² collagen IV and 15 μg/cm² fibronectin (both from Sigma-Aldrich Co.) by placing one drop on each cover slip. After allowing the erythrophores to attach to the substrate for one hour, the cover slips were submerged in complete L15 media and stored at room temperature. Mixed cultures containing both melanophores and erythrophores were prepared by choosing fin tissue that contained both color types of chromatophores. Alternatively, different fish can be used to provide different types of chromatophores and a suitable combination produced by mixing.

Prior to exposure with calcium-modulating chemicals, erythrophores attached to cover slips were loaded into a continuous flow chamber (Warner Instrument Corp., Hamden, Conn., USA) and equilibrated for 5 minutes in physiological saline solution (PSS, 128 mM NaCl, 2.7 mM KCl, 1.8 mM CaCl₂, 1.8 mM MgCl₂, 5.6 mM glucose, 10 mM Tris/HCl, pH 7.2). Added agents were injected directly into the enclosed cell chamber at the given concentration dissolved in 200 μl of PSS. Added chemicals can be flushed out of the cell chamber by continuous upstream flow of PSS.

An assembled erythrophore chamber was mounted on a stage of a Zeiss IM-35 inverted microscope (Oberkochen, Germany) fitted with a Plan 6.3x objective having a 0.16 numerical aperture. A field of view was chosen that included about 200 erythrophores. Digital images were captured every 20 seconds with a Panasonic GP-US502 color 3-CCD camera using a Flashpoint 3D frame grabber and a personal computer based on a Pentium III processor. Image processing was performed using Image Pro 4.1 image analysis software.

Measurements were based on the following steps. Images were segmented to distinguish erythrophores (dark objects) from a bright background and a relative area occupied by erythrophores was calculated. The area was then calculated for all dark objects in the segmented image. The area was calculated in relative size units.

Pigment aggregation and dispersion trends within a sequence of images were determined by calculating a relative aggregation for each image in the sequence. The relative aggregation (Relative Aggr.) was computed using the following formula:
Relative Aggr.=−log₁₀(Pixel Area/Reference Pixel Area)
wherein the pixel area was determined for each image and the reference pixel area was the area for the first image in the sequence.

A dose-response curve was obtained and individual data points in the dose response curves were found by calculating a dispersion ratio for each test. This value was calculated as the following ratio: dispersion ratio=(erythrophore area after 10 min exposure)/(erythrophore area before exposure).

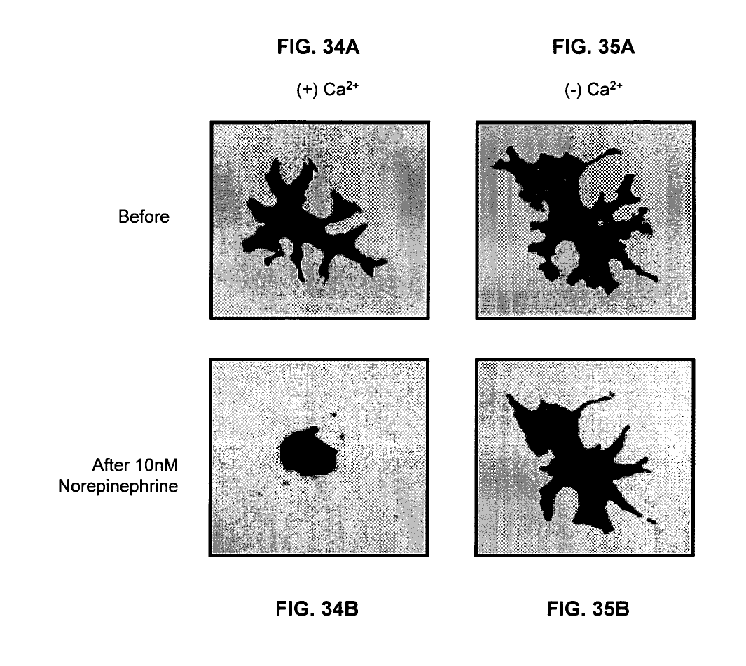

The requirement of calcium for intracellular transport in erythrophores has been hypothesized for erythrophores previously, but the involvement of the key molecular target of membrane acting calcium channel blocking drugs has not been investigated. The calcium requirement for intracellular transport of pigment was confirmed in Betta splendens erythrophores by treating the cells with a common aggregation-inducing stimulus (10 nM norepinephrine) in solution that either contained calcium ion or solution devoid of calcium ion. The results on erythrophores from this treatment are shown in FIGS. 34A-34B and 35A-35B. With reference to FIG. 34B, in calcium containing PSS (1.8 mM added Ca²⁺), treatment of erythrophores with norepinephrine produces apparent aggregation of pigment-containing vesicles with respect to unexposed erythrophores (FIG. 34A). With reference to FIG. 35B, exposure in PSS with 1 mM EGTA and no added calcium produces no apparent aggregation in comparison with unexposed erythrophores (FIG. 35A). It is evident that pigment aggregation was impaired by the lack of calcium. Thus, pigment transport can be altered by pharmacologically perturbing intracellular calcium concentration.

Modulators of plasma membrane calcium channels were initially considered as a means of controlling intracellular calcium levels. The direct application of the calcium channel activator Bay K8644 resulted in neither the aggregation nor dispersion of erythrophore pigment and direct application of a variety of Ca²⁺ channel inhibitors did not produce a direct response in erythrophores. However, pre-treatment with norepinephrine has a two-fold effect on erythrophores. First, norepinephrine operates through adrenergic receptors to open receptor-activated calcium channels on erythrophores. This results in a higher transient intracellular Ca²⁺ concentration. Second, norepinephrine acts as an agonist of α₂-adrenergic receptors that leads to inhibition of adenylate cyclase and a decrease in intracellular cAMP levels, a pathway that is well characterized in association with the aggregation of pigment in all dendritic chromatophores, including erythrophores. The application of inhibitors of plasma membrane calcium channels to erythrophores after pre-treatment with norepinephrine resulted in immediate and rapid dispersion of pigmented vesicles.

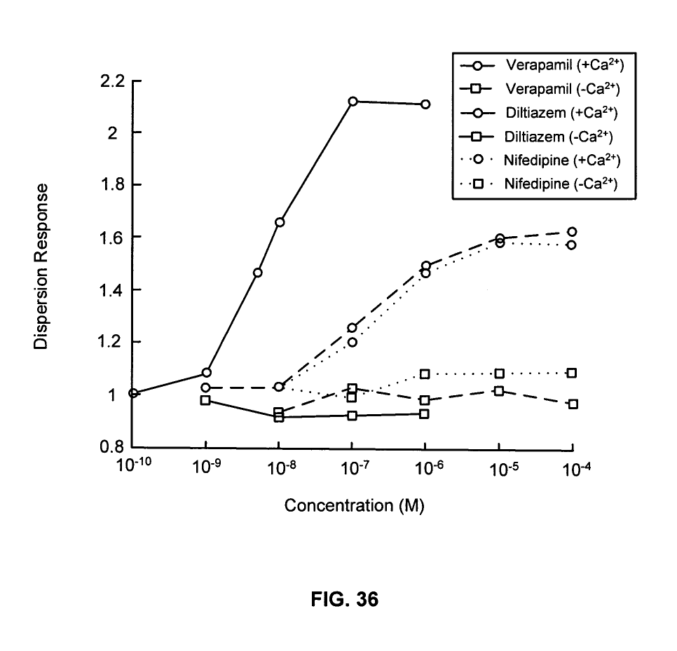

FIG. 36 contains dose response curves for inhibitors verapamil, diltiazem, and nifedipine. Erythrophores were treated for at least 5 minutes with 1 nM NE prior to exposure to the L-type Ca²⁺ channel blockers. Dispersion response was calculated as an area occupied by cells after exposure for 10 minutes divided by an area occupied prior to exposure. Curves labeled with (+Ca²⁺) were performed in PSS containing 1.8 mM added Ca²⁺. Curves labeled with (−Ca²⁺) were performed without added Ca²⁺. Each of the above chemicals added directly to erythrophores produces pigment dispersion but does not alter melanophores. The mode of action for each of these agents is similar in that they work by blocking the passage of calcium ions from extracellular space into the cytoplasm. Each of these chemicals induces this response in a dose dependent manner. Control experiments performed in solutions without added calcium failed to induce pigment dispersion response so that a direct role of intracellular calcium concentration in these cells is implicated.

Erythrophore dispersion caused by Ca²⁺ channel inhibition was examined by imaging continuously during an experiment where two different verapamil concentrations were added in succession. Continuous pre-treatment with norepinephrine caused pigment to aggregate in cells that normally have dispersed pigment. Upon addition of a dose of 1 nM verapamil, rapid dispersion followed until the cell chamber was flushed of verapamil by continued flow of buffered solution with added norepinephrine. This allowed the cells to re-aggregate rapidly until a second, stronger dose (100 nM) of verapamil was added. This stronger dose of verapamil caused the cells to disperse almost as completely as possible and the dispersion was sustained as long as verapamil was present. This demonstrates not only the sensitivity of erythrophores to Ca²⁺ channel inhibitors, but also the ability of these cells to repeatedly and consistently respond.

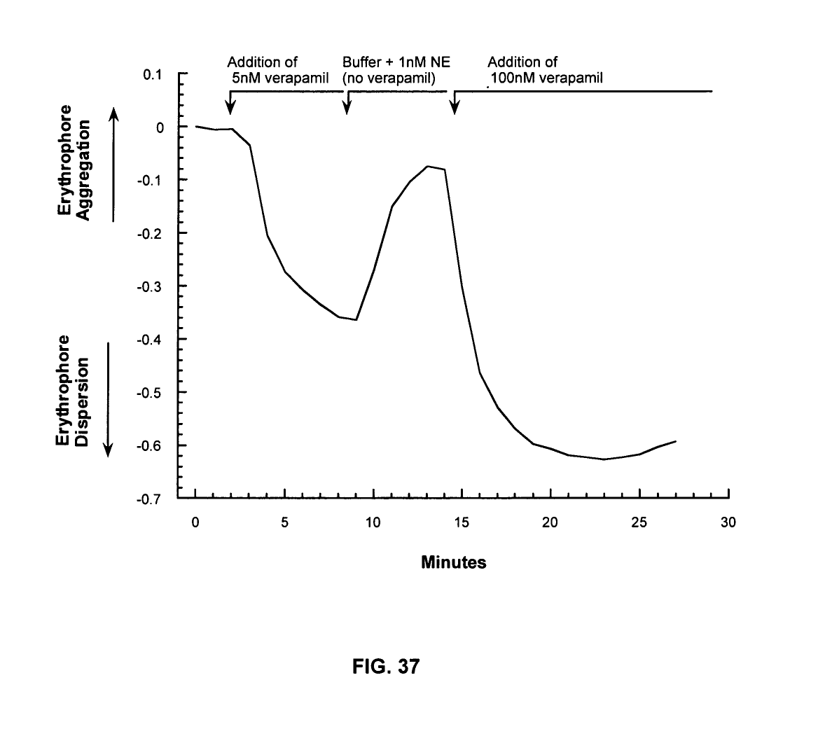

FIG. 37 contains a graph of erythrophore aggregation/dispersion as a function of time during which various doses of verapamil are applied. Initially, the erythrophores are exposed to norepinephrine and then exposed to 5 nM verapamil causing pigment dispersion. During flushing, there was an exposure to 1 nM NE without verapmil, producing re-aggregation. Exposure to 100 nm verapmil produced a larger dispersion.

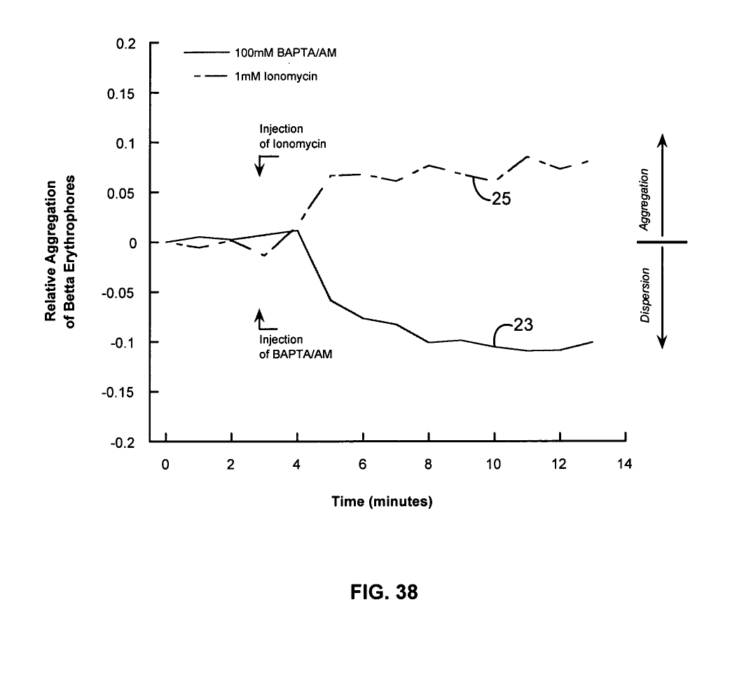

To confirm the results observed by raising intracellular Ca²⁺ concentration with Ca²⁺ channel blockers, the membrane-permeable acetylmethyl (AM) form of the Ca²⁺ chelator BAPTA was added to erythrophores. The lower intracellular Ca²⁺ concentration ensuing from chelation resulted in pigment dispersion as seen in FIG. 38. These results led to the question of whether increasing intracellular Ca²⁺ levels would result in an opposite effect as decreasing levels, namely by aggregating the pigment in erythrophores. Treating erythrophores with the Ca²⁺ ionophore ionomycin tested this idea. The results of this experiment can also be seen in FIG. 38 in graph 25. Upon addition of ionomycin to erythrophores a slight, but sustained aggregation is apparent, confirming that Ca²⁺ concentration can independently modulate both dispersion and aggregation in erythrophores.

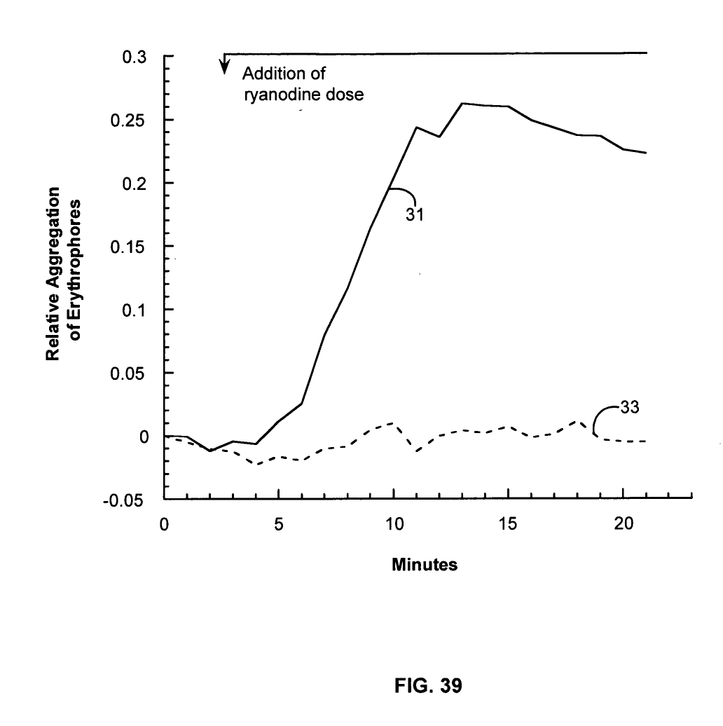

The presence of intracellular Ca²⁺ channels in erythrophores was also investigated. Such ryanodine and IP₃ receptors are present on the endoplasmic and sarcoplasmic reticulum in many other cell types and control the flow of Ca²⁺ from intracellular stores. Again the pharmacological applications of receptor modulators were used on ryanodine receptors. IP₃ receptors were not examined here because of their cross-reactivity in other signaling pathways and because of the lack of specific cell-permeable agonists. Ryanodine receptors were screened for by the application of the agonist ryanodine as seen in FIG. 39. Ryanodine is a membrane permeable molecule that has an unusual biphasic effect on intracellular receptors because at low concentrations (1nM-100 nM) ryanodine acts as an agonist of the receptor. However, at high concentrations (>1 μM) ryanodine acts as an antagonist of the same receptors. Referring to FIG. 39, curve 31 corresponding to 10 nM ryanodine is associated with increasing aggregation while curve 33, corresponding to 10 μM ryanodine is associated with decreasing aggregation. This information is consistent with the data shown in FIG. 39. A 10 μM dose of ryanodine onto erythrophores did not produce any morphological change in the cells, signifying that no Ca²⁺ was released into the cytoplasm from intracellular stores. However, a 10 nM dose of ryanodine onto erythrophores did produce a sustained aggregation response. This result is consistent with the biphasic response profile of ryanodine. Images of erythrophores treated with low and high concentrations of ryanodine are consistent with these results. The aggregation response seen at a low concentration (10 nM) of ryanodine suggests that ryanodine receptors are mediating the release of Ca²⁺ from the endoplasmic reticulum into the cytoplasm.

Ca²⁺ channels are involved in the bi-directional movement of intracellular vesicles in erythrophores. There is no corresponding calcium dependence in melanophores, and so the combined observation of erythrophores and melanophores in response to pharmacological agents can verify or refute the possibility that a given agent acts upon calcium regulating molecular targets within the cells. Such a capability of chromatophores is established by the showing that the requirement for extracellular calcium for pigment movements and the effects of numerous calcium-modulating agents on pigment movements are exclusive to erythrophores and not melanophores.

### Example 6

### Use of Elicitors in Absence of Pattern Recognition Tools

This example demonstrates that the use of elicitors increases the information content of biosensor experiments. Fish chromatophores were exposed to algal extracts. A correlation between biosensor cell activity and pharmacological activity of algal extracts was established. The degree of aggregation and dispersion in chromatophore cells, specifically melanophores, is determined using a melanophore index rating system. The melanophores are placed in contact with an algal extract for twenty-four hours and then tested by exposure with norepinephrine. Norepinephrine is known to cause complete melanophore aggregation. Four basic types of responses were observed as the result of cytosensor exposure to algal extracts.

In a type I response, the extract has no effect on the cytosensor. In a type II response, a direct change in the melanophore index is produced as the result of extract exposure followed by complete aggregation due to norepinephrine exposure. In a type III response there is a direct response to extract exposure followed by impaired aggregation when exposed to the elicitor, norepinephrine. In a type IV response there is no direct response to algal extract exposure and there is impaired aggregation from subsequent exposure to the elictor, norepinephrine.

Extracts were tested along with fractions from extract 1233, and three pure marine toxins: brevetoxin A, saxitoxin, and curacin A. The activity of melanophore aggregation or loss of ability to aggregate suggests that there is a correlation between algal extract activity and biosensor cell activity. This correlation indicates that such a cytosensor is useful in screening active algal extracts for pharmaceutical activity.

Algal extracts were detected using melanophore activity from Nile tilapia scales. A solution of 2.3 mM tricaine methanesulfonate (MS222) and 20 mM Tris-HCl (pH 7.6) in deionized water is used to euthanize the Nile tilapia fish. (Plunging the animal in a cold ice bath can also be used to kill the fish.) The tilapia is removed from the solution and placed in a plucking apparatus. The plucking apparatus consists of a pipette box lid containing a fish tank water filter cut in half. The water filter is pre-moistened with deionized water or PSS (see below). Residual MS222 is removed from the fish by rinsing both sides of the fish. Typically, 60 scales are removed from the dorsal fin area of the tilapia. The scales are placed into 100 mm dishes containing 20 mL of a divalent cation-free “skinning solution” (1 mM EDTA and 10 mM glucose in phosphate-buffered saline, pH 7.4). The skinning process consists of four 30-minute intervals at 70 rpm on the shaker. The time and degree of shaking can be selected according to the efficiency at which the skinning process removes epithelial cell from the surface skin layers of the scale. For Betta splendens scales, a shorter incubation time and gentler agitation can generally be used. After each 30-minute interval, the spent skinning solution is replaced with 20 mL of fresh skinning solution. The final exchange replaces skinning solution with 20 mL of physiological saline solution (PSS) (128 mM NaCl, 2.7 mM KCl, 1.8 mM CaCl₂, 1.8 mM MgCl₂, 5.6 mM glucose, 10 mM Tris-HCl 7.2 pH, 100 U/mL penicillin, and 100 μg/mL streptomycin). Following the skinning process, the scales are stored at 25° C. for 24 hours during which time they undergo a period of spontaneous optical changes. These spontaneous changes are related to the tissue injury caused by their excision, which causes internal bioactive conditions (neurotransmitters and hormones) to temporarily induce optical changes. Scales can be stored for periods of weeks in PSS. A change of the PSS solution each week to serve as a periodic feeding was used, but other feeding regimens that maintain the sensitivity of the chromatophores can be used.

### Example 7

This example concerns an embodiment of a method for testing for bacteria. Defined bacterial strains can be obtained from research labs or from culture collections such as the American Type Tissue Culture. A pure culture of bacteria (one that consists of only one type of bacteria) is cultivated in a culture-specific medium using aseptic technique. This medium can be Luria broth's (LB) or any other conventional, nonconventional, chemically defined (complex) or undefined (complex) medium as described in references such as that published by ATCC (American Type Tissue Culture). In addition to appropriate nutrients, cultivating bacteria requires suitable environmental conditions, including temperature, pH, and oxygen conditions. When necessary, these environmental conditions are adjusted to obtain optimal growth of bacterial cultures.

Bacterial strains can be obtained from natural habitats. Aseptic technique is used to obtain a pure culture of bacteria from its natural habitat. These pure culture isolates are then cultivated as described above.

If desired, the bacterial culture can be filtered or centrifuged to separate bacterial cells from the medium, each of which can then be assayed in FSS (fish saline) or FSL (fish complete medium). Alternatively, the whole suspension (bacteria plus medium) can be diluted into FSS or FSL and used directly in the toxicity assay.

Gram negative and Gram positive bacterial strains have been tested against Betta splendens chromatophores (i.e., biosensor cells). The experimental approach was similar for all bacterial strains tested. Pure cultured bacterial strains were cultivated and approximately 10⁷ bacterial cells were added to FSS (fish saline) or FSL (fish complete medium) containing biosensor cells, and the reaction of the biosensor cells to the bacterial cells was noted microscopically after 5, 10, 15, 30 and 60 minutes. A direct positive reaction was observed when the pigment of the biosensor cells rapidly aggregated (in the first 5 minutes after the addition of the bacterial cells to the biosensor cells) in a manner similar to that observed with norepinephrine (NE). The Gram negative bacterial strain JM101, a non-toxin producing Escherichia coli strain used commonly in molecular genetic manipulations, did not cause the pigment of the biosensor cells to aggregate, thus indicating a negative response. This E. coli strain can be used as a negative control in toxicity assays. The Gram positive bacterial strain ATCC #6051, a non-toxin producing, non-pathogenic Bacillus subtilis strain used commonly in molecular genetic manipulations, does not cause the pigment of the biosensor cells to aggregate, thus indicating a negative response. This B. subtilis strain can be used as a negative control in toxicity assays. Similarly, the Gram positive non-toxin producing, non-pathogenic Lactococcus lactis bacteria used commonly in food fermentations processes does not cause the pigment of the biosensor cells to aggregate, thus indicating a negative response. This Lactococcal strain can also be used as a negative control in toxicity assays. In contrast, the Gram negative bacterial strain ATCC#4931, a toxin-producing, pathogenic Salmonella enteritidis strain, caused the pigment of the biosensor cells to aggregate within the first 5 minutes indicating a direct and rapid response of the biosensor cells to the toxin-producing, pathogenic Salmonella enteritidis. Similarly, the Gram positive bacterial strain ATCC#49064, a toxin producing, pathogenic Bacillus cereus strain, causes the pigment of the biosensor cells to aggregate within the first 5 minutes indicating a direct and rapid response of the biosensor cells to the pathogenic Bacillus cereus. Both the Salmonella enteritidis (ATCC#493 1) and the Bacillus cereus (ATCC#49064) strains were isolated as a result of a human gastroenteritis outbreak. Similar aggregation responses were elicited by a toxin-producing, pathogenic Escherichia coli 0157:H7 strain and a Vibrio cholera strain.

In addition to the defined bacterial strains obtained from research labs or from culture collections such as the American Type Tissue Culture, bacterial strains obtained from natural habitats were also assayed. Of the seven pure bacterial isolates obtained from natural habitats and tested to date, three caused pigment of the biosensor cells to rapidly aggregate (within 5 minutes) indicating the presence of a toxin. The three naturally occurring bacterial isolates have been putatively identified as a Bacillus sp., a Bacillus cereus, and a Clostridium sp. The other four bacterial isolates that did not cause the biosensor cells to rapidly aggregate (thus indicating no presence of toxin) were identified as either a Pseudomonas sp. or a Bacillus subtilis.

### Example 8

This example concerns detecting biological toxins in food and water using a cytosensor. Three classes of purified toxins were tested, including an enterotoxin, a membrane damaging toxin, and a protein synthesis inhibitor. Purified cholera toxin, an enterotoxin from Vibrio cholera, causes hyperdispersion at toxin concentrations of 50 ng/mL down to 1 ng/mL, following a 20 hour incubation of Betta splendens chromatophores with purified toxin. The levels of cAMP are increased with the addition of cholera toxin, and presumably this impact correlates with the observed hyperdispersion. The inhibition of norepinephrine (NE) induced pigment aggregation in Betta splendens chromatophores by cholera toxin is detected at concentrations as low as 1 ng/mL after a 20 hour reaction time. The NE inhibition affect by cholera toxin is more evident at higher cholera toxin concentrations.

A membrane damaging toxin, alpha-hemolysin from Staphylococcus aureus, directly changes Betta splendens chromatophores in a manner similar to norepinephrine. Pigment aggregation in these chromatophores can be observed one hour after the addition of 0.5 or 0.1 μg/mL of alpha hemolysin toxin. A protein synthesis inhibitor, Shiga-like toxin, had no readily observable effect on the Betta splendens chromatophores at the highest dose tested (0.1 mg/mL).

### Example 9

This example concerns using a cellular biosensor that couples the analytical sensitivity of fish chromatophores to the neurotransmitter secretory behavior of nerve cells. Such a system can be applied to detection of the neurological toxins botulinum and tetanus in unknown samples and can used with combinations of several types of chromatophores.

A multi-cell system uses culture procedures that allow chromatophores and PC12 nerve cells to coexist in similar environments of temperature and media composition for an extended period. The system also allows the neuron-chromatophore bioassay to detect the effects of neurotoxins on PC12 cells. This method has been developed to monitor the activity of nerve cell neurotransmitter secretion using changes in chromatophore morphology. The substances examined with this assay include toxins whose pathogenesis is mediated through inhibition of the neurosecretory pathway. In this example embodiment, the Clostridial toxins botulinum and tetanus are selected. In addition, the responses caused by each toxin are characterizable, allowing differentiation of toxins in an unknown sample. For example, toxins including cholera, pertussis, and β-latrotoxin can be measured or detected using a neuron-chromatophore bioassay.

### Example 10

In this example, a population of 1,000 Betta chromatophores contained in a cell chamber was used to detect a known bioactive agent, norepinephrine. The sample of norepinephrine was introduced into the cell chamber via syringe injection. Video images of the optical appearance of the chromatophores were analyzed. A statistically significant change in the appearance of the chromatophores was recognized by an analysis algorithm built into the cytosensor. Thus, at the expected moment when norepinephrine entered the cell chamber, the ensuing optical changes in the chromatophore sensor cells were sensitively reported by the cytosensor instrumentation.

Male fish of a red variety of Betta splendens were used as source of chromatophores that were obtained using the protocol described above. The chromatophores were transferred to a cell chamber and included approximately 1000 chromatophores (90% erythrophores, 9% melanophores, 1% iridophores) that occupied a 1 mm² viewing area. See FIGS. 1A-1B for examples of Betta chromatophore appearance.

A cell chamber was constructed using a laminate construction method as described herein. An outer layer was a transparent material (Melinex 453) for viewing the sensor cells. In this embodiment, the chromatophores were attached to the interior-facing surface of a transparent window layer. A chamber interior was routed with fluidic lines so that entry and exit of fluids and analytes could be accomplished. The fluidics geometry was designed such that the shear tolerance of cells fell below the expected allowable value of 1 N/m² at a height of 350 microns with a flow rate of 10 microliters per minute in a one millimeter wide section.

The cell chamber was sandwiched between the two halves of a fluid interconnect. The interconnect supplied the tubing to the fluid delivery system and the sample injection system. A connector was used that had an inlet tube at right angles to the chamber flow direction. The end of the tube was flanged, with the flanged portion held tight against the inlet port walls so that fluid could escape through the tube into the chamber. The dimensions and hexagonal geometry of the fluid interconnect were chosen so that the light source and camera mechanism could be mounted for recording the optical signals from the sensor cells.

A syringe injection system and syringe pumping system was used to deliver sample and aqueous medium to the cell chamber. A commercially available syringe pump (Hamilton, Reno Nev.) was used to deliver fluids into the chamber. Various flow rate could be selected. The syringe pump and injection system are described in additional detail elsewhere herein.

The optics subsystem imaged a central 1 mm×1 mm area of the 3 mm×3 mm total sample area onto the color CCD array for optimal field of view. A Hastings triplet lens provided an achromatic, flat field of view. The cell chamber was illuminated with white light emitting diodes.

An example analyte sample (two microliters of 100 nM norepinephrine dissolved in FSL medium) was delivered as a uniform front (slug) into the chamber.

A detection and identification algorithm used a time sequence of images acquired every five seconds. The steps were implemented on a general-purpose microprocessor running Windows NT. Screen capture images show detection results using 92 images. An alarm was triggered at a 60th frame, with a delay of two frames. Signature identification used 10 frames (from 58th to 67th). Frames 1 to 57 correspond to slowly changing statistics (null hypothesis). Frames 68 to 92 represent statistical equilibrium. Control injections of a blank analyte (FSL medium with no added norepinephrine) caused no alarm. Thus, upon injection of 100 nM norepinephrine, the analyzer indicated >0.99 probability of a biologically active agent. The response time of the cytosensor was typical of the biological response time to norepinephrine (i.e., less than about 1 minute).

### Example 11

### Use of Ensembles of Cells for Biosensor Applications

This example concerns the use of non-random mixtures or ensembles of cells for biosensing applications. The use of multiple cell types increases the information content of the experiments and facilitates the design of improved biosensor systems.

The coupling of fish chromatophores to the neurotransmitter secretory behavior of nerve cells can be applied to detection of the neurological toxins botulinum and tetanus in unknown samples and can used with combinations of several types of chromatophores. A multi-cell system uses culture procedures that allow chromatophores and PC12 nerve cells to coexist in similar environments of temperature and media composition for an extended period. The system also allows the neuron-chromatophore bioassay to detect the effects of neurotoxins on PC12 cells. This method has been developed to monitor the activity of nerve cell neurotransmitter secretion using changes in chromatophore morphology. The substances examined with this assay include toxins whose pathogenesis is mediated through inhibition of the neurosecretory pathway. In this example embodiment, the Clostridial toxins botulinum and tetanus are selected. In addition, the responses caused by each toxin are characterizable, allowing differentiation of toxins in an unknown sample. For example, toxins including cholera, pertussis, and β-latrotoxin can be measured using a neuron-chromatophore bioassay.

### Example 12

### Use of Ensembles of Cells for Biosensor Applications

This example concerns the use of non-random mixtures or ensembles of cells for biosensing applications. The use of multiple cell types increases the information content of the experiments and facilitates the design of improved biosensor systems.

The multispectral optical changes of chromatophores can be used to evaluate exposure to bioactive agents. Automated cytosensors can be built to utilize this full set of color information. In this example, multiple colors are used to detect or quantify analytes or classes of analytes. The skin and scales of some brightly colored fish generally include patches of iridophores that reflect specific wavelengths of light as well as pigmented chromatophores that absorb various wavelengths of light. As a result, various colors can be produced as regulated by the nervous and endocrine systems of the animal. In this example, such multispectral features are used in a cytosensor for detection of many kinds of biologically active agents.

Scales of the West African Hemichromis bimaculatus include several color classes of chromatophores that are responsive to numerous bioactive conditions. For example, the agent DFP (at a concentration of about 0.5 mM or approximately 100 ppm) triggers optical changes in several color classes. As observed, DFP exposure triggers the reflected coloration of the iridophore patch to change within about 1 minute from predominantly blue to predominately green in appearance. Other chromatophores (e.g., melanophores, erythrophores, xanthophores) change with respect to an area occupied by cell pigment . Other biologically active substances (including norepinephrine, forskolin, and cholera toxin) also change the appearance of the Hemichromis bimaculatus chromatophores.

About 500 light reflecting centers are responsible for the light reflectance from a typical patch of iridophores, enabling the color changes in the iridophore patches to be easily visible. Reflection from an iridophore is produced by a subcellular organelle referred to as an iridosome. When viewed with an electron microscope, iridosomes include stacks of microcrystalline platelets separated by distances of between about ½ to ¼ of the wavelength of visible light. Such microcrystals are typically composed of guanine and other purines. Analyte-induced changes in platelet separations produce shifts in maximum reflectance wavelengths.

Because chromatophores include complex signaling pathways, chromatophores are sensitive to many agents. The class of organophosphate enzyme inhibitors can serve as an example that illustrates how chemical enzyme inhibitors change the appearance of multispectral populations of chromatophores. Organophosphate inhibitors (including biochemistry reagents such as DFP as well as notorious chemical warfare agents such as sarin) generally act via an irreversible reaction with active site amino acid residues of hydrolases.

Exposure of Hemichromis bimaculatus scales to aqueous solutions of DFP produced a pronounced color change from blue to green. This change was quantified with video microscopy and digital image analysis. The color changes and analysis are illustrated in FIGS. 4A-4C. FIG. 4A shows an example in which an iridophore patch of a scale is viewed by a mixture of transmitted and reflected light at low magnification. FIG. 4B shows quantitative changes in red, green, and blue response of the patch as a function of time after exposure to DFP. FIG. 4C represents patch color change in a hue-saturation-value (HSV) color space, showing that the color dimension hue angle (H) changed markedly from blue to yellow after DFP exposure, while color saturation (S) and the color-intensity (I, not shown) did not change significantly. Thus, cytosensor measurements can be based on hue determinations.

Using HSV as an indicator of exposure to DFP (the yellow-hued channel), cytosensor sensitivity can be increased. Digital color segmentation was used isolate the colors of the brilliantly reflective iridophores and to isolate the yellow-hued reflections. The yellow-hue increased with increasing doses of DFP. The threshold dose for triggering a detectable response was about 10 μM DFP.

In addition to hue changes in iridophores, other colored cell types also exhibited color changes with DFP, exposure such as melanophore and erythrophore pigment aggregation, and xanthophore pigment dispersion (not shown). Within 10 minutes of exposure to DFP, a series of changes were underway that were measurable in multiple spectral channels. Simplified illustrations such as those of FIG. 13 illustrate how multispectral chromatophores of Hemichromis bimaculatus scales are affected by DFP, other organophosphates, and several other cholinesterase inhibitors. The different agents induced differing responses in the multispectral set of chromatophores. A further improvement in differential capability was gained by employing a “failure mode” analysis, which determined how a given biological agent interfered with subsequent optical changes when challenged by a well-characterized agent (norepinephrine and forskolin are two examples of challenges shown here, other agents can be used).

The mechanism for organophosphate induced optical changes in multispectral populations of chromatophores is not completely understood. One target of organophosphates is acetylcholinesterase but acetylcholinesterase is not typically involved in the anatomical locale of chromatophores (subdermal skin regions), and so organophosphates are unlikely to affect jewel cicichlid chromatophores via the inhibition of acetylcholinesterase. Indeed, acetylcholine itself caused no color change. Thus, one or more among the many other serine hydrolases of cells could be the enzymatic targets whose inactivation leads to optical changes in chromatophores.

Many other agents and classes of toxic agents can be detected by the multispectral responses of chromatophores. For example, Hemichromis bimaculatus chromatophores changed in response to exposure to cholera toxin (1 nM, 2 hour exposure, purified cholera toxin from Sigma Chemical Co.). After this exposure, the melanophores were unimpaired upon challenge by norepinephrine (i.e., the black pigment aggregated as normal towards the center of the melanophores). However, erythrophore response was impaired as norepinephrine exposure did not produce the typical aggregation of red pigment.

These categories include toxic organophosphates, toxic pollutants (heavy metals, polyaromatic hydrocarbons, and various categories of pharmacological drugs). In addition, the entirely unique discovery has been made showing that chromatophores are methodologically effective in testing the bioactivity of compounds whose composition is entirely unknown at the time they were presented to chromatophores. Such compounds include compounds in extracts from algae (under study because of their potential content of interesting lead compounds for drug discovery) and compounds produced by microbial cells (under investigation as toxic pathogens from water sites, from food contamination, and from environmental contamination). Furthermore, the purity and concentration of the compound(s) under investigation can be unknown at the time of their exposure to a chromatophore-based biosensor as many different biologically active substances are detectable in various purities and concentrations using chromatophores.

### Example 13

### Use of Ensembles of Cells for Biosensor Applications

This example concerns the use of non-random mixtures or ensembles of cells for biosensing applications. The use of multiple cell types increases the information content of the experiments and facilitates the design of improved biosensor systems.

This example focuses specifically on identifying, detecting and □immers□n probiotic potential of commensal organisms using a cytosensor. Chromatophore biosensor cells were preincubated with lactococcal strains proposed to have probiotic potential before exposure to the toxin-producing, pathogenic Bacillus cereus strain. Preincubation with lactococcal strains prevented Bacillus cereus from causing the □immers□nts□e biosensor cells to rapidly aggregate. These results indicate that the chromatophore-based cyctosensor has the ability to identify, detect and analyze the probiotic potential of commensal organisms.

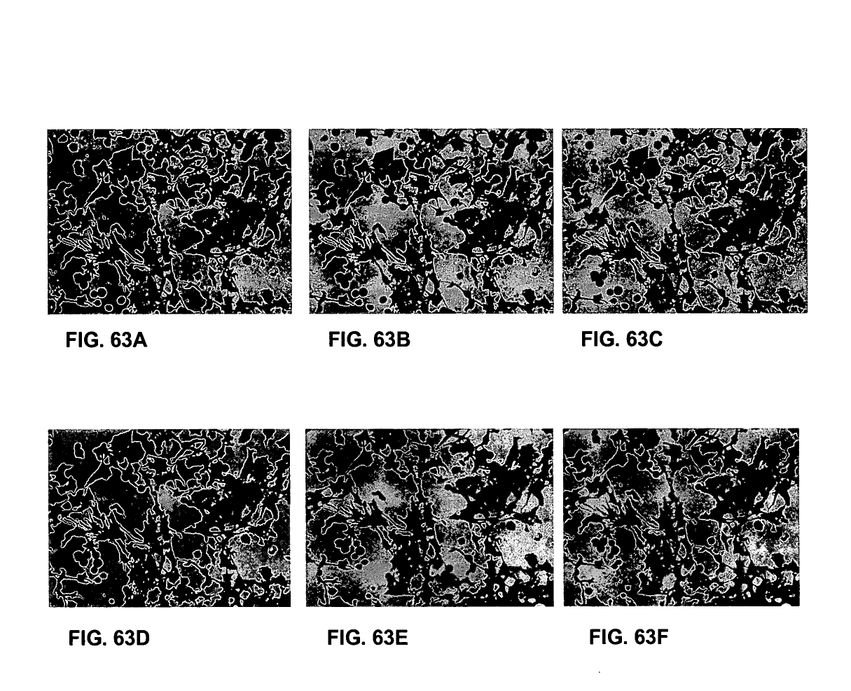

The results are illustrated in FIGS. 63A-63F. FIG. 63A illustrates the pigment pattern of chromatophores before exposure to Lactococcus. FIG. 63B illustrates the pigment pattern of chromatophores 5 minutes after exposure to Lactococcus. FIG. 63C illustrates the pigment pattern of chromatophores 30 minutes after exposure to Lactococcus. There is no obvious pigment redistribution (no aggregation) occurring in the chromatophore cells after exposure to Lactococcus. FIGS. 63D-63F illustrate the response of chromatophores to Bacillus cereus 5, 10, 30 minutes after the chromatophores have been preincubated for 30 minutes with Lactococcus. As illustrated by the data, Bacillus cereus can no longer elicit an aggregation response from the pigment organelles in chromatophores if the chromatophores have been preincubated with Lactoccus.

### Example 14

### Ensembles of Cells in a Microencapsulated Environment

This example demonstrates the encapsulation of non-random populations or ensembles of cells biosensor applications. The on-line creation of microcapsules containing both an environmental sample and chromatophores can be done with an extruder, such as shown in FIG. 18 that receives feed-streams of a gel-forming material to produce sub-millimeter sized spheres containing chromatophores and sample.

Fish cells (chromatophores) from Betta fish are isolated (as described above) from the fish tail and fins and mixed with sterile alginate solution. Polymerized alginate beads of uniform size (˜400 μm) are obtained by extruding the alginate solution through the needle of an air-jet droplet generator (see FIG. 18) and collecting them in the CaCl₂ solution. The beads are subsequently encapsulated within a semipermeable poly-L-lysine (PLL) capsule. The interior of the microcapsules is liquefied with citric acid. This enables a liquid environment for the fish cells within a permeable capsule that will allow the diffusion of nutrients and toxins of certain molecular weight ranges.

The fish cells are pelleted by centrifugation and resuspended in 5 mL of 1.5-2.5% sodium alginate solution that has been filter sterilized using a 0.2-μm filter. The sodium alginate solution is made by dissolving alginate powder into warm saline solution (0.85 g NaCl in 100 mL distilled water). The alginate is sprinkled into the saline solution, a small amount at the time, with gentle mixing. Once it has dissolved (approximately 1-2 h), the viscous solution is allowed to cool, and then it is transferred into plastic tubes, capped, and stored in refrigerator until required.

The fish cells that were resuspended in sodium alginate solution are then extruded using either a syringe or an air-jet droplet generator into 100 mL of a 1.5% CaCl₂ solution. Ferromagnetic particles are added to the cell/alginate mixture prior to extrusion. The ferromagnetic particles help control motion of the microcapsules in the biosensor device using a magnetic field

The resulting beads are then transferred to a sterile 50-mL plastic centrifuge tube with a conical bottom. The beads are washed (10 times) with 30 mL of 0.1% 2-α-cyclohexylamino ethane sulfimic acid. A 0.1% CHES solution is prepared by adding 5 mL of CHES stock solution to 95 mL of 1.1% CaCl₂ solution, CHES stock solution is made by dissolving 2 g of CHES and 0.51 g of NaCl in 90 mL of distilled water, adjusting the pH to 8.2 with NaOH, and increasing the volume to 100 mL. After allowing the beads to settle (for up to 60 s), the volume of the supernatant is reduced with a vacuum aspirator.

A semipermeable capsule membrane is then formed by reacting the gel droplets with 30 mL of a 0.05% (w/v) poly-L-lysine (PLL) solution (PLL hydrobromide—SIGMA (Mw=30,000-70,000) in sterile saline. The resulting capsules are then allowed to settle, the excess PLL solution is aspirated off, and then the capsules are washed with 30 mL each of 0.1% CHES and 1.1% CaCl₂ and with two aliquots of saline. The capsules are then resuspended in 30 mL of 0.03% sodium alginate solution for 4 min to form an outer layer on the capsules and to neutralize free active groups on the PLL membrane.

The interior of the PLL microcapsule is liquefied by suspending 5 mL of the capsules in 30 mL of a 0.05 M sodium citrate solution (2.58 g of sodium citrate and 0.85 g of NaCl in 200 mL of distilled water) for 6 minutes. The capsules are then washed several times in saline to remove any excess citrate and then rocked end-to-end for 30 min to allow the alginate to diffuse out of the capsules, and for the capsules to swell toward their equilibrium state.

The capsules are now ready to be incubated with various test compounds. Incubation permits bacteria to express toxins that are detected by chromatophores.

### Example 15

### Use of Ensembles of Cells in a Microfluidic Environment

This example is directed to using non-random mixtures of biosensor cells. One or more cell chambers were micromachined in polycarbonate. In representative examples, 2 and 5 cell chambers were defined. Each of the chambers can be supplied from a common reservoir. Such a design allows flexibility in fluidic connections as well as in a number and use of chromatophore chambers. Supply lines or inlet channels can be independently controlled with valves or pumps to set desired flow rates for different analytes.

Syringe pumps situated upstream of the chambers can be used to apply the sample to successive chambers. Each chamber can include individual control of sample intake volumes and flow rate using valves or with pump control. A common downstream pumping mechanism can be used to maintain a basal flow of fluid to the bank of chambers. The downstream pump can be a syringe pump, a capillary wick, a negative partial pressure, or some other pumping system. All chambers are typically configured with a basal flow of fluid that can be achieved by either individual pumps or a common downstream pump. Such a pump can be a syringe pump, a wicking type pump mechanism, an electro-osmotic type, or some other pump. A multi-stream arrangement can be used to direct parallel and separate streams through a common chamber to achieve concurrent sampling in a common cell chamber. Multiple streams within a single chamber may be possible and thereby permit introduction of multiple analytes into a common chamber. By setting the proper flow conditions these streams can remain separate within the chamber. A simple two-stream prototype was used to evaluate the extent of fluid mixing by adjacent streams within a single chamber and showed that fluid mixing control is achievable.

An illumination system includes a light source such as a laser diode, light emitting diode, or lamp that emits radiation that is directed to the sensor cells. An optical system can be provided to shape and direct the radiation, and mirrors or beamsplitters can be configured so that a single light source illuminates more than one cell chamber. One or more cameras are arranged to receive radiation from the cell chambers. A single camera can be arranged to image cells in one or more cell chambers using relay optics, or several cameras can be used. Multitasking can be used to enable one or more computer systems to process the images collected from multiple chambers.

This examples concerns using a cytosensor that includes an analyte entry tube that delivers an analyte to a reaction cell that contains selected chromatophores. The reaction cell defines a reaction chamber that includes side walls and one or more transparent windows, and the chromatophores are held stationary in the reaction chamber. In a specific embodiment, the analyte entry tubing is 0.25 mm inside diameter TEFLON tubing, and a 1 microliter analyte volume is introduced into the reaction chamber. The analyte can be introduced with a syringe, a syringe pump, a peristaltic pump, or other pump. Alternatively, the analyte can be introduced by wicking with, for example, cellulose fibers, or using a capillary flow.

Chromatophores introduced into the reaction chamber can be retained in the reaction chamber by permitting the chromatophores to settle onto a chamber surface. Superior chromatophore retention is achieved by treating the chamber surface with an attachment factor such as collagen and/or fibronectin. Attachment of the chromatophores is generally adequate 1-24 hr after introduction, and can be verified by observation with a light microscope. After the chromatophores are attached to the chamber surface, flow rates into and out of the chamber can be increased without dislodging the chromatophores.

FIGS. 26A-26B illustrate one embodiment of a reaction cell. A reaction chamber is defined in a laminated stack of sheets of a transparent, adhesive-backed material such as MELINEX. The reaction chamber is typically rectangular, with representative dimensions of 2 mm by 3 mm by 0.4 mm. The 0.4 mm depth of the chamber is generally selected as an integer multiple of the thickness of the sheets laminated to form the reaction cell. Inlet and outlet apertures of about 0.5 mm diameter are provided for introduction of and extraction of analytes and other reagents.

As shown in FIG. 26A, the reaction cell is mounted to a fluid interconnect. The fluid interconnect includes a top plate and a bottom plate having central apertures to permit illumination of the chromatophores. These plates are illustrated in FIGS. 27 and 28A-28B. Mounting holes are provided for attaching the fluid interconnect to the reaction cell, and fluid inlets/outlets are provided as well. Bolts (not shown) are used to connect the top and bottom plates, sandwiching the reaction cell.

FIG. 29 is a diagram of an alternative reaction cell that defines two reaction chambers. A stack of polyimide layers and copper layers are laminated by heating the stack to that the polyimide layers bond the copper layers. The stack is terminated on a first surface with a first end cap, and on a second surface with a second end cap that includes inlet/output apertures for any necessary analytes or other materials. Reaction cells that include fewer than or more than two reaction chambers can be fabricated similarly.

FIGS. 30A-30C are schematic diagrams of a multi-analyte reaction cells that include analyte reservoirs and FIG. 31 is photograph of a micromold for fabrication of reaction chambers.

One or more light sources such as LEDs, a laser diode or other laser, or lamps are situated to illuminate the chromatophores. In an embodiment, illumination from the light sources is collected with a lens that directs the collected illumination to the chromatophores. Light reflected by or transmitted through the chromatophores is collected with a lens and directed to a imaging system that includes a CCD or other camera. Typically, the chromatophores are imaged onto the CCD. The CCD supplies an electrical image signal (typically an analog NTSC video signal) to a digitizer that produces a digital image of the electrical image signal that is supplied to a computer. Alternatively, an image sensor that provides a digital image directly can be used. The computer receives the digital image and stores the digital image in a tagged image file format (TIFF) or other convenient format for processing. Images are generally obtained at a rate of about 2/sec. The apparatus is configurable for measurement of transmitted or reflected light. Typically, transmitted or reflected light is suitable for melanophores, erythrophores, and xanthophores, but for some chromatophores such as iridophores, reflected light is superior. The apparatus is also configurable for measurements of fluorophores.

Images are typically acquired in a red-green-blue (RGB) color representation in which values (r, g, b) for each of the colors R, G, B are assigned to each pixel. Other representations can be used, such as a hue-saturation-intensity representation, or other representations. After conversion into the selected color representation, the image data is segmented into areas corresponding to one or more predefined colors or color ranges that are based on the selected chromatophores. A color segment is defined as a predetermined number of adjacent pixels having a color within a predetermined range as described herein.

In a specific example, the image sensor is a ½″ interline transfer CCD color camera. Color camera variables include the number of the CCD arrays (e.g., 3-CCD arrays with RGB filters), CCD formats (typically ⅓″, ½″ and 1″), and resolution (e.g., the number of horizontal and vertical pixels N, M, respectively). For example, a CCD camera can have N=768 columns and M=494 rows of pixels, or 379,392 total pixels and the CCD cell size is 8.4 μm (H)×9.8 μm(V). The camera produces an analog RGB signal that is digitized using a multi-channel frame grabber having sampling rates up to 30 MHz and a data transfer rate of 130 MB/second. A digital color camera can also be used. Such a camera typically has a variable speed shutter (1/60 sec. to 1/10,000 sec.) and can be operated both synchronously and asynchronously.

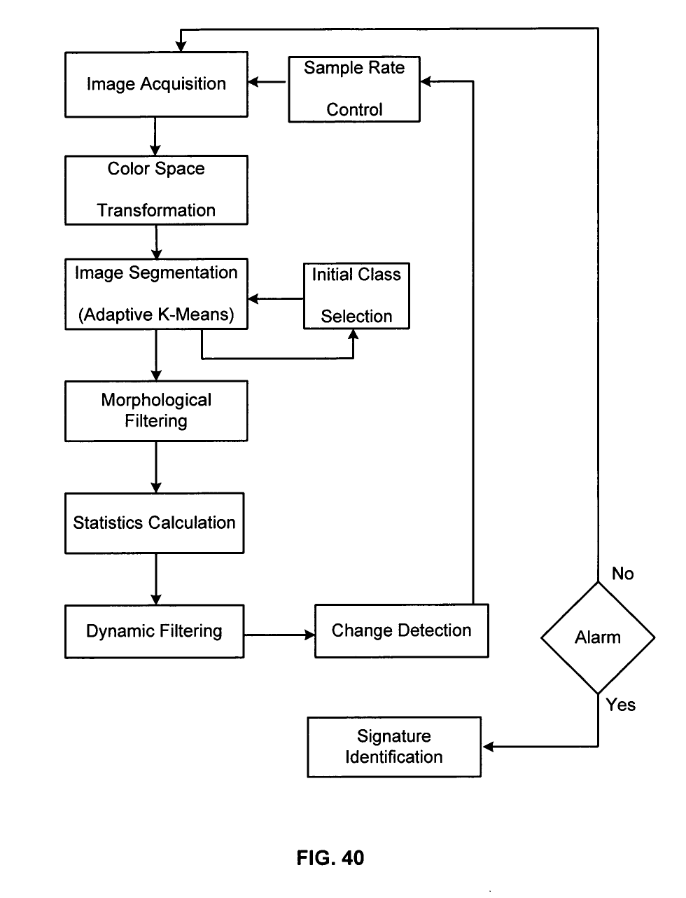

FIG. 40 is a block diagram of a method of processing the digital images that uses a time sequence of images acquired either asynchronously or synchronously. Images are typically collected at about one image (frame) per second, but the collection rate is adjusted based on the rates of change of image parameters calculated from previous image and acquisition rates of up to 30 images per second are achievable with conventional image sensors. An optimal acquisition rate can be selected to match the estimated (on-line) rate of change of the image content.

Each image is represented by N×M array A, whose entries are P dimensional vectors. Such an array can be viewed as P dimensional discrete field. N and M define a grid that divides the image into small rectangular regions called pixels or picture elements, e.g., a field of view of dimensions X by Y is divided into pixels (rectangles) of dimensions (X/N) by (Y/M). The P-dimensional discrete field model represents most digital image formats, including the common RGB format. Thus each entry of A represents an average color from a picture element using a P=3 dimensional vector of intensities of red, green and blue color components. Typically these intensities are normalized to take values between 0 and 1. The generalized color histogram can be then viewed as a map of A into a 3 dimensional unit cube. Since the image is a discrete description of the optical appearance of the field of cells, the analyzer can use colors and shapes to characterize the cell state.

In a color space transformation step, the representation of the image in RGB space is transformed to a suitable color space where the distance between colors can be defined to provide, for example, visual uniformity. In an example, a HSV (Hue, Saturation, and Value) space is used.

After color space transformation, an image segmentation step is executed to divide the image into segments based on pixel color. Color segmentation is performed by selecting a specific color coordinate or combination of coordinates, and characterizing pixels based on this coordinate (or coordinates). This process is based on a generalized K-Means algorithm. A color cluster is a set of colors which are “alike,” and the colors from different clusters are “not alike.” A quantitative description of a cluster is “an aggregation of points in a multi-dimensional space, such that the distance between any two points in the cluster is less than the distance between any point in the cluster and any point not in it”. Intuitively clusters are connected regions of a multi-dimensional space containing a relatively high density of points, separated from other such regions by a region of a relatively low density of points. The segmentation takes place in a color histogram space. The distance D is calculated using a quasi-periodic measure based primarily, in a representative example, on the hue coordinate (H) of the image pixels and secondarily, on a contribution based on the saturation coordinate (S). A number of color classes K is adjusted adaptively using dynamic programming techniques. The smallest K is sought such that a color variance (calculated using D) of each of the clusters does not exceed an a priori specified threshold. The dynamic programming method re-uses the initial segmentation for K classes to perform efficiently (K+1) class segmentation. Typically, the image is divided into 5-10 color classes.

After segmentation, the color clusters are associated with their corresponding image (spatial) coordinates for evaluation of cluster geometrical shape in a morphological filtering step. Morphological filtering is applied to each color class to eliminate small spatially isolated clusters and to merge close clusters. For example, morphologically filtered clusters should correspond to the size expected for an image of a chromatophore or a group of chromatophores. The morphologically filtered image contains areas that can be associated with erythrophores, melanophores, and other chromatophores as well as a background. Statistical and morphological parameters of the filtered clusters are calculated in a statistics calculation step. Representative calculated parameters of the color clusters include area, convex hull, bounding polygon, convex area, equivalent diameter, major and minor axis lengths, solidity, extent, orientation, eccentricity, and nth order moments (both spatial using Euclidean distance, and in color space using, for example, the distance D).

In a dynamic filtering step and a change detection step, changes in filtered color clusters are detected based on an analysis of series of images rather than a single image. Dynamic patterns (trends) of selected statistical or other parameters are estimated (for example, area and 2^{nd} order spatial moments), using a conditionally linear filter, which generalizes traditional Kalman filtering methods. This filter tests sequentially two alternative hypotheses about the parameters of a mixture of two distributions. The null hypothesis assumes that there is no bioagent present and that the cells statistics are slowly varying. The alternative hypothesis assumes that after being exposed to a bioagent the cell statistics reach an equilibrium that is distinct from the initial state equilibrium. In a representative example, a mixture of two Gaussian distributions is used having a slowly varying mean and variance and a second having constant moments. The test control parameters include probability of false alarm and detection delay. The acceptance of the second hypothesis marks the end of detection process and stops the image acquisition.

Dynamic patterns are parametrically identified from the statistics waveform in the transition region (i.e. in the non-causal neighborhood of the time instant signaled by the detection algorithm) using, for example, a BARMAX (Bilinear Auto-Regressive Moving Average) model, but various parameterized models can also be used. The estimated model parameters are matched against a stored library of template signatures. These template signatures represent the results of controlled experiments in which chromatophores are exposed to known (type and concentration) bioactive conditions. Since thus matching problem is of a low dimension (number of model parameters), various statistical (geometric) pattern recognition techniques such as proximity matrix methods can be used here. Some of the cell responses to specific bioactive conditions lend themselves to structural (syntactic) pattern recognition. In this approach the patterns are composed of simple sub-patterns. A sub-pattern can be built from simpler parts with grammatical techniques. Primitives (or the simplest sub-patterns) can be shared among many different experiments to reduce the need for extensive experimentation and the creation of large signature libraries. The outcomes of pattern matching for various statistics represents “votes”, defining the confidence level of agent identification.

Such a detection method is implemented on a general-purpose microprocessor such as an embedded Intel Pentium processor running Windows NT or a dedicated processor. Numerical calculations can use fixed-point arithmetic and are suitable for implementation on DSP type boards. The color segmentation algorithm is multi-threaded and can be easily ported to a multi-processor computer or multi DSP chip board. The identification and pattern recognition algorithms have hierarchical organization suitable for communication between several sensors in order to obtain more reliable detection results.

The representation of the cells state as a mathematical field and the general segmentation algorithm allow use of multi-spectral (not necessarily visual) images. Due to the sequential data processing the storage (i.e. memory) requirements are minimal. The system minimizes energy use by adapting the image sampling rates, which is an essential feature in mobile device operation.

The representation of the cell's state as a mathematical field and the general segmentation algorithm allow use of multi-spectral (not necessarily visual) images. Due to the sequential data processing the storage (i.e. memory) requirements are minimal. The system minimizes energy use by adapting the image sampling rates to permit mobile device operation.

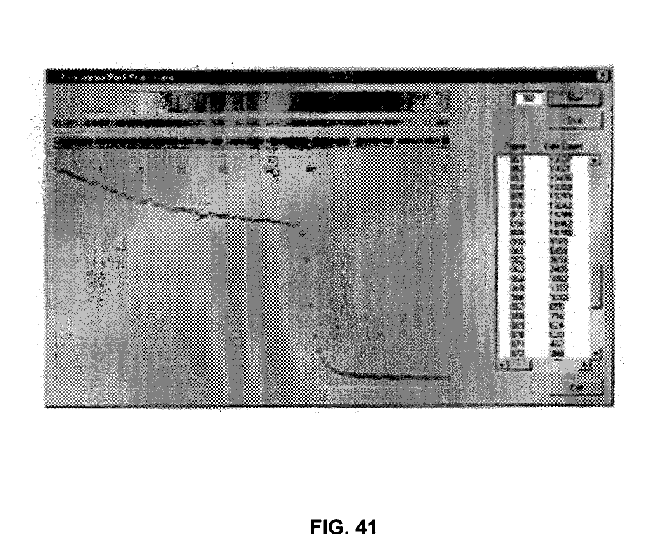

FIG. 41 illustrates results of image processing as described above. The screen image of FIG. 41 is based on 92 images that are acquired at a rate of about 5 images/second. An alarm is triggered at a 60^{th} frame, with a delay of two frames. The signature identification uses 10 frames (from the 58^{th} frame to 67^{th} frame). Frames 1 to 57 correspond to slowly changing statistics (null hypothesis) and frames 68 to 92 represent equilibrium.

For convenience, FIGS. 5A-5B are provided to illustrate a change in an image hue coordinate after exposure to a selected analyte. While RGB color coordinates change (as shown is FIG. 5A), the change in hue of FIG. 5B is more apparent.

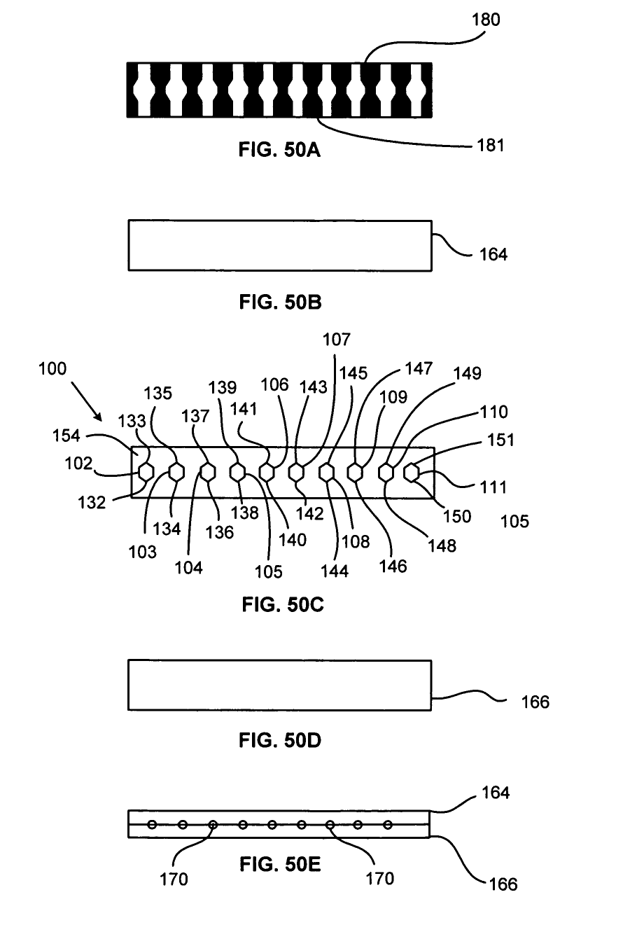

Cytosensor chambers can be formed using conventional machining, laser machining, photolithographic processes, or other methods. Referring to FIGS. 50A-50E, a chamber array 100 includes chambers 102-111 that are defined by recesses in a polycarbonate plate 121. The chambers 102-111 are provided with channels 132-151 that extend toward edges 152, 154 of the plate 121. A chamber assembly 160 includes the plate 121, a top plate 164, and a bottom plate 166. The top plate 164 and the bottom plate 166 are attached to the plate 121 by sonic welding, an adhesive, or with solvent welding. In one example, the plate 121 is exposed to a mixture of methylene chloride, methyl alcohol, and toluene. After exposure, the mixture is blown from the plate 121 and the plate 121 is contacted to the top plate 164 and the bottom plate 166. The plates 121, 164, 166 are pressed together for bonding and can be conveniently aligned using alignment holes configured to fit on an alignment pin made from, for example, drill rod or other material. After bonding, the chamber assembly 160 can be treated to eliminate any solvent residue by heat treating, or other method.

Fluid ports 170 extend from the chambers 102-111 to sides 180, 181 for fluid entry and discharge. The fluid ports 170 can be formed by boring holes from the sides 180 to the channels 132-151. Fluid connection to the channels 132-151 can be made by inserting tubing such as microchannel tubing into the holes so that a leak free seal is formed. Capillary action or gravity can be used to direct fluids into one or more of the chambers through the tubing. In some examples, one of either the top plate or the bottom plate is white polycarbonate to enhance viewing of chromatophores. Typically the top plate is clear to permit viewing contents of the chambers, but some plates can be made of clear or black polycarbonate or other materials.

### Example 16

### High Throughput Screening Biosensor Based on Microencapsulation of Ensembles of Cells

This example concerns selective routing of samples through high-throughput screening devices that can be used in many applications, including biosensor methods. The use of non-random populations of encapsulated cells in microscale devices will increase the efficiency of high throughput screening applications. Bio-capsules, capture dots; and micro-ball-valves that might constitute such a system also are described.

An encapsulation machine can be used to take periodic samples of analytes and package them for optical measurements, using a method of continuous encapsulation of sensor cells. After formation, capsules can be arranged in an order that allows each to be associated with the period of time that a respective sample was fed into the system. Controlled microfluidics and/or movements achieved by the use of magnetic fields on suitably doped capsules can be used to move the capsules to positions within an instrument where they can be optically observed. An optical detection system can be used to measure optical appearance of the chromatophores within the capsules.

Various types of illumination can be used to irradiate the capsules, including a fluorescent lighting arrangement based on innate fluorescence of some chromatophores (including erythrophores), or fluorescence of a marker introduced into the chromatophores.

An analyzer with parallel processing capabilities or other suitable hardware architecture can be used to keep track of the optical appearance of the capsules and determine whether a significant change occurs in any capsule. Given that each capsule is associated with a particular sample entry time, results can be associated with analyzer times at which biologically active agents are introduced.

A sensor cell feed line provides chromatophores in a form that makes them readily available for encapsulation and an analyte feed line receives a liquid sample that is directed to an encapsulation zone. The encapsulation zone is a portion of a cytosensor at which sensor cells and analyte are mixed together and, in conjunction with gel-forming and gel-dissolving components, capsules are formed.

Micro-balls can be used in biomaterial carriers (capsules containing bio and ferromagnetic material) and in micro-ball valves. Apparatus for the immobilization of biomaterial in alginate beads can be adapted for micro-ball production. Several microball compositions have been made and several thousands of micro-balls have been produced. Micro-balls of different sizes, densities, and percent by weight (Wt %) of ferromagnetic material have been produced. Micro-balls having diameters dp in a range of about 150-1000 μm, ferromagnetic Wt % of about 5%-20%, and densities in a range of about 1.05 g/cm³ to 1.35 g/cm³ have been produced.

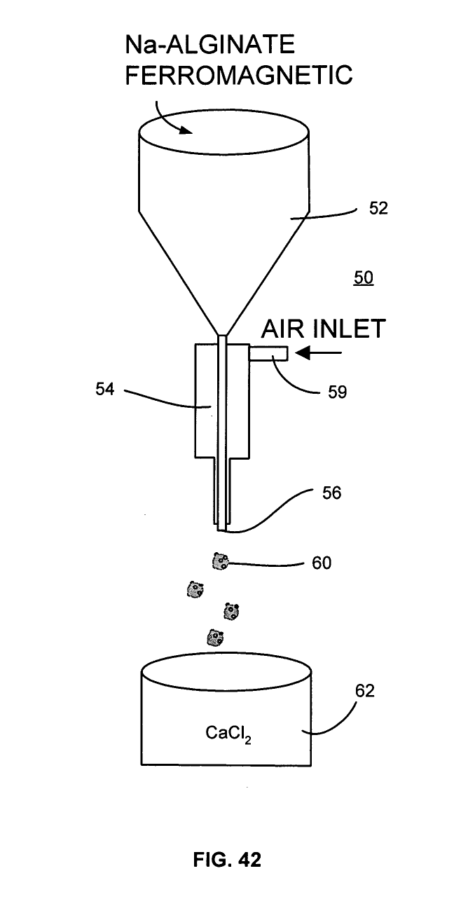

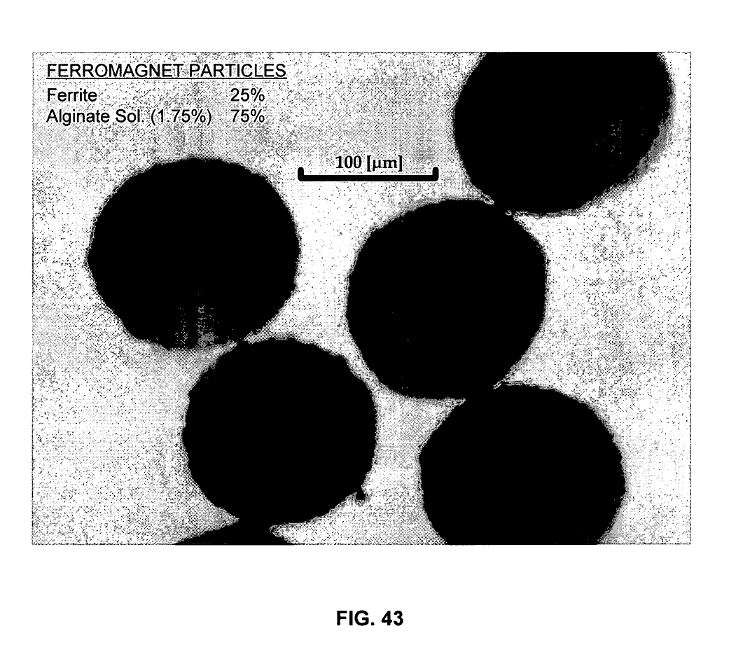

Micro-ball carriers are preferably in a size range of about 100-200 μm and can be produced with an extrusion apparatus such as apparatus 50 of FIG. 42. A mixture of 1.5% sodium alginate and 95% water is combined with a ferromagnetic material and an active substance and placed in a reservoir 52. The mixture is directed through a needle 54 or other channel and air is directed to a tip 56 of the needle 54 through an air inlet 58. Introduction of air into the inlet 58 shears off beads 60 at the tip 56. The beads 60 are directed into a CaCl₂ bath 62 in which Ca²⁺ is exchanged for sodium ions Na⁺ so that the alginate material is polymerized. The duration of exposure to the CaCl₂ bath determines the degree of polymerization. FIG. 43 is a photograph of several micro-balls (dp≈150 μm) produced with the apparatus 50.

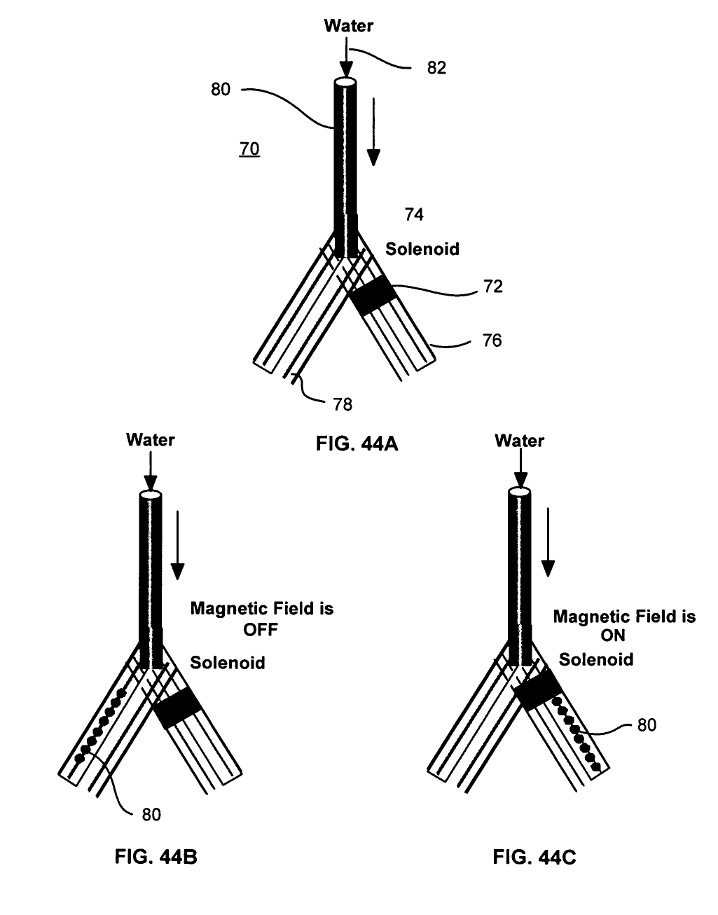

High-throughput screening devices typically require selective immobilization of biomaterial within a maze of micro-channels. Devices incorporating this capability are preferably programmable for a variety of operating configurations. Referring to FIGS. 44A-44C, a Y-branch 70 permits micro-balls containing a ferromagnetic material to be directed to a channel 76 as controlled by a magnetic field generated by a solenoid 72 positioned at an entrance 74 of the channel 76. Micro-balls are directed to one side of the Y-branch 70 by activating a magnetic field for a selected channel (e.g., channels 76, 78). As illustrated in FIG. 44B, with no applied magnetic field (the solenoid 72 in an off state), magnetic particles 80 in a liquid 82 are directed to both channels 76, 78. With an applied magnetic field as shown in FIG. 44C, the particles 80 are directed to the channel 76. In a specific example, micro-balls (the particles 80) of diameter dₚ=500 μm were directed to a Y-branch having internal diameters of about d_{ch}=1000 μm. In other examples, particles of diameter of dₚ≈100 μm were directed into channels of internal diameter d_{ch}≈200 μm.

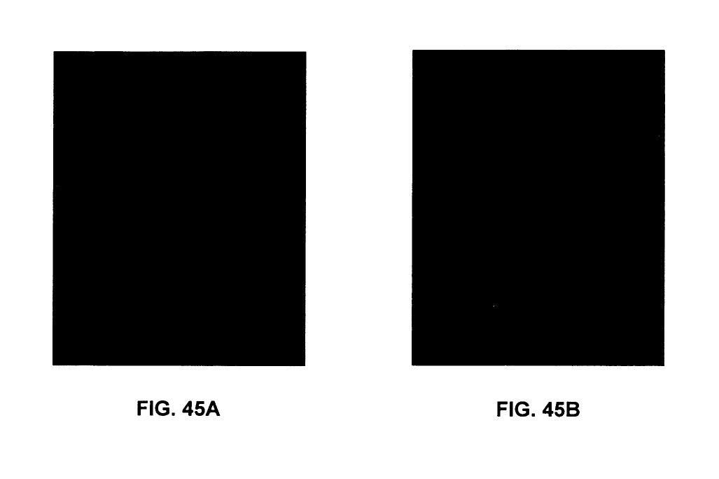

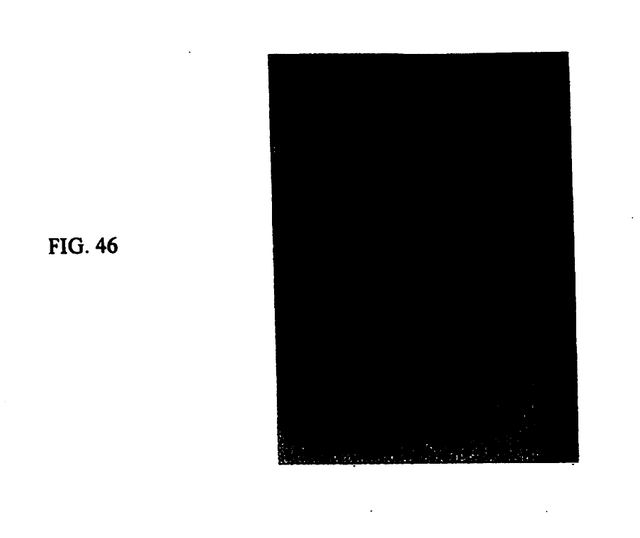

Detailed calculations of magnetic fields from the thin film coils and magnetic forces on ferromagnetic beads can be used to determine capture dot geometry. Capture dots can be produced by electroforming into a lithographic mold. Round and square coils have been produced using various photolithographic (PL) test patterns. These patterns can include an oxide via to bottom conductor layer, a pad layer for electrical contact, a coiled conductor layer, and a test structure layer. Multiple patterns can be formed on a single mask. Some examples of mask patterns are shown in FIGS. 45A-45B and an electroformed coil made from the pattern of FIG. 45A is shown in FIG. 46.

A method for making contact printing masks for photolithography includes evaporation of a 0.5 μm chromium layer onto a glass microscope slide or other substrate. The chromium layer is selectively ablated using a 532 nm Nd:YAG laser. Because glass (SiO₂) is transparent to the 532 nm wavelength, the laser ablates the chromium while leaving the glass surface intact. Linewidths at least as small as 35 μm can be produced. The glass surface is typically somewhat “frosted” indicating that some amount of laser ablation or other effect occurs at the glass surface. Masks can also be made by direct writing with an ultraviolet (UV) on Cr/glass plates or by exposing photoresist/Cr/glass plates for etching of the Cr layer.

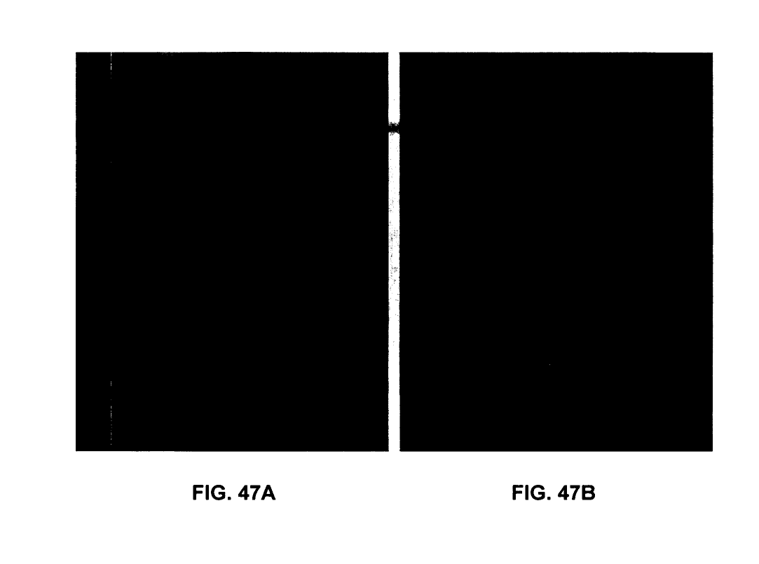

A two-way micro-ball valve based on capture dots technology for actuating a ferromagnetic ball on the order of several hundred microns can be used. A one-way micro-ball valve is shown in FIGS. 47A-47B. The micro-ball valve includes a valve chamber between a valve orifice (FIG. 47A) and a ball catch plate (FIG. 47B). The device is actuated hydraulically and permits flow in one direction. When flowing from the orifice to the catch plate, the ball is caught in the catch plate allowing flow through the valve. When flowing from the catch plate to the orifice, the ball seals the orifice causing flow to stop. The catch plate thus permits flow through the valve while retaining the ball.

The valve shown in FIGS. 47A-47B defines a chamber of approximately 900 μm diameter and has a glass ball of approximately 400 μm diameter. The valve is made of 9 layers, alternating 0.005 inch MELINEX 453 polyester film with Avery Dennison FT8311 double-sided pressure sensitive adhesive film. There are four FT8311 laminae interspersed between five MELINEX laminae. The two outside laminae are patterned as shown with the orifice and catch plate. The inner laminae are patterned with a circle of the diameter of the valve.

Several one-way valves similar to those of FIGS. 47A-47B were built and tested. Leakage within a diode (one-way) valve can be quantified as a diodicity (i.e. a ratio of pressure required to give an intended flow rate in a first direction to the pressure required to create the same flow rate in a second direction, opposite the first (an undesired flow direction). Table 5 summarizes the diodicity results from a particular valve.

TABLE 5
Micro-ball Valve Diodicity

| | Forward flow | Reverse flow | |
|---|---|---|---|
| Flow rate | Pressure | Pressure | |
| (ml/min) | (psi) | (psi) | Diodicity |
| 5 | 1.05 | 0.37 | 2.84 |
| 8 | 1.48 | 0.95 | 1.56 |
| 9 | 1.6 | 1.12 | 1.43 |
| 10 | 2.96 | 1.4 | 2.11 |

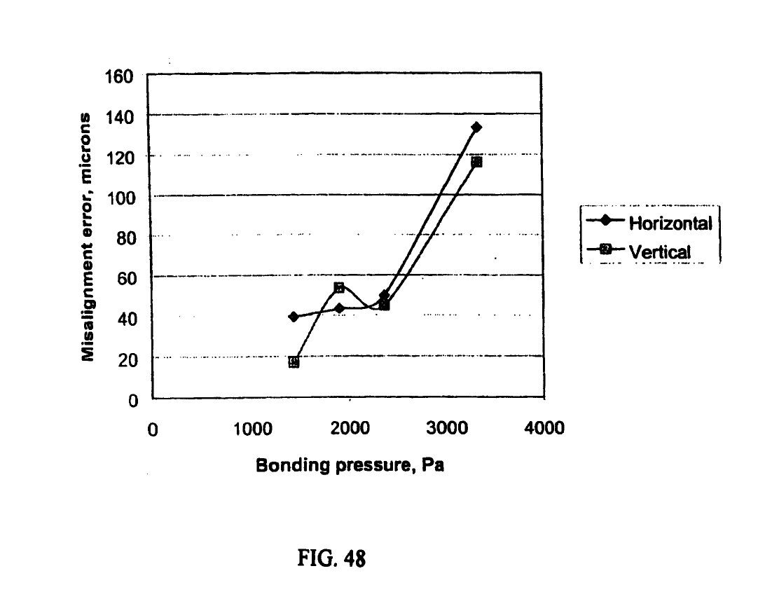

The diodicity of this valve is limited by alignment errors between layers that prevent the ball from sealing the orifice. Misalignment as a function of bonding pressure was measured and is graphed in FIG. 48.

In other examples, adhesive appeared within the chamber causing the ball to stick as the pressure sensitive adhesive oozes under high bonding pressures. Therefore, the design for the middle chamber laminae was changed so that the circles cut in the FT8311 laminae for the chamber were larger than the circles cut in the MELINEX laminae. This was done to keep the ball from being exposed to any excess adhesive that might be squeezed out from between laminae. In addition, misalignment can still occur and alignment holes were provided to reduce misalignment. Use of a 266 nm Nd:YAG laser for patterning MELINEX layers and FT8311 laminae reduces thermal damage and improves chamber geometry.

TABLE 6
Micro-ball Valve Diodicity

| | Forward flow | Reverse flow | |
|---|---|---|---|
| Flow rate | Pressure | Pressure | |
| (ml/min) | (psi) | (psi) | Diodicity |
| Valve diameter = 700 microns | | | |
| 1 | <0.01 | 28 | >2800 |
| 5 | 0.025 | 28 | 1120.0 |
| 7.5 | 0.3 | 28 | 93.3 |
| 10 | 0.305 | 28 | 91.8 |
| 12.5 | 0.52 | 28 | 53.8 |
| Valve diameter = 900 microns | | | |
| 5 | <0.01 | 28 | >2800 |
| 7.5 | 0.13 | 28 | 215.4 |
| 10 | 0.27 | 28 | 103.7 |
| 12.5 | 0.45 | 28 | 62.2 |

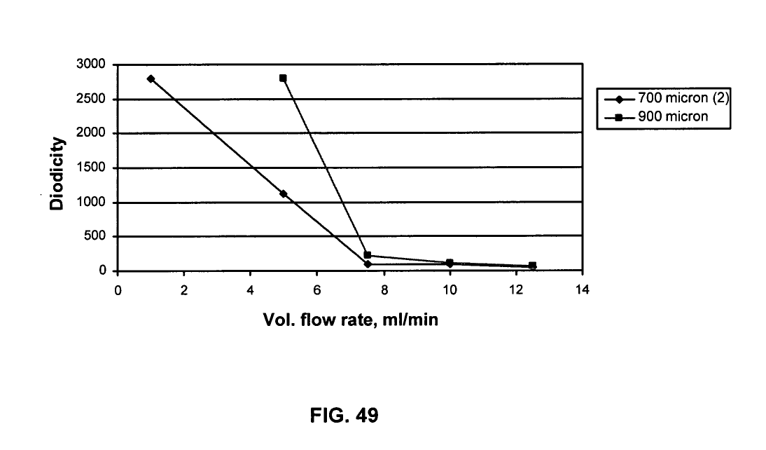

Valve performance with these improvements is illustrated in Table 6 and FIG. 49. All reverse flow pressure drops in Table 6 are 28 psi, limited by a pressure gauge in a measurement system. Therefore, diodicity for the valve at the tabulated flow rates is at least about 2,800. Leakage between the glass ball and the polyester orifice be less than 10 microliters/min at 28 psi. Such valves can include magnetic balls so that the valves are electromagnetically activated.

### Example 17

### High Throughput Screening Biosensor Based on Microfluidic Chambers Populated with Ensembles of Cells

A method for decreasing the variation in cell response by maintaining the cells in a microfluidic environment.

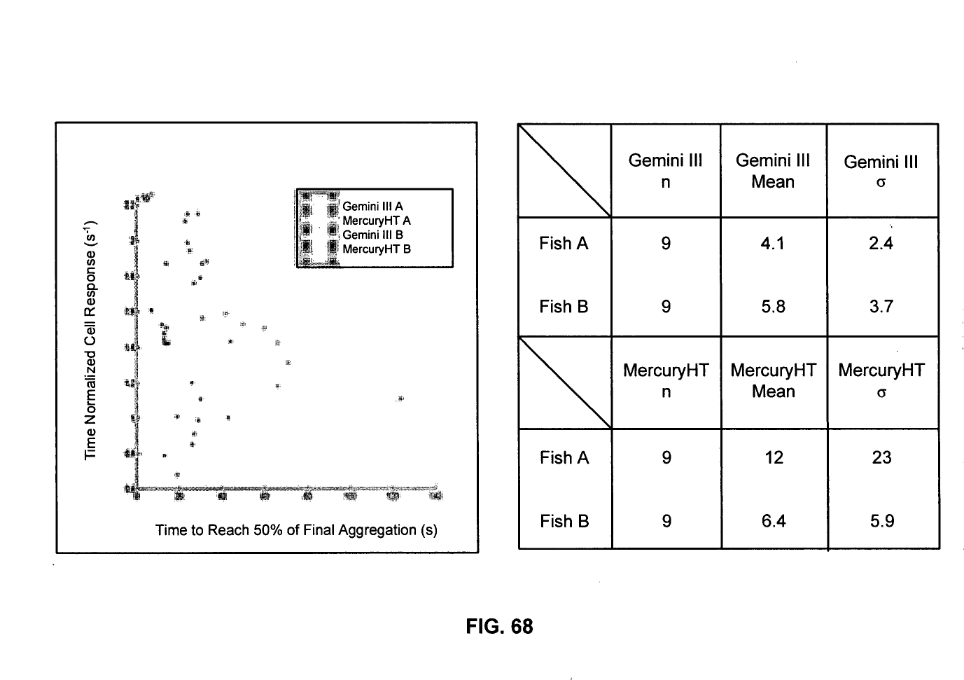

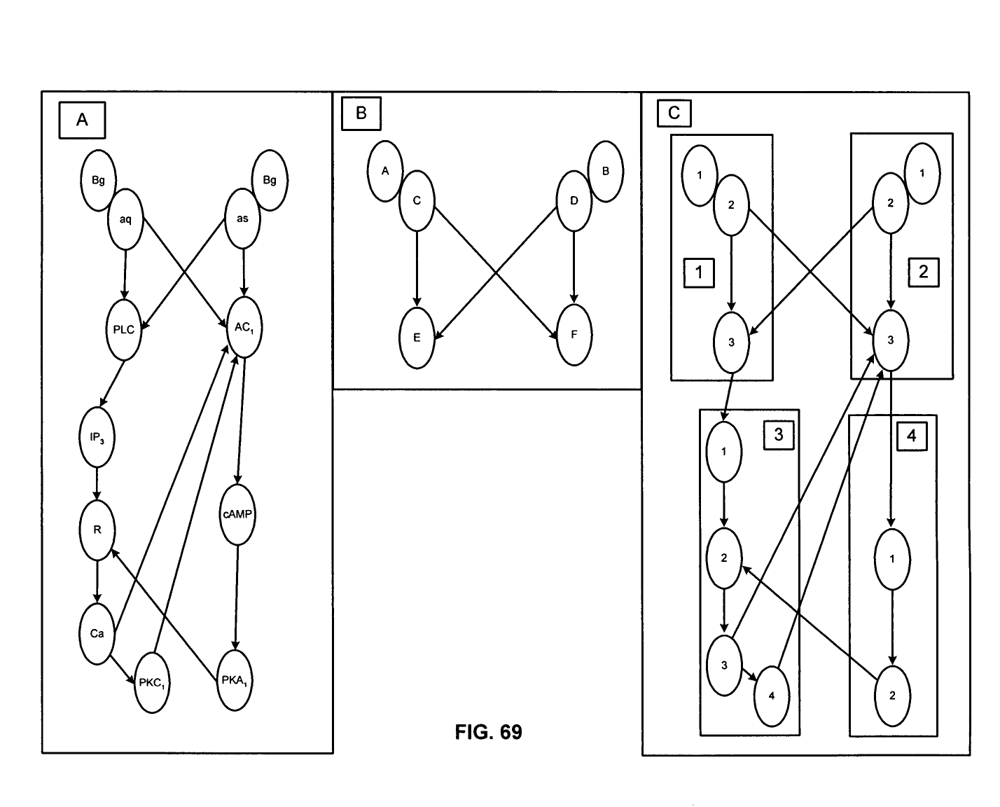

Microfluidic systems inherently provide more tightly controlled mass and momentum transfer environments. Preliminary results demonstrate that this has a significant effect on the quality of information extracted from cell-based biosensor systems. FIG. 68 shows a comparison of results from two different systems. Gemini III is a microfluidic platform developed through DARPA funded research. MercuryHT is the commonly used 24-well tissue culture plate format. Data clusters obtained from the microfluidic device platform show less scatter than those obtained from the tissue culture wells. The effect of genetic variation in fish stocks is also evident from these experiments. Much of the error associated with the tissue culture plates occurs as a consequence of inconsistencies in mixing during the initial exposure to agents. With reference to FIG. 68: Gemini III is a microfluidic chamber with dimensions of 20001 μm×1000 μm×500 μm (l×w×h). Mercury HT is a standard 24-well tissue culture plate. Results for two fish (n=9 experiments from each fish). Fish were anaethesized in ice cold water. Fins were removed, washed in phosphate buffered saline/EDTA and digested with a mixture of collagenase type I and hyaluronidase before plating in microfluidic chambers or tissue culture plate. Each data point represents a single experimental run. Data from each experimental run was in the form of a time series of images. The data from each segment was reduced to two parameters representing the normalized middle value of the response and the time required to reach the middle value point. The normalized middle value represents the point where the cells reach their half aggregation status in the whole process. The data from the nine experiments forms the cluster documented in the figure. Cluster size was captured by measuring the distance from each point to an imaginary center point and calculating an associated standard deviation.

### Example 18

Chromatophores can be used to detect bioactive compounds that act upon specified molecular targets. For example, one important class of compounds includes pharmacological agents that act upon the membrane channels that admit calcium ions into cells. Drugs based on the activation or inhibition of these membrane channels can be useful in treatments of various diseases and syndromes. To provide such chromatophore-based sensors, sensor cells are evaluated for the presence of a calcium ion channel. As a specific example, calcium-dependence of pigment transport in melanophores and erythrophores was evaluated.

The signal transduction pathway initiated by the formation of cAMP by adenylate cyclase has been relatively well characterized in its role on intracellular motility in erythrophores and melanophores. This enzyme is closely connected to the α₂-adrenergic receptor, the stimulation of which leads to the direct modulation of intracellular levels of cAMP. These levels have been directly implicated in both the aggregation and dispersion of pigmented vesicles within the cytoplasm. Decreased intracellular levels of cAMP have been shown to induce rapid aggregation of vesicles while increases in cAMP levels lead to dispersion. An alternate signal transduction pathway that has been implicated in vesicle motility in erythrophores involves the role of calcium concentration in the cytoplasm. The role of calcium in erythrophores is relatively misunderstood compared to the role of cAMP. Conflicting results have been obtained concerning whether calcium has a direct impact on intracellular motility in erythrophores. Example Embodiment 9 includes direct pharmacological evidence for the presence of calcium ion channels on the plasma membrane of erythrophores but not melanophores. The activity of these channels leading to altered intracellular calcium concentrations results in the translocation of red pigmented vesicles in erythrophores, but no corresponding changes in the black pigment of melanophores.

Erythrophore cultures were prepared 3-10 days prior use as described herein. Dorsal, caudal, and anal fins were clipped from euthanized male Betta splendens and diced into 2.0-4.0 mm squares. The diced fins were washed in six solution changes of phosphate buffered saline (PBS) (137 mM NaCl, 2.7 mM KCl, 10 mM Na₂HPO₄, 1.6 mM KH₂PO₄, pH 7.4) containing 1 mM NaEDTA, and transferred into 7 ml of digestion solution (PBS containing 960 unit/ml collagenase I and 230 unit/ml hyalouronidase Worthington Biochemical Corp., Lakewood, N.J., USA). After a 30-minute incubation, dissociated erythrophores were separated from the digestion solution by centrifugation for 3 minutes at 300×g and resuspended in L15 media (Sigma-Aldrich Co., St. Louis, Mo., USA) supplemented with 5% fetal bovine serum. Erythrophores from three successive digestion solution incubations were pooled and suspended in complete L15 media at a concentration of 1×10⁵ cells/ml. Cells were plated on 15 mm diameter glass cover slips coated with 20 μg/cm² collagen IV and 15 μg/cm² fibronectin (both from Sigma-Aldrich Co.) by placing one drop on each cover slip. After allowing the erythrophores to attach to the substrate for one hour, the cover slips were submerged in complete L15 media and stored at room temperature. Mixed cultures containing both melanophores and erythrophores were prepared by choosing fin tissue that contained both color types of chromatophores. Alternatively, different fish can be used to provide different types of chromatophores and a suitable combination produced by mixing.

Prior to exposure with calcium-modulating chemicals, erythrophores attached to cover slips were loaded into a continuous flow chamber (Warner Instrument Corp., Hamden, Conn., USA) and equilibrated for 5 minutes in physiological saline solution (PSS, 128 mM NaCl, 2.7 mM KCl, 1.8 mM CaCl₂, 1.8 mM MgCl₂, 5.6 mM glucose, 10 mM Tris/HCl, pH 7.2). Added agents were injected directly into the enclosed cell chamber at the given concentration dissolved in 200 μl of PSS. Added chemicals can be flushed out of the cell chamber by continuous upstream flow of PSS.

An assembled erythrophore chamber was mounted on a stage of a Zeiss IM-35 inverted microscope (Oberkochen, Germany) fitted with a Plan 6.3x objective having a 0.16 numerical aperture. A field of view was chosen that included about 200 erythrophores. Digital images were captured every 20 seconds with a Panasonic GP-US502 color 3-CCD camera using a Flashpoint 3D frame grabber and a personal computer based on a Pentium III processor. Image processing was performed using Image Pro 4.1 image analysis software.

Measurements were based on the following steps. Images were segmented to distinguish erythrophores (dark objects) from a bright background and a relative area occupied by erythrophores was calculated. The area was then calculated for all dark objects in the segmented image. The area was calculated in relative size units.

Pigment aggregation and dispersion trends within a sequence of images were determined by calculating a relative aggregation for each image in the sequence. The relative aggregation (Relative Aggr.) was computed using the following formula:
Relative Aggr.=−log₁₀(Pixel Area/Reference Pixel Area)
wherein the pixel area was determined for each image and the reference pixel area was the area for the first image in the sequence.

A dose-response curve was obtained and individual data points in the dose response curves were found by calculating a dispersion ratio for each test. This value was calculated as the following ratio: dispersion ratio=(erythrophore area after 10 min exposure)/(erythrophore area before exposure).

The requirement of calcium for intracellular transport in erythrophores has been hypothesized for erythrophores previously, but the involvement of the key molecular target of membrane acting calcium channel blocking drugs has not been investigated. The calcium requirement for intracellular transport of pigment was confirmed in Betta splendens erythrophores by treating the cells with a common aggregation-inducing stimulus (10 nM norepinephrine) in solution that either contained calcium ion or solution devoid of calcium ion. The results on erythrophores from this treatment are shown in FIGS. 34A-34B and 35A-35B. With reference to FIG. 34B, in calcium containing PSS (1.8 mM added Ca²⁺), treatment of erythrophores with norepinephrine produces apparent aggregation of pigment-containing vesicles with respect to unexposed erythrophores (FIG. 34A). With reference to FIG. 35B, exposure in PSS with 1 mM EGTA and no added calcium produces no apparent aggregation in comparison with unexposed erythrophores (FIG. 35A). It is evident that pigment aggregation was impaired by the lack of calcium. Thus, pigment transport can be altered by pharmacologically perturbing intracellular calcium concentration.

Modulators of plasma membrane calcium channels were initially considered as a means of controlling intracellular calcium levels. The direct application of the calcium channel activator Bay K8644 resulted in neither the aggregation nor dispersion of erythrophore pigment and direct application of a variety of Ca²⁺ channel inhibitors did not produce a direct response in erythrophores. However, pre-treatment with norepinephrine has a two-fold effect on erythrophores. First, NE operates through adrenergic receptors to open receptor-activated calcium channels on erythrophores. This results in a higher transient intracellular Ca²⁺ concentration. Second, NE acts as an agonist of α₂-adrenergic receptors that leads to inhibition of adenylate cyclase and a decrease in intracellular cAMP levels, a pathway that is well characterized in association with the aggregation of pigment in all dendritic chromatophores, including erythrophores. The application of inhibitors of plasma membrane calcium channels to erythrophores after pre-treatment with norepinephrine resulted in immediate and rapid dispersion of pigmented vesicles.

FIG. 36 contains dose response curves for inhibitors verapamil, diltiazem, and nifedipine. Erythrophores were treated for at least 5 minutes with 1 nM NE prior to exposure to the L-type Ca²⁺ channel blockers. Dispersion response was calculated as an area occupied by cells after exposure for 10 minutes divided by an area occupied prior to exposure. Curves labeled with (+Ca²⁺) were performed in PSS containing 1.8 mM added Ca²⁺. Curves labeled with (−Ca²⁺) were performed without added Ca²⁺. Each of the above chemicals added directly to erythrophores produces pigment dispersion but does not alter melanophores. The mode of action for each of these agents is similar in that they work by blocking the passage of calcium ions from extracellular space into the cytoplasm. Each of these chemicals induces this response in a dose dependent manner. Control experiments performed in solutions without added calcium failed to induce pigment dispersion response so that a direct role of intracellular calcium concentration in these cells is implicated.

Erythrophore dispersion caused by Ca²⁺ channel inhibition was examined by imaging continuously during an experiment where two different verapamil concentrations were added in succession. Continuous pre-treatment with norepinephrine caused pigment to aggregate in cells that normally have dispersed pigment. Upon addition of a dose of 1 nM verapamil, rapid dispersion followed until the cell chamber was flushed of verapamil by continued flow of buffered solution with added norepinephrine. This allowed the cells to re-aggregate rapidly until a second, stronger dose (100 nM) of verapamil was added. This stronger dose of verapamil caused the cells to disperse almost as completely as possible and the dispersion was sustained as long as verapamil was present. This demonstrates not only the sensitivity of erythrophores to Ca²⁺ channel inhibitors, but also the ability of these cells to repeatedly and consistently respond.

FIG. 37 contains a graph of erythrophore aggregation/dispersion as a function of time during which various doses of verapamil are applied. Initially, the erythrophores are exposed to NE and then exposed to 5 nM verapamil causing pigment dispersion. During flushing, there was an exposure to 1 nM NE without verapmil, producing re-aggregation. Exposure to 100 nm verapmil produced a larger dispersion.

To confirm the results observed by raising intracellular Ca²⁺ concentration with Ca²⁺ channel blockers, the membrane-permeable acetylmethyl (AM) form of the Ca²⁺ cHeLator BAPTA was added to erythrophores. The lower intracellular Ca²⁺ concentration ensuing from cHeLation resulted in pigment dispersion as seen in FIG. 38. These results led to the question of whether increasing intracellular Ca²⁺ levels would result in an opposite effect as decreasing levels, namely by aggregating the pigment in erythrophores. Treating erythrophores with the Ca²⁺ ionophore ionomycin tested this idea. The results of this experiment can also be seen in FIG. 38 in graph 25. Upon addition of ionomycin to erythrophores a slight, but sustained aggregation is apparent, confirming that Ca²⁺ concentration can independently modulate both dispersion and aggregation in erythrophores.

The presence of intracellular Ca²⁺ channels in erythrophores was also investigated. Such ryanodine and IP₃ receptors are present on the endoplasmic and sarcoplasmic reticulum in many other cell types and control the flow of Ca²⁺ from intracellular stores. Again the pharmacological applications of receptor modulators were used on ryanodine receptors. IP₃ receptors were not examined here because of their cross-reactivity in other signaling pathways and because of the lack of specific cell-permeable agonists. Ryanodine receptors were screened for by the application of the agonist ryanodine as seen in FIG. 39. Ryanodine is a membrane permeable molecule that has an unusual biphasic effect on intracellular receptors because at low concentrations (1 nM-100 nM) ryanodine acts as an agonist of the receptor. However, at high concentrations (>1 μM) ryanodine acts as an antagonist of the same receptors. Referring to FIG. 39, curve 31 corresponding to 10 nM ryanodine is associated with increasing aggregation while curve 33, corresponding to 10 μM ryanodine is associated with decreasing aggregation. This information is consistent with the data shown in FIG. 39. A 10 μM dose of ryanodine onto erythrophores did not produce any morphological change in the cells, signifying that no Ca²⁺ was released into the cytoplasm from intracellular stores. However, a 10 nM dose of ryanodine onto erythrophores did produce a sustained aggregation response. This result is consistent with the biphasic response profile of ryanodine. Images of erythrophores treated with low and high concentrations of ryanodine are consistent with these results. The aggregation response seen at a low concentration (10 nM) of ryanodine suggests that ryanodine receptors are mediating the release of Ca²⁺ from the endoplasmic reticulum into the cytoplasm.

Ca²⁺ channels are involved in the bi-directional movement of intracellular vesicles in erythrophores. There is no corresponding calcium dependence in melanophores, and so the combined observation of erythrophores and melanophores in response to pharmacological agents can verify or refute the possibility that a given agent acts upon calcium regulating molecular targets within the cells. Such a capability of chromatophores is established by the showing that the requirement for extracellular calcium for pigment movements and the effects of numerous calcium-modulating agents on pigment movements are exclusive to erythrophores and not melanophores.

Referring to Table 7, cytosensors can be configured for a variety of applications such as identifying, quantifying, or discriminating among and between neurotransmitters, adrenergic agonists, adrenergic antagonists, serotonergic agonists, serotonergic antagonists, hormones, cytoskeletal inhibitors, cAMP and Ca++ signal transduction modulators, membrane voltage modulators, neurotoxins, and protein kinase modulators. Effects of some Ca²⁺ modulating chemicals are listed in Table 7. Pharmacological results using Ca²⁺ channel antagonists indicate the presence of L-type channels in Betta splendens erythrophores.

TABLE 7
Effects of Ca²⁺ modulating chemicals on Betta erythrophores

| | Morphology Effect | | |
|---|---|---|---|
| | Dispersion | Aggregation | Comments |
| Ca²⁺ channel antagonists^{a} | | | |
| Verapamil (@ <1 μM) | Yes | No | Phenylalkylamine, L-type specific |
| Diltiazem (@ <1 μM) | Yes | No | Benzothiazepine, L-type specific |
| Nimodipine (@ <1 μM) | Yes | No | Dihydropyridine, L-type specific |
| Nifedipine (@ <1 μM) | na^{c} | na | Dihydropyridine, L-type specific |
| μ-Conotoxin GVIA | No | No | L-type channels are insensitive |
| Ca²⁺ channel agonists | | | |
| Bay K8644 | Na | na | Dihydropyridine, L-type specific |
| High [K+] (>50 mM) | No | Yes (slight) | |
| Ca²⁺ cHeLating chemicals | | | |
| EGTA (extracell.) | na^{c} | na | |
| BAPTA/AM (intracell.) | Yes | No | Cell permeable, Ca²⁺ specific |
| Ionophores | | | |
| A23187 | No | No | |
| Ionomycin | No | Yes | More effective than A23187 |
| Intracellular Ca²⁺ channel agonist | | | |
| Ryanodine^{b} | | | Mediates Ca²⁺ release from ER |
| Release Ca²⁺ @ nM-μM | No | Yes | |
| Inhibit Ca²⁺ release | No | No | |
| @ >1 μM | | | |
| Intracellular Ca²⁺ channel | | | |
| antagonist | | | |
| Ruthenium red | na^{c} | na | |
| Microsomal Ca²⁺-ATPase inhibitor | | | |
| Thapsigargin (@ <1 μM) | No | Yes | Results in elevated cytoplasmic Ca²⁺ |
| | | | levels |

Additional chromatophore responses to various agents and classes are listed in Table 8. Response times are indicated as X (minutes), XX (tens of minutes), or hours (X=1-6 hr, XX=6-24 hr). Threshold concentrations are indicated in parts per billion (X=1 to 100 PPB, XX=100 to 1000 PPB), parts per million (X=1 to 100 PPM, XX=100 to 1000 PPM), or parts per thousand (X=1 PPT or higher). The nature of the chromatophore response is summarized for melanophores, erythrophores, xanthophores, iridophores, or combinations thereof, or on chromatophores in representative types of 2-cell cytosensor (chromatophore/neuronal cell, chromatophore/bacterial cell, chromatophore/fungal cell, chromatophore/protozoal cell). Pigment aggregation and dispersion are indicated as Aggr. and Disp., respectively. Aggregation followed by impairment is indicated as All and fish scale responses are indicates as FS. Chromatophore impairments is indicated as Imp.

The 2-cell cytosensors associated with Table 8 can include chromatophores and a small inoculum of a selected microbial cell (bacteria, fungus, protozoan) as the second cell type. To test its potency, a potential antibiotic is added to the 2-cell cytosensor. At a later time the chromatophores are evaluated by a known test agent (such as norepinephrine). If the antibiotic is potent, the microbial inoculum will not have thrived and toxified the chromatophores, and so the chromatophores will exhibit a “normal response” to the known test agent. If the antibiotic is not potent, however, the chromatophores will have become toxified by the microbe and the chromatophores will not respond normally to the test agent. Such versions of the 2-cell cytosensor are capable of detecting numerous antibiotics beyond those listed in Table 1B. Moreover, the 2-cell cytosensor is useful because a normal response further indicates that the antibiotic is not harmful to the animal cells (chromatophores). Useful antibiotics such as penicillin, having low animal cell toxicity, are readily detectable in this manner.

Tables 9-10 list additional agents and categories of agents having chromatophore responses.

TABLE 8
Chromatophore Response to Categories of Agents

| | | | | | | | Chromatophore response | | | | |
|---|---|---|---|---|---|---|---|---|---|---|---|
| | | | | | | | | | | | 2-Cell |
| Functional | | Response | | Threshold | | | | | | | Cytosensor |
| Category of | | time | | concentration | | | Melano- | Erythro- | Xantho- | Irido- | (second cell |
| Agent | Specific agent | Min. | Hr. | PPB | PPM | PPT | phores | phores | phores | phores | type included) |
| Neuro- | Norepinephrine | X | | X | | | Aggr. | Aggr. | Disp. | Hue changes (FS) | |
| transmitters | | | | | | | | | | | |
| | Adenosine | X | | | XX | | Disp. | | | | |
| | Dopamine | X | | | XX | | Aggr. (FS) | | | | |
| | Serotonin | X | | | X | | Partial Aggr. | | | | |
| | Acetylcholine | X | | | X | | | | | | Eryth. Aggr. |
| | | | | | | | | | | | (neuronal cells) |
| | ATP | X | | | X | | | | | | Erythr. Aggr. |
| | | | | | | | | | | | (neuronal cells) |
| Adrenergic | Clonidine | X | | XX | | | Aggr. | Aggr. | Disp. | | |
| agonists | | | | | | | | | | | |
| | Naphazoline | X | | XX | | | Aggr. | Aggr. | Disp. | | |
| | Oxymetazoline | X | | XX | | | Aggr. | Aggr. | Disp. | | |
| | Tetrahydrozoline | X | | XX | | | Aggr. | Aggr. | Disp. | | |
| Adrenergic | Phentolamine | X | | XX | | | Blocks norepinephrine | | | | |
| antagonists | | | | | | | | | | | |
| | Yohimbine | X | | X | | | Blocks norepinephrine | | | | |
| | Phenoxy- | X | | X | | | Blocks norepinephrine | | | | |
| | benzamine | | | | | | | | | | |
| Serotonergic | | X | | XX | | | Blocks serotonin | | | | |
| antagonists | | | | | | | | | | | |
| Cholinergic | Carbachol | X | | | X | | | | | | Erythr. Aggr. |
| agonists | | | | | | | | | | | (neuronal cells) |
| Hormones | MCH | X | | X | | | Aggr. | | | | |
| | MSH | X | | X | | | Disp. | Disp. | | Hue changes (isolated | |
| | | | | | | | | | | iridophores) | |
| | Substance P | XX | | | | | | | | Hue changes (FS) | |
| Cytoskeletal | Colchicine | XX | | | XX | | Imp. | | | | |
| inhibitors | | | | | | | | | | | |
| | Cytochalasin D | XX | | | XX | | Imp. | | | | |
| cAMP Signal | CAMP | X | | XX | | | Disp. (microinjected and permeabilized melanophores) | | | | |
| transduction | | | | | | | | | | | |
| modulators | | | | | | | | | | | |
| | Forskolin | X | | XX | | | Disp. | | | | |
| | Caffeine | XX | | XXX | | | Disp. | | | | |
| Ca++ Signal | Verapamil | X | | X | | | | Disp. | | | |
| transduction | | | | | | | | | | | |
| modulators | | | | | | | | | | | |
| | Diltiazem | X | | XX | | | | Disp. | | | |
| | Nifedipine | X | | XX | | | | Disp. | | | |
| | Ryanodine | X | | XX | | | | Aggr. | | | |
| Membrane | Depolarizing K+ | X | | | | X | Aggr. | Aggr. | | | Erythr. Aggr. |
| voltage | in medium | | | | | | (FS) | | | | (neuronal cells) |
| modulators | | | | | | | | | | | |
| | Tricaine | X | | | X | | Disp. | | | | |
| | | | | | | | (FS) | | | | |
| Neurotoxins | Capsaicin | XX | | | X | | Hollow- | | | | |
| | | | | | | | ing (FS) | | | | |
| | Latrotoxin | X | | XX | | | | Aggr | | | Erythrophore |
| | (spider venom) | | | | | | | | | | Aggr. |
| | | | | | | | | | | | (neuronal cells) |
| | w-Conotoxin | XX | | | X | | | Disp. | | | |
| | peptide | | | | | | | | | | |
| | neurotoxin) | | | | | | | | | | |
| | Saxitoxin | XX | | | XX | | Aggr. | Aggr. | | | |
| | (Paralytic | | | | | | (FS) | | | | |
| | shellfish toxin) | | | | | | | | | | |
| Protein | Protein kinase | X | | | XX | | Disp. | | | | |
| kinase | inhibitor H-8 | | | | | | | | | | |
| modulators | | | | | | | | | | | |
| | Protein kinase | X | | | XX | | Disp. | | | | |
| | inhibitor H-9 | | | | | | | | | | |
| | Protein kinase | X | | | X | | Disp. | | | | |
| | inhibitor K252a | | | | | | | | | | |
| Caustic | Salicylate | XX | | | XX | | Imp. (FS) | | | | |
| irritants | | | | | | | | | | | |
| | Formaldehyde | XX | | | XX | | Imp. (FS) | | | | |
| | Ammonium | XX | | | XX | | Imp. (FS) | | | | |
| | Hydrogen | XX | | | XX | | Imp. (FS) | | | | |
| | peroxide | | | | | | | | | | |
| | Cyanide | | X | | X | | Imp. (FS) | | | | |
| Heavy metals | Pb | XX | | | | X | Imp. (FS) | | | | |
| | Cu | XX | | | | X | Imp. (FS) | | | | |
| | Ag | XX | | | | X | Imp. (FS) | | | | |
| | Ni | XX | | | | X | Imp. (FS) | | | | |
| Polyaromatic | Pyrene | XX | | | X | | | Imp. | | | |
| hydrocarbons | | | | | | | | | | | |
| | 1,2- | XX | | | X | | | Imp. | | | |
| | Benzanthracene | | | | | | | | | | |
| | Fluoranthene | XX | | | X | | | Imp. | | | |
| | Acenapthene | XX | | | X | | | Imp. | | | |
| Organo- | DFP | X | | | X | | Imp. | | | | |
| phosphate | | | | | | | | | | | |
| nerve agents | | | | | | | | | | | |
| | DFP | XX | | | X | | A/I (FS) | Aggr. (FS) | | Hue changes (FS) | |
| | Paraoxon | XX | | | XX | | A/I (FS) | Aggr. (FS) | | Hue changes (FS) | |
| | Mipafox | | X | | XX | | A/I (FS) | Aggr. (FS) | | Hue changes (FS) | |
| | EPN | | X | | XX | | | | | | |
| | Mevinphos | | X | | | X | Imp. | Imp. (FS) | | Hue change, then Imp. | |
| | | | | | | | (FS) | | | (FS) | |
| | Dichlorvos | | X | | | X | Imp. | Imp. (FS) | | Hue change, then Imp. | |
| | | | | | | | (FS) | | | (FS) | |
| | Trichlorfon | | X | | | X | Imp. | Imp. (FS) | | Hue change (FS) | |
| | | | | | | | (FS) | | | | |
| Pyschogenic | Cocaine | X | | | | | Aggr. (FS) | | | | |
| agents | | | | | | | | | | | |
| | Cannabinoid | | X | | XX | | Disp. (FS) | | | | |
| | CP-55940 | | | | | | | | | | |
| | Amphetamine | XX | | | X | | Aggr. (FS) | | | | |
| | Desipramine | XX | | | X | | Aggr. (FS) | | | | |
| Various | Pheniramine | XX | | | XX | | Disp. | | | | |
| drugs and | (antihistamine) | | | | | | | | | | |
| agents | | | | | | | | | | | |
| | PMSF (enzyme | XX | | | X | | Imp. (FS) | | | | |
| | inhibitor) | | | | | | | | | | |
| | Hydrocortisone | | XX | | X | | Hyp. D. | HypD. | | | |
| | Bradykinin | X | | | XX | | | | | | Eryth. Aggr. |
| | | | | | | | | | | | (neuronal cells) |
| | Guanethidine | XX | | | XX | | Disp. | | | | |
| | | | | | | | (FS) | | | | |
| | Histamine | X | | | XX | | Aggr. | | | | |
| | | | | | | | (FS) | | | | |
| | Imipramine | X | | | X | | Disp. | | | | |
| | | | | | | | (FS) | | | | |
| Algal toxins | Algal extracts. | X (various | | X (range of | | | Aggr. | | | | |
| (48 sources) | (six different | response | | concentrations, | | | | | | | |
| | species of | times) | | from 0.3 ppm to 10 | | | | | | | |
| | marine algae) | | | ppm) | | | | | | | |
| | Algal extracts. | X (various | | X (range of | | | A/I | | | | |
| | (nineteen | response | | concentrations, | | | | | | | |
| | different species | times) | | from 0.3 ppm to 10 | | | | | | | |
| | of marine algae) | | | ppm) | | | | | | | |
| | Algal extracts. | X (various | | X (range of | | | Imp. | | | | |
| | (seventeen | response | | concentrations, | | | | | | | |
| | different species | times) | | from 0.3 ppm to 10 | | | | | | | |
| | of marine algae) | | | ppm) | | | | | | | |
| | Algal extracts. | No detected response at up to 10 ppm | | | | | | | | | |
| | (six species of | | | | | | | | | | |
| | marine algae) | | | | | | | | | | |
| Algal toxins | Brevetoxin | | X | | X | | Aggr. (FS) | | | | |
| (pure | | | | | | | | | | | |
| sources) | | | | | | | | | | | |
| | Curacin A | | X | | X | | Aggr. and Imp. (FS) | | | | |
| Bacterial | Cholera toxin | | X | | X | | | Imp. | | | |
| toxins | | | | | | | | | | | |
| | Pertussis toxin | | X | | X | | Imp. | | | | |
| | Staph alpha- | | X | | X | | Aggr. | | | | |
| | toxin | | | | | | | | | | |
| Known | Bacillus cereus | X | | | | | Aggr. | Aggr. | | | |
| pathogenic | | | | | | | | | | | |
| bacteria (9 | | | | | | | | | | | |
| tested strains) | | | | | | | | | | | |
| | Shigella dysenteria | | X | | | | Aggr. | Aggr. | | | |
| | Shigella sonnei | | X | | | | Aggr. | Aggr. | | | |
| | Salmonella typhi | | X | | | | Aggr. | Aggr. | | | |
| | Salmonella enteriditis | | X | | | | Aggr. | Aggr. | | | |
| | Escherichia coli | | X | | | | Aggr. | Aggr. | | | |
| | O157: H7 | | | | | | | | | | |
| | Vibrio cholera | | X | | | | Aggr. | Aggr. | | | |
| | Clostridium sp. | X | | | | | Aggr. | Aggr. | | | |
| | Pseudomonas sp. | No detected response | | | | | Imp. | | | | |
| | | | | | | | (FS) | | | | |
| Non- | Escherichia coli | No detected response | | | | | | | | | |
| pathogenic | JM101 | | | | | | | | | | |
| bacteria (3 | | | | | | | | | | | |
| strains) | | | | | | | | | | | |
| | Bacillus subtilis | No detected response | | | | | | | | | |
| | Lactococcus | No detected response | | | | | | | | | |
| | lactis | | | | | | | | | | |
| Bacterial | Twenty-four | X | | X | | | | Various acute responses and impaired responses | | | |
| isolates (34 | natural isolates | | | | | | | | | | |
| tested) | of bacteria, | | | | | | | | | | |
| | collected from | | | | | | | | | | |
| | air and water | | | | | | | | | | |
| | sources | | | | | | | | | | |
| | Six broths of | X (range of | | X (range of concentrations) | | | | Various acute responses and impaired responses | | | |
| | anaerobic | times) | | | | | | | | | |
| | bacteria, | | | | | | | | | | |
| | prepared from | | | | | | | | | | |
| | isolates collected | | | | | | | | | | |
| | from outdoor | | | | | | | | | | |
| | soil samples | | | | | | | | | | |
| | Exposure to | XX | | Single-colony dose | | | | Various acute responses and impaired responses | | | |
| | single bacterial | | | | | | | | | | |
| | colonies | | | | | | | | | | |
| | collected by | | | | | | | | | | |
| | exposing agar | | | | | | | | | | |
| | plates to indoor | | | | | | | | | | |
| | air (four distinct | | | | | | | | | | |
| | colonies) | | | | | | | | | | |
| Antibacterial | Penicillin | | XX | | X | | | | | | Normal |
| agents | | | | | | | | | | | response |
| | | | | | | | | | | | (bacterial |
| | | | | | | | | | | | inoculum) |
| | Streptomycin | | XX | | X | | | | | | Normal |
| | | | | | | | | | | | response |
| | | | | | | | | | | | (bacterial |
| | | | | | | | | | | | inoculum) |
| | Tetracycline | | XX | | X | | | | | | Normal |
| | | | | | | | | | | | response |
| | | | | | | | | | | | (bacterial |
| | | | | | | | | | | | inoculum) |
| | Trimethoprim- | | XX | | X | | | | | | Normal |
| | sulfamethoxazole | | | | | | | | | | response |
| | | | | | | | | | | | (bacterial |
| | | | | | | | | | | | inoculum) |
| | Nalidixic acid | | XX | | X | | | | | | Normal |
| | | | | | | | | | | | response |
| | | | | | | | | | | | (bacterial |
| | | | | | | | | | | | inoculum) |
| Fungi (14 | Ten | X | | X | | | | Various acute responses and impaired responses | | | |
| tested) | uncharacterized | | | | | | | | | | |
| | fungal broths, | | | | | | | | | | |
| | prepared from | | | | | | | | | | |
| | isolates collected | | | | | | | | | | |
| | from outdoor | | | | | | | | | | |
| | soil samples and | | | | | | | | | | |
| | air samples. | | | | | | | | | | |
| | Exposure to | XX | | Single-colony dose | | | | Various acute responses and impaired responses | | | |
| | single fungal | | | | | | | | | | |
| | colonies, | | | | | | | | | | |
| | collected by | | | | | | | | | | |
| | exposing agar | | | | | | | | | | |
| | plates to indoor | | | | | | | | | | |
| | air (four distinct | | | | | | | | | | |
| | colonies | | | | | | | | | | |
| | representing four | | | | | | | | | | |
| | distinct fungal | | | | | | | | | | |
| | species) | | | | | | | | | | |
| Antifungal | Amphotericin B | | XX | | X | | | | | | Normal |
| agents | | | | | | | | | | | response |
| | | | | | | | | | | | (fungal |
| | | | | | | | | | | | inoculum) |
| Antiprotozoal | Metronidazole | | XX | | X | | | | | | Normal |
| agents | | | | | | | | | | | response |
| | | | | | | | | | | | (protozoal |
| | | | | | | | | | | | inoculum) |
| | Chloroquine | | XX | | XX | | | | | | Normal |
| | | | | | | | | | | | response |
| | | | | | | | | | | | (protozoal |
| | | | | | | | | | | | inoculum) |
| | Quinine sulfate | | XX | | XX | | | | | | Normal |
| | | | | | | | | | | | response |
| | | | | | | | | | | | (protozoal |
| | | | | | | | | | | | inoculum) |

TABLE 9
Additional Categories and Agents

| | | |
|---|---|---|
| Categories | Detected agents | Likely capability |

DRUGS ACTING AT SYNAPTIC AND NEUROEFFECTOR JUNCTIONAL SITES

| | |
|---|---|
| Neurotransmission: The autonomic and somatic | Norepinephrine |
| motor nervous systems. | Adenosine |
| | ATP |
| Muscarinic receptor agonists and antagonists. | Acetylcholine |
| | Carbachol |
| Anticholinesterase agents. | DFP, Paraoxon, Mipafox, EPN, |
| | Mevinphos, Dichlorvos, Trichlorfon |
| Agents acting at the neuromuscular junction and | Acetylcholine |
| autonomic ganglia. | |
| Catecholamines, sympathomimetic drugs, and | Norepinephrine, Dopamine, Clonidine, |
| adrenergic receptor antagonists. | Naphazoline, Oxymetazoline |
| 5-Hydroxytryptamine (Serotonin) receptor | Serotonin |
| agonists and antagonists. | Ketanserin |

DRUGS ACTING ON THE CENTRAL NERVOUS SYSTEM

| | | |
|---|---|---|
| Neurotransmission and the central nervous | — | |
| system. | | |
| History and principles of anesthesiology. | — | |
| General anasthetics. | Not tested | Barbiturates |
| | | Benzodiazepines |
| Local anasthetics. | Tricaine, Cocaine, Saxitoxin | |
| Therpapeutic gases. | Not tested | Nitric oxide |
| Hypnotics and sedatives: Ethanol | Not tested | Benzodiazepines |
| Drugs and the treatment of psychiatric disorders: | Clonidine, Reserpine | Benzodiazepines |
| Psychosis and anxiety. | | |
| Drugs and the treatment of psychiatric disorders: | Desipramine | |
| Depression and mania. | Imipramine | |
| Drugs effective in the therapy of the epiliepsies. | Not tested | Barbiturates |
| | | Benzodiazepines |
| Drugs effective in the therapy of migraine. | Ketanserin, Diltiazem, Nifedipine, | |
| | Verapamil | |
| Treatment of central nervous system degenerative | Dopamine, Acetylcholine, Carbachol | |
| disorders. | | |
| Opioid analgesics and antagonists. | Not tested | Morphine |

AUTACOIDS: DRUG THERAPY OF INFLAMMATION

| | | |
|---|---|---|
| Histamine, Bradykinin and their antagonists. | Histamine, Pheniramine, Bradykinin | |
| Lipid-derived autacoids: Eicosanoids and platelet- | Not tested | Unknown |
| activating factor. | | |
| Analgesic-antipyretic and antiinflammatory agents | Colchicine | Vinblstine |
| and drugs employed in the treatment of gout. | | |
| Drugs used in the treatment of asthma. | Caffeine | Theophylline |

DRUGS AFFECTING RENAL AND CARDIOVASCULAR FUNCTION

| | | |
|---|---|---|
| Diuretics. | Not tested | FK453, adenosine receptor |
| | | antagonist |
| Vasopressin and other agents affecting the renal | Protein kinase inhibitors H-8, H-9, | |
| conservation of water. | K252a | |
| Renin and angiotensin. | Not tested | Angiotensin |
| Drugs used in the treatment of myocardial | Diltiazam, Nifedipine | Digoxin |
| ischemia. | Verapamil | |
| Antihypertensive agents and the drug therapy of | Guanethidine, Reserpine, Diltiazem, | |
| hypertension. | Nifedipine, Verapamil | |
| Pharmacological treatment of heart failure. | Phentolamine | |
| | Nifedipine | |
| Antiarrythmic drugs. | Clonidine, Adenosine, Diltiazem, | |
| | Nifedipine, Verapamil ″ | |
| Drugs used in the treatment of | Not tested | Unknown |
| hyperlipoproteinemias. | | |

DRUGS AFFECTING GASTROINTESTINAL FUNCTION

| | | |
|---|---|---|
| Agents for control of gastric acidity and treatment | Metronidazole | |
| of peptic ulcers. | | |
| Agents affecting gastrointestinal water flux and | Cannabinoid CP-55940 | Metoclopramide |
| motility; emesis and antiemetics; bile acids and | | Phenothiazines |
| pancreatic enzymes. | | |

DRUGS AFFECTING UTERINE MOTILITY

| | |
|---|---|
| Agents that cause contraction or relaxation of the | Nifedipine |
| uterus. | |

CHEMOTHERAPY OF PARASITIC INFECTIONS

| | |
|---|---|
| Drugs used in the chemotherapy of protozoal | Chloroquine, Quinine sulfate, |
| infections: Malaria. | Tetracycline, Sulfonamide |
| Drugs used in the chemotherapy of protozoal | Chloroquine, Metronidazole |
| infections: Trypanosomiasis, leishmaniasis, | |
| amebiasis, giardiasis, trichhomoniasis, and other | |
| protozoal infections. | |
| Drugs used in the chemotherapy of helminthiasis. | Not tested |

CHEMOTHERAPY OF MICROBIAL DISEASES

| Antimicrobial agents: General considerations. | | |
|---|---|---|
| Antimicrobial agents: Sulfonamides, | Trimethoprim-sulfamethoxazole, | |
| trimethoprim-sulfamethoxazole, quinolones, and | Nalidixic acid | |
| agents for urinary tract infections. | | |
| Antimicrobial agents: Penicillins, cephalosporins, | Penicillin | Ampicillin |
| and other beta-lactam antibiotics. | | |
| Antimicrobial agents: The aminoglycosides. | Streptomycin | Neomycin |
| | | Kanamycin |
| Antimicrobial agents: Tetracyclines, | Tetracycline | Chloramphenicol, |
| chloramphenicol, erythromycin, and miscellaneous | | Erythromycin, Vancomycin |
| antibacterial agents. | | |
| Antimicrobial agents: Drugs used in the | Streptomycin | Isoniazid |
| chemotherapy of tuberculosis and leprosy. | | Rifampin |
| Antimicrobial agents: Antifungal agents. | Amphotericin B | |
| Antimicrobial agents: Antiviral agents. | Not tested | Unknown |

CHEMOTHERAPY OF NEOPLASTIC DISEASES

| | | |
|---|---|---|
| Antineoplastic agents. | Colchicine | Vinca alkaloids |
| | | Taxol |
| DRUGS USED FOR IMMUNOMODULATION | | |
| Immunomodulators: Immunosuppressive agents | Not tested | Unknown |
| and immunostimulants. | | |

DRUGS ACTING ON THE BLOOD AND THE BLOOD-FORMING ORGANS

| | | |
|---|---|---|
| Hematopoetic agents: Growth factors, minerals, | Not tested | Unknown |
| and vitamins. | | |
| Anticoagulant, thrombolytic, and antiplatelet | Not tested | Unknown |
| drugs. | | |

HORMONES AND HORMONE ANTAGONISTS

| | | |
|---|---|---|
| Adenohypophyseal hormones and their | MSH | |
| hypothalamic releasing factors. | | |
| Thyroid and antithyroid drugs. | Not tested | Unknown |
| Estrogens and progestins. | Not tested | Unknown |
| Androgens. | Not tested | Unknown |
| Adrenocorticotropic hormone; adrenocortical | Hydrocortisone | |
| steroids and their complex analogs; inhibitors of | | |
| the synthesis and actions of adrenocortical | | |
| hormones. | | |
| Insulin, oral hypoglycemic agents, and the | Verapamil | Insulin |
| pharmacology of the endocrine pancreas. | Clonidine | |
| Agents affecting calcification and bone turnover: | Not tested | Unknown |
| Calcium, phosphate, parathyroid hormone, | | |
| vitamin D, calcitonin, and other compounds. | | |

THE VITAMINS

| | | |
|---|---|---|
| Water-soluble vitamins: The vitamin B complex | Not tested | Unknown |
| and ascorbic acid. | | |
| Fat-soluble vitamins: Vitamin A, K and E. | Not tested | Unknown |

DERMATOLOGY

| | | |
|---|---|---|
| Dermatological pharmacology. | Hydrocortisone, Capsaicin, | Topical antibacterial agents |
| | Tetracycline, Colchicine | Topical antifungal agents |

OPTHAMOLOGY

| | |
|---|---|
| Ocular pharmacology. | Cocaine, Carbacol, DFP, Naphazoline, |
| | Tetrahydrozoline |

TOXICOLOGY

| | | |
|---|---|---|
| Heavy metals and heavy-metal antagonists. | Pb, Cu, Ag, Ni | |
| Nonmetallic environmental toxicants: Air | Polyaromatic hydrocarbons | Pyrethrin insecticides |
| pollutants, solvents, vapors, and pesticides. | Organophosphates | |
| | Cyaninde | |

TABLE 10
Additional Classes and Agents.

| | Detection | |
|---|---|---|
| Class/Agent | Demonstrated | Detection Likely |
| ANALGESICS AND | | |
| NONPRESCRIPTION | | |
| PREPARATIONS | | |
| Acetaminophen | | Not known |
| Salicylates | X | |
| Nonsteroidal antiinflammatory | | Not known |
| agents | | |
| Iron | | Not known |
| Vitamins | | Not known |
| Dieting agents and regimens | X | |
| Caffeine | X | |
| PRESCRIPTION | | |
| MEDICATIONS | | |
| Theophylline | | X |
| Hypoglycemic agents | X | |
| Anticonvulsants | X | |
| Antihistamines | X | |
| Antocoagulants | | Not known |
| Antituberculous agents | | X |
| Antimarial agents | X | |
| Ergotamines | | Not known |
| Antibiotics: Penicillin and other | X | |
| substances that cause | | |
| anaphylaxis and anaphylactoid | | |
| reactions | | |
| Antineoplastic agents | X | |
| Digitalis | | X |
| Antihypertensive agents | X | |
| Antidysrhythmic agents | X | |
| SECTION III: | | |
| PSYCHOPHARMACOLOGIC | | |
| MEDICATIONS | | |
| Cyclic antidepressants | X | |
| Neuroleptic agents | | X |
| Monoamine oxidase inhibitors | | X |
| Lithium | | Not known |
| Opioids | | Not known |
| Sedative-hypnotic agents | | X |
| ALCOHOLS AND DRUGS OF | | |
| ABUSE | | |
| Ethanol | | X |
| Methanol, ethylene glycol, and | | X |
| isopropanol | | |
| Cocaine | X | |
| Amphetamines | X | |
| Phencyclidine | | Not Known |
| Lysergic acid diethylamide and | X | |
| other psychedelics | | |
| Marijuana | | X |
| Disulfiram and disulfiramlike | | Not known |
| reactions | | |
| Substance withdrawal | N/A | |
| FOOD POISONING | | |
| Food poisoning | X | |
| Staphylococcus aureus | X | |
| Bacillus cereus | X | |
| Clostridium sp. | X | |
| Salmonella sp. | X | |
| Vibrio cholera | X | |
| Ciguatera seafood poisoning | | X |
| Shellfish poisoning (saxitoxin, | X | |
| brevetoxin) | | |
| Botulism | | X |
| BOTANICALS | | |
| Mushrooms: Toxic and | X | X |
| hallucinogenic | | |
| Herbal preparations | X | X |
| Plants | X | X |
| Nicotine | X | |
| HEAVY METALS | | |
| Arsenic | | Not known |
| Lead | X | |
| Mercury | | Not known |
| Cadmium and other metals and | X | |
| metalloids | | |
| HOUSEHOLD TOXINS | | |
| Antiseptics, disinfectants, and | X | |
| sterilants | | |
| Camphor and mothballs | | Not known |
| PESTICIDES | | |
| Insecticides: Organophosphates | X | |
| and carbamates | | |
| Insecticides: Chlorinated | | Not known |
| hydrocarbons, pyrethrins, and | | |
| DEET | | |
| Rodenticides | | Not known |
| Herbicides: Paraqaut and diquat | | Not known |
| OCCUPATIONAL AND | | |
| ENVIRONMENTAL TOXINS | | |
| Recognition and follow-up of | N/A | |
| workplace poisonings | | |
| Methemoglobinemia | | Not known |
| Simple asphyxiants and | | X |
| pulmonary irritants | | |
| Smoke inhalation | | X |
| Carbon monoxide | | X |
| Cyanide and hydrogen sulfide | X | |
| Hydrocarbons | X | |
| Caustics and batteries | | X |
| Hazardous materials releases | N/A | |
| Farm toxicology | X | |
| Art hazards | | X |
| TOXIC ENVENOMATIONS | | |
| Snakes and other reptiles | | X |
| Arthropods | X | |
| Marine animals | X | |

### Example 19

### Integration of Elicitor Panel Concept with Soft Classification Techniques

Sophisticated informatics is necessary for the development of fieldable cytosensor systems. Field applications add stringent requirements for simplicity and robustness to any analysis software and associated experimental methodologies. Simplicity implies the capacity for extracting useful information without extensive laboratory infrastructures. Robustness refers to accurate scenario matching with low false positive/negative generation rates. Informatics approaches must meet the simplicity and robustness requirements while having the capability to handle sparse data generated through experiments that produce large, highly multi-dimensional data sets. It should be noted that the approaches developed through the research described herein are applicable to cell based systems in general providing there are measurable outputs to generate the numerical feature vector and effectors of cell response are readily available.

The classification techniques integrate both numerical and non-numerical classifiers for optimal interpretation and decision-making processes and are built around four basic concepts: feature space, integrated experts, adaptive expert calibration and soft classification. Feature space consists of a non-numerical description (scenario) and a numerical feature vector extracted from the experimental run. Each experimental run is uniquely identified by the scenario and numerical feature vector. An expert is defined as a mapping from the numerical feature space to the set of scenarios. This mapping is realized through assigning to a numerical vector a set of mixing (weighting) coefficients corresponding to all the non-numerical descriptors (labels). An integrated expert is an arbitrary number of experts working in parallel. A process of adaptive calibration is needed to properly fuse the individual expert (integrated or individual) outputs. A simplex scenario is a special but important case of a scenario for which the numerical feature vector is concentrated on a single label or non-numerical identifier. Labeling induced by simplex scenarios divides the complete numerical feature or system response space into subsets. A complex scenario can be viewed as a combination of simplex scenarios. Soft classification is understood as the a priori creation of a complex scenario.

The elicitor set method is an experimental framework derived from an understanding of the signal transduction networks that underlie cellular function. Elicitor panels are formed from known effectors of checkpoints in the signal transduction pathways. Application of the elicitors in combination with known agents (calibration runs) results in the generation of simplex scenarios that form the basis for the soft classification of unknown agents (operational runs).

Fish chromatophores are plated in a 24-well culture dish 2-5 days prior to testing. The media is changed 1 day after plating. Stock concentrations of standard agents for the elicitor panel are prepared. The day of the test, fish chromatophores are sequentially exposed to the test agent and each elicitor agent in the panel. Time of exposure for the unknown agent was 5 minutes. Time of exposure to each elicitor agent varies, but is sufficient to capture the dynamics of the cell response as evidenced by the movement of the pigment particles (for example, aggregation, dispersion or no change).

The procedure for each experimental replicate consists of mounting the 24-well plate on the microscope stand, selecting a well, finding a field of view with an adequate number of cells (50-100), opening the image acquisition program setting the image capture rate (typically 30 frames/min), adding the unknown agent for a period of 5 minutes, and adding the known agent for the time period necessary to capture the cell response dynamics. A sufficient number of replicates were performed at each condition to develop a meaningful result (n=3-6).

As seen in Table 11, the elicitor agent panel consists of forskolin (100 μM), melanin stimulating hormone (MSH; 10 nM), clonidine (100 nM) and L-15 media (standard composition). The elicitor agents were selected on the basis of interactions with important signal transduction pathway checkpoints. Figure XXX shows cAMP mediated G-protein regulated pathways (Gₛ and Gᵢ) of importance to fish chromatophores. Forskolin is a direct activator of adenyl cyclase, a checkpoint in the Gₛ and Gᵢ pathways; melanin stimulating hormone directly stimulates the Gᵢ receptors; and clonidine directly stimulates Gᵢ receptors. Three strains of Bacillus cereus (BC1-49064, BC5-10987 and BC6-14579) expressing different complements of toxins as detailed in Table 12 were tested against the standard elicitor agent panel as described.

TABLE 11
Elicitor panel concentrations and reaction time

| | | |
|---|---|---|
| Elicitor | Final | Rxn Time¹ |
| Agent | Concentration | (min) |

| | | | |
|---|---|---|---|
| Forskolin | 100 | mM | 15 |
| MSH | 10 | nM | 20 |
| Clonidine | 100 | nM | 10 |

| | | |
|---|---|---|
| L-15 Media | Standard Composition | 10 |

¹Reaction time following exposure to test agent for 5 minutes

TABLE 12
Complements of toxins expressed by each B. cereus strain

| | Hbl | Nhe | BceT | New heat | |
|---|---|---|---|---|---|
| Strain | Toxin | Toxin | Toxin | + | Others |
| BC1-49064 | + | + | − | + | + |
| BC5-10987 | − | + | − | − | + |
| BC6-14579 | + | + | + | − | + |

The data was analyzed as follows. Features of interest (response profiles of different sub-populations of cells, changes in cell area etc.) were extracted. Data reduction was performed using modeling techniques such as parametric non-linear auto-regressive external input models. Model outputs were used to form clusters in feature space. Cluster analysis was performed using integrated experts and adaptive expert calibration as described elsewhere.

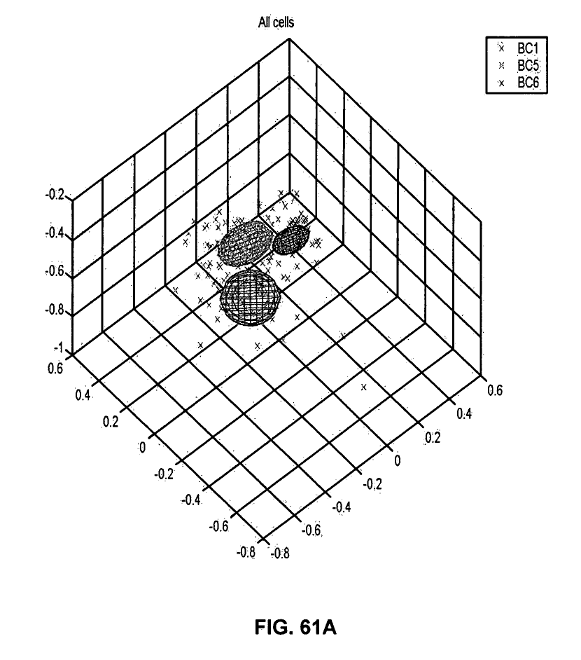

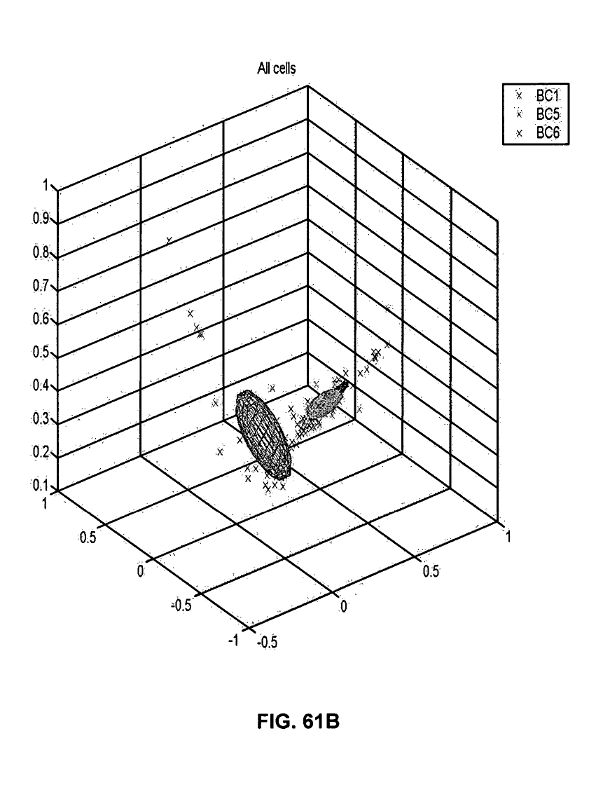

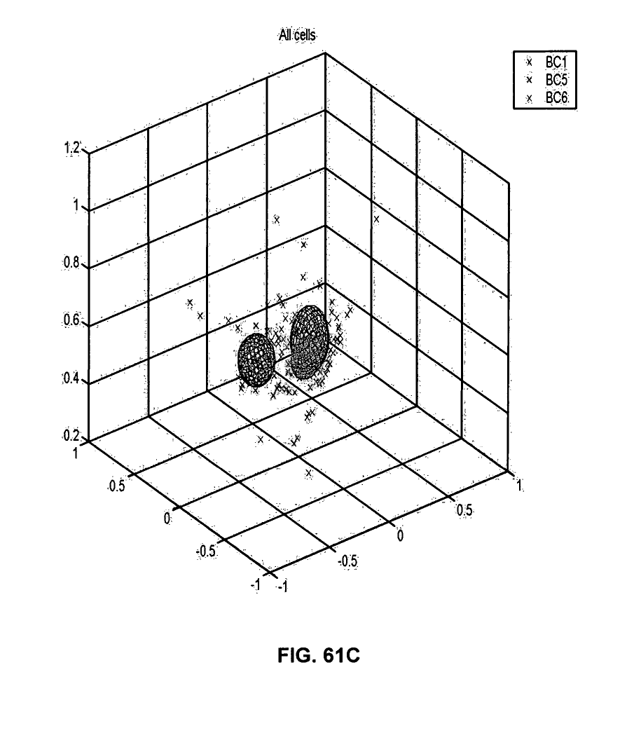

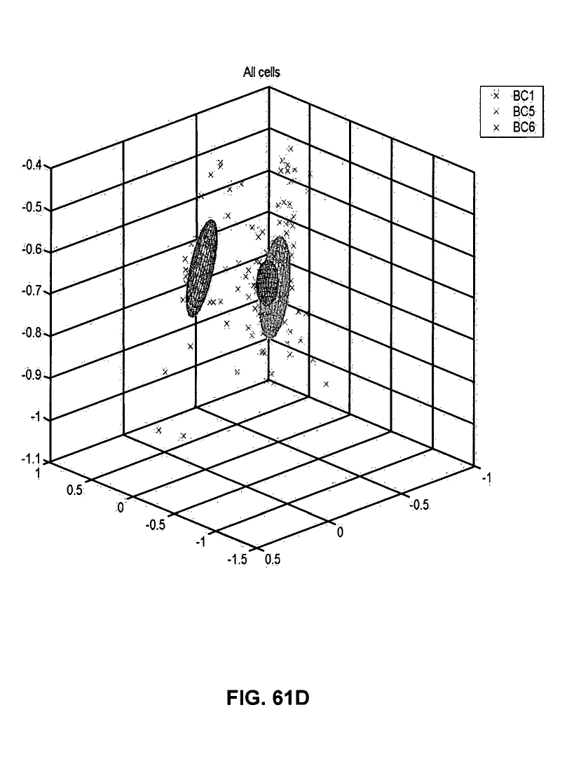

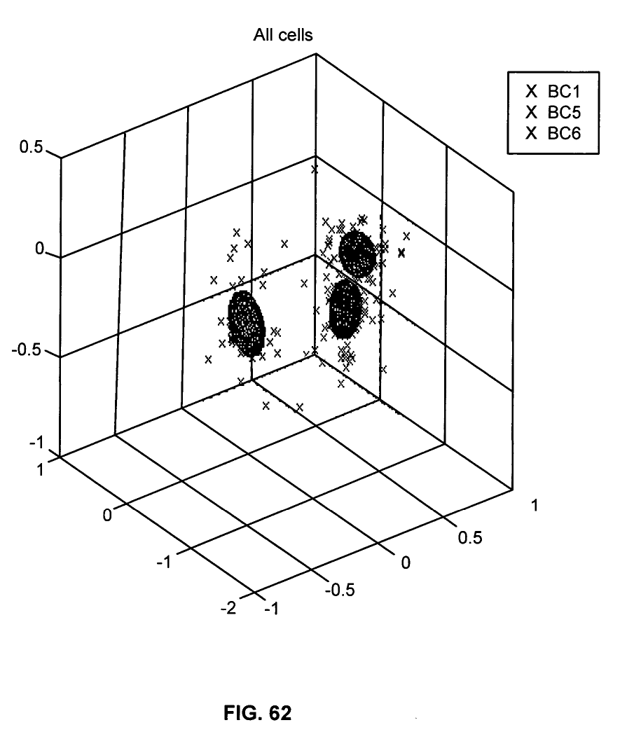

FIG. 61 shows four simplex scenarios corresponding to each of the elicitors present in the elicitor panel. Complex complex scenarios can be created from various combinations of simplex scenarios. A comparison of the complex complex scenarios to the complex scenario shown in FIG. 62 provides a mechanistic interpretation of the results.

Non-verbal classifiers can be used for integrating outputs from orthogonal biosensor arrays. Experiments for each biosensor generate a set of numerical and non-numerical features descriptors (features). Non-numerical features (e.g., nominal and ordinal) are useful for orthogonal biosensor array communication. Different types of agents provide ordinal features that can be nominally classified as strong or weak, for example. Nominal features can be selected to be non-sensor specific. The non-numerical feature plus the nominal value is equivalent to a label. Table 13 defines a lexicon as the underlying collection of “lexemes” or ordinal descriptors for the set of labels that feeds into the different scenarios. The corresponding indicator values dictate the contribution of the individual lexemes each scenario. The final scenario is described by the vectors {Lexicon}+{Indicators}=Scenario.

TABLE 13

| “Lexicon” consists of “lexemes” | “Indicators” |
|---|---|
| {response strength none | { p₁ |
| response strength weak | p₂ |
| response strength medium | p₃ |
| response strength high | p₄ |
| response strength highest ever | p₅ |
| toxicity none | p₆ |
| toxicity weak | p₇ |
| toxicity medium | p₈ |
| toxicity high | p₉ |
| . | . |
| . | . |
| . | . |
| Agent Hg none | pₙ |
| agent Hg low concentration | p ₙ₊₁ |
| agent Hg medium concentration | p ₙ₊₂ . . . } |

### Example 20

### Other Forms of Soft Classification

Graph theoretic modeling approaches including Bayesian networks and dependency networks fit within the soft classification framework

### Directed Graphical Models^{1,2}

Graphical models are defined by a graphical structure, M, which consists of sets of nodes or vertices, V, and edges or arcs, E: S=(V, E). Each node represents a random variable described by a joint probability distribution, fᵢ, with parameters, θᵢ. The presence of an edge between two nodes represents a conditional dependence. For a graph structure S=(V,E), the set of joint probability distributions and associated parameters are reported in vector format where fᵢ is a member of set F with corresponding parameter vector.
¹ Heckerman, D. (1995) A tutorial on learning with Bayesian Networks. Technical Report MSR-TR-95-06. Microsoft Research, Microsoft Corporation.

² Husmeier, D. (2003) Sensitivity and specificity of inferring genetic regulatory interactions from microarray experiments with dynamic Bayesian networks. Bioinformatics 19(17):2271-2282.

Directed Acyclic Graphs

A directed edge allows represents allows information to flow in a single direction where the source node is called the parent and the recipient node is called the child. Take as an example FIG. 69, which is a schematic of interactions between the signal transduction pathways initiated by the Gᵢ and G_{q} receptors showing both non-conditional and conditional interactions. With reference to FIG. 69, solid arrays indicate conditional interactions. Dashed arrows indicate non-conditional interactions. FIG. 69A is a schematic of Gs and Gq pathways. Adapted from Gomperts et al.³; FIG. 69B is an acyclic graph derived from upper segment of Gs and Gq pathways; FIG. 69C illustrates modular breakdown for network structure shown in 69A. Segmentation experiment shown in FIG. 69 uses forskolin to decouple PKA activity from AC mediation (Modules 2 and 4). The abbreviations of FIG. 69 are as follows: βγ, βγ subunit of GPCR; αq, a subunit of Gq GPCR; αs; a subunit of Gs GPCR; PLC, Phospholipase C; AC, adenylate cyclase; IP₃, inositol tri-phosphate; R, calcium ion channel regulatory protein; Ca, calcium; PKC, protein kinase C; cAMP, cyclic adenosine monophosphate; PKA, protein kinase A.
³Gomperts, B. D., Kramer, I. M. and Tatham, P. E. R. (2002) Signal Transduction. Elsevier Academic Press, New York ISBN: 0-12-289632-7.

The upper section of this pathway is reduced to a simple directed acyclic graph (DAG) as shown in FIG. 69B described by the set of vertices, V=(A,B,C,D,E,F) and edges E={(A,C),(B,D),(C,E),(D,F),(C,F),(D,E)}, where A is the parent of C and D is the child of B. A is the ancestor of vertices E, F, who are the descendents of A.

The absence of cyclic structures, a defining property of DAG's or Bayesian networks, indicates that a node is dependent on its descendents but independent of non-descendents. Practically speaking this implies that the flow of information through the graph is in a single direction as shown in FIG. 69B. Bayesian conditional probabilities can be expanded as shown in Eqn. 1, where the set of random variables for S is represented by X=(X₁, X₂, . . . , Xₙ) and pa[Xᵢ] represents the set of parents for node i.

$$P ( {X}_{1} , {X}_{2} , … {X}_{n} \vert {\overline{S}}_{B} ) = {\prod }_{i = 1}^{n} P ( {X}_{i} \vert \overline{pa} [ {X}_{i} ] , {\overline{Θ}}_{B} ) \qquad ( 1 )$$

Nodes are associated with random variables particular to the inference problem being addressed. Random variables of interest for inferring genetic networks might include individual genes or clusters of genes with associated gene expression levels. Random variables of interest for inferring signal propagation patterns through protein networks with chromatophores include perceived changes in cell area or colors⁴.
⁴ Roussel, N. (2003) Advanced image segmentation and data clustering concepts applied to digital image sequences featuring the response of biological materials to toxic agents. Master's Thesis, Oregon State University.

Dependency networks⁵ are related to DAG or Bayesian networks but differ in that the acyclic requirement is relaxed. Consequently, nodes in dependency networks are independent of all other nodes and joint probability distributions are as shown in Equation 2.
P(X₁,X₂, . . . Xₙ|\overline{Paᵢ},\overline{S}_{D})=P(Xᵢ|\overline{X}\Xᵢ,\overline{Θ}_{d}) (2)

⁵ Hulten, G., Chickering, D. M. and Herckerman, D. (2003) Learning Bayesian Networks from Dependency Networks: A preliminary study.

Dependency networks have major advantages relative to Bayesian networks in that the joint probability distribution at each node can be learned prior to network integration allowing for easier integration of modularity. In addition, cyclic motifs in biological networks are the rule rather than the exception meaning that certain modifications are necessary in order to apply the standard Bayesian approach. The major advantage of Bayesian networks is the simple factored form of the joint distribution, which has advantages for inference in situations following model building were the necessary computing resources are limited. This along with the relatively easy experimental approach described herein has important implications for the eventual wide-spread dissemination of these tools.

### Learning Networks

For the purposes of the following discussion we assume that the family of probability distributions, F, is considered known and fixed. The family of probability distributions can be independently derived using dependency networks or other similar approaches as described. The network parameters, Θ, define the conditional probabilities associated with each edge, providing causal links between the various elements in the network. The objective is to learn the network, S_{B}, produced by elicitor set experiments, which requires identifying the network structure that best fits the experimental data. In other words, we must find the structure S_{B}i that maximizes P(S_{B}|D).
S_{B}_{ᵢ}=arg max_{S}_{_{Bi}}{P(S_{B}|D)} (3)
and the parameters Θ that maximize P(Θ|D, S_{B}.). Bayes rule provides Eqn. (4) and (5) with P(S_{B}i) as the

$$P ( {S}_{{B}_{i}} \vert D ) = \frac{P ( D \vert {S}_{{B}_{i}} ) P ( {S}_{{B}_{i}} )}{{\sum }_{{S}_{B}} P ( D \vert {S}_{{B}_{i}} ) P ( {S}_{{B}_{i}} )} \qquad ( 4 )$$

prior probability on network structure \mathring{C}, and P(D|S_{Bi}) is the marginal likelihood for network structures.
P(D|S_{B}_{ᵢ})=∫P(D|Θ,S_{B}_{ᵢ}) PΘ|S_{B}_{ᵢ})dΘ (5)
The integral in Eqn. (5) is tractable for complete data (data for every node in the network) providing closed form relationships, such as the linear Gaussian (continuous data) or multinomial (discrete data) distributions are selected. Many standard approaches have been developed for solving these networks, which are NP-Hard (solution difficult increases exponentially with the number of nodes)⁶. Appropriate selection of initializing network structures and the use of dependency networks as tools for network learning can reduce the computational complexity of the problem significantly.
⁶Cf. Luger, G. F. (2002) Artificial Intelligence: Structures and Strategies for Complex Problem Solving, 4th Ed. Addison Welsey, New York.

Direct sampling of Equation (4) will be possible with small network structures. Larger network require the use of numerical methods, of which the Monte Carlo Markov Chain (MCMC) is widely applied. New network structures are proposed in an iterative process using three elementary proposal moves from a given node—deleting, reversing or adding an edge, and accepted using a modification of the Metropolis-Hastings acceptance criterion as shown in Equation. (6). N is the neighborhood of S_{Bi}, with neighborhood size is defined as the set of diacyclic graphs that can be reached in a single move or iteration. Non-acyclic conditions that result from this algorithm are considered invalid graph structures. Cyclic conditions represent over-lapping candidate graph structures or hypotheses. The Hasting ratio represents the ratio of the neighborhood sizes of the networks involved in the proposed change in graph structure and is used as a criterion for network convergence.

$$\frac{Q ( {S}_{{B}_{OLD}} \vert {S}_{{B}_{NEW}} )}{Q ( {S}_{{B}_{NEW}} \vert {S}_{{B}_{OLD}} )} = \frac{N ( {S}_{{B}_{OLD}} )}{N ( {S}_{{B}_{NEW}} )} \qquad ( 6 )$$

### Example 21

This example concerns monitoring optical changes in chromatophores and analyzing such systems using a detection system configured to receive a light flux from a chromatophore population retained in a cell chamber. Cell chambers can be constructed using a series of layers that are laminated. Alternatively, a cell chamber can be formed using, for example, injection molding, casting, or machining. In a laminated cell chamber, one or more layers that define an interior of the chamber form a biomatrix layer on which the sensor cells are cultured. Some layers are configured to provide fluid interconnect and transport for delivery of samples and other reagents. One or more of the layers is transparent to permit detection of optical changes in the chromatophores. Biocompatible materials are selected for the cell chamber and are generally durable materials that tolerate sterilization processes. Suitable materials include MELINEX 453 polyester because it is clear and hydrolytically stable at room temperature.

The laminated cell chamber is sandwiched between the two halves of a fluid interconnect that is in communication with a fluid delivery system and a sample injection system. Dimensions and hexagonal geometry of the fluid interconnect are selected for convenience in illuminating the sensor cells and recording changes in the sensor cells.

Construction of a cell chamber using a lamination process permits cell chamber geometry and fluid interconnects to be configured as required. In addition, the lamination method permits rapid, inexpensive chamber designs so that cell configurations can be selected for a particular application.

A pressure-sensitive adhesive (PSA) bonds laminated layers together to form the cell chamber. The PSA was selected to be biocompatible and hydrolytically stable. Hydrolytic stability was assessed by immersing PSA samples in water for several days. Some samples became milky in color, indicating that these sample were water permeable and likely to leak after prolonged water exposure. Avery Dension adhesive FT 8311 was used because of its ability to withstand exposure to analytes and other materials. Further, biocompatibility tests were conducted on FT8311. Several chambers were fabricated and populated with cells. The populated chambers were monitored over several weeks to determine how long the cells would survive in the chambers. The only biocompatibility issue associated with the FT 8311 was that occasionally the adhesive would form bubbles that served as small antechambers next to the main cell chamber. If chemicals were used to clean the chambers prior to population, these chemicals can be caught in the antechambers and subsequently diffuse into the main chamber after cell population. The result is a drastic reduction in the shelf life of the cells.

The geometry of the cell chamber was based on the feeding requirements of sensor cells, the shear tolerance of sensor cells, and to afford an optimal field of view of the sensor cells, as described below.

In the first embodiment of a cytosensor the optics subsystem images the center 1 mm×1 mm area of the 3 mm×3 mm total sample area onto the color CCD array for optimal field of view. A Hastings triplet lens provided an achromatic, flat field of view.

The fluidics subsystem is designed such that the shear tolerance of cells is compensated for. In order to maintain cell viability the shear stress for a range of design options (chamber cross sectional area as measured by the chamber height) was calculated assuming viscous channel flow. Results show that the shear stress decreases rapidly with channel height and falls below the expected allowable of 1 N/m² at a height of 350 microns with a flow rate of 10 liters per minute in a one-millimeter wide channel. This result is inversely linear with flow rate (if the flow rate is reduced by a factor of 2 then the maximum channel height that yields the critical shear stress is increased by a factor of two).

The uniformity of delivery of analyte to the chamber and the flow conditions in the chamber depend on the downstream variability of the chamber cross section geometry. The chamber inlet section and the flow connections with the inlet channels influence the chamber flow conditions. These conditions were studied for a range of flow rates using a rapidly diffusing inlet (approximately 30° diffusion angle). Images of the flow within the chamber using a dissolved dye in water injected upstream of the chamber and allowed to flow steadily into the chamber show that a relatively uniform front of analyte can be achieved within the chamber and the frontal concentration gradient will depend on the diffusion coefficient of the analyte. Results were identical for a wide range of flow rates from 1 to 50 μL per minute.

Fluidics connection points were built to specifications described elsewhere herein. The connection of the chamber to the analyte supply reservoir can be made in a variety of ways as long as the connections are bio-compatible and maintain their integrity (leak-proof) under pressures developed within the chamber. A simple connector was used which had an inlet tube at right angles to the chamber flow direction. The end of the tube was flanged, with the flanged portion held tight against the inlet port walls such that fluid could escape through the tube into the chamber. Other possibilities were used with success including o-ring seals and gasket material to prevent leaks.

A syringe injection system and syringe pumping system with the following attributes was used to deliver sample and aqueous medium to the cell chamber. This allowed easy variation of the flow rate ranges and provided long term operation at a set flow rate condition. The syringe pump was not absolutely continuous in that the supply mechanism provided very short duration steps to the syringe injection mode. Due to the very slow flow rates used this resulted in pulsing flow conditions within the chamber. This condition was found not to change the overall operation of the chromatophores.

Response time of the instrument includes sample arrival time at the cell chamber that can be adjusted according to the following considerations. The analyte can be delivered as a uniform front (slug) flow into the chamber. The time for the sample to arrive at the chamber can be varied by altering the set flow rate delivered by the pump or by varying the inlet channel length and cross sectional area. Sample calculations of the dead-volumes used in this embodiment show that the dead-volumes are sufficiently small. The waste stream was discarded, but can be expected to have other uses, such as providing a sample for further analysis to unspecified additional instrumentation, such as a mass spectrometer.

A schematic diagram of an optical system is provided. A lens system was used to magnify the field of view and project it efficiently onto the imaging chip of the specified video camera. The cell chamber was illuminated with Nichia “white” LEDs which are based on a Ce:YAG phosphor over a blue InGaN LED emitter. The resulting spectrum still contains a large amount of the blue light in addition to a broad-band quasi-white emission from the phosphor. Filters or gratings can be used to adjust and/or dynamically alter the wavelength of light in order to accentuate the readouts from given colors of sensor cells. The illumination source can be filtered to test for optimum color illumination. The optimum color can then be matched by either filters or a combination of pure colored LEDs. Certain cells may reflect or transmit certain colors much better and thus provide more efficient detection. LED illumination adds no heat to the cells and requires very simple power of 10 mA at about 2.8 V. A periodically switching circuit can be used to modulate the light source during measurements to permit narrowband or lock-in detection.

A detection and identification algorithm uses a time sequence of images acquired either asynchronously or synchronously. In one embodiment, the images are collected on the average of one frame per second, but this rate is typically based on the rate of content change in the previous images. The system is capable of acquiring up to 30 images per second. The optimal sampling rate should match the estimated (on-line) rate of change of the image content.

Each image is represented by N×M array A, whose entries are P dimensional vectors. Such an array can be viewed as P dimensional discrete field. N and M define the grid, which conceptually divides the camera field of view into small rectangular regions called pixels or picture elements (e.g. field of view X by Y is divided into rectangles of (X/N) by (Y/M)). The P-dimensional discrete field model represents most of the known digital image formats. The image is initially acquired in RGB format. Thus each entry of A represents an average color from a picture element using P=3 dimensional vector of intensities of red, green and blue color components. Typically these intensities are normalized to take the values between 0 and 1. The generalized color histogram can be then viewed as a map of A into a 3 dimensional unit cube. Since the image is a discrete description of the optical appearance of the field of cells, the analyzer uses the colors and shapes to characterize the cell state.

Color segmentation based on reducing the number of distinct colors in the image to the maximum of K color-clusters (in the first embodiment, K is typically between 5 and 10). This reduction process is based on a generalized K-means algorithm. A color cluster is a set of colors which are “alike”, and the colors from different clusters are “not alike”. A quantitative description of a cluster is “an aggregation of points in a multi-dimensional space, such that the distance between any two points in the cluster is less than the distance between any point in the cluster and any point not in it.” Intuitively clusters are connected regions of a multi-dimensional space containing a relatively high density of points, separated from other such regions by a region of a relatively low density of points. The segmentation takes place in a color histogram space. The RGB coordinates are transformed to a suitable color space where the distance between colors can be defined to ensure, for example, visual uniformity. The HSV (hue, saturation, and value) space is used in some examples. The distance D is calculated using quasi-periodic measure. The number of color classes K is adjusted adaptively using dynamic programming techniques. The smallest K is sought such that the variance (calculated using D) of each cluster does not exceed a priori specified threshold. The dynamic programming method allows re-using the initial segmentation for K classes to perform efficiently (K+1) class segmentation.

The segmentation is performed in an abstract color space. The clusters are next mapped onto a 2 dimensional image space where the geometrical shape of clusters is of interest. The morphological filtering is applied to each color class to eliminate small spatially isolated clusters and to merge the close ones. For example the cluster should be of the size expected for a chromatophore or a group of chromatophores.

The image segments represent now the areas filled with erythrophores, melanophores, and so on. The background is also isolated. The temporal statistics and features of filtered clusters are next calculated and include: area, convex hull, bounding polygon, convex area, equivalent diameter, major and minor axis lengths, solidity, extent, orientation, eccentricity, and nth order moments (both spatial using Euclidean distance, and in color space using D measure).

The next two steps determine if the statistics representing various chromatophores change significantly over time based on analysis of series of images. Dynamic patterns (trends) of selected statistics are estimated (for example, area and 2nd order spatial moments), using a conditionally linear filter that generalizes Kalman filtering methods. This filter tests sequentially two alternative hypotheses about the parameters of a mixture of two distributions. The null hypothesis assumes that there is no bioagent present and that the cell's statistics are slowly varying. The alternative hypothesis assumes that after being exposed to a bioagent the cell statistics reach a distinct from the initial state equilibrium. A mixture of two Gaussian distributions is used with a slowly varying mean and variance of the first one and constant moments of the second. The test control parameters include probability of false alarm and detection delay. The acceptance of the second hypothesis marks the end of detection process and stops the image acquisition.

Time-dependent or dynamic pattern behavior is parametrically identified from the statistics waveform in the transition region (i.e. in the non-causal neighborhood of the time instant signaled by the detection algorithm). A BARMAX (Bilinear Auto-Regressive Moving Average) model is used, however various parameterized models can be used. The estimated model parameters are matched against a stored library of template signatures. These template signatures represent the results of controlled experiments when the living cells were exposed to known (type and concentration) bioactive agent. A proximity matrix approach is used but since the matching problem is of a low dimension (number of model parameters), various statistical (geometric) pattern recognition techniques can be used. Some of the cell responses to specific bioactive conditions lend themselves to structural (syntactic) pattern recognition. In this approach the patterns are composed of simple sub-patterns. A sub-pattern can be built from simpler parts with grammatical techniques. Primitives (or the simplest sub-patterns) can be shared among many different experiments just relaxing the need for extensive experimentation and creation of large signature libraries. The outcomes of pattern matching for various statistics represent “votes” that establish a confidence level for agent identification.

Detection methods are conveniently implemented on a general-purpose microprocessor running Windows NT. The numerical calculations use fixed-point arithmetic and are ready for implementations on DSP type boards. The color segmentation algorithm is multi-threaded and can be easily ported to a multi-processor computer or multi DSP chip board. The identification and pattern recognition algorithms have hierarchical organization suitable for communication between several sensors in order to obtain more reliable detection results.

The representation of the cells state as a mathematical field and the general segmentation algorithm allow use of multi-spectral (not necessarily visual) images. Due to the sequential data processing the storage (i.e. memory) requirements are minimal. The system minimizes energy use by adapting the image sampling rates, which is an essential feature in mobile device operation.

Screen capture is shown with reference to the working examples based on detection results using 92 images that are acquired every 5 seconds. An alarm is triggered at a 60th frame, with a delay of two frames. Signature identification uses 10 frames (from frame 58 to 67). Frames 1 to 57 correspond to slowly changing statistics (null hypothesis). Frames 68 to 92 represent statistical equilibrium.

Microlamination techniques for fabricating microtechnology-based devices for micro-scale bio-devices are based on patterning and assembling thin shims and bonding them into a composite assembly. Microlamination typically involves three steps: 1) lamina patterning, 2) laminae registration to form an assembly, and 3) bonding of the assembly. A dual microchannel array fabricated by microlamination methods using polyimide as a thermal adhesive is shown in Example Embodiment 11. Microlamination permits fabrication of metal and/or polymeric devices with high aspect ratios in large production volumes.

Various microlamination schemes can be used. For example, polyester films can be patterned using 266 nm laser micromachining and bonded together using pressure-sensitive adhesives. Polycarbonate films can be patterned with 266 nm laser micromachining and bonded by solvent welding.

In some cell chambers, two laminae are used. One lamina is a flat cover plate that provides optical access into and sealing of the cell chamber. The other lamina is micromolded lamina. Such polymeric microchannels and chamber substrates can be fabricated using soft lithography methods and substrates can be formed in poly(dimethylsiloxane) (PDMS). The PDMS substrates can be formed using a combination of laser-based mask-making and contact photolithography. A mask can be made using selective laser ablation of a chromium-on-glass contact mask. Contact photolithography on a thick photoresist can be used to create a master mold of the microfluidic structure to be used as a mandrel in micromolding. An example of an SU-8 master mold for a particular embodiment is shown in the figures. In addition, bulk silicon etching can be used to create micromolding mandrels. A PDMS pre-polymer can be cast and cured on a mandrel.

Bonding of a cover plate onto a PDMS substrate can be handled conformally or adhesively. If necessary, cover plates can be readily assembled and disassembled by using nonadhesive, conformal contact with the molded PDMS substrate, allowing direct access to sample microchannels after an experiment. PDMS sample channels and chambers may be sealed permanently by oxidizing two PDMS surfaces in a plasma discharge and bringing them into conformal contact. Alternately, UV-curable adhesives may be used to permanently seal chambers. The use of adhesives may require the design and fabrication of adhesive reservoirs in the polymeric substrate.

PDMS substrates can be used as mandrels in a replica molding process to generate modest volumes of polyurethane or other crosslinked polymers. Replica molding is capable of fine-feature (submicron), high-aspect-ratio (20:1) pattern generation inside of microchannels and chambers, if necessary. Bonding between polyurethane and cover slips can be handled by a partial cure method or by UV-curable adhesives. The partial cure method simply involves putting the cover slip in contact with the polymeric substrate before it is fully polymerized and then finish curing the substrate.

High-volume molding and bonding can also be provided through injection molding and ultrasonic welding of cover slips to substrates.

Either tissue explants (scales, fin tissue), isolated chromatophores , or continually dividing populations of chromatophores can be employed in a cell-chamber. Chromatophore populations can also be populated into entrapped forms or encapsulated forms. In a specific embodiment, a cell chamber was populated with isolated Betta chromatophores, comprising approximately 1000 chromatophores (90% erythrophores, 9% melanophores, 1% iridophores) into a 1 mm² viewing area. The ratio of color types of chromatophores in a population can be varied to take advantage of distinct sensitivities of certain color classes of chromatophores. For example, there is evidence of selective sensitivity of erythrophores to cholera toxin, and melanophores to pertussis toxin. The numbers of chromatophores which are populated into the cytosensor can also be varied. Also, additional cell types such as PC12 cells, which can provide additional dimensions of sensitivity by being coupled to chromatophores, can also be included in the sensor cell populations.

The sensor cells are typically incorporated into a chamber or entrapped form at least one to three days prior to testing against an analyte, in order to give time for the cells to become firmly attached and adapted to in vitro conditions. That period can be lengthened to up to the maximum period that Betta chromatophores survive in vitro. During this storage period (the time between initializing the population and when the cells are used to test analytes), the cells have feeding requirements that are attended to according to the general criteria that a population of cells requires a flow rate of medium amounting to an exchange of 50 to 5,000 picoliters per cell per day, and 0.02 to 2 pmol O₂ per cell per day. In a specific embodiment involving 1000 chromatophores in a 1 mm² viewing area of a cell chamber, this degree of medium exchange is accomplished by passive diffusion of medium from a bulk volume in which the chamber inlets and outlets are submerged. The feeding requirement can also be accomplished via active fluid flows, conveyed by capillary action or mechanical pumping.

As described for a specific embodiment, upon injection of 100 nM norepinephrine, the analyzer indicated >0.99 probability of a biologically active agent. Similarly, the optical changes occurring in Betta chromatophores upon exposure to bacterial cells and to PC12 cells show that a cytosensor built according to these specified principles and designs is capable of detecting biologically active agents with strong certainty.

### Example 22

This examples concerns using a cytosensor, one embodiment of which is illustrated in FIG. 19 schematically that includes an analyte entry tube that delivers an analyte to a reaction cell that contains selected chromatophores. The reaction cell defines a reaction chamber that includes side walls and one or more transparent windows, and the chromatophores are held stationary in the reaction chamber. In a specific embodiment, the analyte entry tubing is 0.25 mm inside diameter TEFLON tubing, and a 1 microliter analyte volume is introduced into the reaction chamber. The analyte can be introduced with a syringe, a syringe pump, a peristaltic pump, or other pump. Alternatively, the analyte can be introduced by wicking with, for example, cellulose fibers, or using a capillary flow.

Chromatophores introduced into the reaction chamber can be retained in the reaction chamber by permitting the chromatophores to settle onto a chamber surface. Superior chromatophore retention is achieved by treating the chamber surface with an attachment factor such as collagen and/or fibronectin. Attachment of the chromatophores is generally adequate 1-24 hr after introduction, and can be verified by observation with a light microscope. After the chromatophores are attached to the chamber surface, flow rates into and out of the chamber can be increased without dislodging the chromatophores.

FIGS. 26A-26B illustrate one embodiment of a reaction cell. A reaction chamber is defined in a laminated stack of sheets of a transparent, adhesive-backed material such as MELINEX. The reaction chamber is typically rectangular, with representative dimensions of 2 mm by 3 mm by 0.4 mm. The 0.4 mm depth of the chamber is generally selected as an integer multiple of the thickness of the sheets laminated to form the reaction cell. Inlet and outlet apertures of about 0.5 mm diameter are provided for introduction of and extraction of analytes and other reagents.

As shown in FIG. 26A, the reaction cell is mounted to a fluid interconnect. The fluid interconnect includes a top plate and a bottom plate having central apertures to permit illumination of the chromatophores. These plates are illustrated in FIGS. 27 and 28A-28B. Mounting holes are provided for attaching the fluid interconnect to the reaction cell, and fluid inlets/outlets are provided as well. Bolts (not shown) are used to connect the top and bottom plates, sandwiching the reaction cell.

FIG. 29 is a diagram of an alternative reaction cell that defines two reaction chambers. A stack of polyimide layers and copper layers are laminated by heating the stack to that the polyimide layers bond the copper layers. The stack is terminated on a first surface with a first end cap, and on a second surface with a second end cap that includes inlet/output apertures for any necessary analytes or other materials. Reaction cells that include fewer than or more than two reaction chambers can be fabricated similarly.

FIGS. 30A-30C are schematic diagrams of a multi-analyte reaction cells that include analyte reservoirs and FIG. 31 is photograph of a micromold for fabrication of reaction chambers.

One or more light sources such as LEDs, a laser diode or other laser, or lamps are situated to illuminate the chromatophores. In an embodiment, illumination from the light sources is collected with a lens that directs the collected illumination to the chromatophores. Light reflected by or transmitted through the chromatophores is collected with a lens and directed to a imaging system that includes a CCD or other camera. Typically, the chromatophores are imaged onto the CCD. The CCD supplies an electrical image signal (typically an analog NTSC video signal) to a digitizer that produces a digital image of the electrical image signal that is supplied to a computer. Alternatively, an image sensor that provides a digital image directly can be used. The computer receives the digital image and stores the digital image in a tagged image file format (TIFF) or other convenient format for processing. Images are generally obtained at a rate of about 2/sec. The apparatus is configurable for measurement of transmitted or reflected light. Typically, transmitted or reflected light is suitable for melanophores, erythrophores, and xanthophores, but for some chromatophores such as iridophores, reflected light is superior. The apparatus is also configurable for measurements of fluorophores.

Images are typically acquired in a red-green-blue (RGB) color representation in which values (r, g, b) for each of the colors R, G, B are assigned to each pixel. Other representations can be used, such as a hue-saturation-intensity representation, or other representations. After conversion into the selected color representation, the image data is segmented into areas corresponding to one or more predefined colors or color ranges that are based on the selected chromatophores. A color segment is defined as a predetermined number of adjacent pixels having a color within a predetermined range. This procedure is described in detail below.

In a specific example, the image sensor is a ½″ interline transfer CCD color camera. Color camera variables include the number of the CCD arrays (e.g. 3-CCD arrays with RGB filters), CCD formats (typically ⅓″, ½″ and 1″), and resolution (e.g., the number of horizontal and vertical pixels N, M, respectively). For example, a CCD camera can have N=768 columns and M=494 rows of pixels, or 379,392 total pixels and the CCD cell size is 8.4 μm (H)×9.8 μm(V). The camera produces an analog RGB signal that is digitized using a multi-channel frame grabber having sampling rates up to 30 MHz and a data transfer rate of 130 MB/second. A digital color camera can also be used. Such a camera typically has a variable speed shutter (1/60 sec. to 1/10,000 sec.) and can be operated both synchronously and asynchronously.

FIG. 40 is a block diagram of a method of processing the digital images that uses a time sequence of images acquired either asynchronously or synchronously. Images are typically collected at about one image (frame) per second, but the collection rate is adjusted based on the rates of change of image parameters calculated from previous image and acquisition rates of up to 30 images per second are achievable with conventional image sensors. An optimal acquisition rate can be selected to match the estimated (on-line) rate of change of the image content.

Each image is represented by N×M array A, whose entries are P dimensional vectors. Such an array can be viewed as P dimensional discrete field. N and M define a grid that divides the image into small rectangular regions called pixels or picture elements, e.g., a field of view of dimensions X by Y is divided into pixels (rectangles) of dimensions (X/N) by (Y/M). The P-dimensional discrete field model represents most digital image formats, including the common RGB format. Thus each entry of A represents an average color from a picture element using a P=3 dimensional vector of intensities of red, green and blue color components. Typically these intensities are normalized to take values between 0 and 1. The generalized color histogram can be then viewed as a map of A into a 3 dimensional unit cube. Since the image is a discrete description of the optical appearance of the field of cells, the analyzer can use colors and shapes to characterize the cell state.

In a color space transformation step, the representation of the image in RGB space is transformed to a suitable color space where the distance between colors can be defined to provide, for example, visual uniformity. In an example, a HSV (Hue, Saturation, and Value) space is used.

After color space transformation, an image segmentation step is executed to divide the image into segments based on pixel color. Color segmentation is performed by selecting a specific color coordinate or combination of coordinates, and characterizing pixels based on this coordinate (or coordinates). This process is based on a generalized K-Means algorithm. A color cluster is a set of colors which are “alike,” and the colors from different clusters are “not alike.” A quantitative description of a cluster is “an aggregation of points in a multi-dimensional space, such that the distance between any two points in the cluster is less than the distance between any point in the cluster and any point not in it”. Intuitively clusters are connected regions of a multi-dimensional space containing a relatively high density of points, separated from other such regions by a region of a relatively low density of points. The segmentation takes place in a color histogram space. The distance D is calculated using a quasi-periodic measure based primarily, in a representative example, on the hue coordinate (H) of the image pixels and secondarily, on a contribution based on the saturation coordinate (S). A number of color classes K is adjusted adaptively using dynamic programming techniques. The smallest K is sought such that a color variance (calculated using D) of each of the clusters does not exceed an a priori specified threshold. The dynamic programming method re-uses the initial segmentation for K classes to perform efficiently (K+1) class segmentation. Typically, the image is divided into 5-10 color classes.

After segmentation, the color clusters are associated with their corresponding image (spatial) coordinates for evaluation of cluster geometrical shape in a morphological filtering step. Morphological filtering is applied to each color class to eliminate small spatially isolated clusters and to merge close clusters. For example, morphologically filtered clusters should correspond to the size expected for an image of a chromatophore or a group of chromatophores. The morphologically filtered image contains areas that can be associated with erythrophores, melanophores, and other chromatophores as well as a background. Statistical and morphological parameters of the filtered clusters are calculated in a statistics calculation step. Representative calculated parameters of the color clusters include area, convex hull, bounding polygon, convex area, equivalent diameter, major and minor axis lengths, solidity, extent, orientation, eccentricity, and nth order moments (both spatial using Euclidean distance, and in color space using, for example, the distance D).

In a dynamic filtering step and a change detection step, changes in filtered color clusters are detected based on an analysis of series of images rather than a single image. Dynamic patterns (trends) of selected statistical or other parameters are estimated (for example, area and 2nd order spatial moments), using a conditionally linear filter, which generalizes traditional Kalman filtering methods. This filter tests sequentially two alternative hypotheses about the parameters of a mixture of two distributions. The null hypothesis assumes that there is no bioagent present and that the cells statistics are slowly varying. The alternative hypothesis assumes that after being exposed to a bioagent the cell statistics reach an equilibrium that is distinct from the initial state equilibrium. In a representative example, a mixture of two Gaussian distributions is used having a slowly varying mean and variance and a second having constant moments. The test control parameters include probability of false alarm and detection delay. The acceptance of the second hypothesis marks the end of detection process and stops the image acquisition.

Dynamic patterns are parametrically identified from the statistics waveform in the transition region (i.e. in the non-causal neighborhood of the time instant signaled by the detection algorithm) using, for example, a BARMAX (Bilinear Auto-Regressive Moving Average) model, but various parameterized models can also be used. The estimated model parameters are matched against a stored library of template signatures. These template signatures represent the results of controlled experiments in which chromatophores are exposed to known (type and concentration) bioactive conditions. Mercury and Gemini use. Since thus matching problem is of a low dimension (number of model parameters), various statistical (geometric) pattern recognition techniques such as proximity matrix methods can be used here. Some of the cell responses to specific bioactive conditions lend themselves to structural (syntactic) pattern recognition. In this approach the patterns are composed of simple sub-patterns. A sub-pattern can be built from simpler parts with grammatical techniques. Primitives (or the simplest sub-patterns) can be shared among many different experiments to reduce the need for extensive experimentation and the creation of large signature libraries. The outcomes of pattern matching for various statistics represents “votes”, defining the confidence level of agent identification.

Such a detection method is implemented on a general-purpose microprocessor such as an embedded Intel Pentium processor running Windows NT or a dedicated processor. Numerical calculations can use fixed-point arithmetic and are suitable for implementation on DSP type boards. The color segmentation algorithm is multi-threaded and can be easily ported to a multi-processor computer or multi DSP chip board. The identification and pattern recognition algorithms have hierarchical organization suitable for communication between several sensors in order to obtain more reliable detection results.

The representation of the cell's state as a mathematical field and the general segmentation algorithm allow use of multi-spectral (not necessarily visual) images. Due to the sequential data processing the storage (i.e. memory) requirements are minimal. The system minimizes energy use by adapting the image sampling rates to permit mobile device operation.

FIG. 41 illustrates results of image processing as described above. The screen image of FIG. 41 is based on 92 images that are acquired at a rate of about 5 images/second. An alarm is triggered at a 60th frame, with a delay of two frames. The signature identification uses 10 frames (from the 58^{th} frame to 67^{th} frame). Frames 1 to 57 correspond to slowly changing statistics (null hypothesis) and frames 68 to 92 represent equilibrium.

For convenience, FIGS. 5A-5B are provided to illustrate a change in an image hue coordinate after exposure to a selected analyte. While RGB color coordinates change (as shown is FIG. 5A), the change in hue of FIG. 5B is more apparent.

### Example 23

This example concerns selective routing of samples through high-throughput screening devices that can be used in many applications, including cytosensor methods. Bio-capsules; capture dots; and micro-ball-valves also are described.

Micro-balls can be used in biomaterial carriers (capsules containing bio and ferromagnetic material) and in micro-ball valves. Apparatus for the immobilization of biomaterial in alginate beads can be adapted for micro-ball production. Several microball compositions have been made and several thousands of micro-balls have been produced. Micro-balls of different sizes, densities, and percent by weight (Wt %) of ferromagnetic material have been produced. Micro-balls having diameters dp in a range of about 150-1000 μm, ferromagnetic Wt % of about 5%-20%, and densities in a range of about 1.05 g/cm³ to 1.35 g/cm³ have been produced.

Micro-ball carriers are preferably in a size range of about 100-200 μm and can be produced with an extrusion apparatus such as apparatus 51 of FIG. 42. A mixture of 1.5% sodium alginate and 95% water is combined with a ferromagnetic material and an active substance and placed in a reservoir 52. The mixture is directed through a needle 54 or other channel and air is directed to a tip 56 of the needle 54 through an air inlet 58. Introduction of air into the inlet 58 shears off beads 60 at the tip 56. The beads 60 are directed into a CaCl₂ bath 62 in which Ca²⁺ is exchanged for sodium ions Na⁺ so that the alginate material is polymerized. The duration of exposure to the CaCl₂ bath determines the degree of polymerization. FIG. 43 is a photograph of several micro-balls (dp≈150 μm) produced with the apparatus 50.

High-throughput screening devices typically require selective immobilization of biomaterial within a maze of micro-channels. Devices incorporating this capability are preferably programmable for a variety of operating configurations. Referring to FIGS. 44A-44C, a Y-branch 70 permits micro-balls containing a ferromagnetic material to be directed to a channel 76 as controlled by a magnetic field generated by a solenoid 72 positioned at an entrance 74 of the channel 76. Micro-balls are directed to one side of the Y-branch 70 by activating a magnetic field for a selected channel (e.g., channels 76, 78). As illustrated in FIG. 44B, with no applied magnetic field (the solenoid 72 in an off state), magnetic particles 80 in a liquid 82 are directed to both channels 76, 78. With an applied magnetic field as shown in FIG. 44C, the particles 80 are directed to the channel 76. In a specific example, micro-balls (the particles 80) of diameter dₚ=500 μm were directed to a Y-branch having internal diameters of about d_{ch}=1000 μm. In other examples, particles of diameter of dₚ≈100 μm were directed into channels of internal diameter d_{ch}≈200 μm.

Detailed calculations of magnetic fields from the thin film coils and magnetic forces on ferromagnetic beads can be used to determine capture dot geometry. Capture dots can be produced by electroforming into a lithographic mold. Round and square coils have been produced using various photolithographic (PL) test patterns. These patterns can include an oxide via to bottom conductor layer, a pad layer for electrical contact, a coiled conductor layer, and a test structure layer. Multiple patterns can be formed on a single mask. Some examples of mask patterns are shown in FIGS. 45A-45B and an electroformed coil made from the pattern of FIG. 45A is shown in FIG. 46.

A method for making contact printing masks for photolithography includes evaporation of a 0.5 μm chromium layer onto a glass microscope slide or other substrate. The chromium layer is selectively ablated using a 532 nm Nd:YAG laser. Because glass (SiO₂) is transparent to the 532 nm wavelength, the laser ablates the chromium while leaving the glass surface intact. Linewidths at least as small as 35 μm can be produced. The glass surface is typically somewhat “frosted” indicating that some amount of laser ablation or other effect occurs at the glass surface. Masks can also be made by direct writing with an ultraviolet (UV) on Cr/glass plates or by exposing photoresist/Cr/glass plates for etching of the Cr layer.

A two-way micro-ball valve based on capture dots technology for actuating a ferromagnetic ball on the order of several hundred microns can be used. A one-way micro-ball valve is shown in FIGS. 40A-40B. The micro-ball valve includes a valve chamber between a valve orifice (FIG. 47A) and a ball catch plate (FIG. 47B). The device is actuated hydraulically and permits flow in one direction. When flowing from the orifice to the catch plate, the ball is caught in the catch plate allowing flow through the valve. When flowing from the catch plate to the orifice, the ball seals the orifice causing flow to stop. The catch plate thus permits flow through the valve while retaining the ball.

The valve shown in FIGS. 47A-47B defines a chamber of approximately 900 μm diameter and has a glass ball of approximately 400 μm diameter. The valve is made of 9 layers, alternating 0.005 inch MELINEX 453 polyester film with Avery Dennison FT8311 double-sided pressure sensitive adhesive film. There are four FT8311 laminae interspersed between five MELINEX laminae. The two outside laminae are patterned as shown with the orifice and catch plate. The inner laminae are patterned with a circle of the diameter of the valve.

Several one-way valves similar to those of FIGS. 47A-47B were built and tested. Leakage within a diode (one-way) valve can be quantified as a diodicity (i.e. a ratio of pressure required to give an intended flow rate in a first direction to the pressure required to create the same flow rate in a second direction, opposite the first (an undesired flow direction). Table 14 summarizes the diodicity results from a particular valve.

TABLE 14
Micro-ball Valve Diodicity

| | Forward flow | Reverse flow | |
|---|---|---|---|
| Flow rate | Pressure | Pressure | |
| (ml/min) | (psi) | (psi) | Diodicity |
| 5 | 1.05 | 0.37 | 2.84 |
| 8 | 1.48 | 0.95 | 1.56 |
| 9 | 1.6 | 1.12 | 1.43 |
| 10 | 2.96 | 1.4 | 2.11 |

The diodicity of this valve is limited by alignment errors between layers that prevent the ball from sealing the orifice. Misalignment as a function of bonding pressure was measured and is graphed in FIG. 48.

In other examples, adhesive appeared within the chamber causing the ball to stick as the pressure sensitive adhesive oozes under high bonding pressures. Therefore, the design for the middle chamber laminae was changed so that the circles cut in the FT8311 laminae for the chamber were larger than the circles cut in the MELINEX laminae. This was done to keep the ball from being exposed to any excess adhesive that might be squeezed out from between laminae. In addition, misalignment can still occur and alignment holes were provided to reduce misalignment. Use of a 266 nm Nd:YAG laser for patterning MELINEX layers and FT8311 laminae reduces thermal damage and improves chamber geometry.

Valve performance with these improvements is illustrated in Table 3 and FIG. 49. All reverse flow pressure drops in Table 15 are 28 psi, limited by a pressure gauge in a measurement system. Therefore, diodicity for the valve at the tabulated flow rates is at least about 2,800. Leakage between the glass ball and the polyester orifice be less than 10 microliters/min at 28 psi. Such valves can include magnetic balls so that the valves are electromagnetically activated.

TABLE 15
Micro-ball Valve Diodicity

| | Forward flow | Reverse flow | |
|---|---|---|---|
| Flow rate | Pressure | Pressure | |
| (ml/min) | (psi) | (psi) | Diodicity |
| Valve diameter = 700 microns | | | |
| 1 | <0.01 | 28 | >2800 |
| 5 | 0.025 | 28 | 1120.0 |
| 7.5 | 0.3 | 28 | 93.3 |
| 10 | 0.305 | 28 | 91.8 |
| 12.5 | 0.52 | 28 | 53.8 |
| Valve diameter = 900 microns | | | |
| 5 | <0.01 | 28 | >2800 |
| 7.5 | 0.13 | 28 | 215.4 |
| 10 | 0.27 | 28 | 103.7 |
| 12.5 | 0.45 | 28 | 62.2 |

### Example 24

Cytosensor chambers can be formed using conventional machining, laser machining, photolithographic processes, or other methods. Referring to FIGS. 50A-50E, a chamber array 100 includes chambers 102-111 that are defined by recesses in a polycarbonate plate 121. The chambers 102-111 are provided with channels 132-151 that extend toward edges 152, 154 of the plate 121. A chamber assembly 160 includes the plate 121, a top plate 164, and a bottom plate 166, The top plate 164 and the bottom plate 166 are attached to the plate 121 by sonic welding, an adhesive, or with solvent welding. In one example, the plate 121 is exposed to a mixture of methylene chloride, methyl alcohol, and toluene. After exposure, the mixture is blown from the plate 121 and the plate 121 is contacted to the top plate 164 and the bottom plate 166. The plates 121, 164, 166 are pressed together for bonding and can be conveniently aligned using alignment holes configured to fit on an alignment pin made from, for example, drill rod or other material. After bonding, the chamber assembly 160 can be treated to eliminate any solvent residue by heat treating, or other method.

Fluid ports 170 extend from the chambers 102-111 to sides 180, 181 for fluid entry and discharge. The fluid ports 170 can be formed by boring holes from the sides 180 to the channels 132-151. Fluid connection to the channels 132-151 can be made by inserting tubing such as microchannel tubing into the holes so that a leak free seal is formed. Capillary action or gravity can be used to direct fluids into one or more of the chambers through the tubing. In some examples, one of either the top plate or the bottom plate is white polycarbonate to enhance viewing of chromatophores. Typically the top plate is clear to permit viewing contents of the chambers, but some plates can be made of clear or black polycarbonate or other materials.

### Example 25

### Improved Classification of Biologically Active Agents with Chromatophores

A method for improved classification of biologically active agents predicated on induced changes in the cell response profile of chromatophores to a standard elicitor set.

Fish chromatophores were plated in a 24-well culture dish 2-5 days prior to testing. The media was changed 1 day after plating. Stock concentrations of standard agents for the elicitor panel were prepared. The day of the test, fish chromatophores were sequentially exposed to the test agent and each elicitor agent in the panel. Time of exposure for the unknown agent was 5 minutes. Time of exposure to each elicitor agent varied, but was sufficient to capture the dynamics of the cell response as evidenced by the movement of the pigment particles (for example, aggregation, dispersion or no change).

The procedure for each experimental replicate consisted of mounting the 24-well plate on the microscope stand, selecting a well, finding a field of view with an adequate number of cells (50-100), opening the image acquisition program setting the image capture rate (typically 30 frames/min), adding the unknown agent for a period of 5 minutes, and adding the known agent for the time period necessary to capture the cell response dynamics. A sufficient number of replicates were performed at each condition to develop a meaningful result (n=3-6).

As seen in Table 16, the elicitor agent panel consisted of forskolin (100 μM), melanin stimulating hormone (MSH; 10 nM), clonidine (100 nM) and L-15 media (standard composition). Three strains of Bacillus cereus (BC1-49064, BC5-10987 and BC6-14579) expressing different complements of toxins as detailed in Table 17 were tested against the standard elicitor panel as described.

TABLE 16
Elicitor panel concentrations and reaction time

| | | |
|---|---|---|
| Elicitor Agent | Final Concentration | Rxn Time¹ (min) |

| | | | |
|---|---|---|---|
| Forskolin | 100 | mM | 15 |
| MSH | 10 | nM | 20 |
| Clonidine | 100 | nM | 10 |

| | | |
|---|---|---|
| L-15 Media | Standard Composition | 10 |

¹Reaction time following exposure to test agent for 5 minutes

TABLE 17
Complements of toxins expressed by each B. cereus strain

| | | | | New heat | |
|---|---|---|---|---|---|
| Strain | Hbl Toxin | Nhe Toxin | BceT Toxin | + | Others |
| BC1-49064 | + | + | − | + | + |
| BC5-10987 | − | + | − | − | + |
| BC6-14579 | + | + | + | − | + |

The data was analyzed as follows. Features of interest (response profiles of different sub-populations of cells, changes in cell area etc.) were extracted. Data reduction was performed using modeling techniques such as parametric non-linear auto-regressive external input models. Model outputs were used to form clusters in feature space. Cluster analysis was performed using integrated experts and adaptive expert calibration.

Analysis of the data indicates that incorporation of elicitors into experimental methods results in a non-linear resealing of feature space. The consequences of this are better cluster separation and easier classification based on cell response (FIG. 51). FIG. 51 demonstrates poor cluster separation for BC 5 and BC 6 in the absence of elicitors. FIG. 52 demonstrates that the use of the elicitor panel results in better cluster separation for BC5 and BC6.

### Example 26

### Generation of Libraries of Well-Characterized Effectors of Cell Function for Elicitor Panel Experiments

### Rationale and Scope

Chromatophores are dermal cells that develop from the neural crests of animals such as fish, cephalopods, and amphibians^{7,8,9,10}. Within these cells are organelles called pigment granules or chromatosomes¹¹. The movement of the chromatosomes within a pigment cell alters the cell appearance. When the pigment granules are evenly dispersed, the cell appears dark; when the pigment is aggregated at the cell center, the cell appears pale¹². Pigment granules are transported along the system of microtubules and actin filaments within the chromatophore¹³. In many species, changes in coloration can be controlled by the nervous and endocrine systems, with the response mediated by the chromatophore cell membrane receptors (Chaplen et al, 2002). There are a number of different chromatophore types, which contain different pigments.
⁷ Potenza, M. N. et al. (1992) A method for evaluating the effects of ligands upon Gₛ protein-coupled receptors using a recombinant melanophore-based bio-assay. Anal. Biochem. 206:315-322

⁸ McClintock, T. S. (1996) Melanophore pigment dispersion responses to agonists show two patterns of sensitivity to inhibitors of cAMP-dependent protein kinase and protein kinase C. J. Cell Physiol. 167:1-7

⁹ Hadley M. E. and Bagnara J. T. (1969) Integrated nature of chromatophore responses in the in vitro frog skin bioassay. Endocrinology 84:69-82.

¹⁰ Daniolos A. et al. (1990) Action of light on frog pigment cells in culture. Pigment Cell Research 3:38-43.

¹¹ Fujii, R. (1993). Cytophysiology of Fish Chromatophores. International Review of Cytology, 143, 191-256

¹² Thaler, C. D., and Haimo, L. T. (1992). Control of Organelle Transport in Melanophores: Regulation of Ca²⁺ and cAMP Levels. Cell Motility and the Cytoskeleton, 22, 175-184

¹³ Tuma, M. C., and Gelfand, V. I. (1999). Molecular Mechanisms of Pigment Transport in Melanophores. Pigment Cell Research, 12, 283-294

Early research used simple comparisons of paired digital images to measure pigment dispersion and aggregation in melanophores from the African clawed frog, X. laevis. While this approach results in basic information about the responsiveness of the cells to a given stimulus, an examination of the kinetics of chromatosome movement is more useful for characterizing the strength of a reaction and the nature of the response. Later research by Chaplen et al.¹⁴ demonstrated that measurement of the dynamics of chromatosome movement provided a wealth of information about the biological response of cells to systemic perturbations. The current system acquires digital images at a rate of up to one frame per second throughout the course of an experiment, and includes sophisticated image segmentation software that is capable of identifying individual cells and tracking features such as apparent cell area⁴.
¹⁴ Chaplen, F. W. R., Upson, R. H., McFadden, P. N., and Kolodziej, W. (2002) Fish Chromatophores as Cytosensors in a Microscale Device: Detection of Environmental Toxins and Bacterial Pathogens. Pigment Cell Research 15:19-26. Appendix A.

⁴ Roussel, N. (2003) Advanced image segmentation and data clustering concepts applied to digital image sequences featuring the response of biological materials to toxic agents. Master's Thesis, Oregon State University.

Cells isolated from Xenopus laevis will be utilized. This species has been used for the investigation of signal transduction pathways for many years and for which there exists a large body of prior knowledge. Dividing primary cell lines are straightforward to isolate. Transformed lines will take an additional 6 months. Furthermore, Xenopus has been sequenced¹⁵ and has a large molecular cell biology toolset.
¹⁵ Xenbase: A Xenopus web resource. http://www.xenbase.org/ January, 2004

### Research Design

The presence of putative signal transduction pathways will be tested directly using cell surface pathway effectors. Major classes of cell-surface receptors relevant for monitoring nanoparticle interactions with cells have been identified for initial testing. These include the G-protein coupled receptors (GPCR), Toll-like receptors (TLR), tumor necrosis factor receptors (TNFR), Fas signaling pathway receptors (FSPR) and Integrin Signaling Pathway Receptors (ISPR). Signal transduction pathway maps will be modified on the basis of these effector tests. Potential effectors for cell surface and sub-surface signaling events in our selected cell lines will be identified and tested to determine their utility. An example of the effector testing approach is outlined below for the GPCR regulatory pathways are described herein.

a. Receptors.

Receptors include those associated with the Gᵢ, Gₛ and G_{q} signal transduction pathways. For example, in order to demonstrate that norepinephrine (NE) stimulates a putative α₁-adrenergic receptor, blocking the α₂-adrenergic receptors with yohimbine, and then stimulating with NE, which stimulates both α₁ and α₂ receptors should result in an aggregation of erythrophore pigment granules. NE can stimulate both α₁ (G_{q}) and α₂ (Gᵢ) receptors. Yohimbine is a selective α₂-adrenergic blocker.

b. Adenyl Cyclase Activators.

Forskolin directly activates adenyl cyclase, and thus can directly mimic Gₛ-linked receptor signal transduction cascades. However, the response seen to forskolin is different, perhaps because receptor-mediated signal transduction involves both G_{α} and G_{βγ} subunits, as well as signal attenuation and other activities at the cell surface.

c. Inhibitors of Protein Kinase A,

H8 and H9, or H89 (Calbiochem Corporation), are membrane permeable. They inhibit by binding the ATP binding site of PKA. RP-cAMPS is a cell-permeable analog of cAMP resistant to hydrolysis by phosphodiesterases, which competitively inhibits PKA by preventing the dissociation of the holoenzyme.

d. Activators of Protein Kinase A,

SP-8-C1-cAMP and dibutyrycAMP are membrane permeable analogs of cAMP that artificially raise cAMP levels and thereby activate PKA

e. Inhibitors of Protein Kinase C.

Ro-31-8220 (Roche Products, Ltd.) is a membrane-permiable inhibitor of protein kinase C. The mechanism of inhibition has not been precisely elucidated.

f. Activators of Protein Kinase C.

Certain types of phorbol esters (4□PMA, 4□PDBu) have a long history of use in activation of PKC. 1,2-dioctanoylglycerol is a synthetic diacylglycerol that mimics endogenous stimulators of PKC.

g. Calcium Ionophores.

Ionomycin is a membrane permeable Ca⁺⁺ ionophore.

h. Calcium/Calmodulin Activated Phosphatase 2B.

Okadaic acid is a marine sponge toxin that inhibits certain phosphatases, including this one.

i. RNA Technology.

RNA regulation of cellular activities is well established. RNAi is a mechanism found in plants and animals whereby small RNA fragments are utilized to suppress the expression of specific proteins. Recent work has demonstrated that RNAi can be utilized to elucidate signal transduction networks. An increasingly more detailed knowledge of signaling pathways will be used to select appropriate RNA-derived elicitors. Such elicitors could block the production of specific proteins involved in the signal transduction networks or interfere with RNA regulation of various cellular activities.

Sequencing efforts underway for Xenopus will allow us to make use of some powerful tools for inhibiting specific protein synthesis. Anti-sense technologies target very specific sequences on the mRNA for a protein and prevent its transcription. RNAi is a mechanism found in plants and animals whereby small RNA fragments are utilized to suppress the expression of specific proteins. Recent work has demonstrated that RNAi can be utilized to elucidate signal transduction networks in insect cell culture systems with similar outcomes¹⁶. Other work has demonstrated the feasibility of this approach in Xenopus¹⁷. We will use the increasingly more detailed knowledge of signaling pathways to select appropriate targets that will allow us to not only observe the effect of blocking a particular protein's action, but also inactivate certain signal transduction pathways in order to simplify the system. Cross talk between various pathways can be a source of unwanted complexity in data analysis, and this strategy has the potential to provide more robust signals.
16 Lum, L. et al. (2003) Identification of Hedgehog Pathway Components by RNAi in Drosphilia Cultured Cells. Science 299:2039-2045.

¹⁷ Fjose A. et al. (2001) RNA interference: Mechanisms and actions. Biotechnol. Ann. Rev. 7:31-57.

### Expected Outcome and Significance

Chromatophores are particularly useful as a model system for a number of reasons. Cell response to stimulation often results in the intracellular migration of pigment granules¹⁸. This means that fluorescent markers or other assays are unnecessary; visual parameters can be tracked through digital image analysis in order to characterize how the chromatophores react when they are exposed to different agents. The relatively large sizes of both chromatophores (20-30 μm for melanophores in B. splendens) and chromatosomes (100-300 nm for melanosomes in B. splendens) make them a useful system for studying organelle transport, and a number of published studies describe signal transduction pathways which result in pigment granule transport.
¹⁸ Haimo, L. T., and Thaler, C. D. (1994). Regulation of organelle transport: Lessons from color change in fish. BioEssays, 16(10), 727-733

The systematic perturbation at different points along the signal transduction pathway through the action of elicitors, may allow us to better understand how environmental or pharmaceutical agents affect the biology of the pigment cells. For example, epinephrine binds with two different types of membrane receptors, which may generate conflicting signals for aggregation and dispersal, leading to a reduced rate of aggregation¹². Certain organophosphate pesticides can cause a ‘freezing’ of the chromatophores, where only partial pigment aggregation occurs¹⁹. By comparing the cell response to a new substance to a library of responses in which the signal cascade was disrupted at specific points, we can gain some insight into which cell mechanisms were impaired or bypassed. This strategy may be useful for predicting whether an unknown substance is toxic or otherwise biologically active and requires the development of extensive effector libraries.
¹² Thaler, C. D., and Haimo, L. T. (1992). Control of Organelle Transport in Melanophores: Regulation of Ca²⁺ and cAMP Levels. Cell Motility and the Cytoskeleton, 22, 175-184

¹⁹ Danosky, T. R. and McFadden, P. N. (1997). Biosensors based on the chromatic activities of living, naturally pigmented cells: digital image processing of the dynamics of fish melanophores. Biosensors and Bioelectronics, 12, 925-936

### Example 27

### Identification of Sub-Sets of Elicitors with Properties of Interest

A method for identifying sub-sets of elicitors of particular interest for classifying different classes of biologically active agents derived from the response profile of chromatophores to a standard elicitor set.

Fish chromatophores were plated in a 24-well culture dish 2-5 days prior to testing. The media was changed 1 day after plating. Stock concentrations of standard agents for the elicitor panel were prepared. The day of the test, fish chromatophores were sequentially exposed to the test agent and each elicitor agent in the panel. Time of exposure for the unknown agent was 5 minutes. Time of exposure to each elicitor agent varied, but was sufficient to capture the dynamics of the cell response as evidenced by the movement of the pigment particles (for example, aggregation, dispersion or no change).

The procedure for each experimental replicate consisted of mounting the 24-well plate on the microscope stand, selecting a well, finding a field of view with an adequate number of cells (50-100), opening the image acquisition program, setting the image capture rate (typically 30 frames/min), adding the unknown agent for a period of 5 minutes, and adding the known agent for the time period necessary to capture the cell response dynamics. A sufficient number of replicates were performed at each condition to develop a meaningful result (n=3-6).

As seen in Table 18, the elicitor agent panel consisted of forskolin (100 μM), melanin stimulating hormone (MSH; 10 nM), clonidine (100 nM) and L15 media (standard composition). Three strains of Bacillus cereus (BC1-49064, BC5-10987 and BC6-14579) expressing different complements of toxins as detailed in Table 19 were tested against the standard elicitor panel as described.

TABLE 18
Elicitor panel concentrations and reaction time

| | | |
|---|---|---|
| Elicitor Agent | Final Concentration | Rxn Time¹ (min) |

| | | | |
|---|---|---|---|
| Forskolin | 100 | mM | 15 |
| MSH | 10 | nM | 20 |
| Clonidine | 100 | nM | 10 |

| | | |
|---|---|---|
| L-15 Media | Standard Composition | 10 |

¹Reaction time following exposure to test agent for 5 minutes

TABLE 19
Complements of toxins expressed by each B. cereus strain

| | | | | New heat | |
|---|---|---|---|---|---|
| Strain | Hbl Toxin | Nhe Toxin | BceT Toxin | + | Others |
| BC1-49064 | + | + | − | + | + |
| BC5-10987 | − | + | − | − | + |
| BC6-14579 | + | + | + | − | + |

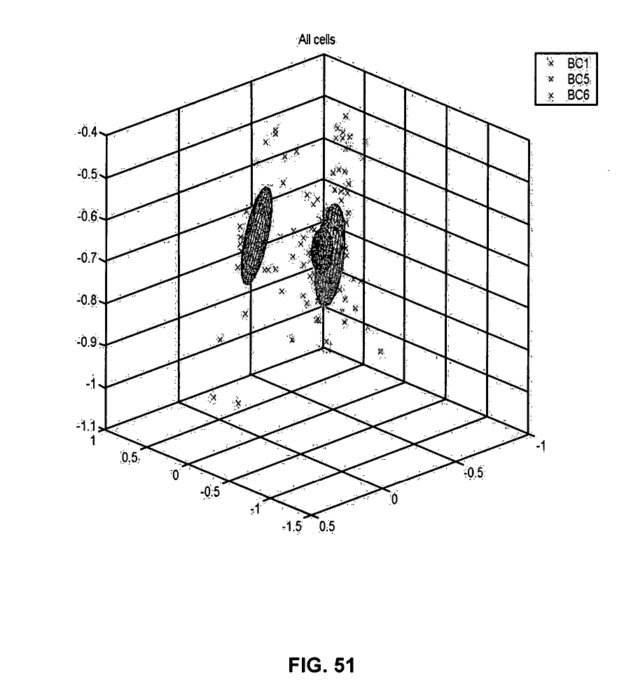

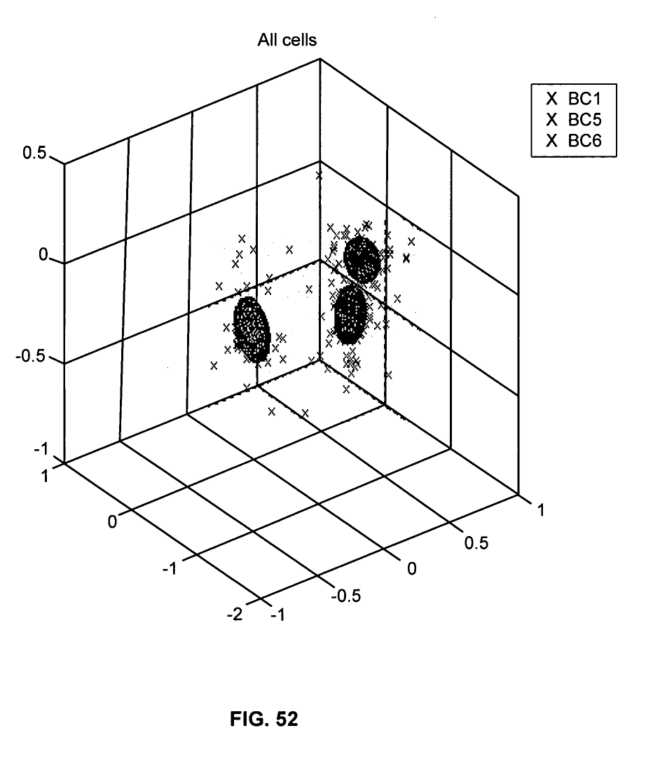

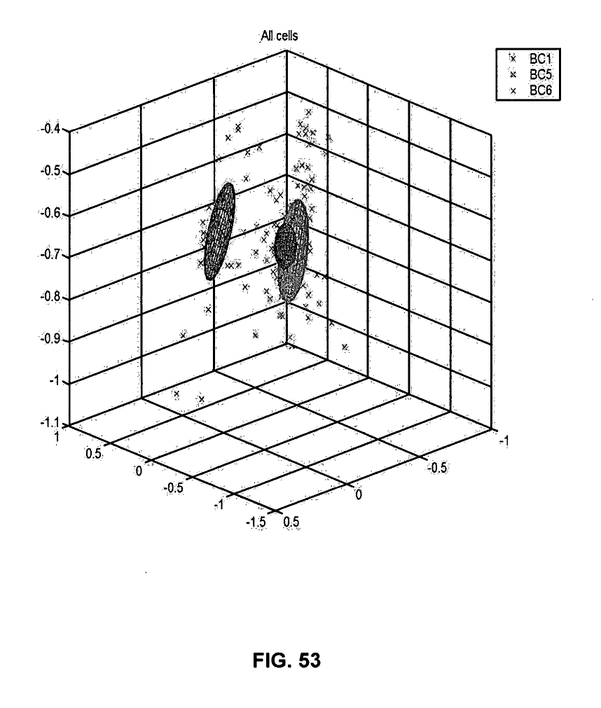

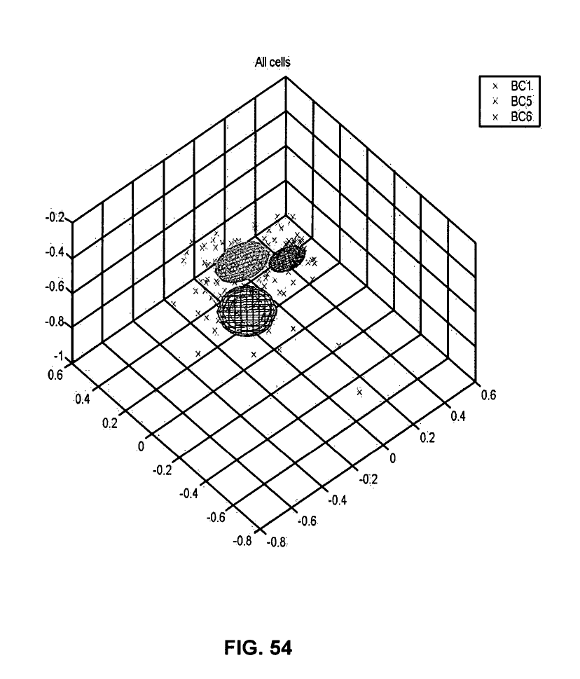

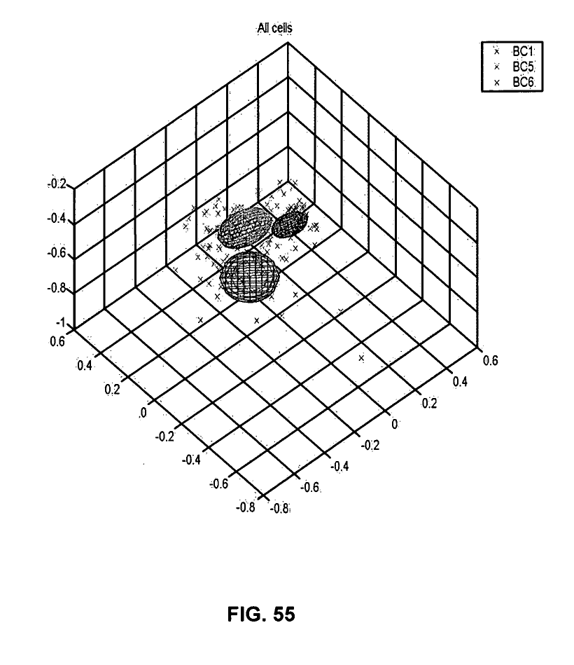

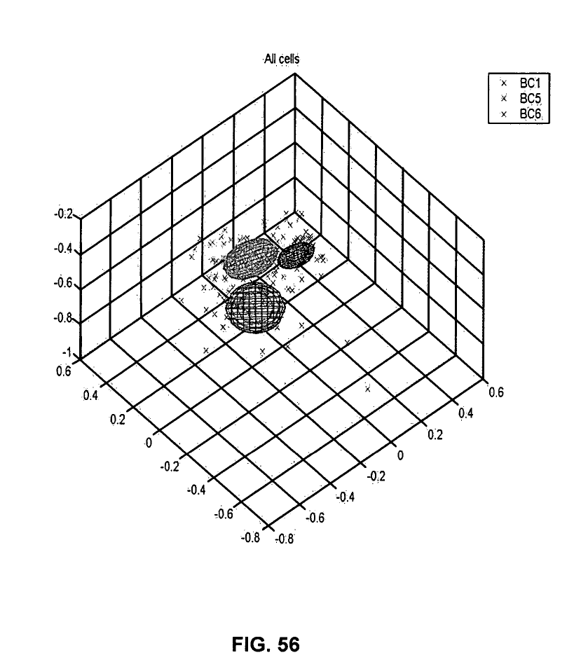

The data was analyzed as follows. Features of interest (response profiles of different sub-populations of cells, changes in cell area etc.) were extracted. Data reduction was performed using modeling techniques such as parametric non-linear auto regressive external input models. Model outputs were used to form clusters in feature space. Cluster analysis of cell response in the presence of each individual elicitor was performed using integrated experts and adaptive expert calibration. FIG. 53 indicates that cluster separation in the presence of L-15 for bacterial strains BC 5 and BC 6 is poor. FIG. 54. demonstrates that cluster separation for BC 5 and BC 6 in the presence of MSH alone is almost as good as is evident in FIG. 1B for the entire elicitor set.

### Example 28

### Mechanistic Classification of Biologically Active Agents with Chromatophores

A method for assigning biological mechanisms to biologically active agents predicated on induced changes in the color response profile of chromatophores to a standard panel of elicitor agents selected on the basis of interactions with important signal transduction pathway checkpoints.

Fish chromatophores are plated in a 24-well culture dish 2-5 days prior to testing. The media is changed 1 day after plating. Stock concentrations of standard agents for the elicitor panel are prepared. The day of the test, fish chromatophores are sequentially exposed to the test agent and each elicitor agent in the panel. Time of exposure for the unknown agent was 5 minutes. Time of exposure to each elicitor agent varies, but is sufficient to capture the dynamics of the cell response as evidenced by the movement of the pigment particles (for example, aggregation, dispersion or no change).

The procedure for each experimental replicate consists of mounting the 24-well plate on the microscope stand, selecting a well, finding a field of view with an adequate number of cells (50-100), opening the image acquisition program setting the image capture rate (typically 30 frames/min), adding the unknown agent for a period of 5 minutes, and adding the known agent for the time period necessary to capture the cell response dynamics. A sufficient number of replicates were performed at each condition to develop a meaningful result (n=3-6).

As seen in Table 20, the elicitor agent panel consisted of forskolin (100 μM), melanin stimulating hormone (MSH; 10 nM), clonidine (100 nM) and L-15 media (standard composition). The elicitor agents were selected on the basis of interactions with important signal transduction pathway checkpoints. FIG. 3 shows cAMP mediated G-protein regulated pathways (Gₛ and Gᵢ) of importance to fish chromatophores. Forskolin is a direct activator of adenyl cyclase, a checkpoint in the Gₛ and Gᵢ pathways; melanin stimulating hormone directly stimulates the Gₛ receptors; and clonidine directly stimulates Gᵢ receptors. Three strains of Bacillus cereus (BC1-49064, BC5-10987 and BC6-14579) expressing different complements of toxins as detailed in Table 21 were tested against the standard elicitor agent panel as described.

TABLE 20
Elicitor panel concentrations and reaction time

| | | |
|---|---|---|
| Elicitor Agent | Final Concentration | Rxn Time¹ (min) |

| | | | |
|---|---|---|---|
| Forskolin | 100 | mM | 15 |
| MSH | 10 | nM | 20 |
| Clonidine | 100 | nM | 10 |

| | | |
|---|---|---|
| L-15 Media | Standard Composition | 10 |

¹Reaction time following exposure to test agent for 5 minutes

TABLE 21
Complements of toxins expressed by each B. cereus strain

| | | | | New heat | |
|---|---|---|---|---|---|
| Strain | Hbl Toxin | Nhe Toxin | BceT Toxin | + | Others |
| BC1-49064 | + | + | − | + | + |
| BC5-10987 | − | + | − | − | + |
| BC6-14579 | + | + | + | − | + |

The data was analyzed as follows. Features of interest (response profiles of different sub-populations of cells, changes in cell area etc.) were extracted. Data reduction was performed using modeling techniques such as parametric non-linear auto-regressive external input models. Model outputs were used to form clusters in feature space. Cluster analysis was performed using integrated experts and adaptive expert calibration as described elsewhere.

A mechanistic interpretation is derived from the differences in cell responses to elicitors in the presence or absence of biologically active agents of interest. FIG. 54 demonstrates that cell responses to MSH in the presence of BC 5 and BC 6 are different indicating that the complement of toxins expressed by each bacterial strain affects the cell signaling networks in a dissimilar manner. A mechanistic interpretation of this observation is that the Gₛ signal transduction pathway in specifically affected. FIG. 55 shows cluster distribution with MSH as an elicitor. FIG. 56 demonstrates that forskolin also allows differentiation of BC 5 and BC 6 but not as efficiently as MSH. This suggests that the different complements of toxins are specifically impacting the Gₛ signal transduction pathway between the receptor and adenyl cyclase.

### Example 29

### To Identify a Library of Signal Transduction Network Modules Using the Framework Provided by Structural Motifs Present in Cellular Regulatory Systems

### Rationale and Scope

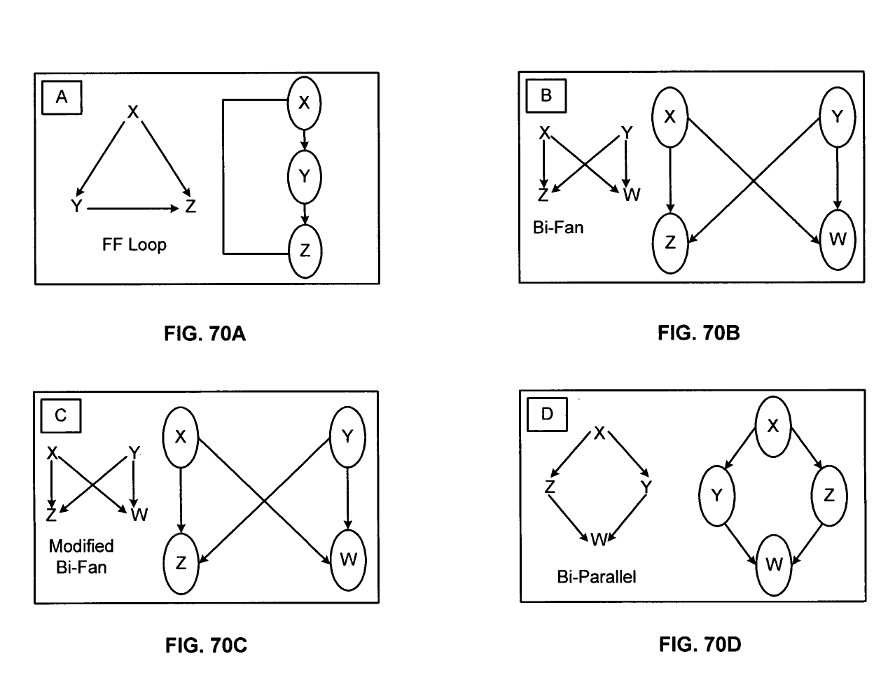

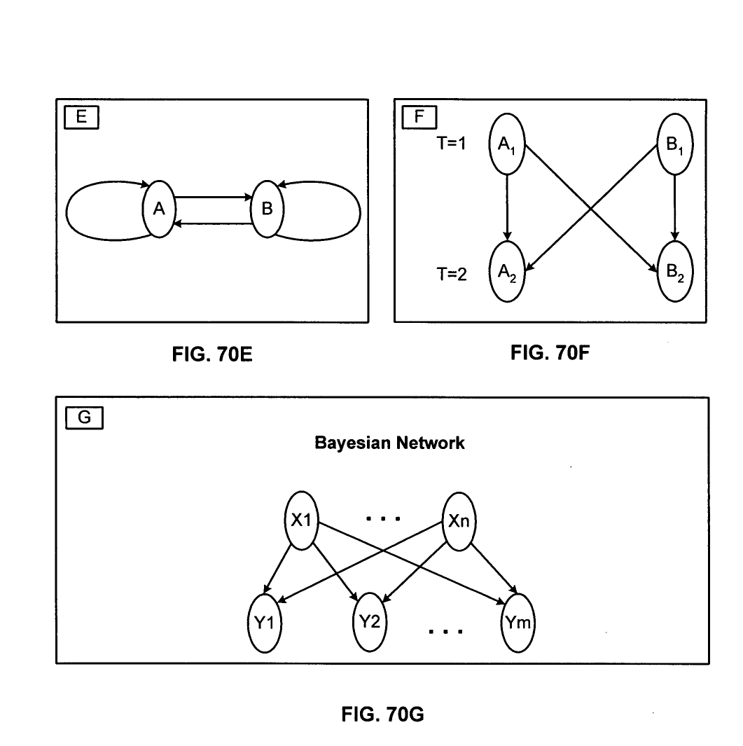

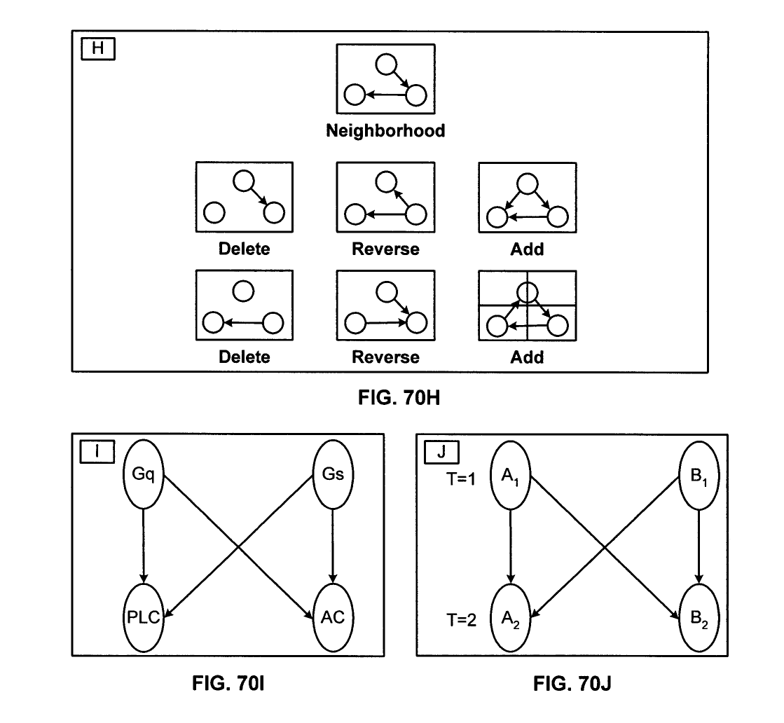

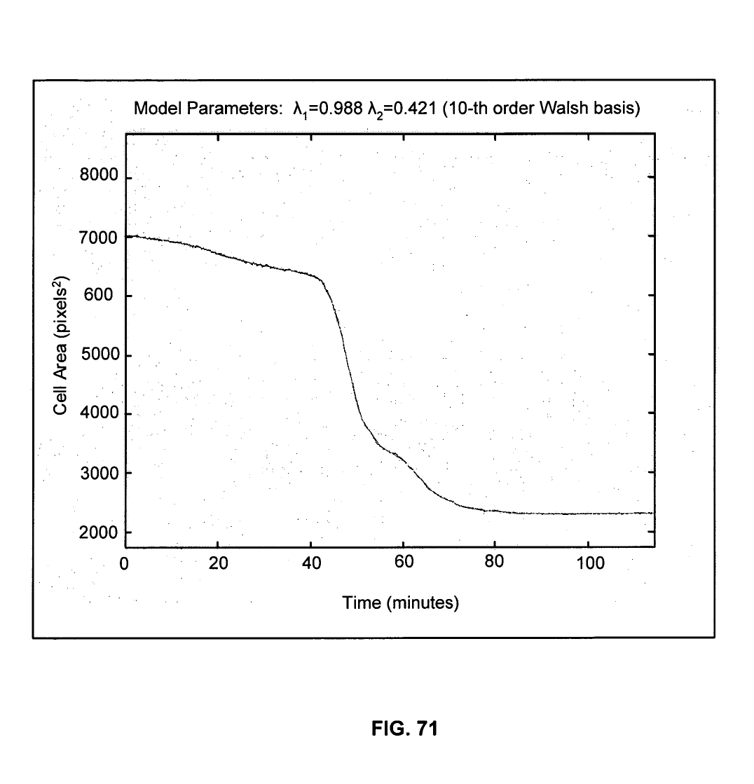

Segmentation experiments similar are representative of necessary experiments. For that segmentation scheme, AC is acting as the primary node with forskolin acting as a direct effector. The α₁-adenergic receptor (cirrazoline), α₂-adenergic receptor (clonidine) and PKA (H89) are secondary nodes. Stimulus of the system at secondary nodes provides additional information regarding the unknown agent's mechanism-of-action within the module created through the action of forskolin. Additional experiments with well characterized effectors as “unknown” agents will be utilized to identify primary nodes specific for segmentation. The structural motifs evident in FIG. 70 will aid in the identification of reference node candidates such as AC and PLC.

Recent statistical analyses of genetic and neural network topologies have identified three and four-node structural motifs present in the genomic networks of Saccharomyces cerevisiae and Escherichia coli and the synaptic connection patterns of neurons in Caenorhabditis elegans²⁰. FIG. 70 shows the structure of four of these sub-graphs (Feed-Forward Loop, Bi-Fan, Modified Bi-Fan and Bi-Parallel). Sub-graphs represent discretized information network processes projected onto the gene sequence. FIG. 70E is a graphical representation of the core bistability of the phage λ switch, which consists of two antagonistic feed-back loops²¹. FIG. 70G shows the general structure of a Bayesian network, which qualitatively matches the topology of the structural motifs shown in FIG. 70A-D and demonstrates the applicability of Bayesian networks to modeling biological cell signaling networks. The Bi-Fan structure matches the hidden Markov model equivalent of the genetic regulatory motif shown in FIG. 70E. Note the interesting parallels between the edge selection proposal moves given through the Metropolis algorithm (FIG. 70H) and the 3-node structural elements that form the basis for the motifs in FIG. 70A-D. Information flows through cellular protein interaction networks may be organized around simple structural motifs as the Bi-Fan structure is also found as a recurrent element in the G-protein regulated pathways shown in FIG. 70A-B.
²⁰ Milo, R., Shen-Orr, S., Itzkovitz, S. et al. (2002) Network motifs: Simple building blocks of complex networks. Science 298:824-827.

²¹ Rao, C. V. and Arkin, A. P. (2001) Control Motifs for Intracellular Regulatory Networks. Annu. Rev. Biomed. Eng. 3:391-419.

### Research Design

Despite the large amounts of data available from global analyses of cell signaling networks, the major challenge remains incomplete data. The objective is to learn the Bayesian network from experimental data gathered using literature data and elicitor experiments. The initial protein interaction network structure can be formulated using the literature. However, the species variation associated with cell signaling networks will inevitability require experimental verification of edge assignment. Elicitor experiments provide lumped network data which is inherently discrete and complete. The discrete nature of the data arises from the fact the system state is being sampled at a particular time period. The state is then captured as the eigenvalues of the cell response curve as shown in FIG. 70. The inherent completeness is because elicitors define the network, and each elicitor experiment is effectively an output measurement from the sub-graph represented by the segmented mechanism space.

Lumping cellular activities using a modular format will reduce the number of random variables that must be considered as network structures are elucidated. Identified modules may not map to naturally occurring functional modules but will recognize the structural motifs evident in analyses of genomic information (See FIG. 70 and Milo et al.²⁰).
²⁰ Milo, R., Shen-Orr, S., Itzkovitz, S. et al. (2002) Network motifs: Simple building blocks of complex networks. Science 298:824-827.

Analytically, the number of edges converging on a node can be limited, which has the practical advantage of improving convergence of the MCMC simulation. Scale-free network descriptors will be used to introduce scale-free clustering and modularity to the network model. Two descriptors are useful in determining whether this network is similar in structure to a scale-free network, with or without embedded modularity. The distribution of network connections within a scale-free network (k) can be described with a power law relationship, where N(k)˜k^{-γ}, 2<γ<3 and a clustering coefficient as defined in Equation 7, where n is the number of direct links between the ki nearest neighbors of node, I is equal to one for a highly interconnected hub, and is zero for a node that is part of a loosely connected group.

$${C}_{i} = \frac{2 n}{{k}_{i} ( {k}_{i} - 1 )} \qquad ( 7 )$$

In a purely scale-free network, the average clustering coefficient decreases proportionally to N^{-0.75}, but in modular metabolic networks the clustering coefficient followed a power law dependent on k:
C(k)k˜⁻¹ (8)

### Expected Outcome and Significance

Learning algorithms derived from Eqn. (3) are likely to lead to multiple network structures as initial data is analyzed due to lack of preliminary experimental data. An iterative experimental/analytical approach will be necessary to learn which network structures are applicable for given environmental conditions. Preliminary work has demonstrated the decoupling segments of the signal transduction network through identification of reference nodes and applications of primary elicitors. Other studies have demonstrated that removal of PKC activity failed to prevent pigment dispersion indicating that signals had been rerouted²².Reference nodes form a non-conditional framework for analysis under a particular set of conditions. Conditionality is a working definition based on the objective function of the study and issues associated with efficiently²³ identifying acceptable network structures. Preliminary results the demonstrate network edge reduction through removal of the edge between adenylate cyclase and protein kinase A with forskolin, an inhibitor of adenylate cyclase. Absence of identified edges reduces interactions between two signal transduction pathways and reduces the complexity of analysis. Edges could also be removed through the use of gene knockout or modification of gene translation/transcription through anti-sense or other approaches as described herein.
²² Reilin, A. R., Tint, I. S., Peunova, N. I., Enikolopov, G. N., and Gelfand, V. I. (1998). Regulation of Organelle Movement in Melanophores by Protein Kinase A (PKA), Protein Kinase C (PKC), and Protein Phosphatase 2A. Journal of Cell Biology, 142(3), 803-813.

²³ “Efficient” refers both to computational and experimental efficiency.

### Example 30

### Improved Classification of Biologically Active Agents with Cells Other than Chromatophores

An improved method for classifying biologically active agents predicated on induced changes in the response profile of different cell types to a standard panel of elicitor agents.

The response profile of the cells might be measured based on fluorescent probe technology, expression of recombinant markers, such as green fluorescent protein or luciferase, or the use of appropriately labeled semiconductor nanoparticles.

The cells will be plated in a 24-well culture dish some days prior to testing. Stock concentrations of standard agents for the elicitor panel are prepared. The day of the test, the cells are sequentially exposed to the test agent and each elicitor agent in the panel. The time of exposure to the test agent is sufficient to affect the biological system. Time of exposure to each elicitor agent varies, but is sufficient to capture the dynamics of the cell response.

The data is analyzed as follows. Features of interest (response profiles of different sub-populations of cells, changes in cell area etc.) are extracted. Data reduction is performed using modeling techniques such as parametric non-linear auto-regressive external input models. Model outputs are used to form clusters in feature space. Cluster analysis is performed using integrated experts and adaptive expert calibration.

### Example 31

### Mechanistic Classification of Biologically Active Agents with Cells Other than Chromatophores

An method for assigning a mechanistic interpretation to interactions between biologically active agents and cells predicated on induced changes in the response profile of different cell types to a standard panel of elicitor agents.

The response profile of the cells might be measured based on fluorescent probe technology, expression of recombinant markers, such as green fluorescent protein or luciferase, or the use of appropriately labeled semiconductor nanoparticles.

The cells will be plated in a 24-well culture dish some days prior to testing. Stock concentrations of standard agents for the elicitor panel are prepared. The day of the test, the cells are sequentially exposed to the test agent and each elicitor agent in the panel. The time of exposure to the test agent is sufficient to affect the biological system. Time of exposure to each elicitor agent varies, but is sufficient to capture the dynamics of the cell response.

The elicitor agent panel consists of agents selected on the basis of interactions with important signal transduction pathway checkpoints. For example, FIG. 3 shows cAMP mediated G-protein regulated pathways (Gₛ and Gᵢ) of importance to fish chromatophores. Forskolin is a direct activator of adenyl cyclase, a checkpoint in the Gₛ and Gᵢ pathways; melanin stimulating hormone directly stimulates the Gₛ receptors; and clonidine directly stimulates Gᵢ receptors. Similar panels are developed for cell types of interest.

The data is analyzed as follows. Features of interest (response profiles of different sub-populations of cells, changes in cell area etc.) are extracted. Data reduction is performed using modeling techniques such as parametric non-linear auto-regressive external input models. Model outputs are used to form clusters in feature space. Cluster analysis is performed using integrated experts and adaptive expert calibration.

A mechanistic interpretation is derived from the differences in cell responses to elicitors in the presence or absence of biologically active agents of interest

### Example 32

### Classification of Biologically Active Agents with Microbial Communities

A method for classifying biologically active agents predicated on induced changes in the response profile of microbial communities to a standard panel of elicitor agents.

The response profile of the microbial community might be measured based on fluorescent probe technology, expression of recombinant markers, such as green fluorescent protein or luciferase, the use of appropriately labeled semiconductor nanoparticles, or standard techniques, such as lipid membrane composition or 16S RNA profile.

Samples are taken from the region of interest (water, swab of surface). Stock concentrations of standard agents for the elicitor panel are prepared. The day of the test, the sample is sequentially exposed to the test agent and each elicitor agent in the panel. The time of exposure to the test agent is sufficient to affect the biological system. Time of exposure to each elicitor agent varies, but is sufficient to capture the dynamics of the microbial community response.

The data is analyzed as follows. Features of interest (response profiles of different sub-populations of cells, changes in cell area etc.) are extracted. Data reduction is performed using modeling techniques such as parametric non-linear auto-regressive external input models. Model outputs are used to form clusters in feature space. Cluster analysis is performed using integrated experts and adaptive expert calibration.

### Example 33

### Orthogonal Methods for Increasing the Size of the Feature Space Vector

A method for increasing the size of the feature space vector predicated on orthogonal measurements of biological system response. Experiments are performed as described in Examples 1, 2, 3, 4 and 5. Gene transcription patterns are assessed using technologies such as microarray technology. Gene expression patterns are assessed using technologies such as 2-D gel electrophoresis. Changes in cell regulation associated with protein phosphorylation are measured with technologies such as 2-D gel electrophoresis. The expression patterns of extracellular proteins such as are produced by microbial pathogens are measured in growth medium using 2-D gel electrophoresis. Media component (sugars, amino acids, growth factors, salts) consumption patterns and the by-products of cell metabolism are measured using liquid and gas chromatographic techniques. Intracellular metabolic fluxes are assessed using nuclear magnetic resonance in combination with modeling techniques, such as metabolic flux analysis.

This data is integrated into the feature space vector along with the features extracted from the time series of images captured during the experimental. Data analysis occurs as described elsewhere.

### Example 34

### Microfluidic Systems for Better Control of Cell Response in the Presence of Biologically Active Agents or Elicitors

A method for decreasing the variation in cell response by maintaining the cells in a microfluidic environment.

Fish chromatophores were plated in a 24-well culture dish or in microscale (GIII) chambers 2-5 days prior to testing. The media was changed 1 day after plating. Stock concentrations of clonidine (100 nM) or overnight cultures of B. cereus Strain 1 were prepared. The day of the test, fish chromatophores in the 24-well plate or microfluidic chamber were exposed to the test agents. Time of exposure was 2 minutes.

The procedure for each experimental replicate consisted of mounting the 24-well plate on the microscope stand, selecting a well, finding a field of view with an adequate number of cells (50-100), opening the image acquisition program setting the image capture rate (typically 30 frames/min), adding the unknown agent for a period of 5 minutes, and adding the known agent for the time period necessary to capture the cell response dynamics. A sufficient number of replicates were performed at each condition to develop a meaningful result (n=3-6).

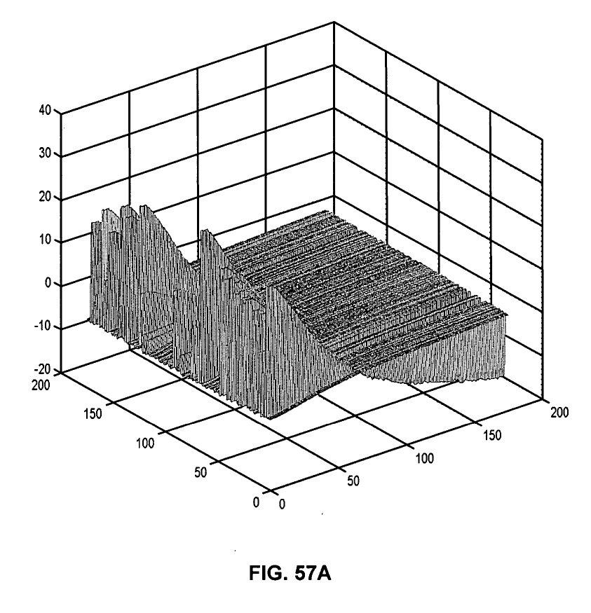

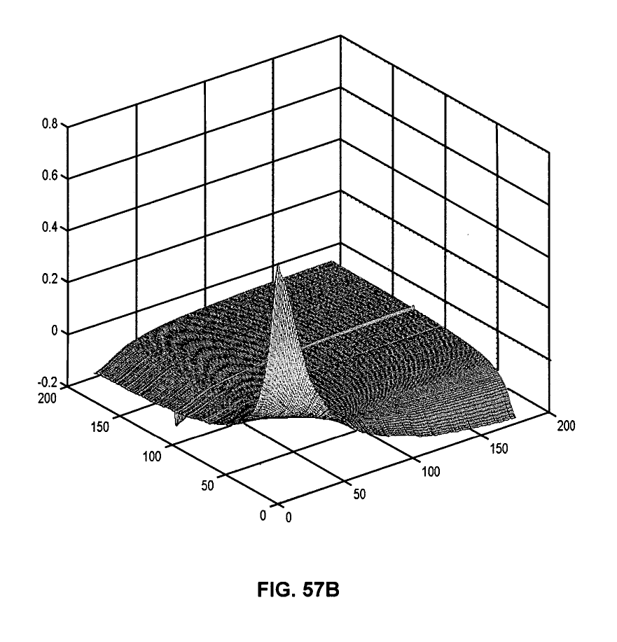

A qualitative assessment of the data FIG. 57 indicates that the cell response profile to clonidine was smoother for the microfluidic system.

### Example 35

### Signature Classification of Biologically Active Agents with Chromatophores

A method for assigning signatures to biologically active agents predicated on induced changes in the cell response profile of chromatophores to a standard panel of elicitors agents.

Fish chromatophores were plated in a 24-well culture dish 2-5 days prior to testing. The media was changed 1 day after plating. Stock concentrations of standard agents for the elicitor panel were prepared. The day of the test, fish chromatophores were sequentially exposed to the test agent and each elicitor agent in the panel. Time of exposure for the unknown agent was 5 minutes. Time of exposure to each elicitor agent varied, but was sufficient to capture the dynamics of the cell response as evidenced by the movement of the pigment particles (for example, aggregation, dispersion or no change).

The procedure for each experimental replicate consisted of mounting the 24-well plate on the microscope stand, selecting a well, finding a field of view with an adequate number of cells (50-100), opening the image acquisition program setting the image capture rate (typically 30 frames/min), adding the unknown agent for a period of 5 minutes, and adding the known agent for the time period necessary to capture the cell response dynamics. A sufficient number of replicates were performed at each condition to develop a meaningful result (n=3-6).

As seen in Table 22, the elicitor agent panel consisted of forskolin (100 μM), melanin stimulating hormone (MSH; 10 nM), clonidine (100 μM) and L-15 media (standard composition). Three strains of Bacillus cereus (BC1-49064, BC5-10987 and BC6-14579) expressing different complements of toxins as detailed in Table 23 were tested against the standard elicitor panel as described.

The data was analyzed as follows. Features of interest (response profiles of different sub-populations of cells, changes in cell area etc.) were extracted. Data reduction was performed using modeling techniques such as parametric non-linear auto-regressive external input models.

TABLE 22
Elicitor panel concentrations and reaction time

| | | |
|---|---|---|
| Elicitor Agent | Final Concentration | Rxn Time¹ (min) |

| | | | |
|---|---|---|---|
| Forskolin | 100 | mM | 15 |
| MSH | 10 | nM | 20 |
| Clonidine | 100 | nM | 10 |

| | | |
|---|---|---|
| L-15 Media | Standard Composition | 10 |

¹Reaction time following exposure to test agent for 5 minutes

TABLE 23
Complements of toxins expressed by each B. cereus strain

| | | | | New heat | |
|---|---|---|---|---|---|
| Strain | Hbl Toxin | Nhe Toxin | BceT Toxin | + | Others |
| BC1-49064 | + | + | − | + | + |
| BC5-10987 | − | + | − | − | + |
| BC6-14579 | + | + | + | − | + |

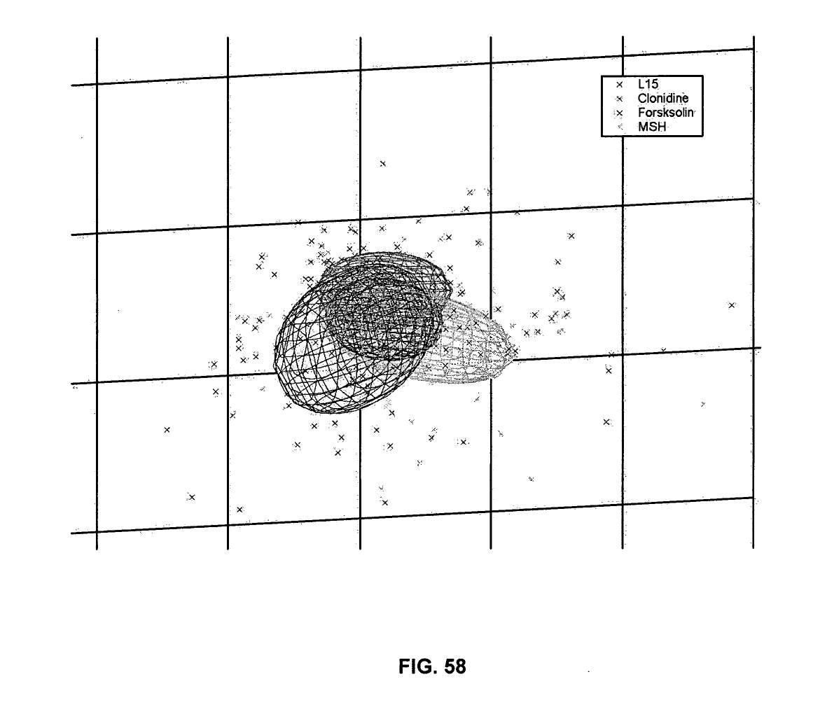

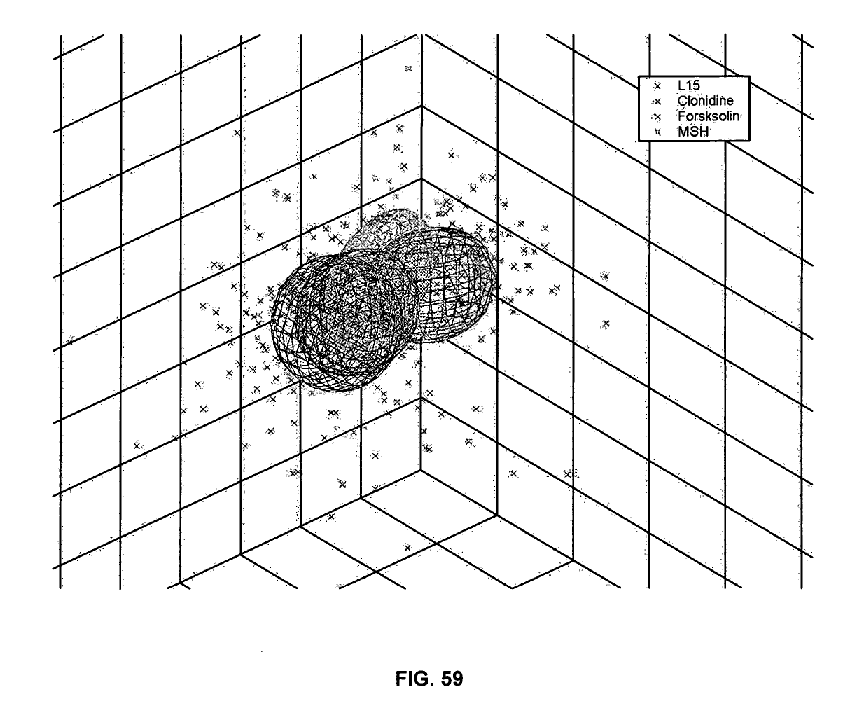

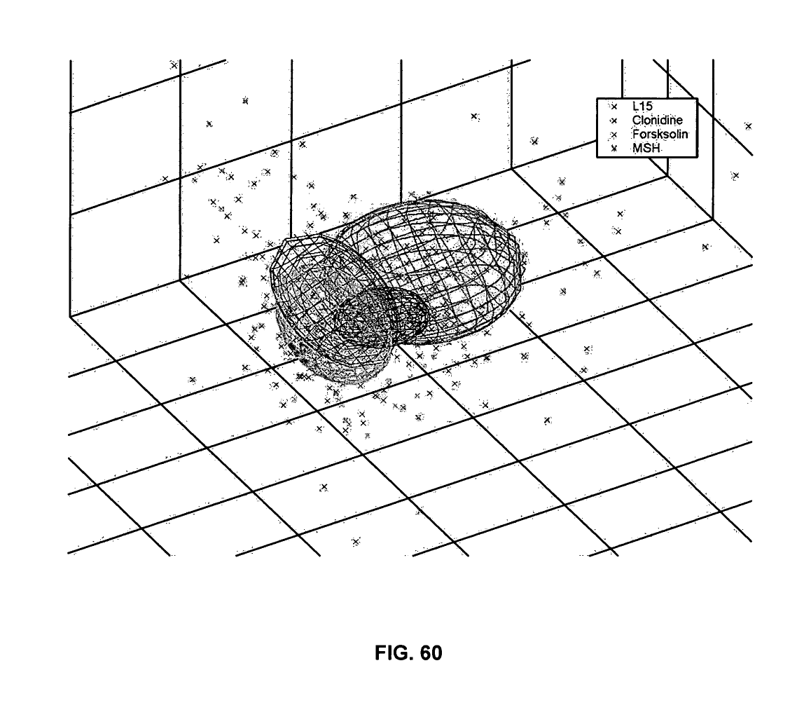

Model outputs were used to form clusters in feature space. Cluster analysis was performed using integrated experts and adaptive expert calibration as described elsewhere. FIGS. 58-60 shows that the each bacterial strain demonstrates has a different signature (cluster pattern) based on interactions with the elicitor panel.

### Example 36

### Signature Classification of Biologically Active Agents with Cells Other than Chromatophores

A method for assigning signatures to biologically active agents predicated on induced changes in the response profile of cells to a standard panel of elicitors agents.

The response profile of the cells might be measured based on fluorescent probe technology, expression of recombinant markers, such as green fluorescent protein or luciferase, or the use of appropriately labeled semiconductor nanoparticles.

The cells will be plated in a 24-well culture dish some days prior to testing. Stock concentrations of standard agents for the elicitor panel are prepared. The day of the test, the cells are sequentially exposed to the test agent and each elicitor agent in the panel. The time of exposure to the test agent is sufficient to affect the biological system. Time of exposure to each elicitor agent varies, but is sufficient to capture the dynamics of the cell response.

The data is analyzed as follows. Features of interest (response profiles of different sub-populations of cells, changes in cell area etc.) are extracted. Data reduction is performed using modeling techniques such as parametric non-linear auto-regressive external input models. Model outputs are used to form clusters in feature space. Cluster analysis is performed using integrated experts and adaptive expert calibration.

Each biologically active agent will have a unique signature based on the cluster map of cell responses derived from the elicitor panel.

### Example 37

### Integration of Elicitor Panel Concept with Soft Classification Techniques

Sophisticated informatics is necessary for the development of fieldable cytosensor systems. Field applications add stringent requirements for simplicity and robustness to any analysis software and associated experimental methodologies. Simplicity implies the capacity for extracting useful information without extensive laboratory infrastructures. Robustness refers to accurate scenario matching with low false positive/negative generation rates. Informatics approaches must meet the simplicity and robustness requirements while having the capability to handle sparse data generated through experiments that produce large, highly multi-dimensional data sets. It should be noted that the approaches developed through the research described herein are applicable to cell based systems in general providing there are measurable outputs to generate the numerical feature vector and effectors of cell response are readily available.

The classification techniques integrate both numerical and non-numerical classifiers for optimal interpretation and decision-making processes and are built around four basic concepts: feature space, integrated experts, adaptive expert calibration and soft classification. Feature space consists of a non-numerical description (scenario) and a numerical feature vector extracted from the experimental run. Each experimental run is uniquely identified by the scenario and numerical feature vector. An expert is defined as a mapping from the numerical feature space to the set of scenarios. This mapping is realized through assigning to a numerical vector a set of mixing (weighting) coefficients corresponding to all the non-numerical descriptors (labels). An integrated expert is an arbitrary number of experts working in parallel. A process of adaptive calibration is needed to properly fuse the individual expert (integrated or individual) outputs. A simplex scenario is a special but important case of a scenario for which the numerical feature vector is concentrated on a single label or non-numerical identifier. Labeling induced by simplex scenarios divides the complete numerical feature or system response space into subsets. A complex scenario can be viewed as a combination of simplex scenarios. Soft classification is understood as the a priori creation of a complex scenario.

The elicitor set method is an experimental framework derived from an understanding of the signal transduction networks that underlie cellular function. Elicitor panels are formed from known effectors of checkpoints in the signal transduction pathways. Application of the elicitors in combination with known agents (calibration runs) results in the generation of simplex scenarios that form the basis for the soft classification of unknown agents (operational runs).

Fish chromatophores are plated in a 24-well culture dish 2-5 days prior to testing. The media is changed 1 day after plating. Stock concentrations of standard agents for the elicitor panel are prepared. The day of the test, fish chromatophores are sequentially exposed to the test agent and each elicitor agent in the panel. Time of exposure for the unknown agent was 5 minutes. Time of exposure to each elicitor agent varies, but is sufficient to capture the dynamics of the cell response as evidenced by the movement of the pigment particles (for example, aggregation, dispersion or no change).

The procedure for each experimental replicate consists of mounting the 24-well plate on the microscope stand, selecting a well, finding a field of view with an adequate number of cells (50-100), opening the image acquisition program setting the image capture rate (typically 30 frames/min), adding the unknown agent for a period of 5 minutes, and adding the known agent for the time period necessary to capture the cell response dynamics. A sufficient number of replicates were performed at each condition to develop a meaningful result (n=3-6).

As seen in Table 24, the elicitor agent panel consists of forskolin (100 μM), melanin stimulating hormone (MSH; 10 nM), clonidine (100 nM) and L-15 media (standard composition). The elicitor agents were selected on the basis of interactions with important signal transduction pathway checkpoints. Forskolin is a direct activator of adenyl cyclase, a checkpoint in the Gₛ and Gᵢ pathways; melanin stimulating hormone directly stimulates the Gₛ receptors; and clonidine directly stimulates Gᵢ receptors. Three strains of Bacillus cereus (BC1-49064, BC5-10987 and BC6-14579) expressing different complements of toxins as detailed in Table 245 were tested against the standard elicitor agent panel as described.

TABLE 24
Elicitor panel concentrations and reaction time

| | | |
|---|---|---|
| Elicitor | Final | Rxn Time¹ |
| Agent | Concentration | (min) |

| | | | |
|---|---|---|---|
| Forskolin | 100 | mM | 15 |
| MSH | 10 | nM | 20 |
| Clonidine | 100 | nM | 10 |

| | | |
|---|---|---|
| L-15 Media | Standard Composition | 10 |

¹Reaction time following exposure to test agent for 5 minutes

TABLE 25
Complements of toxins expressed by each B. cereus strain

| | Hbl | Nhe | BceT | New heat | |
|---|---|---|---|---|---|
| Strain | Toxin | Toxin | Toxin | + | Others |
| BC1-49064 | + | + | − | + | + |
| BC5-10987 | − | + | − | − | + |
| BC6-14579 | + | + | + | − | + |

The data was analyzed as follows. Features of interest (response profiles of different sub-populations of cells, changes in cell area etc.) were extracted. Data reduction was performed using modeling techniques such as parametric non-linear auto-regressive external input models. Model outputs were used to form clusters in feature space. Cluster analysis was performed using integrated experts and adaptive expert calibration as described elsewhere.

FIG. 61 shows four simplex scenarios corresponding to each of the elicitors present in the elicitor panel. Complex complex scenarios can be created from various combinations of simplex scenarios. A comparison of the complex complex scenarios to the complex scenario shown in FIG. 62 provides a mechanistic interpretation of the results.

### Example 38

### Demonstrating Module Integration Using Bayesian Network and Other Probabilistic Modeling Approaches

### Rationale and Scope

The distribution of conditional probabilities through the network represents a signature for biologically active compounds. Mechanistic models (systems of ordinary differential equations) can provide additional information once the structure of the network is determined for a given situation. ODE model development will follow a simple to complex approach. Once the network structure is identified using the described approaches, the steps in the pathway will be linked together to yield a model which accepts an elicitor type and concentration as inputs, and predicts how the apparent cell area of the chromatophores will change in response to the given treatment. A representative sample of 10-20 chromatophores will be selected for each experiment of interest using image segmentation software developed specifically for chromatophore applications. This data will be used as a training set for parameterization with a genetic algorithm. The relative success of the genetic algorithm in fitting parameters for the mechanistic model will be used for sensitivity analyses, in order to determine which sections of the model are over-simplified. The modeling approach for each section is described in more detail below.

### Research Design

Mechanistic model development will follow a simple to complex approach. For each of the processes listed in the objectives section, the investigation will start with a basic model, using simplified cell geometry and chemical kinetics. The steps in the pathway will be linked together to yield a model which accepts an elicitor type and concentration as inputs, and predicts how the apparent cell area of the chromatophores will change in response to the given treatment. A representative sample of 10-20 chromatophores will be selected for each experiment of interest using image segmentation software developed specifically for chromatophore applications^{4,24}. This data will be used as a training set for parameterization with a genetic algorithm. The relative success of the genetic algorithm in fitting parameters for the mechanistic model will be used for sensitivity analyses, in order to determine which sections of the model are over-simplified. The modeling approach for each section is described in more detail below.
⁴ Roussel, N. (2003) Advanced image segmentation and data clustering concepts applied to digital image sequences featuring the response of biological materials to toxic agents. Master's Thesis, Oregon State University.

²⁴ Teng, A. (2002) Process Segmentation and Multivariate Time Series Modeling, Final Year Project, Oregon State University.

Agent Delivery to the Cell Surface

Agents are delivered by injecting into a well of cell media, with the chromatophores fixed to the bottom of the well. The initial simplifying assumption is that the turbulence of the injection causes efficient mixing of the agent with the cell media, and that any concentration boundary layer surrounding the cells can be neglected. Under this assumption, the agent concentration at the outer cell surface would be equal to the bulk concentration in the well. Dye tests, using a dye with molecular weight and chemical characteristics similar to the elicitors, suggest that this is a reasonable approach. Thus, the concentration of agent A just outside the cell membrane, C_{A,,o} is equivalent to the bulk concentration of agent I, C_{A,∞}.
C_{A,,o}=C_{A,∞} (10)

If the assumption of uniform agent distribution is incorrect, then two factors must be considered. First, if the bulk solution is not thoroughly mixed by the injection process, the unequal distribution of the agent must be addressed by investigating the effects of diffusion. In addition, a poorly stirred concentration layer adjacent to the chromatophore membranes may exist, in which the localized concentration will be lower than that of the bulk concentration.

Passive Diffusion Through a Cell Membrane

The passive diffusion of a compound through a cell membrane can be described with an adaptation of Fick's equation:
J=kₚA(c_{1,out}−c_{1,in}) (11)
where J is the net flux across the membrane (molecules per second), kₚ is the permeability coefficient (cm/s), A is the surface area of the cell membrane (cm²), c_{1,out} is the mass concentration of compound C₁ immediately outside the membrane (molecules/cm³), and c_{1,in} is the mass concentration of C₁ just inside the membrane (molecules/cm³). For the simplified case of a spherical cell, where R is the distance from the center of the cell to the inner surface of the cell membrane, and the membrane thickness is Δr, the surface area A can be calculated using basic geometry:
A=4π(R+Δr)² (12)
General Diffusion Within the Cell

A governing equation for the concentration of a compound as a function of time and radial distance from the cell center is simplified by the assumption of spherical symmetry²⁵. Taking mass diffusion and a generic reaction term into account, with D₁ as the mass diffusion coefficient, and q₁ as the net generation/removal of the species due to reaction

$$\frac{ⅆ {C}_{1}}{ⅆ t} = {D}_{1} ( \frac{{ⅆ}^{2} {C}_{1}}{ⅆ {r}^{2}} + \frac{2}{r} \frac{ⅆ {C}_{1}}{ⅆ r} ) + {q}_{1} \qquad ( 13 )$$

²⁵ Rashevsky, N. (1960). Mathematical biophysics: physico-mathematical foundations of biology, vol. 1, 3^{rd} edition. New York: Dover Publications, Inc

Receptor-Ligand Binding to a G-Protein Receptor

Several models of G-protein signal transduction have been proposed. The collision coupling model includes the binding of a ligand (L) to a membrane receptor \mathring{C} to form a complex (C), which associates with an effector (E) to form an intermediate (E-C)²⁶. In the case of pigment dispersion, the ligand is a compound such as MSH, which binds to the MSH receptor (C), and then associates with the pre-coupled G-protein/adenylate cyclase complex (E). This association is presumed to be a collision coupling mechanism, where the ligand-receptor complex C physically encounters the effector complex E by diffusion within the cell membrane layer. The intermediate complex E-C is catalyzed by the exchange of GDP for GTP by the G-protein, dissociating to an activated adenylate cyclase/G-protein complex (E*) and the ligand-receptor complex (C). The enzyme complex can be deactivated with rate constant kd. These interactions are displayed in Eqn 14 and 15.
²⁶ Lauffenburger, D. A., and Linderman, J. J. (1993). Receptors: models for binding, trafficking, and signalling. New York: Oxford University Press.

$$\begin{matrix}L + R {⇔}_{{k}_{r}}^{{K}_{f}} C & ( 14 ) \\ E + C {⇔}_{{k}_{u}}^{{k}_{c}} E - C {⇒}^{{k}_{a}} {E}^{*} + C {⇒}^{{k}_{d}} E + C & ( 15 )\end{matrix}$$

In analyzing this model, Lauffenburger and Linderman²⁶ make the following assumptions. The free ligand concentration, L, is assumed to be constant, and receptor/ligand binding is taken to be at quasi-equilibrium. The amount of the intermediate complex E-C is assumed to be small compared to C, R, and E, so the receptor/ligand binding at equilibrium simplifies to the basic monovalent case of

$$C = \frac{{R}_{T} L}{{K}_{D} + L} , \qquad ( 16 )$$

where RT is the total number of receptors and K_{D}=kᵣ/k_{f}. The kinetic equations describing the amount of active adenylate cyclase (E*) are

$$\begin{matrix}\frac{ⅆ {E}^{*}}{ⅆ t} = {k}_{a} E - C & ( 17 ) \\ \frac{ⅆ E - C}{ⅆ t} = {k}_{c} EC - ( {k}_{u} + {k}_{a} ) E - C & ( 18 )\end{matrix}$$

²⁶ Lauffenburger, D. A., and Linderman, J. J. (1993). Receptors: models for binding, trafficking, and signalling. New York: Oxford University Press.

Using the pseudo-steady state assumption that equation (18) is approximately equal to 0, and using the conservation of mass approximation for the G-protein/adenylate cyclase complex, with total E_{T}≈E+E*, the amount of activated adenylate cyclase can be expressed as a function of time,
E*(t)=E_{T}[1−exp(−k_{obs}t)] (19)
where the overall first order rate constant k_{obs} is defined by

$${k}_{obs} = ( \frac{{k}_{c} {k}_{a}}{{k}_{u} + {k}_{a}} ) ( \frac{L}{{K}_{D} + L} ) {R}_{T} \qquad ( 20 )$$

As adenylate cyclase is a transmembrane protein, fixed at the membrane surface, there is no need to consider its radial distribution²⁷.
²⁷ Schwartz, J. H. (2001). The many dimensions of cAMP signaling. Proceedings of the National Academy of Science USA, 98(24), 13482-13484

Other models of G-protein activation include more explicit tracking of the active and inactive states of various system components. Shea and Linderman (1997) suggest a pre-coupling mechanism for the receptor-agonist complex C, in which the unbound receptor is assumed to be in the inactive state, but the receptor-agonist complex may take either an inactive or active state. The activation of the G-protein in this case can occur by collision coupling, where the active receptor-agonist complex interacts with the G-protein due to a collision in the plasma membrane, or through pre-coupling, where the G-protein is already associated with the receptor when the agonist and receptor are bound.

Pigment Dispersion by cAMP Activation

When a stimulatory G-protein is activated, it signals adenylate cyclase to convert ATP to cyclic AMP. The AC system involves more than nine related enzymes which are regulated in different ways. These include activation by Ca²⁺/calmodulin, inhibition by low concentrations of Ca²⁺, and inhibition by calcineurin, the Ca²⁺-dependent protein phosphatase²⁷. Cell surface receptors such as MSH receptors and β-adrenergic receptors are linked to G_{S}^{28,18}. The conversion of ATP to cAMP can be modeled as a Michaelis-Menten reaction²⁹.
²⁷ Schwartz, J. H. (2001). The many dimensions of cAMP signaling. Proceedings of the National Academy of Science USA, 98(24), 13482-13484

²⁸ Tuma, M. C., and Gelfand, V. I. (1999). Molecular Mechanisms of Pigment Transport in Melanophores. Pigment Cell Research, 12, 283-294

¹⁸ Haimo, L. T., and Thaler, C. D. (1994). Regulation of organelle transport: Lessons from color change in fish. BioEssays, 16(10), 727-733

²⁹ Goldbeter, A. and Segel, L. A. (1977). Unified mechanism for relay and oscillation of cyclic AMP in Dictyostelium discoideum. Proceedings of the National Academy of Science USA, 74(4), 1543-1547

$$\begin{matrix}{E}^{*} + ATP {⇔}_{{k}_{r2}}^{{k}_{f2}} {E}^{*} - ATP complex & ( 21 ) \\ {E}^{*} - ATP complex {⇔}^{{k}_{f3}} cAMP + {E}^{*} ( + pyrophosphate ) & ( 22 )\end{matrix}$$

Increased cAMP production activates the tetrameric protein kinase A by binding to its regulatory subunits, which causes the PKA to release its two catalytic subunits³⁰. The catalytic subunits then phosphorylate a target protein, and this phosphorylation directly or indirectly leads to the activation of the motor proteins kinesin II and myosin V, which are responsible for chromatosome dispersion³¹. The concentration of cAMP declines due to enzymatic degradation by phosphodiesterase²⁷.
³⁰ Chin, K.-V., Yang, W.-L., Ravatn, R., Kita, T., Reitman, E., Vettori, D., et al. (2001). Part I—Protein Kinase A: Signaling, Model Systems, Pathophysiology, and Genetics—Reinventing the Wheel of Cyclic AMP: Novel Mechanisms of cAMP Signaling. Annals of the New York Academy of Sciences, 968, 49-64

³¹ Rogers, S. L., Tint, I. S., Fanapour, P. C., and Gelfand, V. I. (1997). Regulated bidirectional motility of melanophore pigment granules along microtubules in vitro. Proceedings of the National Academy of Science USA, 94, 3720-3725

²⁷ Schwartz, J. H. (2001). The many dimensions of cAMP signaling. Proceedings of the National Academy of Science USA, 98(24), 13482-13484

The initial steps in the adenylate cyclase/cAMP pathway can be modeled using a mechanistic approach. Thomsen and Neubig³² suggest that the kinetics of activation and inhibition of AC in the presence of G-proteins is extremely rapid, and that the resulting cAMP production is linear with time. A Michaelis-Menten model of enzyme-substrate interaction was used by Juska and Farndale³³ to describe the time-course of cAMP accumulation. I will use a similar model to simulate the binding of cAMP to the regulatory subunits of PKA, which results in the dissociation of the catalytic subunits:
cAMP+PKA_{R2C} [symbol] cAMP−PKA_{R2C2} (23)
cAMP−PKA_{R2C2} [symbol] PKA_{C2}+cAMP−PKA_{R2} (24)

³² Thomsen, W. J., and Neubig, R. R. (1989). Rapid Kinetics of α₂-Adrenergic Inhibition of Adenylate Cyclase. Evidence for a Distal Rate-Limiting Step. Biochemistry, 28, 8778-8786

³³ Juska, A., and Farndale, R. W. (1999). Inhibition of human platelet adenylate cyclase activity by adrenaline, thrombin and collagen: analysis and reinterpretation of experimental data. Biochemical Journal, 340, 245-253

Since the explicit pathway connecting PKA to the activation of the motor proteins has not been determined yet, a mechanistic model for the final step linking the cAMP signal cascade to the microtubule transport system cannot be formulated. Zeroth and first order

The Levenberg-Marquardt method will be utilized for multiple nonlinear regression³⁴ to fit the model parameters. Since this is a variation of chi-square fitting, it should be possible to use chi-square boundaries to estimate confidence intervals for the model parameters, so that comparisons can be made between the models for each experimental treatment.
³⁴ Press, W. H., Teukolsky, S. A., Vetterling, W. T., and Flannery, B. P. (2002). Numerical Recipes in C++: The Art of Scientific Computing (2^{nd} ed.). New York:

### Expected Outcome and Significance

This approach can be adapted to the chromatophore signal transduction network. In order to test the ability of the Bayesian network ability to select an appropriate sequence of modules for a given data series, a suite of modules will be constructed. This suite will include the mechanistic models which mirror the biological activity within the cells, as well as several control modules which contain simple numerical functions unrelated to the cell processes. A testing algorithm will search for high-scoring models among all possible module configurations. Ideally, the module configurations for the experimental data will differ for each agent. This comparison of the model parameters is of great interest, since the goal of the study is to determine how the different mechanisms for cell pigment aggregation and dispersion affect the kinetic rate constants of the mechanistic model.

### Example 39

This example concerns identifying, detecting and analyzing probiotic potential of commensal organisms using a cytosensor. Chromatophore biosensor cells were preincubated with lactococcal strains proposed to have probiotic potential before exposure to the toxin-producing, pathogenic Bacillus cereus strain. Preincubation with lactococcal strains prevented Bacillus cereus from causing the chromatophore biosensor cells to rapidly aggregate. These results indicate that the chromatophore-based cyctosensor has the ability to identify, detect and analyze the probiotic potential of commensal organisms.

The results of this example are illustrated in FIGS. 63A-63F. FIG. 63A illustrates the pigment pattern of chromatophores before exposure to Lactococcus. FIG. 63B illustrates the pigment pattern of chromatophores 5 minutes after exposure to Lactococcus. FIG. 63C illustrates the pigment pattern of chromatophores 30 minutes after exposure to Lactococcus. There is no obvious pigment redistribution (no aggregation) occurring in the chromatophore cells after exposure to Lactococcus. FIGS. 63D-63F illustrate the response of chromatophores to Bacillus cereus 5, 10, 30 minutes after the chromatophores have been preincubated for 30 minutes with Lactococcus. As illustrated by the data, Bacillus cereus can no longer elicit an aggregation response from the pigment organelles in chromatophores if the chromatophores have been preincubated with Lactoccus.

### Example 40

This example concerns using non-verbal classifiers for integrating outputs from orthogonal biosensor arrays. Experiments for each biosensor generate a set of numerical and non-numerical features descriptors (features). Non-numerical features (e.g., nominal and ordinal) are useful for orthogonal biosensor array communication. Different types of agents provide ordinal features that can be nominally classified as strong or weak, for example. Nominal features can be selected to be non-sensor specific. The non-numerical feature plus the nominal value is equivalent to a label. Table 26 defines a lexicon as the underlying collection of “lexemes” or ordinal descriptors for the set of labels that feeds into the different scenarios. The corresponding indicator values dictate the contribution of the individual lexemes each scenario. The final scenario is described by the vectors {Lexicon}+{Indicators}=Scenario.

TABLE 26

| “Lexicon” consists of “lexemes” | “Indicators” |
|---|---|
| {response strength none | { p₁ |
| response strength weak | p₂ |
| response strength medium | p₃ |
| response strength high | p₄ |
| response strength highest ever | p₅ |
| toxicity none | p₆ |
| toxicity weak | p₇ |
| toxicity medium | p₈ |
| toxicity high | p₉ |
| . | . |
| . | . |
| . | . |
| Agent Hg none | pₙ |
| agent Hg low concentration | p ₙ₊₁ |
| agent Hg medium concentration | p ₙ₊₁ . . . } |

The present invention has been described with reference to particular embodiments. It will be understood that the scope of the invention should not be limited to the particular embodiments described but instead is considered with reference to the following claims.

1. A method for classifying a scenario, comprising:
exposing a system to a bioactive condition;
representing a response of the system, or portion thereof, to the bioactive condition; and
attempting to classify a scenario by database comparison.

2. The method according to claim 1 where the system comprises living cells.

3. The method according to claim 1 where the system comprises living organisms.

4. The method according to claim 1 where the system is a living organism.

5. The method according to claim 1 where the system is a microbial community.

6. The method according to claim 1 where the system is tissue.

7. The method according to claim 1 where representing a response of the system comprises determining data sufficient to determine a numerical feature space vector, and the method further comprises providing a database for comparison by exposing a system to known scenarios to determine a numerical feature space vector, and transforming the data.

8. The method according to claim 7 further comprising transforming the data by:
determining software expert parameters based on extracted data; and
weighting the expert parameters.

9. The method according to claim 7 where transforming the data comprises:
determining software expert parameters based on extracted data;
weighting the expert parameters; and
tuning the integrated expert.

10. The method according to claim 9 where tuning the integrated expert comprises adaptive expert calibration.

11. The method according to claim 7 where the known scenarios are simplex scenarios.

12. The method according to claim 1 where attempting to classify the bioactive condition by database comparison comprises:
exposing the system to one or more scenarios to provide sufficient data to generate a characteristic signature for the bioactive condition;
extracting data;
calculating a location of data clusters in feature space representing the characteristic signature of the bioactive condition;
comparing location of data clusters in feature space representing the characteristic signature of the bioactive condition relative to data clusters representing known bioactive conditions; and
determining a likelihood that a bioactive condition is a known bioactive condition.

13. The method according to claim 12 where calculating a relative location of data clusters is done with software experts.

14. The method according to claim 1 where attempting to classify the bioactive condition comprises:
exposing the system to one or more scenarios to provide sufficient data to generate a characteristic signature for the bioactive condition;
calculating a location of data clusters in feature space representing the characteristic signature of the bioactive condition;
comparing the location of data clusters representing the characteristic signature of the bioactive condition relative to data clusters representing known bioactive conditions; and
determining a likelihood that an unknown bioactive condition is a known bioactive condition.

15. The method according to claim 14 where calculating a relative location of data clusters is done with software experts.

16. The method according to claim 1 where attempting to classify a complex scenario comprises:
synthesizing a complex scenario from known scenarios present in the database;
comparing the scenario generated by the bioactive condition to the complex scenario; and
determining a likelihood that a complex scenario is the scenario.

17. The method according to claim 16 where the known scenarios are simplex scenarios.

18. The method according to claim 17 where each simplex scenario is an elicitor.

19. The method according to claim 18 where the elicitor consists of bioactive conditions and the protocol utilized to apply the bioactive conditions to the system.

20. The method according to claim 19 where the bioactive conditions have known effects on the system.

21. The method according to claim 19 where the bioactive conditions have unknown effects on the system.

22. The method according to claim 18 comprising repeatedly applying an unknown bioactive agent in combination with one or more elicitors.

23. The method according to claim 18 where each simplex scenario contains information regarding effect on the system of the bioactive conditions.

24. The method according to claim 16 where determining the probability that the complex scenario is a complex scenario provides information about the manner in which the bioactive condition affects the system.

25. The method according to claim 22 where each repeated step consists of application of an unknown bioactive agent in combination with one or more elicitors.

26. The method according to claim 21 where each simplex scenario contains information regarding the effect on the system of the bioactive conditions.

27. The method according to claim 20 where determining the likelihood that the complex scenario is a complex scenario provides information about the manner in which the bioactive condition affects the system.

28. The method according to claim 7 where the system comprises living cells and the numerical feature space vector contains parameters selected from the group consisting of nucleic acid composition, membrane lipid fatty acid composition, metabolic activity, cellular secretions, chemical measurements, physical measurements, and combinations thereof.

29. The method according to claim 28 where nucleic acid composition is determined by 16S-RNA profiling.

30. The method according to claim 28 where membrane lipid fatty acid composition is determined by phospholipids fatty acid profiling.

31. The method according to claim 28 where metabolic activity is determined by community level physiological profiling.

32. The method according to claim 28 where chemical measurements are selected from the group consisting of organic pollutants, nutrient discharges, heavy metals, dissolved oxygen, pH, redox, chlorine ion concentration and combinations thereof.

33. The method according to claim 28 where physical measurements are selected from the group consisting of temperature, ultra-violet radiation intensity, light intensity, and combinations thereof.

34. The method according to claim 28 where physical measurement provides a measure of cell morphology and turbidity changes.

35. The methods according to claim 1 where the system comprises living cells and a numerical feature space vector contains measurements of changes in physical properties of semiconductor nanoparticles.

36. The method according to claim 1 where the system is a live cell, and a numerical feature space vector contains measurements of cellular processes selected from the group consisting of gene expression, cell regulation, metabolism, and combinations thereof.

37. The method according to claim 36 where changes in cellular processes are characterized using semiconductor nanoparticles.

38. The method according to claim 36 where gene expression is characterized using a technique selected from the group consisting of DNA microarray analysis, recombinant marker analysis, green fluorescent protein analysis, enzymatic activity, semiconductor nanoparticle analysis, and combinations thereof.

39. The method according to claim 38 where the enzymatic activity is recombinant luciferase.

40. The method according to claim 38 where analyzing gene expression comprises detecting protein expression using immunodetection.

41. The method according to claim 40 where immunodetection comprises western blotting, lateral flow immunodetection, immune precipitation, and combinations thereof

42. The method according to claim 38 where cell regulation is characterized using changes in protein phosphorylation patterns.

43. The method according to claim 42 where changes in protein phosphorylation patterns are measured using 2-D gel electrophoresis.

44. The method according to claim 38 where cell regulation is characterized through changes in gene expression patterns characterized using DNA microarray analysis.

45. The method according to claim 38 where changes in cellular processes are determined by measuring growth medium composition.

46. The method according to claim 38 where changes in cellular processes are determined by measuring metabolite secretion patterns.

47. The method according to claim 38 where changes in cellular processes are determined by measuring protein secretion patterns.

48. The method according to claim 40 where measurements are made using methods selected from the group consisting of gas chromatography, liquid chromatography, mass spectrometry, nuclear magnetic resonance spectrometry, gel electrophoresis, and combinations thereof.

49. The method according to claim 38 where changes in cell metabolism are determined by intracellular measurements of metabolite levels.

50. The method according to claim 49 where intracellular measurements are made using metabolite secretion patterns provided by gas chromatography, liquid chromatography, mass spectrometry, nuclear magnetic resonance spectrometry, and combinations thereof.

51. The method according to claim 1 where the system is a live chromatophore.

52. The method according to claim 51 where the chromatophore is a fish chromatophore.

53. The method according to claim 52 where the fish chromatophore is a Betta chromatophore.

54. The method according to claim 51 where the chromatophore is a frog chromatophore.

55. The method according to claim 54 where the frog chromatophore is a Xenopus chromatophore.

56. The method according to claim 2 where the living cells are immortalized.

57. The method according to claim 56 where the living cells are frog chromatophores.

58. The method according to claim 7 where the living system is a chromatophore and the numerical feature space vector includes color change measurements.

59. The method according to claim 58 where the measurements are of changes in the refracted wavelength of light, color intensity, cell morphology, cell area, cell motility or any combination thereof.

60. The method according to claim 58 where color change measurements are made by:
acquiring images of the chromatophore, such images reflecting response of the chromatophore to a scenario;
converting response changes to hue, saturation, value histograms;
applying probabilistic segmentation to assign a probability that a histogram data point belongs to a particular group of data points in the histogram to isolate data points representing response of the chromatophore to the scenario from extraneous information, thereby providing probabilistic clusters for chromatophore responses of interest for segmenting each of the images into image classes;
dividing the images into plural subfields;
extracting numerical information for each subfield from the image segments, such numerical information representing features of the images having information concerning the chromatophore response to the scenario;
generating numerical data for each subfield for each chromatophore response monitored;
applying a model fitting procedure to the set of numerical data and identifying model parameters; and
using at least a portion of the model parameters to define a feature model space having scenario clusters for known materials.

61. The method according to claim 51 where exposing the chromatophore to a known scenario comprises providing an apparatus comprising a digital camera for recording images, a receiver for receiving live cells, an injection port for injecting a material into contact with the chromatophore, and a computer for processing information concerning response of the chromatophore to the material.

62. The method according to claim 1 where the system comprises a live cell, and the method comprises:
exposing the live cell to a scenario;
imaging the live cell to provide images of cellular response to the scenario;
repeating steps as necessary for providing sufficient data for possibly classifying the scenario; and
attempting to classify the scenario by database comparison to known scenarios using software experts.

63. The method according to claim 62 where the system comprises plural live cells.

64. The method according to claim 62 where no information concerning the scenario is included in the database information.

65. The method according to claim 63 where the known scenario is a complex scenario.

66. The method according to claim 65 where the complex scenario comprises multiple simplex scenarios.

67. The method according to claim 63 where each simplex scenario is an elicitor.

68. The method according to claim 67 where the elicitor consists of a known bioactive condition and the protocol utilized to apply the bioactive conditions to the system.

69. The method according to claim 68 where the bioactive condition has known effects on biological pathways.

70. The method according to claim 68 where the bioactive conditions have unknown effects on biological pathways.

71. The method according to claim 70 where the biological pathways are cell signaling pathways that control cell function.

72. The method according to claim 55 where each repeated step consists of application of an unknown bioactive agent in combination with one or more elicitors.

73. The method according to claim 62 where the complex scenario comprises simplex scenarios that represent information regarding the mechanism of action of known bioactive conditions.

74. The method according to claim 62 where classifying the scenario with respect to the complex scenario provides information about mechanism of action of the bioactive condition.

75. The method according to claim 62 where classification involves determining the likelihood that the scenario is a known scenario.

76. The method according to claim 62 where the system comprises plural live cells and database information is obtained by:
serially exposing plural live cell to a known scenario;
acquiring images of the cells reflecting response of the cells to the scenario;
converting response changes to hue, saturation, value histograms;
applying probabilistic segmentation to assign a probability that a histogram data point belongs to a particular group of data points in the histogram to isolate data points representing response of the cells to the scenario from extraneous information, thereby providing probabilistic clusters for cell responses of interest for segmenting each of the images into image classes;
dividing the images into plural subfields;
extracting numerical data sets for each subfield from the image segments, such numerical information representing features of the images having information concerning the cellular response to the scenario;
applying a model fitting procedure to each data set and extracting corresponding model parameters; and
using at least a portion of the model parameters to define a feature model space having scenario clusters for known materials.

77. The method according to claim 76 where attempting to classify the scenario comprises:
repeatedly exposing the cells to an unknown scenario, and repeating steps required for providing a model space having statistical expert information concerning the unknown scenario;
calculating distances between clusters representing unknown scenarios and known scenarios; and
determining a likelihood that an unknown scenario is a known scenario by combining such distances by expert voting.

78. The method according to claim 1 useful for comparing orthogonal biological system responses within a numerical feature space vector.

79. The method according to claim 49 where feature space dimensionality is controlled by elicitor set size.

80. The method according to claim 1 useful for classifying unknown drug candidates.

81. The method according to claim 1 useful for classifying unknown toxins.

82. The method according to claim 81, comprising:
identifying cell type and cell response most useful for providing information concerning cellular response to a particular scenario;
generating a database of scenarios; and
classifying unknowns by comparison with numerical feature space vector created by known scenarios.

83. The method according to claim 1 the system comprises non-pigmented cells.

84. The method according to claim 76 where exposing comprises using an apparatus comprising a digital camera for recording images, a receiver for receiving live cells, an injection port for injecting a material into contact with the cells, and a computer for processing information concerning response of the cells to the material.

85. The method according to claim 84 where the apparatus comprises plural cameras.

86. The method according to claim 85 where the scenario comprises exposing the cells to a chemical or biological agent.

87. The method according to claim 86 where the scenario comprises a chemical agent, and the cells are exposed to the chemical agent at varying concentrations.

88. The method according to claim 87 where the data-fitting model is a parametric nonlinear dynamic model.

89. The method according to claim 84 where the model parameter represents a single cell response.

90. The method according to claim 84 where the model parameter represents plural cell responses.

91. The method according to claim 85 where the model parameters are time dependent.

92. The method according to claim 84 where the model parameters are time independent.

93. The method according to claim 84 where the expert parameter represents a cellular state prior to exposure to the material and subsequent to exposure to the material.

94. The method according to claim 84 where determining a probability that the unknown scenario is a known scenario comprises determining a Mahalanobis distance for the unknown scenario relative to at least one known scenario cell reaction model space.

95. A method for analyzing data, comprising:
providing an apparatus comprising a digital camera for recording images, a receiver for receiving live cells, an injection port for injecting a material into contact with the cells, and a computer for processing information concerning response of the cells to the material;
exposing the cells to a known material;
acquiring images of the cells at a first time to a second time through a predetermined time period;
analyzing the images to isolate cell features from extraneous information by converting response changes to saturation value histograms versus time, applying probabilistic segmentation to assign the probability that a histogram data point belongs to a particular group of data points in the histogram to isolate data points representing response of the cells to the material from extraneous information, thereby providing probabilistic clusters versus time for cell responses of interest for segmenting each of the images into image segments;
dividing the images into plural subfields;
extracting numerical information for each subfield from the image segments, such numerical information representing features of the images having information concerning response of the cells to the material;
generating numerical information versus time for each subfield for each cell response monitored to provide plural expert curves representing cellular response to the material versus time;
applying parametric nonlinear auto-regressive external input models to each of the plural expert curves to describe such curves using a predetermined number of parameters;
determining at least a portion of the parameters to define a feature model space having scenario clusters for known materials;
exposing the cells to an unknown material, and repeating steps required for determining expert parameters;
weighting the expert parameters;
normalizing expert parameters to provide a virtual expert; and
determining a likelihood that an unknown scenario is a known scenario.

96. A method for detecting bioactivity of a test compound, comprising
placing at least one Betta chromatophore in functional contact with the test compound; and
detecting color of the Betta chromatophore.

97. The method according to claim 96 further comprising providing a cytosensor apparatus useful for placing the Betta chromatophore in functional contact with the test compound and detecting the color of the Betta chromatophore.

98. The method according to claim 96 further comprising encapsulating the Betta chromatophore.

99. The method according to claim 96 where the test compound is selected from the group consisting of bacteria, fungi, viruses, plants and animals.

100. The method according to claim 96 where the test compound is selected from the group consisting of neurotransmitters, hormones, intracellular signal transduction agents, pharmaceutically active agents, toxic agents, agricultural chemicals, chemical toxins in ingested materials, biological toxins in ingested materials, microbes and animal cells.

101. A test kit for detecting bioactive compounds comprising the following separately packaged solutions:
a nutrient solution comprising at least one Betta chromatophore; and
a solution containing a positive control solution.

102. The test kit according to claim 101 where the positive control solution contains a compound selected from the group consisting of norepinephrine, serotonin, forskolin, caffeine, adenosine, dopamine, melanocyte stimulating hormone, melanophore concentrating hormone, and structural and pharmacological analogs, agonists, and antagonists of such compounds.

103. The test kit of claim 101 where the Betta chromatophore is selected from the group consisting of B. splendens, B. schaumnestbauer, B. bellica, B. coccina, B. farciata, B. foerrchi, B. rmaradgina, B. maulbruter, B. anabatoidcr, B. balunga, B. brederi, B. macractoma, B. picta, B. pugrrax, B. rubra, B. taeniata, and B. unimaculata.

104. A cytosensor, comprising:
a vessel defining an inlet for cells that provides an inlet for at least one bioactive unit or at least one test compound for functionally contacting the at least one bioactive unit and at least one test compound;
a detector for detecting changes in the bioactive unit; and
a computer for controlling the apparatus and analyzing changes detected by the detector.

105. The apparatus according to claim 104 where the bioactive unit is a Betta chromatophore.

106. The apparatus according to claim 104 further comprising a signal processing system coupled to the detector.

107. The apparatus according to claim 106 further comprising an analyzer that converts a digital output from the signal processing system into a result.

108. A computer program encoding the method of claim 1.

109. A computer programmed with the computer program of claim 108.

110. A computer-readable medium on which is stored a computer program having instructions for executing the method of claim 1.

111. The method according to claim 1 where the system is a living cell that experiences a detectable change in response to an interaction with a bioactive condition.

112. The method according to claim 11 where the living cell is selected from the group consisting of a PC12 pheochromocytoma cell, a HeLa cell, and combinations thereof.

113. The method according to claim 111 where the living cells are cancer cells.

114. The method according to claim 113 where the living cells are selected from the group consisting of HeLa cells, 3T3 cells, COS7 cells, HEPG2 cells, and combinations thereof.

115. The method according to claim 2 where the change detected in the living cells is cytoplasmic streaming.

116. The method according to claim 1 where the system is a mammal.

117. The method according to claim 1 where the system is a fish.
